(19) United States
(12) Patent Application Publication
(10) Pub. No.: US 2018/0334436 A1
(43) Pub. Date: Nov. 22, 2018

(54) TETRAHYDROISOQUINOLINE DERIVATIVES

(71) Applicant: VIIV HEALTHCARE UK LIMITED, Brentford, Middlesex (GB)

(72) Inventors: Brian Alvin JOHNS, Research Triangle Park, NC (US); Emile Johann VELTHUISEN, Research Triangle Park, NC (US); Jason Gordon WEATHERHEAD, Research Triangle Park, NC (US)

(21) Appl. No.: 15/776,532

(22) PCT Filed: Dec. 1, 2016
(86) PCT No.: PCT/IB2016/057270

§ 371 Date: May 16, 2018

Related U.S. Application Data
(60) Provisional application No. 62/262,934, filed on Dec. 4, 2015.

(51) Int. Cl. C07D 217/04; C07D 217/06; C07D 401/06; C07D 401/12; C07D 217/08; C07D 405/04; C07D 413/04; C07D 413/14; C07D 217/24; A61P 31/18

(52) U.S. Cl. CPC C07D 217/04; C07D 217/06; C07D 401/06; C07D 401/12; A61P 31/18; C07D 405/04; C07D 413/04; C07D 413/14; C07D 217/24; C07D 217/08

(57) ABSTRACT

Compounds of Formula I are disclosed and methods of treating viral infections with compositions comprising such compounds.

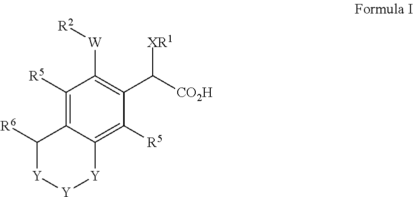

### FIELD OF THE INVENTION

The present invention relates to substituted tetrahydroisoquinoline compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

For example, over the last decade HIV inhibitors have been reported to target the protein-protein interaction between HIV-1 integrase and Lens Epithelium Derived Growth Factor/p75 (“LEDGF”). LEDGF is a cellular transcriptional cofactor of HIV-1 integrase that promotes viral integration of reverse transcribed viral cDNA into the host cell's genome by tethering the preintegration complex to the chromatin. Because of its crucial role in the early steps of HIV replication, the interaction between LEDGF and integrase represents another attractive target for HIV drug therapy.

U.S. provisional patent application 62/027,359 discloses certain isoindoline compounds having the following formula:

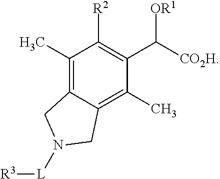

### SUMMARY OF THE INVENTION

Briefly, in one aspect, the present invention discloses compounds of Formula I:

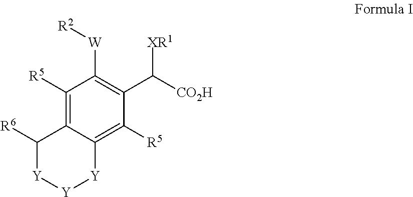

wherein:

each Y is independently C═O, CH—R⁶, or N-L-R³ with the proviso that at least one Y must be CH—R⁶;

X is O or CH₂;

R¹ is C₁₋₆ alkyl wherein said alkyl may contain cycloalkyl portions;

W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂(CO)—, or —NHC(O)CH₂—, wherein each W is optionally substituted by 1 or 2 methyl groups;

R² is H, C₁₋₆ alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆ alkyl, C₁₋₆ hetereoalkyl, or C₁₋₆ alkylene or C₁₋₆ hetereoalklylene wherein said C₁₋₆alkylene or C₁₋₆hetereoalklylene is bonded to adjacent carbon atoms on said C₅₋₁₄ aryl, C₃₋₇cycloalkyl, C₃₋₇cycloalkenyl, C₃₋₉heterocycle, or C₅₋₉heteroaryl to form a fused ring;

each L is independently a bond, —CH₂(CO)—, —C₁₋₃alkylene-, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;

each R³ is independently H, CN, oxo, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, or an R³ may join together with an R⁶ or an R³ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;

R⁴ is CN, halo, —OC₁₋₆alkyl, C₁₋₆alkyl, C₃₋₇cycloalkyl, C₃₋₉heterocycle, or C₅₋₁₄aryl;

each R⁵ is independently H, C₁₋₃alkyl, C₃₋₆cycloalkyl, CH₂F, CHF₂, or CF₃;

each R⁶ is independently H, oxo, C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system, or R⁶ may represent a gem di-C₁₋₃alkyl.

and wherein each heterocycle, heteroaryl, heteroalkyl, and heteroalkylene comprises one to three heteroatoms selected from S, N, B, or O.

In another aspect the present invention discloses pharmaceutically acceptable salts of the compounds of Formula I.

In another aspect, the present invention discloses pharmaceutical compositions comprising a compound of Formula I or a pharmaceutically acceptable salt thereof.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

In accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

### DETAILED DESCRIPTION OF THE INVENTION

Preferably W is a bond.

Preferably one Y is N-L-R³.

Preferably R¹ is C₁₋₆ alkyl. Most preferably, R¹ is t-butyl.

Preferably X is O.

Preferably R² is optionally substituted phenyl. Most preferably, R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

Preferably each R³ is independently C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alky, C₁₋₃fluoroalkyl, or phenyl.

Preferably each R⁵ is methyl.

Preferably each R⁶ is H.

Preferably the stereochemistry on the carbon to which XR¹ is bound is as depicted below.

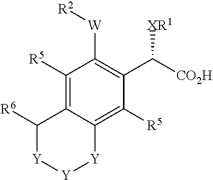

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

### Examples

The compounds of this invention may be made by a variety of methods, including well-known standard synthetic methods. Illustrative general synthetic methods are set out below and then specific compounds of the invention are prepared in the working examples.

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
BINAP=(2,2′-bis(diphenylphosphino)-1,1′-binaphthyl)
boc=tert-butoxycarbonyl
br=broad
Cbz=benzyloxycarbonyl
d=doublet
δ=chemical shift
° C.=degrees celcius
DCE=1,2-dichloroethane
DCM=dichloromethane
dd=doublet of doublets
DIEA=N,N-diisopropylethylamine
DMEM=Dulbeco's Modified Eagle's Medium
DMF=N,N-dimethylformamide
DMSO=dimethylsulfoxide
EtOAc=ethyl acetate
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H+
mg=milligram
min=minutes
mL=milliliter
mM=millimolar
mmol=millimole
MS=mass spectrum
nm=nanomolar
ppm=parts per million
q.s.=sufficient amount
singlet
RT=room temperature
sat.=saturated
triplet
THF=tetrahydrofuran
TFA=trfluoroacetic acid
TMS=trimethylsilyl
T3P=propylphosphonic anhydride
TFA=trifluoroacetic acid
benzyloxycarbonyl

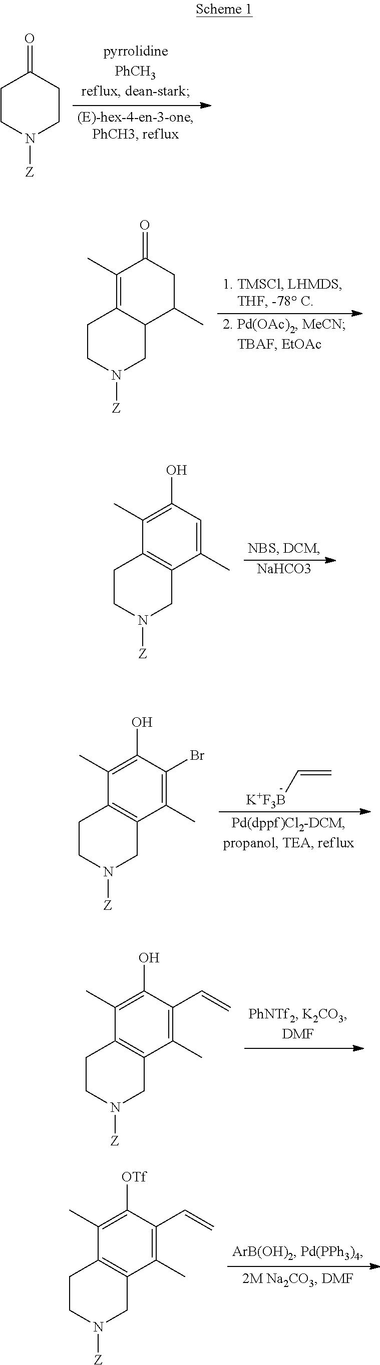

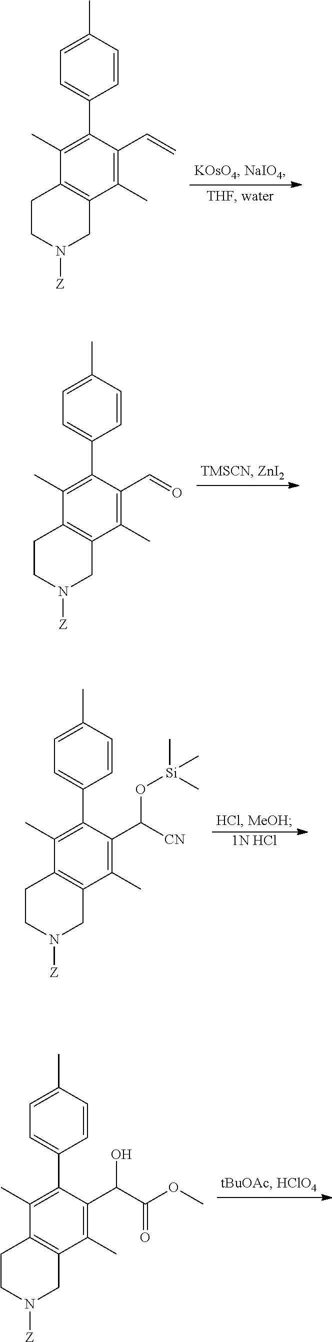

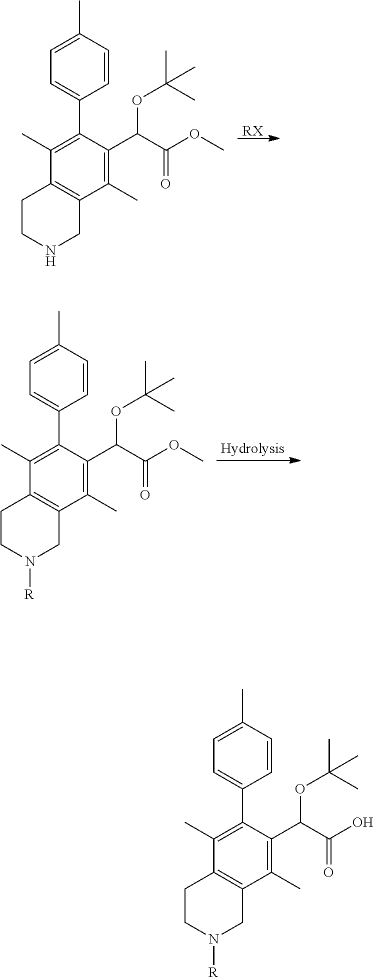

### Example 1: 2-(tert-Butoxy)-2-(2-(3-fluorobenzyl)-5,8-dimethyl-6-(p-tolyl)-1,2,3,4-tetrahydroisoquinolin-7-yl)acetic Acid

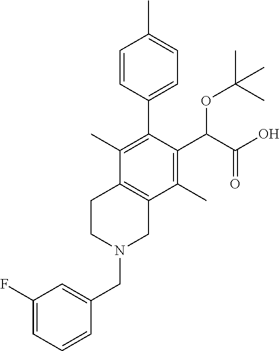 [SMILES: Cc1ccc(-c2c(C)c3c(c(C)c2C(OC(C)(C)C)C(=O)O)CN(Cc2cccc(F)c2)CC3)cc1]

### Step 1: Benzyl 5,8-dimethyl-6-oxo-3,4,6,7,8,8a-hexahydroisoquinoline-2(1H)-carboxylate

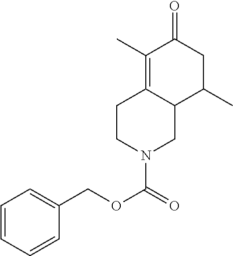 [SMILES: CC1=C2CCN(C(=O)OCc3ccccc3)CC2C(C)CC1=O]

A solution of benzyl 4-oxopiperidine-1-carboxylate (25 g, 107 mmol) and pyrrolidine (44.3 mL, 536 mmol) in 60 mL toluene was reluxed with a dean stark trap for 48 hours. The solvent was removed, using toluene to azeotrope pyrrolidine. The amber oil was redissolved in 60 mL toluene and (E)-hex-4-en-3-one (12.26 mL, 107 mmol) and hydroquinone (0.118 g, 1.072 mmol) were added. The solution was heated to reflux and stirred overnight, cooled to room temperature, diluted with EtOAc and 1M HCl, extracted with EtOAc, dried over Na₂SO₄ and the solvent was removed. The brown oil was purified by silica gel chromatography (0-100% EtOAc/Hexanes gradient elution) to yield the title compound as a yellow oil (23.25 g, 74.2 mmol, 69.2% yield). LCMS (ES+)(m/z): 314.28 (M+1).

### Step 2: Benzyl 6-hydroxy-5,8-dimethyl-3,4-dihydroisoquinoline-2(1H)-carboxylate

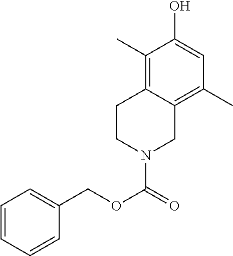 [SMILES: Cc1cc(O)c(C)c2c1CN(C(=O)OCc1ccccc1)CC2]

To a solution of benzyl 5,8-dimethyl-6-oxo-1,4,4a,5,6,8a-hexahydroisoquinoline-2(3H)-carboxylate (12.37 g, 39.5 mmol) in 60 mL THF at −78° C. was added LHMDS (53.1 mL, 53.1 mmol) dropwise. The solution was stirred for 30 minutes then TMSCl (6.05 mL, 47.4 mmol) was added dropwise. The solution was stirred for 2 hours then warmed to room temperature and stirred for 15 mins, diluted with di-ethyl ether, poured over ice and NaHCO₃ and extracted with diethyl ether. The solution was concentrated, dissolved in 300 mL acetonitrile, and palladium(II) acetate (18.32 g, 82 mmol) was added. The suspension was stirred overnight and a color change was observed; clear brown to black cloudy. The solution was filtered over celite and the solvent was removed. The brown oil was redissolved in 300 mL EtOAc and TBAF (148 mL, 148 mmol) was added dropwise. The solution was stirred for 2 hours then diluted with H₂O, EtOAc and 1M HCl, extracted with EtOAc, dried over Na₂SO₄, and the solvent was removed. The brown oil was purified by silica gel chromatography (0-30% EtOAc/Hexanes gradient elution) to yield the title compound as a white solid (10.3 g, 33.1 mmol, 44.6% yield). ¹H NMR (400 MHz, CDCl₃) δ 7.46-7.29 (m, 5H), 6.53 (s, 1H), 5.20 (s, 2H), 4.70 (s, 1H), 4.49 (s, 2H), 3.73 (m, 2H), 2.74 (m, 2H), 2.23-2.05 (m, 6H). LCMS (ES+)(m/z): 312.27 (M+1); 334.30 (M+23).

### Step 3: Benzyl 7-bromo-6-hydroxy-5,8-dimethyl-3,4-dihydroisoquinoline-2(1H)-carboxylate

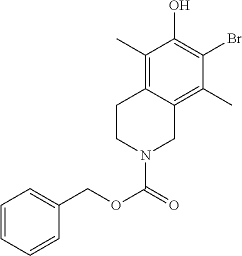 [SMILES: Cc1c(O)c(Br)c(C)c2c1CCN(C(=O)OCc1ccccc1)C2]

To a solution of benzyl 6-hydroxy-5,8-dimethyl-3,4-dihydroisoquinoline-2(1H)-carboxylate (4 g, 12.85 mmol) in 250 mL DCM was added sodium bicarbonate (1.403 g, 16.70 mmol) followed by NBS (2.52 g, 14.13 mmol). A color change was observed, off white to yellow. The solution was stirred for 30 minutes then diluted with DCM and 10% sodium thiosulfate, extracted with DCM, dried over Na₂SO₄ and the solvent was removed. Yellow foam was purified by silica gel chromatography (0-20% EtOAc/Hexanes gradient elution) to yield the title compound as an off white solid (4.15 g, 10.63 mmol, 83% yield). ¹H NMR (400 MHz, CDCl₃) δ 7.45-7.29 (m, 5H), 5.64 (s, 1H), 5.19 (s, 2H), 4.53 (s, 2H), 3.71 (m, 2H), 2.72 (m, 2H), 2.41-2.11 (m, 6H). LCMS (ES+)(m/z): 390.34 (M+1); 412.23 (M+23); 801.34 (2M+23).

### Step 4: Benzyl 6-hydroxy-5,8-dimethyl-7-vinyl-3,4-dihydroisoquinoline-2(1H)-carboxylate

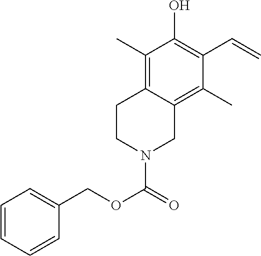 [SMILES: C=Cc1c(C)c2c(c(C)c1O)CCN(C(=O)OCc1ccccc1)C2]

A solution of benzyl 7-bromo-6-hydroxy-5,8-dimethyl-3,4-dihydroisoquinoline-2(1H)-carboxylate (6.82 g, 17.48 mmol) in 170 mL of 1-propanol was degassed for 10 minutes. Vinyl trifluoroborate (9.36 g, 69.9 mmol), TEA (17.05 mL, 122 mmol) and PdCl₂(dppf)-CH₂Cl₂ adduct (1.613 g, 1.975 mmol) were added and the solution was refluxed at 130° C. for 1 hour. The solution was cooled to room temperature, diluted with EtOAc and water, extracted with EtOAc, dried over Na₂SO₄ and the solvent was removed. The brown oil was purified by silica gel chromatography (0-100% EtOAc/Hexanes gradient elution) to yield the title compound as a yellow oil (4.776 g, 14.15 mmol, 81% yield). ¹H NMR (400 MHz, CDCl₃) δ 7.45-7.29 (m, 5H), 6.71 (m, 1H), 5.75 (m, 1H), 5.64 (s, 1H), 5.50 (m, 2H), 5.19 (s, 2H), 4.51 (s, 2H), 3.72 (m, 2H), 2.75 (m, 2H), 2.22-2.01 (m, 6H). LCMS (ES+)(m/z): 338.41 (M+1); 360.36 (M+23); 697.56 (2M+23).

### Step 5: Benzyl 5,8-dimethyl-6-(((trifluoromethyl)sulfonyl)oxy)-7-vinyl-3,4-dihydroisoquinoline-2(1H)-carboxylate

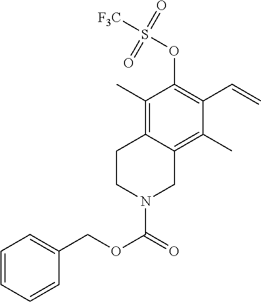 [SMILES: C=Cc1c(C)c2c(c(C)c1OS(=O)(=O)C(F)(F)F)CCN(C(=O)OCc1ccccc1)C2]

To a solution of benzyl 6-hydroxy-5,8-dimethyl-7-vinyl-3,4-dihydroisoquinoline-2(1H)-carboxylate (4.776 g, 14.15 mmol) in 50 mL DMF was added K₂CO₃ (3.91 g, 28.3 mmol) followed by PhNTf₂ (10.62 g, 29.7 mmol). The suspension was stirred for 1 hour, diluted with di-ethyl ether and water, extracted with di-ethyl ether, dried over Na₂SO₄ and the solvent was removed. The resulting yellow oil was purified by silica gel chromatography (0-20% EtOAc/Hexanes gradient elution) to yield the title compound as a clear oil (5.54 g, 11.80 mmol, 83% yield). ¹H NMR (400 MHz, CDCl₃) δ 7.45-7.29 (m, 5H), 6.66 (m, 1H), 5.68 (m, 1H), 5.37 (m, 1H), 5.20 (s, 2H), 4.56 (s, 2H), 3.75 (m, 2H), 2.76 (m, 2H), 2.33-2.12 (m, 6H). LCMS (ES+)(m/z): 470.28 (M+1); 492.28 (M+23).

### Step 6: Benzyl 5,8-dimethyl-6-(p-tolyl)-7-vinyl-3,4-dihydroisoquinoline-2(1H)-carboxylate

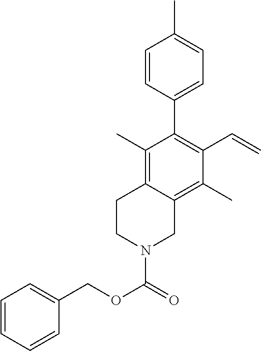 [SMILES: C=Cc1c(C)c2c(c(C)c1-c1ccc(C)cc1)CCN(C(=O)OCc1ccccc1)C2]

A solution of benzyl 5,8-dimethyl-6-(((trifluoromethyl)sulfonyl)oxy)-7-vinyl-3,4-dihydroisoquinoline-2(1H)-carboxylate (1.84 g, 3.92 mmol), p-tolylboronic acid (1.332 g, 9.80 mmol) in 12 mL DMF was degassed for 5 mins. Pd(PPh₃)₄ (0.906 g, 0.784 mmol), and Na₂CO₃ (5.88 ml, 11.76 mmol) were added, then the solution was heated to 120° C. in a microwave while stirring for 30 minutes. The solution was diluted with diethyl ether and water, extracted with diethyl ether, washed with brine, dried over Na₂SO₄, and the solvent was removed. The resulting oil was purified by silica gel chromatography (0-100% EtOAc/Hexanes gradient elution) to yield the title compound as a yellow oil (1.34 g, 3.26 mmol, 83% yield). ¹H NMR (400 MHz, CDCl₃) δ 7.46-7.29 (m, 5H), 7.18 (m, 2H), 6.96 (m, 2H), 6.32 (m, 1H), 5.28-5.16 (m, 3H), 4.94 (m, 1H), 4.65 (s, 2H), 3.79 (m, 2H), 2.80 (m, 2H), 2.40 (s, 3H), 2.28 (s, 3H), 1.91 (s, 3H). LCMS (ES+)(m/z): 412.35 (M+1); 434.35 (M+23).

### Step 7: Benzyl 7-formyl-5,8-dimethyl-6-(p-tolyl)-3,4-dihydroisoquinoline-2(1H)-carboxylate

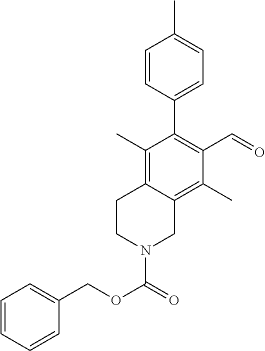 [SMILES: Cc1ccc(-c2c(C)c3c(c(C)c2C=O)CN(C(=O)OCc2ccccc2)CC3)cc1]

To a solution of benzyl 5,8-dimethyl-6-(p-tolyl)-7-vinyl-3,4-dihydroisoquinoline-2(1H)-carboxylate (4 g, 9.72 mmol) in 266 mL 3:1 THF/H₂O was added potassium osmate dihydrate (1.074 g, 2.92 mmol) followed by sodium periodate (12.47 g, 58.3 mmol). The solution was stirred for 1 hour, diluted with EtOAc and 10% sodium thiosulfate, extracted with EtOAc, dried over Na₂SO₄, and the solvent was removed. The resulting oil was purified by silica gel chromatography (0-20% EtOAc/Hexanes gradient elution) to yield the title compound as a white solid (1.81 g, 4.38 mmol, 45.0% yield). ¹H NMR (400 MHz, CDCl₃) δ 9.73 (s, 1H), 7.44-7.31 (m, 5H), 7.24 (m, 2H), 7.04 (m, 2H), 5.22 (s, 2H), 4.68 (s, 2H), 3.78 (m, 2H), 2.84 (m, 2H), 2.54-2.37 (m, 6H), 1.96 (s, 3H). LCMS (ES+)(m/z): 414.44 (M+1); 436.43 (M+23); 827.60 (2M+1).

### Step 8: Benzyl 7-(cyano((trimethylsilyl)oxy)methyl)-5,8-dimethyl-6-(p-tolyl)-3,4-dihydroisoquinoline-2(1H)-carboxylate

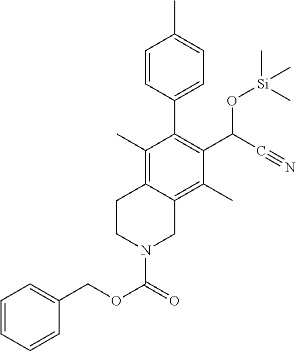 [SMILES: Cc1ccc(-c2c(C)c3c(c(C)c2C(C#N)O[Si](C)(C)C)CN(C(=O)OCc2ccccc2)CC3)cc1]

To a solution of benzyl 7-formyl-5,8-dimethyl-6-(p-tolyl)-3,4-dihydroisoquinoline-2(1H)-carboxylate (1.81 g, 4.38 mmol) in 35 mL DCM at 0° C. was added zinc iodide (0.699 g, 2.189 mmol) followed by TMS-CN (5.87 mL, 43.8 mmol). The solution was stirred for 1 hour, diluted with DCM, poured over ice, extracted with DCM, dried over Na₂SO₄ and the solvent was removed. The resulting yellow oil was carried on to the next step without further purification.

¹H NMR (400 MHz, CDCl₃) δ 7.44-7.21 (m, 7H), 7.01 (m, 2H), 5.35 (s, 1H), 5.21 (s, 2H), 4.65 (s, 2H), 3.76 (m, 2H), 2.78 (m, 2H), 2.58-2.37 (m, 6H), 1.87 (s, 3H), 0.00 (s, 9H). LCMS (ES+)(m/z): 513.37 (M+1); 535.49 (M+23); 1026.18 (2M+1).

### Step 9: Methyl 2-(5,8-dimethyl-6-(p-tolyl)-1,2,3,4-tetrahydroisoquinolin-7-yl)-2-hydroxyacetate

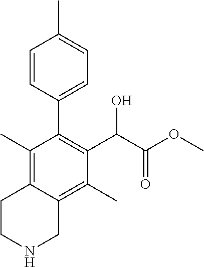 [SMILES: COC(=O)C(O)c1c(C)c2c(c(C)c1-c1ccc(C)cc1)CCNC2]

HCl gas was bubbled through MeOH at 0° C. for 20 minutes to make a saturated HCl solution. 200 mL of this solution was added to a flask with benzyl 7-(cyano((trimethylsilyl)oxy)methyl)-5,8-dimethyl-6-(p-tolyl)-3,4-dihydroisoquinoline-2(1H)-carboxylate (2.29 g, 4.47 mmol) at 0° C. The solution was stirred for 5 minutes at 0° C., warmed to room temperature and stirred for 2 hours, cooled back to 0° C. and HCl was bubbled through solution for 1 hour, stirred at 0° C. for 1 hour, warmed to room temperature and stirred for 1 hour. MeOH was removed by rotary evaporation without dipping into the water bath. The resulting clear oil was suspended in 150 mL 1M HCl and heated to 90° C. for 2.5 hours, poured over ice/water, neutralized with 50% NaOH & 1M NaOH, extracted with 1:3 IPA/CHCl₃, the solvent was removed and the clear colorless oil was carried on to the next step without further purification. ¹H NMR (400 MHz, CDCl₃) δ 7.38 (m, 1H), 7.22 (m, 2H), 7.09 (m, 1H), 5.08 (s, 1H), 4.71 (s, 1H), 3.96 (s, 2H), 3.70 (s, 3H), 3.15 (m, 2H), 2.68 (m, 2H), 2.41 (s, 3H), 2.11 (s, 3H), 1.86 (s, 3H). LCMS (ES+)(m/z): 340.47 (M+1); 679.65 (2M+1).

### Step 10: Methyl 2-(tert-butoxy)-2-(5,8-dimethyl-6-(p-tolyl)-1,2,3,4-tetrahydroisoquinolin-7-yl)acetate

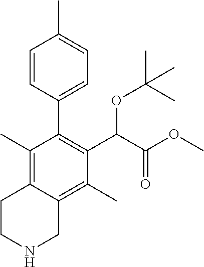 [SMILES: COC(=O)C(OC(C)(C)C)c1c(C)c2c(c(C)c1-c1ccc(C)cc1)CCNC2]

To a solution of methyl 2-(5,8-dimethyl-6-(p-tolyl)-1,2,3,4-tetrahydroisoquinolin-7-yl)-2-hydroxyacetate (330 mg, 0.972 mmol) in tert-butyl acetate (50 mL, 370 mmol) was added perchloric acid (1 mL, 16.62 mmol). The solution was stirred for 30 minutes then diluted with EtOAc and water, made basic with 1M NaOH and 50% NaOH, extracted with EtOAc, washed with brine, dried over Na₂SO₄ and the solvent was removed. The tan solid was purified by silica gel chromatography (0-10% MeOH/DCM gradient elution) to yield the title compound as a white solid (700 mg, 1.770 mmol, 41.1% yield). ¹H NMR (400 MHz, CDCl₃) δ 7.21 (m, 3H), 7.05 (m, 1H), 5.03 (s, 1H), 4.00 (s, 2H), 3.67 (s, 3H), 3.20 (m, 2H), 2.71 (m, 2H), 2.43 (s, 3H), 2.23 (s, 3H), 1.84 (s, 3H), 0.95 (s, 9H). LCMS (ES+)(m/z): 396.44 (M+1); 791.69 (2M+1).

### Step 11: Methyl 2-(tert-butoxy)-2-(2-(3-fluorobenzyl)-5,8-dimethyl-6-(p-tolyl)-1,2,3,4-tetrahydroisoquinolin-7-yl)acetate

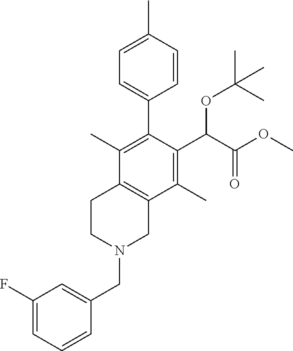 [SMILES: COC(=O)C(OC(C)(C)C)c1c(C)c2c(c(C)c1-c1ccc(C)cc1)CCN(Cc1cccc(F)c1)C2]

To a solution of methyl 2-(tert-butoxy)-2-(5,8-dimethyl-6-(p-tolyl)-1,2,3,4-tetrahydroisoquinolin-7-yl)acetate (20 mg, 0.051 mmol) in 2 mL DCE was added 3-fluorobenzaldehyde (8.05 μl, 0.076 mmol). The solution was stirred for 10 minutes then sodium triacetoxyborohydride (16.92 mg, 0.076 mmol) was added. After 30 minutes sodium triacetoxyborohydride (5.64 mg, 0.025 mmol) was added and the solution was stirred for 30 minutes, diluted with DCM and NaHCO₃, extracted with DCM, dried over Na₂SO₄ and the solvent was removed. The purple oil was carried on to the next step without further purification.

¹H NMR (400 MHz, CDCl₃) δ 7.35-6.92 (m, 8H), 5.02 (s, 1H), 3.74 (s, 2H), 3.65 (s, 3H), 3.60 (s, 2H), 2.82-2.63 (m, 4H), 2.42 (s, 3H), 2.19 (s, 3H), 1.83 (s, 3H), 0.94 (s, 9H). LCMS (ES+)(m/z): 504.51 (M+1); 526.45 (2M+1).

### Step 12: 2-(tert-Butoxy)-2-(2-(3-fluorobenzyl)-5,8-dimethyl-6-(p-tolyl)-1,2,3,4-tetrahydroisoquinolin-7-yl)acetic Acid

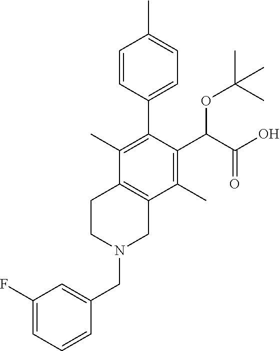 [SMILES: Cc1ccc(-c2c(C)c3c(c(C)c2C(OC(C)(C)C)C(=O)O)CN(Cc2cccc(F)c2)CC3)cc1]

To a solution of methyl 2-(tert-butoxy)-2-(2-(3-fluorobenzyl)-5,8-dimethyl-6-(p-tolyl)-1,2,3,4-tetrahydroisoquinolin-7-yl)acetate (25 mg, 0.050 mmol) in 4 mL 1,4-dioxane was added LiOH (0.745 mL, 0.745 mmol). The solution was stirred at 70° C. overnight. The solution was concentrated and EtOAc was added, washed with 1M HCl, brine, the solvent was removed and the oil was purified by reverse phase HPLC to yield the title compound as a white solid (13.2 mg, 0.027 mmol, 54.3% yield). ¹H NMR (400 MHz, CDCl₃) δ 7.47 (m, 1H), 7.39-7.15 (m, 6H), 7.08 (m, 1H), 5.15 (s, 1H), 4.72-2.84 (m, 8H), 2.42 (s, 3H), 2.11 (s, 3H), 1.90 (s, 3H), 0.98 (s, 9H). LCMS (ES+)(m/z): 490.70 (M+1); 979.86 (2M+1).

### Example 2: 2-(tert-Butoxy)-2-(2-cyclohexyl-5,8-dimethyl-6-(p-tolyl)-1,2,3,4-tetrahydroisoquinolin-7-yl)acetic Acid

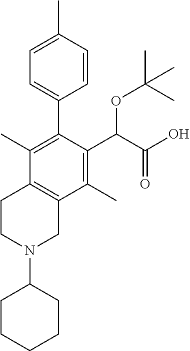 [SMILES: Cc1ccc(-c2c(C)c3c(c(C)c2C(OC(C)(C)C)C(=O)O)CN(C2CCCCC2)CC3)cc1]

The title compound was made in a manner similar to that described in example 1 from cyclohexanone and isolated as a white solid after reverse phase HPLC. ¹H NMR (400 MHz, CDCl₃) δ 7.35-7.08 (m, 4H), 5.15 (s, 1H), 4.53 (m, 1H), 4.09 (m, 1H), 3.74 (m, 1H), 3.47-3.13 (m, 3H), 2.92 (m, 1H), 2.42 (s, 3H), 2.30-2.14 (m, 5H), 2.00 (m, 2H), 1.90 (s, 3H), 1.83-1.56 (m, 2H), 1.50-1.19 (m, 4H), 0.98 (s, 9H). LCMS (ES+)(m/z): 464.51 (M+1).

### Example 3: 2-(tert-Butoxy)-2-(2-(cyclohexylmethyl)-5,8-dimethyl-6-(p-tolyl)-1,2,3,4-tetrahydroisoquinolin-7-yl)acetic Acid

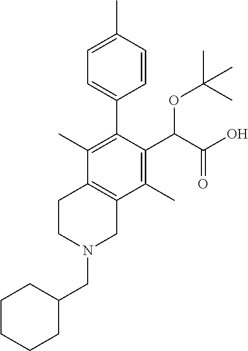 [SMILES: Cc1ccc(-c2c(C)c3c(c(C)c2C(OC(C)(C)C)C(=O)O)CN(CC2CCCCC2)CC3)cc1]

The title compound was made in a manner similar to that described in example 1 from cyclohexanecarbaldehyde and isolated as a white solid after reverse phase HPLC. ¹H NMR (400 MHz, CDCl₃) δ 7.33 (m, 1H), 7.26 (m, 2H), 7.09 (m, 1H), 5.16 (m, 1H), 4.72 (m, 1H), 3.99-3.76 (m, 1H), 3.25-2.82 (m, 3H), 2.43 (s, 3H), 2.18 (s, 3H), 1.99-1.66 (m, 9H), 1.39-1.05 (m, 8H), 0.97 (s, 9H). LCMS (ES+)(m/z): 479.49 (M+1); 955.95 (2M+1).

### Example 4: 2-(tert-Butoxy)-2-(2-(cyclohexanecarbonyl)-5,8-dimethyl-6-(p-tolyl)-1,2,3,4-tetrahydroisoquinolin-7-yl)acetic Acid

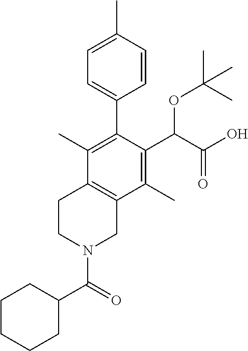 [SMILES: Cc1ccc(-c2c(C)c3c(c(C)c2C(OC(C)(C)C)C(=O)O)CN(C(=O)C2CCCCC2)CC3)cc1]

### Step 1: Methyl 2-(tert-butoxy)-2-(2-(cyclohexanecarbonyl)-5,8-dimethyl-6-(p-tolyl)-1,2,3,4-tetrahydroisoquinolin-7-yl)acetate

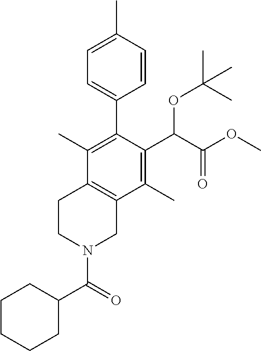 [SMILES: COC(=O)C(OC(C)(C)C)c1c(C)c2c(c(C)c1-c1ccc(C)cc1)CCN(C(=O)C1CCCCC1)C2]

To a solution of cyclohexanecarbonyl chloride (0.034 mL, 0.253 mmol) in 2 mL DCM was added TEA (0.042 mL, 0.303 mmol) followed by a solution of methyl 2-(tert-butoxy)-2-(5,8-dimethyl-6-(p-tolyl)-1,2,3,4-tetrahydroisoquinolin-7-yl)acetate (20 mg, 0.051 mmol) in 1 mL DCM. The solution was stirred for 24 hours, diluted with DCM and 1M HCl, washed with brine and the solvent was removed. The resulting oil was purified by silica gel chromatography (0-100% EtOAc/Hexanes gradient elution) to yield the title compound as a white solid (12.5 mg, 0.025 mmol, 48.9% yield). ¹H NMR (400 MHz, CDCl₃) δ 7.21 (m, 3H), 7.05 (m, 1H), 5.04 (s, 1H), 4.66 (m, 2H), 4.04-3.59 (m, 5H), 2.87-2.54 (m, 3H), 2.42 (s, 3H), 2.30 (s, 3H), 1.94-1.20 (m, 13H), 0.97 (s, 9H). LCMS (ES+)(m/z): 506.56 (M+1); 1012.00 (2M+1).

### Step 2: 2-(tert-Butoxy)-2-(2-(cyclohexanecarbonyl)-5,8-dimethyl-6-(p-tolyl)-1,2,3,4-tetrahydroisoquinolin-7-yl)acetic Acid

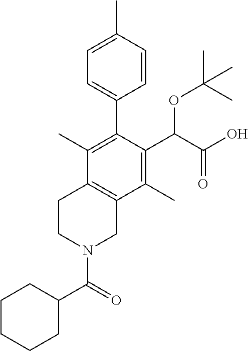 [SMILES: Cc1ccc(-c2c(C)c3c(c(C)c2C(OC(C)(C)C)C(=O)O)CN(C(=O)C2CCCCC2)CC3)cc1]

To a solution of methyl 2-(tert-butoxy)-2-(2-(cyclohexanecarbonyl)-5,8-dimethyl-6-(p-tolyl)-1,2,3,4-tetrahydroisoquinolin-7-yl)acetate (12.5 mg, 0.025 mmol) in 2 mL 1,4-dioxane was added KOTMS (12.68 mg, 0.099 mmol). The solution was heated to 100° C. and stirred for 2 hours, then diluted with EtOAc and 1M HCl, extracted with EtOAc, dried over Na₂SO₄ and the solvent was removed. The resulting brown oil purified by reverse phase HPLC to yield the title compound as a white solid (6.4 mg, 0.013 mmol, 52.7% yield). ¹H NMR (400 MHz, CDCl₃) δ 7.36 (m, 1H), 7.23 (m, 2H), 7.07 (m, 1H), 5.18 (m, 1H), 4.89 (m, 1H), 4.63-4.43 (m, 1H), 3.99-3.59 (m, 1H), 2.87-2.69 (m, 2H), 2.61 (m, 1H), 2.42 (s, 3H), 2.23 (s, 3H), 1.93-1.67 (m, 9H), 1.67-1.49 (m, 2H), 1.31 (m, 3H), 0.98 (s, 9H). LCMS (ES+)(m/z): 492.57 (M+1); 984.02 (2M+1).

### Example 5: 2-(tert-Butoxy)-2-(5,8-dimethyl-2-(2-oxo-2-(piperidin-1-yl)acetyl)-6-(p-tolyl)-1,2,3,4-tetrahydroisoquinolin-7-yl)acetic Acid

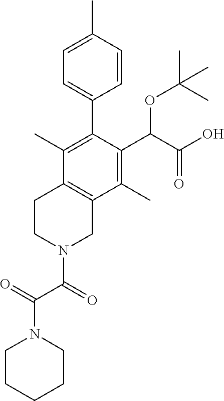 [SMILES: Cc1ccc(-c2c(C)c3c(c(C)c2C(OC(C)(C)C)C(=O)O)CN(C(=O)C(=O)N2CCCCC2)CC3)cc1]

### Step 1: Methyl 2-(tert-butoxy)-2-(5,8-dimethyl-2-(2-oxo-2-(piperidin-1-yl)acetyl)-6-(p-tolyl)-1,2,3,4-tetrahydroisoquinolin-7-yl)acetate

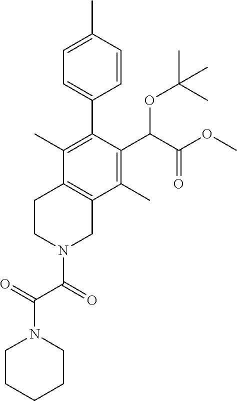 [SMILES: COC(=O)C(OC(C)(C)C)c1c(C)c2c(c(C)c1-c1ccc(C)cc1)CCN(C(=O)C(=O)N1CCCCC1)C2]

To a solution of 2-oxo-2-(piperidin-1-yl)acetic acid (15.89 mg, 0.101 mmol) in 1 mL EtOAc was added TEA (0.021 mL, 0.152 mmol) and T3P (0.075 mL, 0.126 mmol). The solution was stirred for 10 minutes then methyl 2-(tert-butoxy)-2-(5,8-dimethyl-6-(p-tolyl)-1,2,3,4-tetrahydroisoquinolin-7-yl)acetate (20 mg, 0.051 mmol) was added. The reaction was stirred at room temperature overnight, diluted with EtOAc and NaHCO₃, washed with brine and the solvent was removed. The purple oil was carried on to the next step without further purification.

¹H NMR (400 MHz, CDCl₃) δ (mixture of rotamers) 7.22 (m, 3H), 7.05 (m, 1H), 5.03 (m, 1H), 4.81-4.42 (m, 2H), 3.80-3.57 (m, 6H), 3.41 (m, 2H), 2.83 (m, 2H), 2.42 (m, 3H), 2.35-2.18 (m, 3H), 1.86 (m, 3H), 1.77-1.55 (m, 6H), 0.95 (m, 9H). LCMS (ES+)(m/z): 557.57 (M+23); 1070.02 (2M+1).

### Step 2: 2-(tert-Butoxy)-2-(5,8-dimethyl-2-(2-oxo-2-(piperidin-1-yl)acetyl)-6-(p-tolyl)-1,2,3,4-tetrahydroisoquinolin-7-yl)acetic Acid

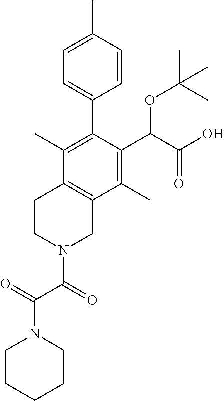 [SMILES: Cc1ccc(-c2c(C)c3c(c(C)c2C(OC(C)(C)C)C(=O)O)CN(C(=O)C(=O)N2CCCCC2)CC3)cc1]

The title compound was made in a manner similar to that described in example 4 step 2 and isolated as a white solid after reverse phase HPLC. ¹H NMR (400 MHz, CDCl₃) δ (mixture of rotamers) 7.33 (m, 1H), 7.23 (m, 2H), 7.06 (m, 1H), 5.16 (m, 1H), 4.90-4.40 (m, 2H), 4.05-3.16 (m, 6H), 2.83 (m, 2H), 2.42 (m, 3H), 2.30-2.13 (m, 3H), 1.88 (m, 3H), 1.67 (m, 7H), 0.98 (m, 9H). LCMS (ES+)(m/z): 521.51 (M+1); 543.44 (M+23); 1041.98 (2M+1).

### Example 6: 2-(tert-Butoxy)-2-(2-(3-fluorobenzyl)-5,8-dimethyl-6-(p-tolyl)-1,2,3,4-tetrahydroisoquinolin-7-yl)acetic Acid

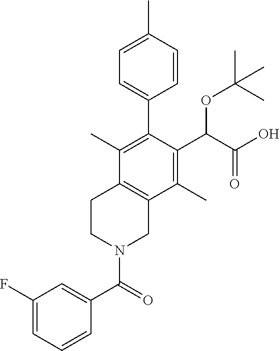 [SMILES: Cc1ccc(-c2c(C)c3c(c(C)c2C(OC(C)(C)C)C(=O)O)CN(C(=O)c2cccc(F)c2)CC3)cc1]

The title compound was made in a manner similar to that described in example 5 from 3-fluorobenzoic acid and isolated as a white solid after reverse phase HPLC. ¹H NMR (400 MHz, CDCl₃) δ (mixture of rotamers) 7.59-6.85 (m, 8H), 5.15 (br. s., 1H), 4.93 (m, 1H), 4.56 (m, 1H), 3.99 (m, 1H), 3.76-3.43 (m, 1H), 3.21 (m, 1H), 2.90-2.66 (m, 2H), 2.40 (s, 3H), 2.28 (br. s., 2H), 1.99 (m, 1H), 1.87 (s, 3H), 0.98 (s, 9H) LCMS (ES+)(m/z): 504.34 (M+1).

### Example 7: 2-(tert-Butoxy)-2-(5,8-dimethyl-2-(2-oxo-2-(piperidin-1-yl)ethyl)-6-(p-tolyl)-1,2,3,4-tetrahydroisoquinolin-7-yl)acetic Acid

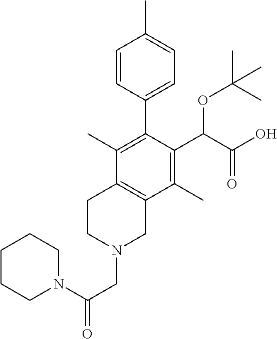 [SMILES: Cc1ccc(-c2c(C)c3c(c(C)c2C(OC(C)(C)C)C(=O)O)CN(CC(=O)N2CCCCC2)CC3)cc1]

### Step 1: Methyl 2-(tert-butoxy)-2-(2-(2-methoxy-2-oxoethyl)-5,8-dimethyl-6-(p-tolyl)-1,2,3,4-tetrahydroisoquinolin-7-yl)acetate

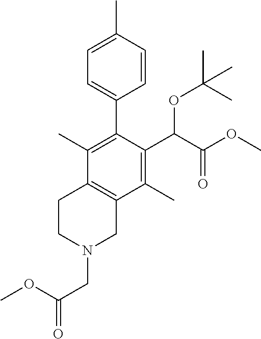

To a solution of methyl 2-(tert-butoxy)-2-(5,8-dimethyl-6-(p-tolyl)-1,2,3,4-tetrahydroisoquinolin-7-yl)acetate (10 mg, 0.025 mmol) in 2 mL DMF was added methyl 2-bromoacetate (4.31 μl, 0.046 mmol) and cesium carbonate (12.36 mg, 0.038 mmol). The solution was stirred overnight at room temperature, diluted with diethyl ether and water, extracted with diethyl ether, dried over Na₂SO₄ and the solvent was removed. The resulting oil was purified by silica gel chromatography (0-20% EtOAc/Hexanes gradient elution) to yield the title compound as a white solid (11.4 mg, 0.024 mmol, 96% yield).

¹H NMR (400 MHz, CDCl₃) δ 7.20 (m, 3H), 7.04 (m, 1H), 5.01 (s, 1H), 3.89-3.58 (m, 8H), 3.49 (s, 2H), 2.96-2.75 (m, 4H), 2.42 (s, 3H), 2.21 (s, 3H), 1.82 (s, 3H), 0.94 (s, 9H). LCMS (ES+)(m/z): 468.50 (M+1); 935.87 (2M+1).

### Step 2: 2-(7-(1-(tert-Butoxy)-2-methoxy-2-oxoethyl)-5,8-dimethyl-6-(p-tolyl)-3,4-dihydroisoquinolin-2(1H)-yl)acetic Acid

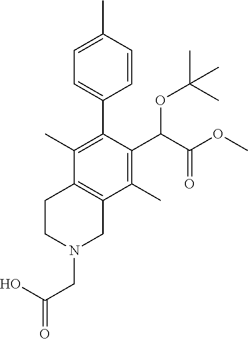 [SMILES: COC(=O)C(OC(C)(C)C)c1c(C)c2c(c(C)c1-c1ccc(C)cc1)CCN(CC(=O)O)C2]

To a solution of methyl 2-(tert-butoxy)-2-(2-(2-methoxy-2-oxoethyl)-5,8-dimethyl-6-(p-tolyl)-1,2,3,4-tetrahydroisoquinolin-7-yl)acetate (11.6 mg, 0.025 mmol) in 2 mL 1,4-dioxane was added LiOH (0.099 mL, 0.099 mmol), the solution was stirred at room temperature for 1 hour, more LiOH (0.099 mL, 0.099 mmol) was added and stirred for 1 hour, diluted with 1M HCl, extracted with EtOAc, washed with brine and the solvent was removed. The resulting oil was purified by silica gel chromatography (0-20% MeOH/DCM gradient elution) to yield the title compound as a clear oil (8 mg, 0.018 mmol, 71.1% yield). ¹H NMR (400 MHz, CDCl₃) δ 7.24-7.09 (m, 3H), 7.02 (m, 1H), 4.99 (s, 1H), 4.38 (m, 2H), 3.92 (m, 2H), 3.74-3.34 (m, 5H), 3.00 (m, 2H), 2.41 (s, 3H), 2.24 (s, 3H), 1.83 (s, 3H), 0.92 (s, 9H). LCMS (ES+)(m/z): 454.49 (M+1); 907.83 (2M+1).

### Step 3: Methyl 2-(tert-butoxy)-2-(5,8-dimethyl-2-(2-oxo-2-(piperidin-1-yl)ethyl)-6-(p-tolyl)-1,2,3,4-tetrahydroisoquinolin-7-yl)acetate

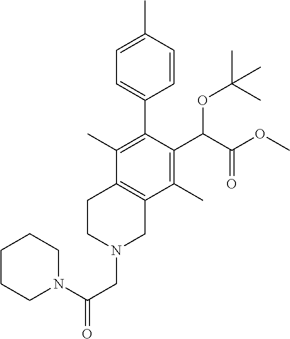 [SMILES: COC(=O)C(OC(C)(C)C)c1c(C)c2c(c(C)c1-c1ccc(C)cc1)CCN(CC(=O)N1CCCCC1)C2]

To a solution of 2-(7-(1-(tert-butoxy)-2-methoxy-2-oxoethyl)-5,8-dimethyl-6-(p-tolyl)-3,4-dihydroisoquinolin-2(1H)-yl)acetic acid (25 mg, 0.055 mmol), TEA (0.023 mL, 0.165 mmol), and T3P (0.082 mL, 0.138 mmol) in 1 mL EtOAc was added piperidine (10.89 μl, 0.110 mmol). The purple solution was stirred overnight, quenched with NaHCO₃, and extracted with EtOAc, washed with brine, dried over Na₂SO₄ and the solvent was removed. The purple oil was purified by silica gel chromatography (0-100% EtOAc/Hexanes gradient elution) to yield the title compound as a white solid (9 mg, 0.017 mmol, 31.4% yield).

¹H NMR (400 MHz, CDCl₃) δ 7.26-7.16 (m, 3H), 7.05 (m, 1H), 5.04 (s, 1H), 3.76-3.33 (m, 11H), 2.97-2.66 (m, 3H), 2.42 (s, 3H), 2.23 (s, 3H), 1.84 (s, 3H), 1.74-1.45 (m, 7H), 0.95 (s, 9H). LCMS (ES+)(m/z): 521.69 (M+1); 1064.21 (2M+23).

### Step 4: 2-(tert-Butoxy)-2-(5,8-dimethyl-2-(2-oxo-2-(piperidin-1-yl)ethyl)-6-(p-tolyl)-1,2,3,4-tetrahydroisoquinolin-7-yl)acetic Acid

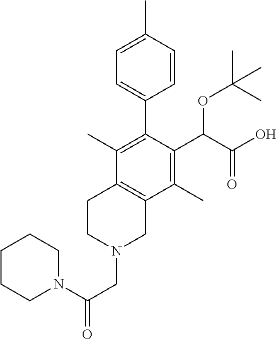 [SMILES: Cc1ccc(-c2c(C)c3c(c(C)c2C(OC(C)(C)C)C(=O)O)CN(CC(=O)N2CCCCC2)CC3)cc1]

The title compound was made in a manner similar to that described in example 4 step 2 and isolated as a white solid after reverse phase HPLC. ¹H NMR (400 MHz, CDCl₃) δ 7.31 (m, 3H), 7.08 (m, 1H), 5.16 (m, 1H), 4.75 (m, 1H), 4.46 (m, 1H), 4.23 (m, 2H), 3.97-2.80 (m, 8H), 2.43 (s, 3H), 2.16 (s, 3H), 1.90 (s, 3H), 1.63 (m, 6H), 0.98 (s, 9H). LCMS (ES+)(m/z): 507.52 (M+1).

### Example 8: 2-(tert-Butoxy)-2-(5,8-dimethyl-2-phenyl-6-(p-tolyl)-1,2,3,4-tetrahydroisoquinolin-7-yl)acetic Acid

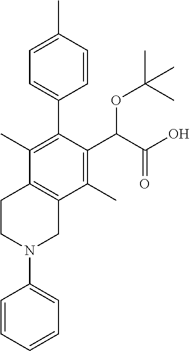 [SMILES: Cc1ccc(-c2c(C)c3c(c(C)c2C(OC(C)(C)C)C(=O)O)CN(c2ccccc2)CC3)cc1]

### Step 1: Methyl 2-(tert-butoxy)-2-(5,8-dimethyl-2-phenyl-6-(p-tolyl)-1,2,3,4-tetrahydroisoquinolin-7-yl)acetate

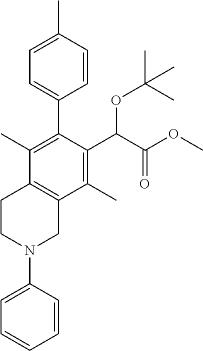 [SMILES: COC(=O)C(OC(C)(C)C)c1c(C)c2c(c(C)c1-c1ccc(C)cc1)CCN(c1ccccc1)C2]

To a solution of methyl 2-(tert-butoxy)-2-(5,8-dimethyl-6-(p-tolyl)-1,2,3,4-tetrahydroisoquinolin-7-yl)acetate (25 mg, 0.063 mmol) in 2 ml THF was added Ruphos Palladacycle (5.16 mg, 6.32 μmol) and iodobenzene (0.021 mL, 0.190 mmol). The solution stirred for 5 minutes under N₂ atmosphere then LHMDS (0.158 mL, 0.158 mmol) added dropwise. A color change occurred, and the solution was stirred for 30 minutes, cooled to 0° C., quenched with sat NH₄Cl, extracted with EtOAc, dried over Na₂SO₄ and the solvent was removed. The resulting brown oil was carried on to the next step without further purification.

LCMS (ES+)(m/z): 472.51 (M+1); 494.48 (M+23).

### Step 2: 2-(tert-Butoxy)-2-(5,8-dimethyl-2-phenyl-6-(p-tolyl)-1,2,3,4-tetrahydroisoquinolin-7-yl)acetic Acid

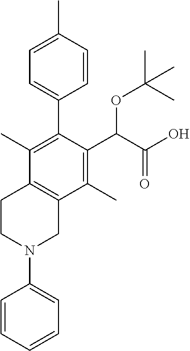 [SMILES: Cc1ccc(-c2c(C)c3c(c(C)c2C(OC(C)(C)C)C(=O)O)CN(c2ccccc2)CC3)cc1]

The title compound was made in a manner similar to that described in example 4 step 2 and isolated as a white solid after reverse phase HPLC. ¹H NMR (400 MHz, CDCl₃) δ 7.50-7.33 (m, 5H), 7.25 (m, 3H), 7.10 (m, 1H), 5.19 (m, 1H), 4.69 (m, 1H), 4.38 (m, 1H), 3.87-3.62 (m, 2H), 3.12-2.83 (m, 2H), 2.42 (s, 3H), 2.22 (s, 3H), 1.91 (s, 3H), 0.99 (s, 9H). LCMS (ES+)(m/z): 458.03 (M+1); 915.79 (2M+1).

### Example 9: 2-(tert-Butoxy)-2-(5,8-dimethyl-2-(piperidine-1-carbonyl)-6-(p-tolyl)-1,2,3,4-tetrahydroisoquinolin-7-yl)acetic Acid

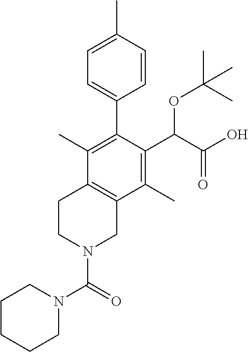 [SMILES: Cc1ccc(-c2c(C)c3c(c(C)c2C(OC(C)(C)C)C(=O)O)CN(C(=O)N2CCCCC2)CC3)cc1]

The title compound was made in a manner similar to that described in example 4 from piperidine-1-carbonyl chloride and isolated as a white solid after reverse phase HPLC. ¹H NMR (400 MHz, CDCl₃) δ 7.35 (m, 1H), 7.24 (m, 2H), 7.06 (m, 1H), 5.17 (m, 1H), 4.35 (m, 2H), 3.51 (m, 2H), 3.27 (m, 4H), 2.81 (m, 2H), 2.42 (s, 3H), 2.22 (s, 3H), 1.88 (s, 3H), 1.61 (m, 6H), 1.50-1.19 (m, 4H), 0.98 (s, 9H). LCMS (ES+)(m/z): 493.53 (M+1); 985.99 (2M+1).

### Example 10: 2-(tert-Butoxy)-2-(2-(cyclohexylcarbamoyl)-5,8-dimethyl-6-(p-tolyl)-1,2,3,4-tetrahydroisoquinolin-7-yl)acetic Acid

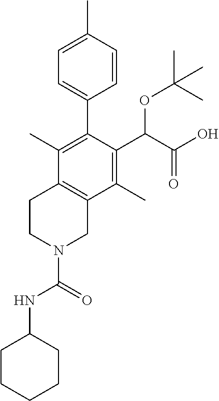 [SMILES: Cc1ccc(-c2c(C)c3c(c(C)c2C(OC(C)(C)C)C(=O)O)CN(C(=O)NC2CCCCC2)CC3)cc1]

### Step 1: Methyl 2-(tert-butoxy)-2-(2-(cyclohexylcarbamoyl)-5,8-dimethyl-6-(p-tolyl)-1,2,3,4-tetrahydroisoquinolin-7-yl)acetate

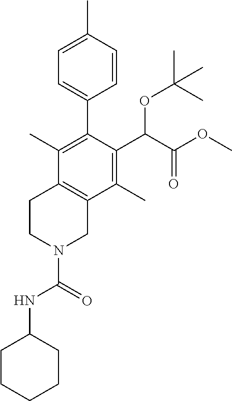 [SMILES: COC(=O)C(OC(C)(C)C)c1c(C)c2c(c(C)c1-c1ccc(C)cc1)CCN(C(=O)NC1CCCCC1)C2]

To a solution of methyl 2-(tert-butoxy)-2-(5,8-dimethyl-6-(p-tolyl)-1,2,3,4-tetrahydroisoquinolin-7-yl)acetate (10 mg, 0.025 mmol) in 1 mL DCE was added TEA (10.57 μl, 0.076 mmol) followed by isocyanatocyclohexane (6.46 μl, 0.051 mmol). The solution was stirred at room temperature for 30 minutes, quenched with 1M HCl, extracted with DCM, washed with brine, dried over Na₂SO₄ and the solvent was removed. The resulting purple oil was carried on to the next step without further purification. LCMS (ES+)(m/z): 543.47 (M+23); 1041.99 (2M+1).

### Step 2: 2-(tert-Butoxy)-2-(2-(cyclohexylcarbamoyl)-5,8-dimethyl-6-(p-tolyl)-1,2,3,4-tetrahydroisoquinolin-7-yl)acetic Acid

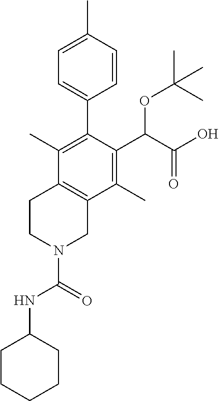 [SMILES: Cc1ccc(-c2c(C)c3c(c(C)c2C(OC(C)(C)C)C(=O)O)CN(C(=O)NC2CCCCC2)CC3)cc1]

The title compound was made in a manner similar to that described in example 1 step 12 and isolated as a white solid after reverse phase HPLC. ¹H NMR (400 MHz, CDCl₃) δ 7.39-7.01 (m, 4H), 5.17 (s, 1H), 4.55 (m, 1H), 4.36 (m, 1H), 3.78-3.50 (m, 3H), 2.78 (m, 2H), 2.41 (s, 3H), 2.23 (s, 3H), 2.00 (m, 2H), 1.88 (s, 3H), 1.79-1.57 (m, 3H), 1.47-1.07 (m, 5H), 0.97 (s, 9H). LCMS (ES+)(m/z): 507.46 (M+1); 1014.02 (2M+1).

### Example 11: 2-(tert-Butoxy)-2-(2-((3-fluorophenyl)sulfonyl)-5,8-dimethyl-6-(p-tolyl)-1,2,3,4-tetrahydroisoquinolin-7-yl)acetic Acid

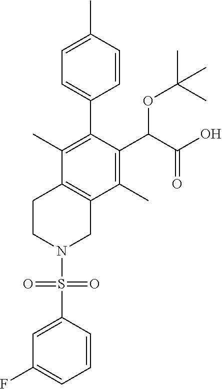 [SMILES: Cc1ccc(-c2c(C)c3c(c(C)c2C(OC(C)(C)C)C(=O)O)CN(S(=O)(=O)c2cccc(F)c2)CC3)cc1]

The title compound was made in a manner similar to that described in example 4 from 3-fluorobenzene-1-sulfonyl chloride and isolated as a white solid after reverse phase HPLC.

¹H NMR (400 MHz, CDCl₃) δ 7.67 (m, 1H), 7.57 (m, 2H), 7.38-7.18 (m, 4H), 7.04 (m, 1H), 5.14 (m, 1H), 4.43-3.98 (m, 2H), 2.83 (m, 2H), 2.41 (s, 3H), 2.17 (s, 3H), 1.83 (s, 3H), 0.97 (s, 9H). LCMS (ES+)(m/z): 562.47 (M+23); 1101.77 (2M+23).

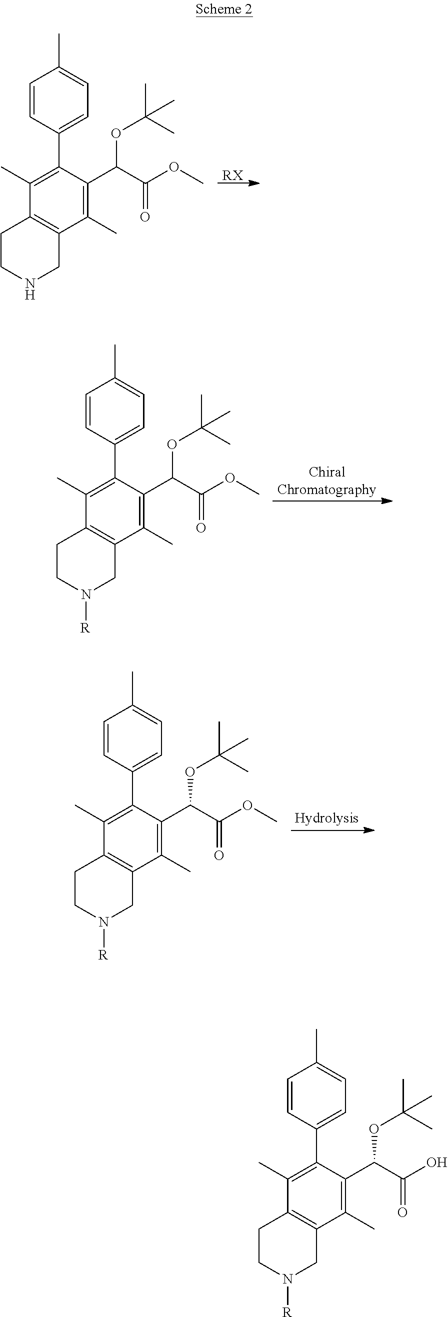

### Example 12: (S)-2-(tert-Butoxy)-2-(5,8-dimethyl-2-(piperidine-1-carbonyl)-6-(p-tolyl)-1,2,3,4-tetrahydroisoquinolin-7-yl)acetic Acid

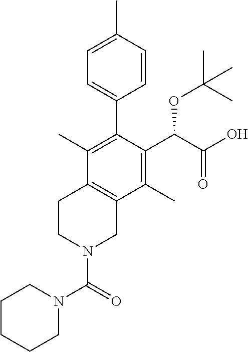 [SMILES: Cc1ccc(-c2c(C)c3c(c(C)c2[C@H](OC(C)(C)C)C(=O)O)CN(C(=O)N2CCCCC2)CC3)cc1]

### Step 1: (S)-Methyl 2-(tert-butoxy)-2-(5,8-dimethyl-2-(piperidine-1-carbonyl)-6-(p-tolyl)-1,2,3,4-tetrahydroisoquinolin-7-yl)acetate

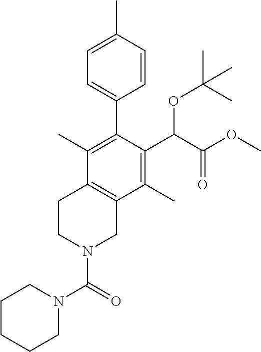 [SMILES: COC(=O)C(OC(C)(C)C)c1c(C)c2c(c(C)c1-c1ccc(C)cc1)CCN(C(=O)N1CCCCC1)C2]

The title compound was made in a manner similar to that described in example 4 step 1 from piperidine-1-carbonyl chloride. ¹H NMR (400 MHz, CDCl₃) δ 7.22 (m, 3H), 7.05 (m, 1H), 5.05 (s, 1H), 4.35 (s, 2H), 3.67 (s, 3H), 3.62-3.40 (m, 2H), 3.26 (m, 4H), 2.80 (m, 2H), 2.42 (s, 3H), 2.29 (s, 3H), 1.85 (s, 3H), 1.61 (m, 6H), 0.96 (s, 9H). LCMS (ES+)(m/z): 507.53 (M+1); 1013.98 (2M+1).

### Step 2: (S)-Methyl 2-(tert-butoxy)-2-(5,8-dimethyl-2-(piperidine-1-carbonyl)-6-(p-tolyl)-1,2,3,4-tetrahydroisoquinolin-7-yl)acetate

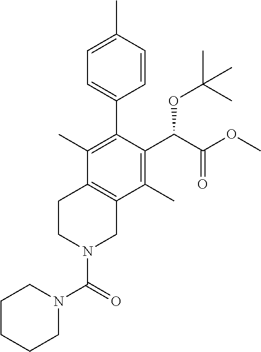 [SMILES: COC(=O)[C@@H](OC(C)(C)C)c1c(C)c2c(c(C)c1-c1ccc(C)cc1)CCN(C(=O)N1CCCCC1)C2]

(S)-methyl 2-(tert-butoxy)-2-(5,8-dimethyl-2-(piperidine-1-carbonyl)-6-(p-tolyl)-1,2,3,4-tetrahydroisoquinolin-7-yl)acetate (40 mg, 0.079 mmol) was purified using chiral chromatography to give (S)-methyl 2-(tert-butoxy)-2-(5,8-dimethyl-2-(piperidine-1-carbonyl)-6-(p-tolyl)-1,2,3,4-tetrahydroisoquinolin-7-yl)acetate (8.5 mg, 0.017 mmol, 21.25% yield) as a white solid. The purification was performed under the following conditions: Whelk-O (S,S) column (250×30 mm i.d., 5 μm; Regis Technologies, Morton Grove, Ill.) under high pressure conditions maintained at 23° C., with 45% EtOH, 55% Hexanes delivered at a combined flow rate of 42.5 ml/min on an Agilent HPLC 1100/1200 system (Agilent Technologies; Santa Clara, Calif.) equipped with a DAD detector and monitored at 280 nm. Retention time of the title compound under these conditions was 6.77 minutes and retention time of the undesired enantiomer was 7.75 minutes.

Chiral purity was determined by chiral analytical HPLC using a a Whelk-O (S,S) column (250×4.6 mm i.d., 5 μm; Regis Technologies, Morton Grove, Ill.) under high pressure conditions maintained at 23° C., with 50% EtOH, 50% Hexanes delivered at a combined flow rate of 1 ml/min on an Agilent HPLC 1100/1200 system (Agilent Technologies; Santa Clara, Calif.) equipped with a DAD detector and monitored at 280 nm. Retention time of the title compound under these conditions was 6.67 minutes and retention time of the undesired enantiomer was 7.56 minutes. ¹H NMR (400 MHz, CDCl₃) δ 7.22 (m, 3H), 7.05 (m, 1H), 5.05 (s, 1H), 4.35 (s, 2H), 3.67 (s, 3H), 3.62-3.40 (m, 2H), 3.26 (m, 4H), 2.80 (m, 2H), 2.42 (s, 3H), 2.29 (s, 3H), 1.85 (s, 3H), 1.61 (m, 6H), 0.96 (s, 9H). LCMS (ES+)(m/z): 507.52 (M+1); 1014.02 (2M+1).

### Step 3: (S)-2-(tert-Butoxy)-2-(5,8-dimethyl-2-(piperidine-1-carbonyl)-6-(p-tolyl)-1,2,3,4-tetrahydroisoquinolin-7-yl)acetic Acid

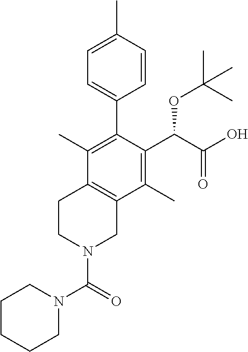 [SMILES: Cc1ccc(-c2c(C)c3c(c(C)c2[C@H](OC(C)(C)C)C(=O)O)CN(C(=O)N2CCCCC2)CC3)cc1]

The title compound was made in a manner similar to that described in example 4 step 2 and isolated as a white solid after reverse phase HPLC. ¹H NMR (400 MHz, CDCl₃) δ 7.36 (m, 1H), 7.24 (m, 2H), 7.06 (m, 1H), 5.18 (m, 1H), 4.35 (m, 2H), 3.49 (m, 2H), 3.26 (m, 4H), 2.80 (m, 2H), 2.41 (s, 3H), 2.21 (s, 3H), 1.88 (s, 3H), 1.61 (m, 6H), 0.98 (s, 9H). LCMS (ES+)(m/z): 493.44 (M+1); 985.89 (2M+1).

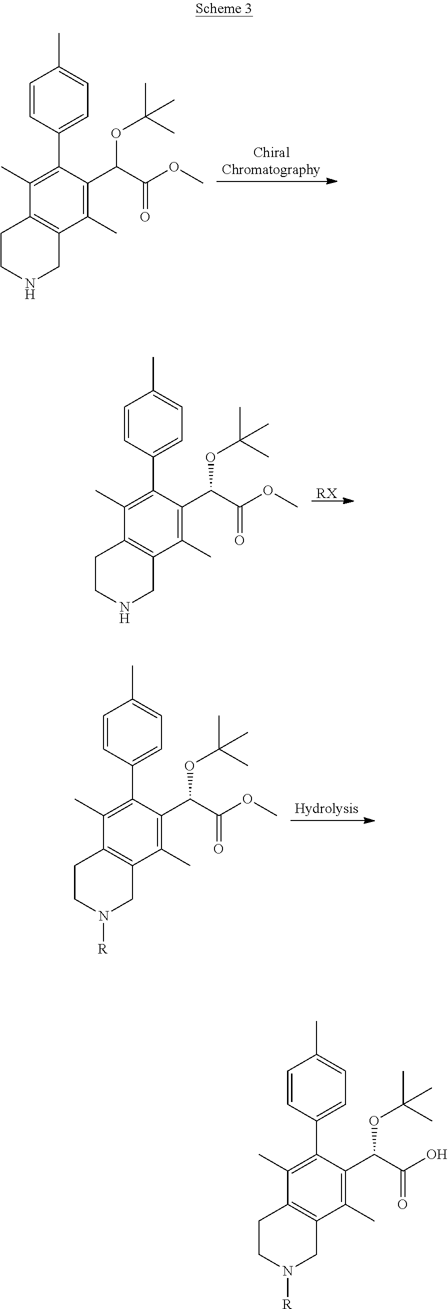

### Example 13: (S)-2-(tert-Butoxy)-2-(2-(4,4-dimethylpiperidine-1-carbonyl)-5,8-dimethyl-6-(p-tolyl)-1,2,3,4-tetrahydroisoquinolin-7-yl)acetic Acid

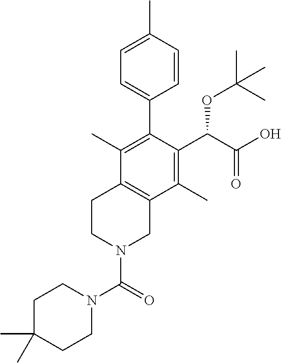 [SMILES: Cc1ccc(-c2c(C)c3c(c(C)c2[C@H](OC(C)(C)C)C(=O)O)CN(C(=O)N2CCC(C)(C)CC2)CC3)cc1]

### Step 1: (S)-Methyl 2-(tert-butoxy)-2-(5,8-dimethyl-6-(p-tolyl)-1,2,3,4-tetrahydroisoquinolin-7-yl)acetate

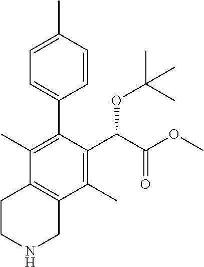 [SMILES: COC(=O)[C@@H](OC(C)(C)C)c1c(C)c2c(c(C)c1-c1ccc(C)cc1)CCNC2]

Methyl 2-(tert-butoxy)-2-(5,8-dimethyl-6-(p-tolyl)-1,2,3,4-tetrahydroisoquinolin-7-yl)acetate (650 mg, 1.643 mmol) was purified using chiral SFC chromatography to give (S)-methyl 2-(tert-butoxy)-2-(5,8-dimethyl-6-(p-tolyl)-1,2,3,4-tetrahydroisoquinolin-7-yl)acetate (229.1 mg, 0.579 mmol, 35.2% yield) as a white solid. The purification was performed under the following conditions: Chiral Tech IC column (250×20 mm i.d., 5 μm; Chiral Tech, West Chester, Pa.) under supercritical conditions maintained at 40° C., 100 bar, with methanol modified CO₂ (24.9% MeOH, 0.1% NH₄OH, 75% CO₂) delivered at a combined flow rate of 45 ml/min on a PIC prep SFC system (PIC Solution; Avignon, France). Triggered collections were made using a Knauer selectable wavelength UV-Vis dectector at 220 nm. Retention time of the title compound under these conditions was 12.84 minutes and retention time of the undesired enantiomer was 10.45 minutes.

Chiral purity was determined by chiral analytical SFC on a Chiral Tech IC column (250×4.6 mm i.d., 5 μm; Chiral Tech, West Chester, Pa.) under supercritical conditions maintained at 40° C., 140 bar, with methanol modified CO₂ (24.9% MeOH, 0.1% NH₄OH, 75% CO₂) delivered at a combined flow rate of 2 ml/min on a PIC Solution Analytical SFC system (Avignon, France) equipped with a DAD detector and monitored at 215 nm. Retention time of the title compound under these conditions was 9.38 minutes and retention time of the undesired enantiomer was 8.16 minutes. ¹H NMR (400 MHz, CDCl₃) δ 7.22 (m, 3H), 7.06 (m, 1H), 5.04 (s, 1H), 3.96 (s, 2H), 3.66 (s, 3H), 3.15 (m, 2H), 2.66 (m, 2H), 2.42 (s, 3H), 2.23 (s, 3H), 1.84 (s, 3H), 0.95 (s, 9H). LCMS (ES+)(m/z): 396.48 (M+1); 791.81 (2M+1).

### Step 2: (S)-2-(tert-Butoxy)-2-(2-(4,4-dimethylpiperidine-1-carbonyl)-5,8-dimethyl-6-(p-tolyl)-1,2,3,4-tetrahydroisoquinolin-7-yl)acetic Acid

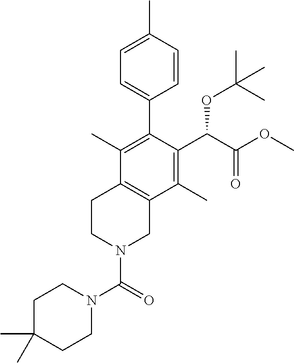

The title compound was made in a manner similar to that described in example 4 step 1 from 4,4-dimethylpiperidine-1-carbonyl chloride. ¹H NMR (400 MHz, CDCl₃) δ 7.22 (m, 3H), 7.05 (m, 1H), 5.04 (s, 1H), 4.34 (s, 2H), 3.68 (s, 3H), 3.61-3.40 (m, 2H), 3.28 (m, 4H), 2.80 (m, 2H), 2.42 (s, 3H), 2.29 (s, 3H), 1.85 (s, 3H), 1.40 (m, 4H), 0.97 (s, 15H). LCMS (ES+)(m/z): 535.53 (M+1); 557.53 (M+23).

### Step 3: (S)-2-(tert-Butoxy)-2-(2-(4,4-dimethylpiperidine-1-carbonyl)-5,8-dimethyl-6-(p-tolyl)-1,2,3,4-tetrahydroisoquinolin-7-yl)acetic Acid

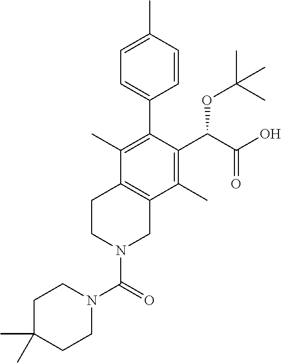 [SMILES: Cc1ccc(-c2c(C)c3c(c(C)c2[C@H](OC(C)(C)C)C(=O)O)CN(C(=O)N2CCC(C)(C)CC2)CC3)cc1]

The title compound was made in a manner similar to that described in example 4 step 2 and isolated as a white solid after reverse phase HPLC. ¹H NMR (400 MHz, CDCl₃) δ 7.35 (m, 1H), 7.23 (m, 2H), 7.06 (m, 1H), 5.17 (s, 1H), 4.34 (m, 2H), 3.49 (m, 2H), 3.29 (m, 4H), 2.80 (m, 2H), 2.41 (s, 3H), 2.22 (s, 3H), 1.88 (s, 3H), 1.40 (m, 4H), 0.98 (s, 15H). LCMS (ES+)(m/z): 521.50 (M+1); 1042.09 (2M+1).

### Example 14: (S)-2-(tert-Butoxy)-2-(5,8-dimethyl-2-(pyrrolidine-1-carbonyl)-6-(p-tolyl)-1,2,3,4-tetrahydroisoquinolin-7-yl)acetic Acid

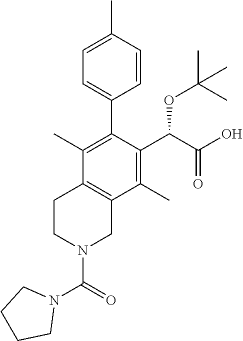 [SMILES: Cc1ccc(-c2c(C)c3c(c(C)c2[C@H](OC(C)(C)C)C(=O)O)CN(C(=O)N2CCCC2)CC3)cc1]

The title compound was made in a manner similar to that described in example 4 from pyrrolidine-1-carbonyl chloride and isolated as a white solid after reverse phase HPLC.

¹H NMR (400 MHz, CDCl₃) δ 7.36 (m, 1H), 7.24 (m, 2H), 7.06 (m, 1H), 5.17 (s, 1H), 4.40 (m, 2H), 3.55 (m, 2H), 3.46 (m, 4H), 2.81 (m, 2H), 2.42 (m, 3H), 2.22 (s, 3H), 1.88 (m, 7H), 0.98 (s, 9H). LCMS (ES+)(m/z): 501.63 (M+23); 958.21 (2M+1).

### Example 15: (S)-2-(tert-Butoxy)-2-(2-((4,4-dimethylpiperidin-1-yl)sulfonyl)-5,8-dimethyl-6-(p-tolyl)-1,2,3,4-tetrahydroisoquinolin-7-yl)acetic Acid

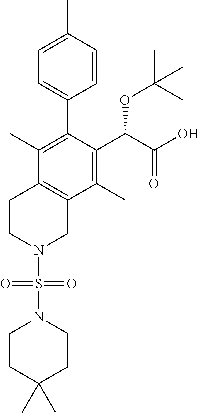 [SMILES: Cc1ccc(-c2c(C)c3c(c(C)c2[C@H](OC(C)(C)C)C(=O)O)CN(S(=O)(=O)N2CCC(C)(C)CC2)CC3)cc1]

The title compound was made in a manner similar to that described in example 4 from 4,4-dimethylpiperidine-1-sulfonyl chloride and isolated as a white solid after reverse phase HPLC as a TFA salt. ¹H NMR (400 MHz, CDCl₃) δ 9.72 (m, 1H), 7.35 (m, 1H), 7.23 (m, 2H), 7.06 (m, 1H), 5.17 (s, 1H), 4.33 (m, 2H), 3.50 (m, 2H), 3.27 (m, 4H), 2.82 (m, 2H), 2.41 (s, 3H), 2.19 (s, 3H), 1.88 (s, 3H), 1.43 (m, 4H), 0.97 (m, 15H). LCMS (ES+)(m/z): 557.45 (M+1); 1135.97 (2M+23).

### Example 16: (S)-2-(tert-Butoxy)-2-(5,8-dimethyl-2-(piperidin-1-ylsulfonyl)-6-(p-tolyl)-1,2,3,4-tetrahydroisoquinolin-7-yl)acetic Acid

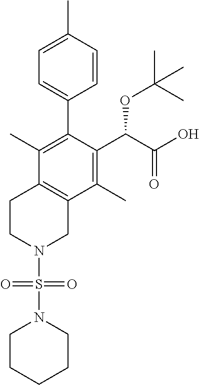 [SMILES: Cc1ccc(-c2c(C)c3c(c(C)c2[C@H](OC(C)(C)C)C(=O)O)CN(S(=O)(=O)N2CCCCC2)CC3)cc1]

The title compound was made in a manner similar to that described in example 4 from piperidine-1-sulfonyl chloride and isolated as a white solid after reverse phase HPLC as a TFA salt. ¹H NMR (400 MHz, CDCl₃) δ 9.74 (m, 1H), 7.35 (m, 1H), 7.24 (m, 2H), 7.06 (m, 1H), 5.16 (s, 1H), 4.32 (m, 2H), 3.50 (m, 2H), 3.26 (m, 4H), 2.81 (m, 2H), 2.41 (s, 3H), 2.19 (s, 3H), 1.88 (s, 3H), 1.68-1.45 (m, 6H), 0.98 (s, 9H). LCMS (ES+)(m/z): 529.53 (M+1); 1057.98 (2M+1).

### Example 17: (S)-2-(tert-Butoxy)-2-(2-(tert-butoxycarbonyl)-5,8-dimethyl-6-(p-tolyl)-1,2,3,4-tetrahydroisoquinolin-7-yl)acetic Acid

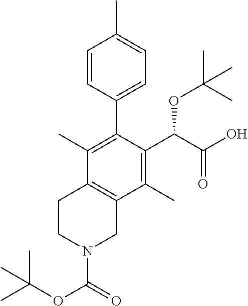 [SMILES: Cc1ccc(-c2c(C)c3c(c(C)c2[C@H](OC(C)(C)C)C(=O)O)CN(C(=O)OC(C)(C)C)CC3)cc1]

### Step 1: (S)-tert-Butyl 7-(1-(tert-butoxy)-2-methoxy-2-oxoethyl)-5,8-dimethyl-6-(p-tolyl)-3,4-dihydroisoquinoline-2(1H)-carboxylate

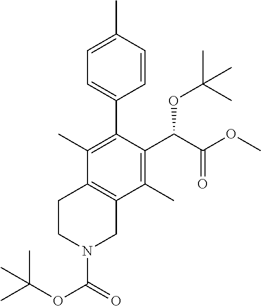 [SMILES: COC(=O)[C@@H](OC(C)(C)C)c1c(C)c2c(c(C)c1-c1ccc(C)cc1)CCN(C(=O)OC(C)(C)C)C2]

To a solution of (S)-methyl 2-(tert-butoxy)-2-(5,8-dimethyl-6-(p-tolyl)-1,2,3,4-tetrahydroisoquinolin-7-yl)acetate (10 mg, 0.025 mmol) in 2 mL MeOH was added TEA (10.57 μl, 0.076 mmol) followed by di-tert-butyl dicarbonate (11.04 mg, 0.051 mmol). The solution was stirred for 30 minutes then diluted with EtOAc and 1M HCl, extracted with EtOAc, washed with brine, the solvent was removed and the resulting purple oil was carried on to the next step without further purification. ¹H NMR (400 MHz, CDCl₃) δ 7.21 (m, 1H), 7.05 (m, 1H), 5.04 (s, 1H), 4.51 (m, 2H), 3.72 (s, 2H), 3.68 (s, 3H), 2.74 (m, 2H), 2.42 (s, 3H), 2.28 (s, 3H), 1.85 (s, 3H), 1.50 (m, 9H), 0.96 (s, 9H). LCMS (ES+)(m/z): 518.54 (M+23); 992.00 (2M+1).

### Step 2: (S)-2-(tert-Butoxy)-2-(2-(tert-butoxycarbonyl)-5,8-dimethyl-6-(p-tolyl)-1,2,3,4-tetrahydroisoquinolin-7-yl)acetic Acid

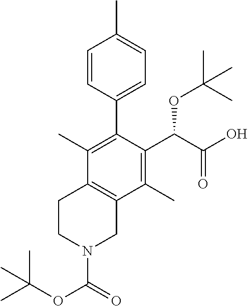 [SMILES: Cc1ccc(-c2c(C)c3c(c(C)c2[C@H](OC(C)(C)C)C(=O)O)CN(C(=O)OC(C)(C)C)CC3)cc1]

The title compound was made in a manner similar to that described in example 4 step 2 and isolated as a white solid after reverse phase HPLC. ¹H NMR (400 MHz, CDCl₃) δ 7.36 (m, 1H), 7.23 (m, 2H), 7.07 (m, 1H), 5.17 (m, 1H), 4.51 (m, 2H), 3.69 (m, 2H), 2.74 (m, 2H), 2.42 (s, 3H), 2.21 (s, 3H), 1.88 (s, 3H), 1.51 (s, 9H), 0.99 (s, 9H).

LCMS (ES+)(m/z): 504.43 (M+23).

### Example 18: (S)-2-(tert-Butoxy)-2-(2-((cyclohexylmethyl)sulfonyl)-5,8-dimethyl-6-(p-tolyl)-1,2,3,4-tetrahydroisoquinolin-7-yl)acetic Acid

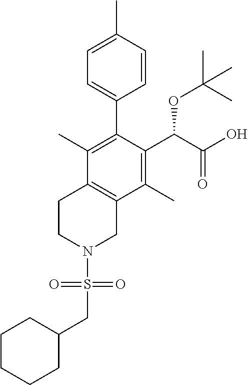 [SMILES: Cc1ccc(-c2c(C)c3c(c(C)c2[C@H](OC(C)(C)C)C(=O)O)CN(S(=O)(=O)CC2CCCCC2)CC3)cc1]

The title compound was made in a manner similar to that described in example 4 from cyclohexylmethanesulfonyl chloride and isolated as a white solid after reverse phase HPLC as a TFA salt. ¹H NMR (400 MHz, CDCl₃) δ 9.72 (m, 1H), 7.35 (m, 1H), 7.24 (m, 2H), 7.06 (m, 1H), 5.17 (s, 1H), 4.57-4.17 (m, 2H), 3.78-3.34 (m, 2H), 2.86 (m, 4H), 2.42 (s, 3H), 2.20 (s, 3H), 2.02 (m, 3H), 1.89 (s, 3H), 1.72 (m, 3H), 1.40-1.05 (m, 5H), 0.98 (s, 9H). LCMS (ES+)(m/z): 564.42 (M+23); 1106.74 (2M+23).

### Example 19: (S)-2-(tert-Butoxy)-2-(2-(cyclohexylsulfonyl)-5,8-dimethyl-6-(p-tolyl)-1,2,3,4-tetrahydroisoquinolin-7-yl)acetic Acid

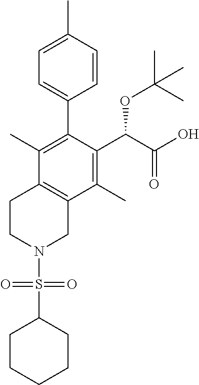 [SMILES: Cc1ccc(-c2c(C)c3c(c(C)c2[C@H](OC(C)(C)C)C(=O)O)CN(S(=O)(=O)C2CCCCC2)CC3)cc1]

The title compound was made in a manner similar to that described in example 4 from cyclohexanesulfonyl chloride and isolated as a white solid after reverse phase HPLC as a TFA salt. ¹H NMR (400 MHz, CDCl₃) δ 9.73 (m, 1H), 7.35 (m, 1H), 7.24 (m, 2H), 7.07 (m, 1H), 5.17 (s, 1H), 4.57-4.29 (m, 2H), 3.78-3.49 (m, 2H), 3.77-3.47 (m, 2H), 3.09-2.71 (m, 3H), 2.42 (s, 3H), 2.25-2.09 (m, 5H), 1.96-1.84 (m, 5H), 1.66-1.47 (m, 6H), 0.98 (s, 9H). LCMS (ES+)(m/z): 550.41 (M+23); 1077.97 (2M+23).

### Example 20: (S)-2-(tert-Butoxy)-2-(2-(3,3-dimethylbutanoyl)-5,8-dimethyl-6-(p-tolyl)-1,2,3,4-tetrahydroisoquinolin-7-yl)acetic Acid

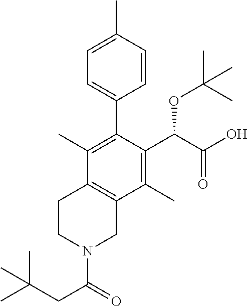 [SMILES: Cc1ccc(-c2c(C)c3c(c(C)c2[C@H](OC(C)(C)C)C(=O)O)CN(C(=O)CC(C)(C)C)CC3)cc1]

The title compound was made in a manner similar to that described in example 4 from 3,3-dimethylbutanoyl chloride and isolated as a white solid after reverse phase HPLC.

¹H NMR (400 MHz, CDCl₃) δ (mixture of rotamers) 7.36 (m, 1H), 7.24 (m, 2H), 7.07 (m, 1H), 5.17 (m, 1H), 4.98-4.44 (m, 2H), 4.01-3.59 (m, 2H), 2.78 (m, 2H), 2.49-2.14 (m, 8H), 1.88 (s, 3H), 1.16-0.92 (m, 18H). LCMS (ES+)(m/z): 480.60 (M+1); 960.14 (2M+1).

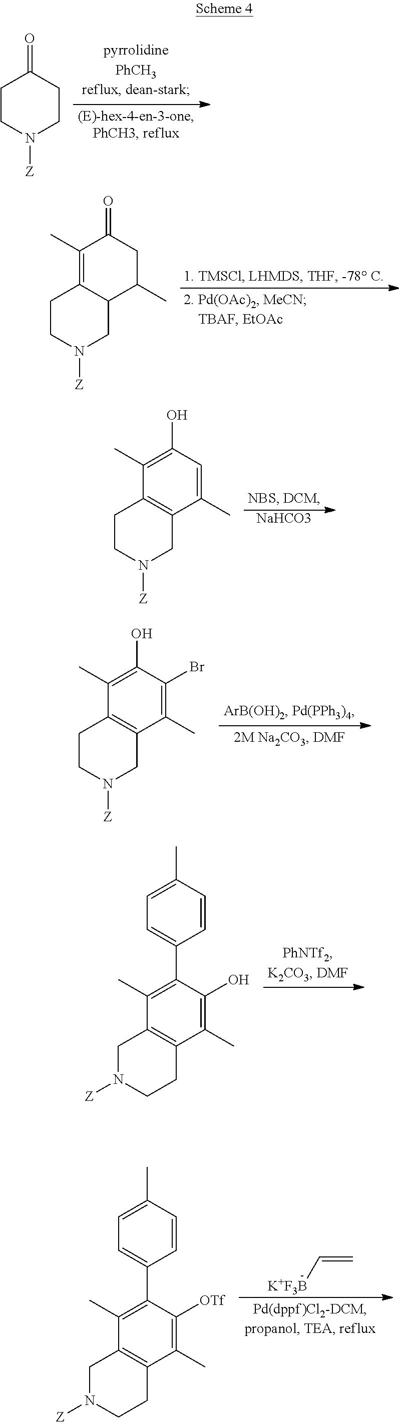

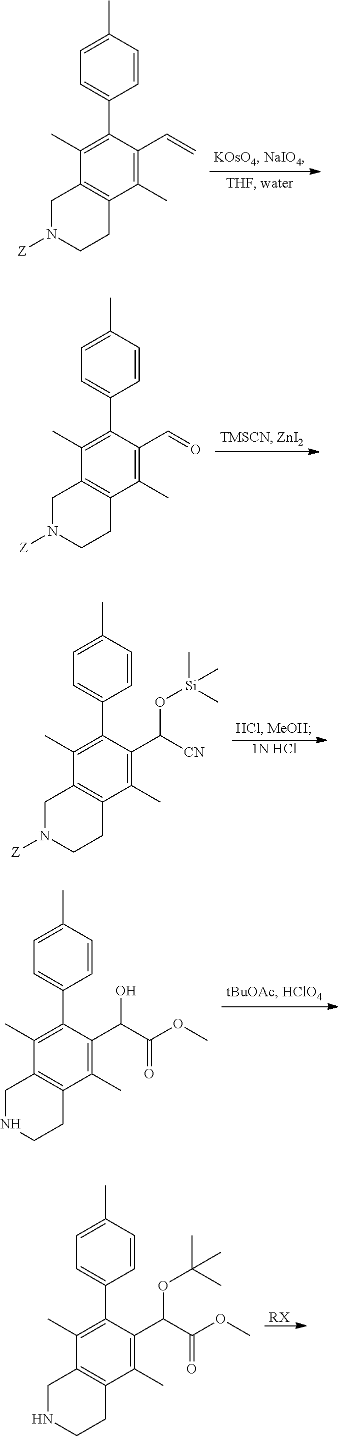

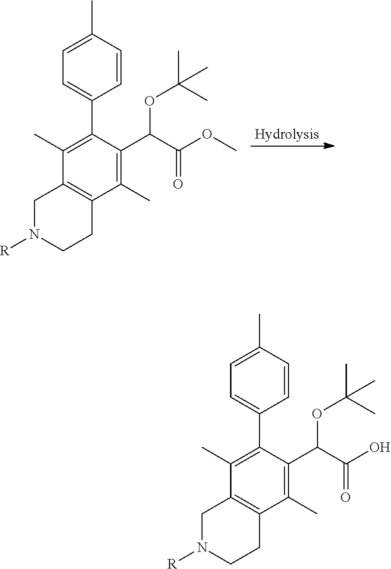

### Example 21: 2-(tert-Butoxy)-2-(2-cyclohexyl-5,8-dimethyl-7-(p-tolyl)-1,2,3,4-tetrahydroisoquinolin-6-yl)acetic Acid

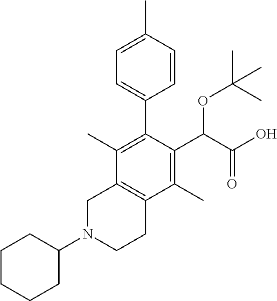 [SMILES: Cc1ccc(-c2c(C)c3c(c(C)c2C(OC(C)(C)C)C(=O)O)CCN(C2CCCCC2)C3)cc1]

### Step 1: Benzyl 6-hydroxy-5,8-dimethyl-7-(p-tolyl)-3,4-dihydroisoquinoline-2(1H)-carboxylate

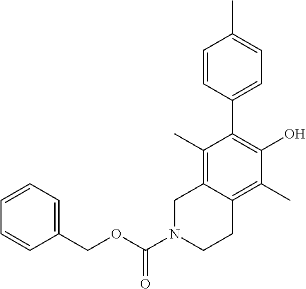 [SMILES: Cc1ccc(-c2c(C)c3c(c(C)c2O)CCN(C(=O)OCc2ccccc2)C3)cc1]

A solution of benzyl 7-bromo-6-hydroxy-5,8-dimethyl-3,4-dihydroisoquinoline-2(1H)-carboxylate (270 mg, 0.692 mmol) in 100 mL DMF was degassed for 5 minutes, p-tolylboronic acid (235 mg, 1.730 mmol), Pd(Ph₃P)₄ (160 mg, 0.138 mmol) and Na₂CO₃ (0.899 mL, 1.799 mmol) were added, the solution was heated to 80° C. and stirred for 1.5 hours. The solution was cooled to room temperature, diluted with di-ethyl ether and water, extracted with di-ethyl ether, dried over Na₂SO₄, and the solvent was removed. The resulting oil was purified by silica gel chromatography (0-20% EtOAc/Hexanes gradient elution) to yield the title compound as a yellow oil (21 g, 52.3 mmol, 91% yield). ¹H NMR (400 MHz, CDCl₃) δ 7.45-7.28 (m, 7H), 7.14 (m, 2H), 5.21 (s, 2H), 4.73 (s, 1H), 4.54 (s, 2H), 3.77 (m, 2H), 2.81 (m, 2H), 2.44 (s, 3H), 2.17 (s, 3H), 1.92 (s, 3H). LCMS (ES+)(m/z): 402.43 (M+1); 803.79 (2M+1).

### Step 2: Benzyl 5,8-dimethyl-7-(p-tolyl)-6-(((trifluoromethyl)sulfonyl)oxy)-3,4-dihydroisoquinoline-2(1H)-carboxylate

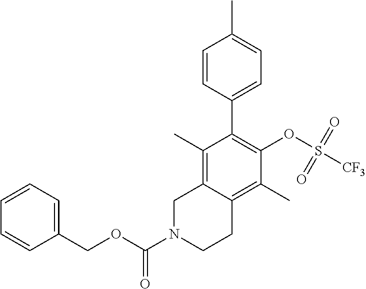 [SMILES: Cc1ccc(-c2c(C)c3c(c(C)c2OS(=O)(=O)C(F)(F)F)CCN(C(=O)OCc2ccccc2)C3)cc1]

To a solution of benzyl 6-hydroxy-5,8-dimethyl-7-(p-tolyl)-3,4-dihydroisoquinoline-2(1H)-carboxylate (2.94 g, 7.32 mmol) was added K₂CO₃ (2.226 g, 16.11 mmol) and PhNTf₂ (5.76 g, 16.11 mmol). The suspension was stirred at room temperature for 2 hours, diluted with diethyl ether and water, extracted with diethyl ether, dried over Na₂SO₄ and the solvent was removed. The resulting yellow oil was purified by silica gel chromatography (0-15% EtOAc/Hexanes gradient elution) to yield the title compound as a white foam (3.49 g, 6.54 mmol, 89% yield).

¹H NMR (400 MHz, CDCl₃) δ 7.45-7.29 (m, 5H), 7.24 (m, 2H), 7.08 (m, 2H), 5.21 (s, 2H), 4.58 (s, 2H), 3.79 (m, 2H), 2.82 (m, 2H), 2.41 (s, 3H), 2.29 (s, 3H), 1.98 (s, 3H). LCMS (ES+)(m/z): 534.43 (M+1); 1067.83 (2M+1).

### Step 3: Benzyl 5,8-dimethyl-7-(p-tolyl)-6-vinyl-3,4-dihydroisoquinoline-2(1H)-carboxylate

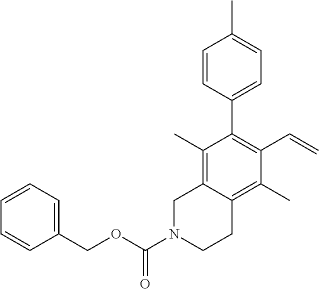 [SMILES: C=Cc1c(C)c2c(c(C)c1-c1ccc(C)cc1)CN(C(=O)OCc1ccccc1)CC2]

A solution of benzyl 5,8-dimethyl-7-(p-tolyl)-6-(((trifluoromethyl)sulfonyl)oxy)-3,4-dihydroisoquinoline-2(1H)-carboxylate (3.49 g, 6.54 mmol) in 100 mL 1-propanol was degassed with N₂ for 10 minutes. Vinyltrifluoroborate (3.50 g, 26.2 mmol), TEA (6.38 mL, 45.8 mmol), PdCl₂(dppf)-CH₂Cl₂ adduct (0.604 g, 0.739 mmol) were added and the suspension was heated to 135° C. for 2 hours, diluted with EtOAc and water, poured over ice water, extracted with EtOAc, dried over Na₂SO₄, and the solvent was removed. The resulting red oil was purified by silica gel chromatography (0-20% EtOAc/Hexanes gradient elution) to yield the title compound as a yellow oil (2.48 g, 6.03 mmol, 92% yield). ¹H NMR (400 MHz, CDCl₃) δ 7.47-7.29 (m, 5H), 7.18 (m, 2H), 6.96 (m, 2H), 6.34 (m, 1H), 5.29-5.14 (m, 3H), 4.95 (m, 1H), 4.60 (s, 2H), 3.79 (m, 2H), 2.84 (m, 2H), 2.40 (s, 3H), 2.28 (s, 3H), 1.91 (s, 3H). LCMS (ES+)(m/z): 412.45 (M+1); 823.73 (2M+1).

### Step 4: Benzyl 6-formyl-5,8-dimethyl-7-(p-tolyl)-3,4-dihydroisoquinoline-2(1H)-carboxylate

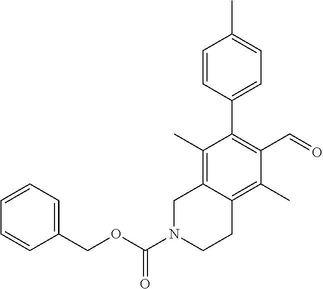 [SMILES: Cc1ccc(-c2c(C)c3c(c(C)c2C=O)CCN(C(=O)OCc2ccccc2)C3)cc1]

To a solution of benzyl 5,8-dimethyl-7-(p-tolyl)-6-vinyl-3,4-dihydroisoquinoline-2(1H)-carboxylate (2.68 g, 6.51 mmol) in 13 mL 3:1 THF/H₂O was added potassium osmate dihydrate (0.720 g, 1.954 mmol) followed by sodium periodate (8.36 g, 39.1 mmol). The solution was stirred for 1 hour, diluted with EtOAc and sodium thiosulfate, extracted with EtOAc, dried over Na₂SO₄ and the solvent was removed. The resulting brown oil was purified by silica gel chromatography (0-100% EtOAc/Hexanes gradient elution) to yield the title compound as a white solid (1.446 g, 3.50 mmol, 53.7% yield). ¹H NMR (400 MHz, CDCl₃) δ 9.73 (s, 1H), 7.46-7.29 (m, 5H), 7.24 (m, 2H), 7.04 (m, 2H), 5.21 (s, 2H), 4.63 (s, 2H), 3.79 (m, 2H), 2.86 (m, 2H), 2.48 (s, 3H), 2.42 (s, 3H), 1.96 (s, 3H). LCMS (ES+)(m/z): 414.41 (M+1); 827.67 (2M+1).

### Step 5: Benzyl 6-(cyano((trimethylsilyl)oxy)methyl)-5,8-dimethyl-7-(p-tolyl)-3,4-dihydroisoquinoline-2(1H)-carboxylate

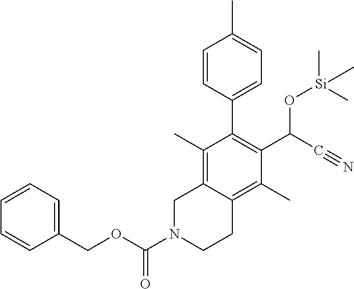 [SMILES: Cc1ccc(-c2c(C)c3c(c(C)c2C(C#N)O[Si](C)(C)C)CCN(C(=O)OCc2ccccc2)C3)cc1]

To a solution of benzyl 6-formyl-5,8-dimethyl-7-(p-tolyl)-3,4-dihydroisoquinoline-2(1H)-carboxylate (1.446 g, 3.50 mmol) in 50 mL DCM at 0° C. was added zinc iodide (0.558 g, 1.748 mmol) followed by TMS-CN (6.56 mL, 49.0 mmol). The solution was stirred for 1 hour, diluted with DCM, poured over ice, extracted with DCM, dried over Na₂SO₄ and the solvent was removed. The resulting clear yellow oil was carried on to the next step without further purification. ¹H NMR (400 MHz, CDCl₃) δ 7.44-7.31 (m, 5H), 7.26 (m, 2H), 7.01 (m, 2H), 5.36 (s, 1H), 5.21 (s, 2H), 4.56 (s, 2H), 3.88-3.64 (m, 2H), 2.84 (m, 2H), 2.52 (s, 3H), 2.44 (s, 3H), 1.87 (s, 3H), 0.00 (s, 9H). LCMS (ES+)(m/z): 535.44 (M+23).

### Step 6: Methyl 2-(5,8-dimethyl-7-(p-tolyl)-1,2,3,4-tetrahydroisoquinolin-6-yl)-2-hydroxyacetate

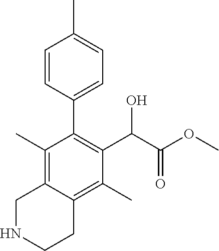 [SMILES: COC(=O)C(O)c1c(C)c2c(c(C)c1-c1ccc(C)cc1)CNCC2]

HCl gas was bubbled through MeOH at 0° C. for 20 minutes to make a saturated HCl solution. 150 mL of the solution was added to a flask with benzyl 6-(cyano((trimethylsilyl)oxy)methyl)-5,8-dimethyl-7-(p-tolyl)-3,4-dihydroisoquinoline-2(1H)-carboxylate (1.519 g, 2.96 mmol) at 0° C. The solution was stirred for 5 minutes at 0° C., warmed to room temperature and stirred for 2 hours, cooled back to 0° C. and HCl was bubbled through solution for 1 hour, stirred at 0° C. for 1 hour, warmed to room temperature and stirred for 1 hour. MeOH was removed by rotary evaporation without dipping into the water bath. The resulting clear oil was suspended in 100 mL 1M HCl and heated to 90° C. for 2.5 hours, poured over ice/water, neutralized with 50% NaOH & 1M NaOH, extracted with 1:3 IPA/CHCl₃, the solvent was removed and the clear colorless oil was carried on without further purification. ¹H NMR (400 MHz, CDCl₃) δ 7.43-6.98 (m, 4H), 5.09 (s, 1H), 4.70 (s, 1H), 3.96 (s, 2H), 3.70 (s, 3H), 3.18 (m, 2H), 2.72 (m, 2H), 2.41 (s, 3H), 2.16 (s, 3H), 1.81 (s, 3H). LCMS (ES+)(m/z): 340.39 (M+1), 679.65 (2M+1).

### Step 7: Methyl 2-(tert-butoxy)-2-(5,8-dimethyl-7-(p-tolyl)-1,2,3,4-tetrahydroisoquinolin-6-yl)acetate

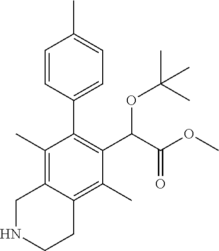 [SMILES: COC(=O)C(OC(C)(C)C)c1c(C)c2c(c(C)c1-c1ccc(C)cc1)CNCC2]

To a solution of methyl 2-(5,8-dimethyl-7-(p-tolyl)-1,2,3,4-tetrahydroisoquinolin-6-yl)-2-hydroxyacetate (1 g, 2.95 mmol) in tert-butyl acetate (200 mL, 1481 mmol) was added perchloric acid (0.886 mL, 14.73 mmol). The solution was stirred for 30 minutes then diluted with EtOAc and water, cooled to 0° C. and made basic (pH10) with 1M NaOH and 50% NaOH, extracted with EtOAc, dried over Na₂SO₄ and the solvent was removed. The resulting tan solid was purified by silica gel chromatography (0-10% MeOH/DCM gradient elution) to yield the title compound as a white solid (618 mg, 1.562 mmol, 53.0% yield). ¹H NMR (400 MHz, CDCl₃) δ 7.21 (m, 3H), 7.05 (m, 1H), 5.03 (s, 1H), 4.00 (s, 2H), 3.67 (s, 3H), 3.24 (m, 2H), 2.74 (m, 2H), 2.43 (s, 3H), 2.28 (s, 3H), 1.791 (s, 3H), 0.95 (s, 9H). LCMS (ES+)(m/z): 396.50 (M+1), 791.95 (2M+1).

### Step 8: Methyl 2-(tert-butoxy)-2-(2-cyclohexyl-5,8-dimethyl-7-(p-tolyl)-1,2,3,4-tetrahydroisoquinolin-6-yl)acetate

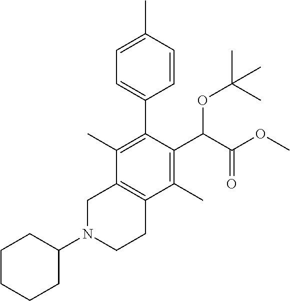 [SMILES: COC(=O)C(OC(C)(C)C)c1c(C)c2c(c(C)c1-c1ccc(C)cc1)CN(C1CCCCC1)CC2]

The title compound was made in a manner similar to that described in example 1 step 11 from cyclohexanone. ¹H NMR (400 MHz, CDCl₃) δ 7.20 (m, 3H), 7.05 (m, 1H), 5.00 (s, 1H), 3.76-3.57 (m, 5H), 2.93-2.72 (m, 4H), 2.58-2.31 (m, 5H), 2.25 (s, 3H), 2.03-1.52 (m, 12H), 0.94 (s, 9H). LCMS (ES+)(m/z): 478.58 (M+1); 500.56 (M+23).

### Step 9: 2-(tert-Butoxy)-2-(2-cyclohexyl-5,8-dimethyl-7-(p-tolyl)-1,2,3,4-tetrahydroisoquinolin-6-yl)acetic Acid

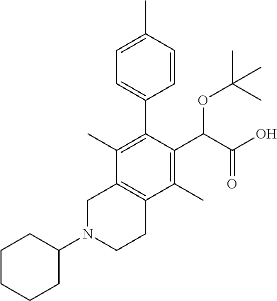 [SMILES: Cc1ccc(-c2c(C)c3c(c(C)c2C(OC(C)(C)C)C(=O)O)CCN(C2CCCCC2)C3)cc1]

The title compound was made in a manner similar to that described in example 1 step 12 and isolated as a white solid after reverse phase HPLC. ¹H NMR (400 MHz, CDCl₃) δ 7.32 (m, 1H), 7.25 (m, 2H), 7.10 (m, 1H), 5.17 (m, 1H), 4.49 (m, 1H), 4.02 (m, 1H), 3.76 (m, 1H), 3.45-3.11 (m, 3H), 2.96 (m, 1H), 2.42 (s, 3H), 2.23 (m, 5H), 1.99 (m, 2H), 1.87 (s, 3H), 1.81-1.32 (m, 6H), 0.94 (s, 9H). LCMS (ES+)(m/z): 464.31 (M+1); 927.93 (2M+1).

### Example 22: 2-(tert-Butoxy)-2-(2-(3-fluorobenzoyl)-5,8-dimethyl-7-(p-tolyl)-1,2,3,4-tetrahydroisoquinolin-6-yl)acetic Acid

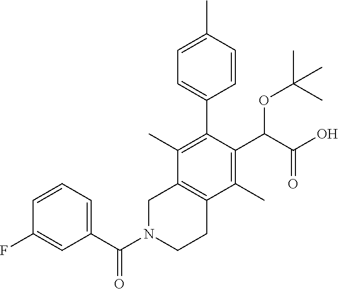 [SMILES: Cc1ccc(-c2c(C)c3c(c(C)c2C(OC(C)(C)C)C(=O)O)CCN(C(=O)c2cccc(F)c2)C3)cc1]

The title compound was made in a manner similar to that described in example 5 from 3-fluorobenzoic acid and isolated as a residue after reverse phase HPLC. ¹H NMR (400 MHz, CDCl₃) δ (mixture of rotamers) 7.61-6.62 (m, 8H), 5.34-5.04 (m, 1H), 5.02-4.81 (m, 1H), 4.74-3.20 (m, 4H), 3.05-2.57 (m, 2H), 2.45-1.79 (m, 9H), 0.97 (s, 9H). LCMS (ES+)(m/z): 526.31 (M+23).

### Example 23: 2-(tert-Butoxy)-2-(5,8-dimethyl-2-(piperidine-1-carbonyl)-7-(p-tolyl)-1,2,3,4-tetrahydroisoquinolin-6-yl)acetic Acid

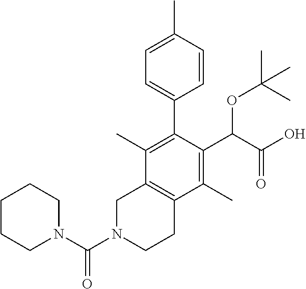 [SMILES: Cc1ccc(-c2c(C)c3c(c(C)c2C(OC(C)(C)C)C(=O)O)CCN(C(=O)N2CCCCC2)C3)cc1]

The title compound was made in a manner similar to that described in example 4 from piperidine-1-carbonyl chloride and isolated as a white solid after reverse phase HPLC. ¹H NMR (400 MHz, CDCl₃) δ 7.35 (m, 1H), 7.23 (m, 2H), 7.07 (m, 1H), 5.17 (m, 1H), 4.31 (m, 2H), 3.50 (m, 2H), 3.27 (m, 3H), 2.82 (m, 2H), 2.41 (s, 3H), 2.23 (s, 3H), 1.87 (s, 3H), 1.60 (m, 6H), 0.98 (s, 9H). LCMS (ES+)(m/z): 493.52 (M+1); 985.94 (2M+1).

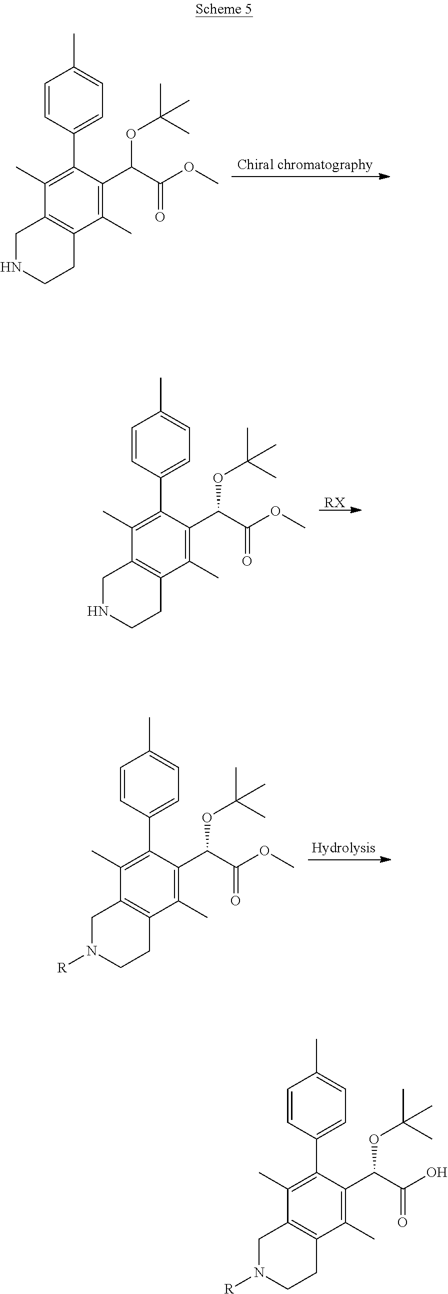

### Example 24: (S)-2-(tert-Butoxy)-2-(5,8-dimethyl-2-pivaloyl-7-(p-tolyl)-1,2,3,4-tetrahydroisoquinolin-6-yl)acetic Acid

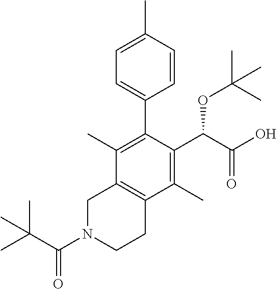 [SMILES: Cc1ccc(-c2c(C)c3c(c(C)c2[C@H](OC(C)(C)C)C(=O)O)CCN(C(=O)C(C)(C)C)C3)cc1]

### Step 1: (S)-Methyl 2-(tert-butoxy)-2-(5,8-dimethyl-7-(p-tolyl)-1,2,3,4-tetrahydroisoquinolin-6-yl)acetate

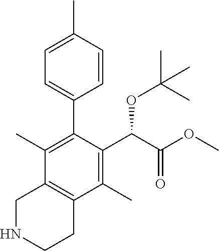 [SMILES: COC(=O)[C@@H](OC(C)(C)C)c1c(C)c2c(c(C)c1-c1ccc(C)cc1)CNCC2]

Methyl 2-(tert-butoxy)-2-(5,8-dimethyl-6-(p-tolyl)-1,2,3,4-tetrahydroisoquinolin-7-yl)acetate (550 mg, 1.391 mmol) was purified using chiral SFC chromatography to give (S)-methyl 2-(tert-butoxy)-2-(5,8-dimethyl-7-(p-tolyl)-1,2,3,4-tetrahydroisoquinolin-6-yl)acetate (200.1 mg, 0.506 mmol, 36.4% yield) as a white solid. The purification was performed under the following conditions: Chiral Tech CC₄ column (250×30 mm i.d., 10 μm; Chiral Tech, West Chester, Pa.) under supercritical conditions maintained at 40° C., 140 bar, with methanol modified CO₂ (19.9% MeOH, 0.1% NH₄OH, 80% CO₂) delivered at a combined flow rate of 90 ml/min on a PIC prep SFC system (PIC Solution; Avignon, France). Triggered collections were made using a Knauer selectable wavelength UV-Vis dectector at 220 nm. Retention time of the title compound under these conditions was 11.58 minutes and retention time of the undesired enantiomer was 9.17 minutes.

Chiral purity was determined by chiral analytical SFC on a Chiral Tech CC₄ column (250×4.6 mm i.d., 5 μm; Chiral Tech, West Chester, Pa.) under supercritical conditions maintained at 40° C., 140 bar, with methanol modified CO₂ (19.9% MeOH, 0.1% NH₄OH, 80% CO₂) delivered at a combined flow rate of 2 ml/min on PIC Solution Analytical SFC system (Avignon, France) equipped with a DAD detector and monitored at 220 nm. Retention time of the title compound under these conditions was 8.12 minutes and retention time of the undesired enantiomer was 6.88 minutes. ¹H NMR (400 MHz, CDCl₃) δ 7.22 (m, 3H), 7.06 (m, 1H), 5.03 (s, 1H), 4.04-3.56 (m, 5H), 3.40-3.60 (m, 5H), 2.42 (s, 3H), 2.28 (s, 3H), 1.80 (s, 3H), 0.95 (s, 9H).

LCMS (ES+)(m/z): 396.39 (M+1); 791.88 (2M+1).

### Step 2: (S)-Methyl 2-(tert-butoxy)-2-(5,8-dimethyl-2-pivaloyl-7-(p-tolyl)-1,2,3,4-tetrahydroisoquinolin-6-yl)acetate

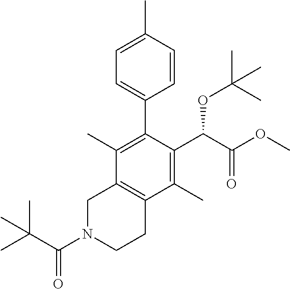 [SMILES: COC(=O)[C@@H](OC(C)(C)C)c1c(C)c2c(c(C)c1-c1ccc(C)cc1)CN(C(=O)C(C)(C)C)CC2]

The title compound was made in a manner similar to that described in example 4 step 1 from pivaloyl chloride. ¹H NMR (400 MHz, CDCl₃) δ 7.21 (m, 3H), 7.04 (m, 1H), 5.03 (s, 1H), 4.66 (m, 2H), 4.03-3.76 (m, 2H), 3.67 (s, 3H), 2.80 (m, 2H), 2.42 (s, 3H), 2.29 (s, 3H), 1.86 (s, 3H), 1.34 (s, 9H), 0.95 (s, 9H). LCMS (ES+)(m/z): 480.47 (M+1); 502.47 (M+23).

### Step 3: (S)-2-(tert-Butoxy)-2-(5,8-dimethyl-2-pivaloyl-7-(p-tolyl)-1,2,3,4-tetrahydroisoquinolin-6-yl)acetic Acid

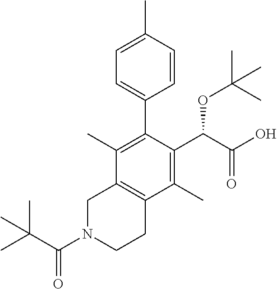 [SMILES: Cc1ccc(-c2c(C)c3c(c(C)c2[C@H](OC(C)(C)C)C(=O)O)CCN(C(=O)C(C)(C)C)C3)cc1]

The title compound was made in a manner similar to that described in example 4 step 2 and isolated as a white solid after reverse phase HPLC. ¹H NMR (400 MHz, CDCl₃) δ 7.35 (m, 1H), 7.24 (m, 2H), 7.06 (m, 1H), 5.17 (s, 1H), 4.91-4.41 (m, 2H), 4.08-3.68 (m, 2H), 2.82 (m, 2H), 2.42 (s, 3H), 2.23 (s, 3H), 1.89 (s, 3H), 1.35 (s, 9H), 0.97 (s, 9H). LCMS (ES+)(m/z): 466.65 (M+1); 488.61 (M+23).

### Example 25: (S)-2-(tert-Butoxy)-2-(2-(3,3-dimethylbutanoyl)-5,8-dimethyl-7-(p-tolyl)-1,2,3,4-tetrahydroisoquinolin-6-yl)acetic Acid

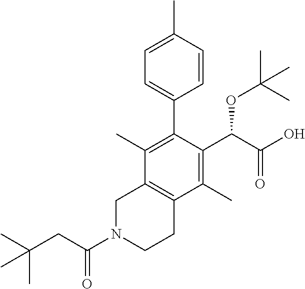 [SMILES: Cc1ccc(-c2c(C)c3c(c(C)c2[C@H](OC(C)(C)C)C(=O)O)CCN(C(=O)CC(C)(C)C)C3)cc1]

The title compound was made in a manner similar to that described in example 4 from 3,3-dimethylbutanoyl chloride and isolated as a white solid after reverse phase HPLC. ¹H NMR (400 MHz, CDCl₃) δ (mixture of rotamers) 7.35 (m, 1H), 7.23 (m, 2H), 7.07 (m, 1H), 5.18 (m, 1H), 4.98-4.38 (m, 2H), 4.03-3.60 (m, 2H), 2.80 (m, 2H), 2.48-2.29 (m, 5H), 2.23 (m, 3H), 1.90 (m, 3H), 1.17-0.90 (m, 18H). LCMS (ES+)(m/z): 480.58 (M+1); 960.12 (2M+1).

### Example 26: (S)-2-(tert-Butoxy)-2-(2-(3-fluoro-2-methoxybenzoyl)-5,8-dimethyl-7-(p-tolyl)-1,2,3,4-tetrahydroisoquinolin-6-yl)acetic Acid

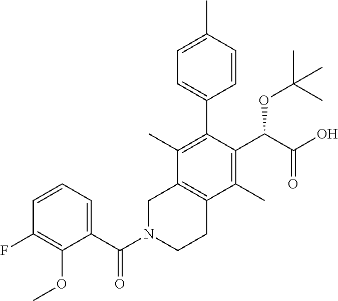 [SMILES: COc1c(F)cccc1C(=O)N1CCc2c(C)c([C@H](OC(C)(C)C)C(=O)O)c(-c3ccc(C)cc3)c(C)c2C1]

The title compound was made in a manner similar to that described in example 5 from 3-fluoro-2-methoxybenzoic acid and isolated as a white solid after reverse phase HPLC. ¹H NMR (400 MHz, CDCl₃) δ (mixture of rotamers) 7.42-6.95 (m, 7H), 5.18 (m, 1H), 5.05-4.61 (m, 1H), 4.44-3.80 (m, 4H), 3.53 (m, 2H), 2.96-2.62 (m, 2H), 2.41 (m, 3H), 2.24 (m, 3H), 1.96 (m, 3H), 0.99 (m, 9H). LCMS (ES+)(m/z): 534.59 (M+1); 1068.03 (2M+1).

### Example 27: (S)-2-(tert-Butoxy)-2-(2-(tert-butoxycarbonyl)-5,8-dimethyl-7-(p-tolyl)-1,2,3,4-tetrahydroisoquinolin-6-yl)acetic Acid

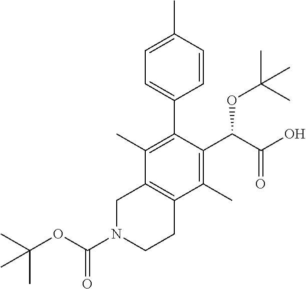 [SMILES: Cc1ccc(-c2c(C)c3c(c(C)c2[C@H](OC(C)(C)C)C(=O)O)CCN(C(=O)OC(C)(C)C)C3)cc1]

The title compound was made in a manner similar to that described in example 17 from di-tert-butyl dicarbonate and isolated as a white solid after reverse phase HPLC as a TFA salt.

¹H NMR (400 MHz, CDCl₃) δ 9.77 (m, 1H), 7.35 (m, 1H), 7.23 (m, 2H), 7.06 (m, 1H), 5.18 (m, 1H), 4.53 (m, 2H), 3.67 (m, 2H), 2.76 (m, 2H), 2.41 (s, 3H), 2.23 (s, 3H), 1.87 (s, 3H), 1.50 (s, 9H), 0.98 (s, 9H). LCMS (ES+)(m/z): 504.41 (M+23).

### Example 28: (S)-2-(tert-Butoxy)-2-(5,8-dimethyl-2-(tetrahydro-2H-pyran-4-yl)-7-(p-tolyl)-1,2,3,4-tetrahydroisoquinolin-6-yl)acetic Acid

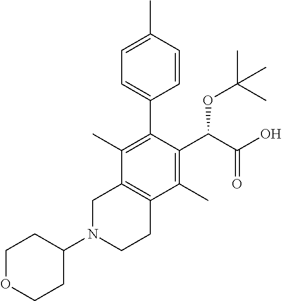 [SMILES: Cc1ccc(-c2c(C)c3c(c(C)c2[C@H](OC(C)(C)C)C(=O)O)CCN(C2CCOCC2)C3)cc1]

The title compound was made in a manner similar to that described in example 1 from dihydro-2H-pyran-4(3H)-one and isolated as a white solid after reverse phase HPLC as a TFA salt. ¹H NMR (400 MHz, CDCl₃) δ 12.79 (m, 1H), 7.39-7.21 (m, 3H), 7.09 (m, 1H), 5.17 (s, 1H), 4.52 (m, 1H), 4.24-3.94 (m, 1H), 3.88-3.57 (m, 2H), 3.49 (m, 2H), 3.22 (m, 2H), 2.99 (m, 1H), 2.42 (s, 3H), 2.24 (s, 3H), 2.05 (m, 4H), 1.87 (s, 3H), 0.96 (s, 9H). LCMS (ES+)(m/z): 466.54 (M+1); 932.00 (2M+1).

### Example 29: (S)-2-(tert-Butoxy)-2-(2-cyclohexyl-5,8-dimethyl-7-(p-tolyl)-1,2,3,4-tetrahydroisoquinolin-6-yl)acetic Acid

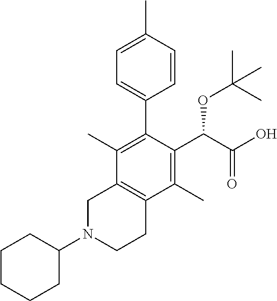 [SMILES: Cc1ccc(-c2c(C)c3c(c(C)c2[C@H](OC(C)(C)C)C(=O)O)CCN(C2CCCCC2)C3)cc1]

The title compound was made in a manner similar to that described in example 1 from cyclohexanone and isolated as a white solid after reverse phase HPLC. ¹H NMR (400 MHz, CDCl₃) δ 7.32 (m, 1H), 7.26 (m, 2H), 7.10 (m, 1H), 5.16 (m, 1H), 4.48 (m, 1H), 4.09-2.88 (m, 5H), 2.43 (s, 3H), 2.23 (m, 5H), 1.98 (m, 2H), 1.87 (s, 3H), 1.81-1.18 (m, 7H), 0.95 (s, 9H).

LCMS (ES+)(m/z): 464.52 (M+1).

### Example 30: (S)-2-(tert-Butoxy)-2-(2-(3-fluorobenzyl)-5,8-dimethyl-7-(p-tolyl)-1,2,3,4-tetrahydroisoquinolin-6-yl)acetic Acid

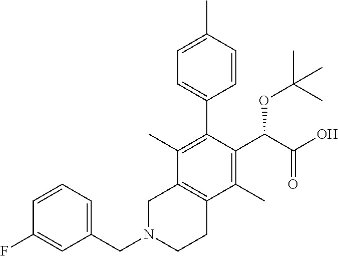 [SMILES: Cc1ccc(-c2c(C)c3c(c(C)c2[C@H](OC(C)(C)C)C(=O)O)CCN(Cc2cccc(F)c2)C3)cc1]

The title compound was made in a manner similar to that described in example 1 from 3-fluorobenzaldehyde and isolated as a white solid after reverse phase HPLC. ¹H NMR (400 MHz, CDCl₃) δ 7.47 (m, 1H), 7.36-7.15 (m, 6H), 7.06 (m, 1H), 5.17 (m, 1H), 4.63-2.92 (m, 6H), 2.42 (s, 3H), 2.24 (s, 3H), 1.79 (s, 3H), 0.96 (s, 9H). LCMS (ES+)(m/z): 490.41 (M+1); 512.41 (M+23).

### Example 31: (S)-2-(tert-Butoxy)-2-(2-(cyclohexylmethyl)-5,8-dimethyl-7-(p-tolyl)-1,2,3,4-tetrahydroisoquinolin-6-yl)acetic Acid

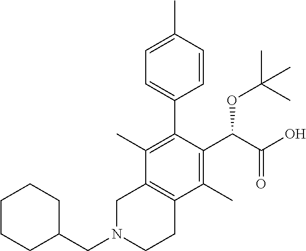 [SMILES: Cc1ccc(-c2c(C)c3c(c(C)c2[C@H](OC(C)(C)C)C(=O)O)CCN(CC2CCCCC2)C3)cc1]

The title compound was made in a manner similar to that described in example 1 from cyclohexanecarbaldehyde and isolated as a white solid after reverse phase HPLC as a TFA salt. ¹H NMR (400 MHz, CDCl₃) δ 8.12 (m, 1H), 7.35 (m, 1H), 7.21 (m, 2H), 7.03 (m, 1H), 5.11 (s, 1H), 3.95-3.58 (m, 2H), 3.05 (m, 2H), 2.87 (m, 2H), 2.75-2.52 (m, 2H), 2.41 (s, 3H), 2.25 (s, 3H), 1.98-1.63 (m, 10H), 1.39-0.80 (m, 13H). LCMS (ES+)(m/z): 478.48 (M+1).

### Example 32: (S)-2-(tert-Butoxy)-2-(2-cyclopentyl-5,8-dimethyl-7-(p-tolyl)-1,2,3,4-tetrahydroisoquinolin-6-yl)acetic Acid

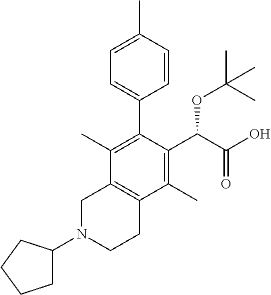 [SMILES: Cc1ccc(-c2c(C)c3c(c(C)c2[C@H](OC(C)(C)C)C(=O)O)CCN(C2CCCC2)C3)cc1]

The title compound was made in a manner similar to that described in example 1 from cyclopentanone and isolated as a white solid after reverse phase HPLC as a TFA salt.

¹H NMR (400 MHz, CDCl₃) δ 12.29 (m, 1H), 7.36-7.23 (m, 3H), 7.09 (m, 1H), 5.17 (s, 1H), 4.63 (m, 1H), 4.03-3.76 (m, 2H), 3.57 (m, 1H), 3.23 (m, 1H), 2.96 (m, 1H), 2.42 (s, 3H), 2.23 (s, 3H), 2.11 (m, 4H), 1.99-1.79 (m, 5H), 1.68 (m, 2H), 0.96 (s, 9H). LCMS (ES+)(m/z): 450.56 (M+1).

### Example 33: (S)-2-(tert-Butoxy)-2-(5,8-dimethyl-2-neopentyl-7-(p-tolyl)-1,2,3,4-tetrahydroisoquinolin-6-yl)acetic Acid

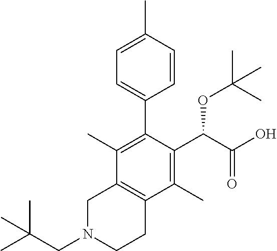 [SMILES: Cc1ccc(-c2c(C)c3c(c(C)c2[C@H](OC(C)(C)C)C(=O)O)CCN(CC(C)(C)C)C3)cc1]

The title compound was made in a manner similar to that described in example 1 from pivalaldehyde and isolated as a white solid after reverse phase HPLC. ¹H NMR (400 MHz, CDCl₃) δ 7.36 (m, 1H), 7.22 (m, 2H), 7.07 (m, 1H), 5.17 (s, 1H), 3.60 (m, 2H), 2.95-2.06 (m, 12H), 1.80 (m, 3H), 1.07-0.82 (m, 18H). LCMS (ES+)(m/z): 452.47 (M+1); 474.48 (2M+1).

### Example 34: (S)-2-(tert-Butoxy)-2-(5,8-dimethyl-2-phenethyl-7-(p-tolyl)-1,2,3,4-tetrahydroisoquinolin-6-yl)acetic Acid

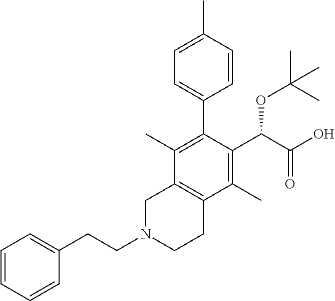 [SMILES: Cc1ccc(-c2c(C)c3c(c(C)c2[C@H](OC(C)(C)C)C(=O)O)CCN(CCc2ccccc2)C3)cc1]

The title compound was made in a manner similar to that described in example 1 from 2-phenylacetaldehyde and isolated as a white solid after reverse phase HPLC as a TFA salt.

¹H NMR (400 MHz, CDCl₃) δ 8.21 (s, 1H), 7.41-7.18 (m, 7H), 7.05 (m, 1H), 5.16 (s, 1H), 4.14-3.89 (m, 2H), 337-2.81 (m, 8H), 2.42 (s, 3H), 2.24 (s, 3H), 1.80 (s, 3H), 0.97 (s, 9H). LCMS (ES+)(m/z): 486.45 (M+1); 508.47 (M+23).

### Example 35: (S)-2-(tert-Butoxy)-2-(2-(cyclohexylcarbamoyl)-5,8-dimethyl-7-(p-tolyl)-1,2,3,4-tetrahydroisoquinolin-6-yl)acetic Acid

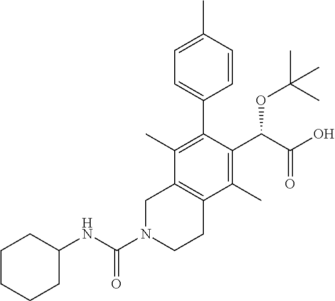 [SMILES: Cc1ccc(-c2c(C)c3c(c(C)c2[C@H](OC(C)(C)C)C(=O)O)CCN(C(=O)NC2CCCCC2)C3)cc1]

The title compound was made in a manner similar to that described in example 10 from isocyanatocyclohexane and isolated as a white solid after reverse phase HPLC as a TFA salt.

¹H NMR (400 MHz, CDCl₃) δ 9.67 (m, 1H), 7.36 (m, 1H), 7.24 (m, 2H), 7.06 (m, 1H), 5.17 (m, 1H), 4.61-4.23 (m, 2H), 3.78-3.53 (m, 3H), 3.99-3.59 (m, 1H), 3.31 (m, 2H), 2.81 (m, 2H), 2.42 (s, 3H), 2.24 (s, 3H), 2.09-1.94 (m, 4H), 1.90 (s, 3H), 1.79-1.58 (m, 3H), 1.47-1.22 (m, 3H), 0.98 (s, 9H). LCMS (ES+)(m/z): 507.66 (M+1); 1014.27 (2M+1).

### Example 36: (S)-2-(tert-Butoxy)-2-(5,8-dimethyl-2-phenyl-7-(p-tolyl)-1,2,3,4-tetrahydroisoquinolin-6-yl)acetic Acid

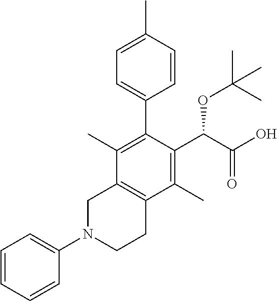 [SMILES: Cc1ccc(-c2c(C)c3c(c(C)c2[C@H](OC(C)(C)C)C(=O)O)CCN(c2ccccc2)C3)cc1]

The title compound was made in a manner similar to that described in example 8 from iodobenzene and isolated as a white solid after reverse phase HPLC. ¹H NMR (400 MHz, CDCl₃) δ 7.50-7.33 (m, 5H), 7.27 (m, 3H), 7.10 (s, 1H), 5.20 (m, 1H), 4.78-4.24 (m, 2H), 3.92-3.55 (m, 2H), 3.21-2.86 (m, 2H), 2.42 (s, 3H), 2.26 (s, 3H), 1.89 (s, 3H), 0.98 (s, 9H). LCMS (ES+)(m/z): 458.42 (M+1); 480.37 (M+23).

### Example 37: (S)-2-(tert-butoxy)-2-(2-(2-cyclohexylacetyl)-5,8-dimethyl-6-(p-tolyl)-1,2,3,4-tetrahydroisoquinolin-7-yl)acetic Acid

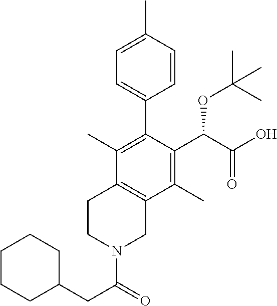 [SMILES: Cc1ccc(-c2c(C)c3c(c(C)c2[C@H](OC(C)(C)C)C(=O)O)CN(C(=O)CC2CCCCC2)CC3)cc1]

The title compound was made in a manner similar to that described in example 4 from 2-cyclohexylacetyl chloride and isolated as a white solid after reverse phase HPLC.

¹H NMR (CDCl₃) δ: 7.35 (m, 1H), 7.24 (m, 2H), 7.07 (m, 1H), 5.17 (m, 1H), 4.97-4.41 (m, 2H), 3.99-3.57 (m, 2H), 2.77 (m, 2H), 2.47-2.15 (m, 8H), 1.97-1.55 (m, 2H), 1.46-0.78 (m, 14H). LCMS ES+ (m/z): 506.60 (M+1), 1012.15 (2M+1).

### Example 38: (S)-2-(tert-butoxy)-2-(5,8-dimethyl-2-(piperidin-1-ylsulfonyl)-7-(p-tolyl)-1,2,3,4-tetrahydroisoquinolin-6-yl)acetic Acid

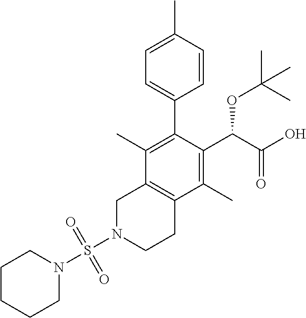 [SMILES: Cc1ccc(-c2c(C)c3c(c(C)c2[C@H](OC(C)(C)C)C(=O)O)CCN(S(=O)(=O)N2CCCCC2)C3)cc1]

The title compound was made in a manner similar to that described in example 4 from piperidine-1-sulfonyl chloride and isolated as a white solid after reverse phase HPLC.

¹H NMR (CDCl₃) ∂: 7.35 (m, 1H), 7.24 (m, 2H), 7.07 (m, 1H), 5.18 (s, 1H), 4.29 (m, 2H), 3.52 (m, 2H), 3.25 (m, 4H), 2.85 (m, 2H), 2.42 (m, 3H), 2.23 (m, 3H), 1.85 (m, 3H), 1.68-1.52 (m, 6H), 0.98 (m, 9H). LCMS ES+ (m/z): 529.59 (M+1), 1058.08 (2M+1).

### Example 39: (S)-2-(tert-butoxy)-2-(2-(2-cyclohexylethyl)-5,8-dimethyl-7-(p-tolyl)-1,2,3,4-tetrahydroisoquinolin-6-yl)acetic Acid

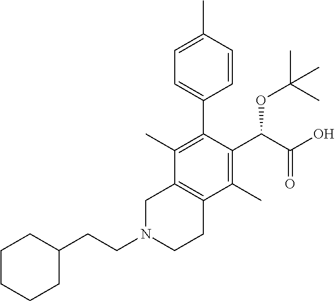 [SMILES: Cc1ccc(-c2c(C)c3c(c(C)c2[C@H](OC(C)(C)C)C(=O)O)CCN(CCC2CCCCC2)C3)cc1]

The title compound was made in a manner similar to that described in example 1 from 2-cyclohexylacetaldehyde and isolated as a white solid after reverse phase HPLC. ¹H NMR (CDCl₃) ∂: 7.36-7.22 (m, 3H), 7.08 (m, 1H), 5.17 (m, 1H), 4.59 (m, 1H), 3.83 (m, 2H), 3.19 (m, 4H), 2.95 (m, 1H), 2.42 (m, 3H), 2.24 (m, 3H), 1.85 (m, 3H), 1.72 (m, 7H), 1.43-1.10 (m, 6H), 0.96 (m, 9H). LCMS ES+ (m/z): 492.63 (M+1), 984.19 (2M+1).

### Example 40: (S)-2-(tert-butoxy)-2-(2-(4,4-dimethylcyclohexyl)-5,8-dimethyl-7-(p-tolyl)-1,2,3,4-tetrahydroisoquinolin-6-yl)acetic Acid

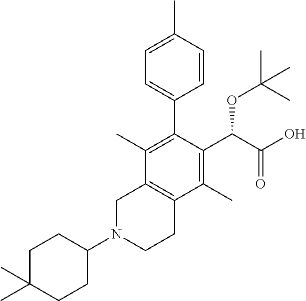 [SMILES: Cc1ccc(-c2c(C)c3c(c(C)c2[C@H](OC(C)(C)C)C(=O)O)CCN(C2CCC(C)(C)CC2)C3)cc1]

The title compound was made in a manner similar to that described in example 1 from 4,4-dimethyl cyclohexanone and isolated as a white solid after reverse phase HPLC.

¹H NMR (CDCl₃) ∂: 7.36-7.22 (m, 3H), 7.09 (m, 1H), 5.16 (m, 1H), 4.49 (m, 1H), 4.01 (m, 1H), 3.76 (m, 1H), 3.24 (m, 3H), 2.94 (m, 1H), 2.41 (m, 3H), 2.23 (m, 3H), 2.02 (m, 2H), 1.86 (m, 5H), 1.59 (m, 4H), 0.96 (m, 15H). LCMS ES+ (m/z): 492.64 (M+1).

### Example 41: (S)-2-(tert-butoxy)-2-(5,8-dimethyl-2-(phenylsulfonyl)-7-(p-tolyl)-1,2,3,4-tetrahydroisoquinolin-6-yl)acetic Acid

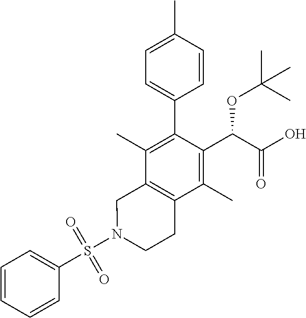 [SMILES: Cc1ccc(-c2c(C)c3c(c(C)c2[C@H](OC(C)(C)C)C(=O)O)CCN(S(=O)(=O)c2ccccc2)C3)cc1]

The title compound was made in a manner similar to that described in example 4 from benzenesulfonyl chloride and isolated as a white solid after reverse phase HPLC. ¹H NMR (CDCl₃) ∂: 7.87 (m, 2H), 7.66-7.51 (m, 3H), 7.37-7.18 (m, 3H), 7.04 (m, 1H), 5.14 (m, 1H), 4.33 (m, 1H), 3.95 (m, 1H), 3.62 (m, 1H), 3.13 (m, 1H), 2.86 (m, 2H), 2.41 (m, 3H), 2.18 (m, 3H), 1.82 (m, 3H), 0.95 (m, 9H). LCMS ES+ (m/z): 522.38 (M+1), 1065.90 (2M+23).

### Example 42: (S)-2-(tert-butoxy)-2-(5,8-dimethyl-2-picolinoyl-7-(p-tolyl)-1,2,3,4-tetrahydroisoquinolin-6-yl)acetic Acid

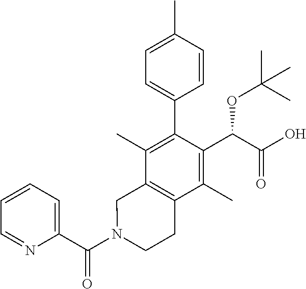 [SMILES: Cc1ccc(-c2c(C)c3c(c(C)c2[C@H](OC(C)(C)C)C(=O)O)CCN(C(=O)c2ccccn2)C3)cc1]

The title compound was made in a manner similar to that described in example 5 from picolinic acid and isolated as a white solid after reverse phase HPLC. ¹H NMR (CDCl₃) ∂: 8.69 (m, 1H), 7.89 (m, 1H), 7.73 (m, 1H), 7.52-6.95 (m, 5H), 5.26-4.50 (m, 3H), 4.14-3.54 (m, 2H), 3.02-2.70 (m, 2H), 2.41 (m, 3H), 2.24 (m, 3H), 2.01-1.62 (m, 3H), 0.98 (m, 9H). LCMS ES+ (m/z): 487.44 (M+1), 973.98 (2M+1).

### Example 43: (S)-2-(tert-butoxy)-2-(5,8-dimethyl-2-nicotinoyl-7-(p-tolyl)-1,2,3,4-tetrahydroisoquinolin-6-yl)acetic Acid

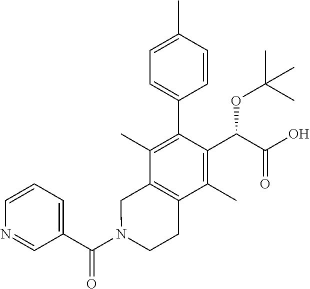 [SMILES: Cc1ccc(-c2c(C)c3c(c(C)c2[C@H](OC(C)(C)C)C(=O)O)CCN(C(=O)c2cccnc2)C3)cc1]

The title compound was made in a manner similar to that described in example 5 from nicotinic acid and isolated as a white solid after reverse phase HPLC. ¹H NMR (CDCl₃) ∂: 8.86 (m, 1H), 8.17 (m, 1H), 7.71 (m, 1H), 7.46-7.17 (m, 4H), 7.08 (m, 1H), 5.19 (m, 1H), 5.07-4.44 (m, 2H), 4.18-3.54 (m, 2H), 2.88 (m, 2H), 2.42 (m, 3H), 2.25 (m, 3H), 2.03-1.59 (m, 3H), 0.99 (m, 9H). LCMS ES+ (m/z): 487.53 (M+1).

### Example 44: (S)-2-(tert-butoxy)-2-(2-isonicotinoyl-5,8-dimethyl-7-(p-tolyl)-1,2,3,4-tetrahydroisoquinolin-6-yl)acetic Acid

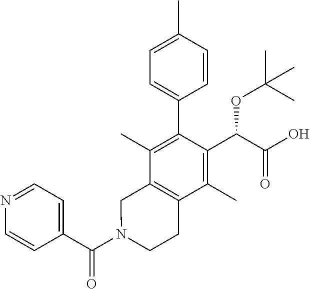 [SMILES: Cc1ccc(-c2c(C)c3c(c(C)c2[C@H](OC(C)(C)C)C(=O)O)CCN(C(=O)c2ccncc2)C3)cc1]

The title compound was made in a manner similar to that described in example 5 from isonicotinic acid and isolated as a white solid after reverse phase HPLC. ¹H NMR (CDCl₃) ∂: 8.87 (m, 2H), 7.67 (m, 2H), 7.38-7.25 (m, 3H), 7.04 (m, 1H), 5.18 (m, 1H), 4.99-4.65 (m, 2H), 4.11-3.50 (m, 2H), 2.97-2.76 (m, 2H), 2.41 (m, 3H), 2.24 (m, 3H), 1.98-1.59 (m, 3H), 0.98 (m, 9H). LCMS ES+ (m/z): 487.54 (M+1).

### Example 45: (S)-2-(tert-butoxy)-2-(2-(cyclohexylsulfonyl)-5,8-dimethyl-7-(p-tolyl)-1,2,3,4-tetrahydroisoquinolin-6-yl)acetic Acid

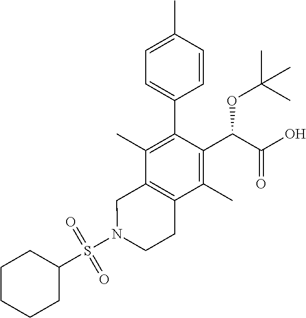 [SMILES: Cc1ccc(-c2c(C)c3c(c(C)c2[C@H](OC(C)(C)C)C(=O)O)CCN(S(=O)(=O)C2CCCCC2)C3)cc1]

The title compound was made in a manner similar to that described in example 4 from cyclohexanesulfonyl chloride and isolated as a white solid after reverse phase HPLC.

¹H NMR (CDCl₃) ∂: 7.35 (m, 1H), 7.24 (m, 2H), 7.07 (m, 1H), 5.18 (m, 1H), 4.57-4.21 (m, 2H), 3.81-3.40 (m, 3H), 3.08-2.74 (m, 3H), 2.42 (m, 3H), 2.27-2.07 (m, 5H), 1.96-1.79 (m, 5H), 1.66-1.44 (m, 5H), 0.98 (m, 9H). LCMS ES+ (m/z): 528.26 (M+1), 550.11 (M+23), 1077.88 (2M+23).

### Example 46: (S)-2-(tert-butoxy)-2-(5,8-dimethyl-2-(2-oxo-2-(piperidin-1-yl)acetyl)-7-(p-tolyl)-1,2,3,4-tetrahydroisoquinolin-6-yl)acetic Acid

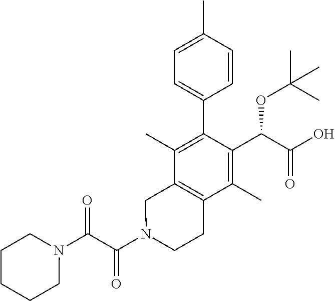 [SMILES: Cc1ccc(-c2c(C)c3c(c(C)c2[C@H](OC(C)(C)C)C(=O)O)CCN(C(=O)C(=O)N2CCCCC2)C3)cc1]

The title compound was made in a manner similar to that described in example 5 from 2-oxo-2-(piperidin-1-yl)acetic acid and isolated as a white solid after reverse phase HPLC. ¹H NMR (400 MHz, METHANOL-d₄) (mixture of rotamers) δ ppm 7.37-7.20 (m, 3H), 7.13-7.00 (m, 1H), 5.11-5.02 (m, 1H), 4.76-4.43 (m, 2H), 4.00-3.50 (m, 4H), 3.47-3.21 (m, 2H), 2.88 (t, J=5.4 Hz, 2H), 2.42 (s, 3H), 2.29 (br. s., 3H), 1.96-1.79 (m, 3H), 1.78-1.50 (m, 5H), 1.44-1.26 (m, 1H), 0.92 (s, 9H); LCMS (m/z) ES⁻=519.1 (M−1).

### Example 47: (S)-2-(tert-butoxy)-2-(2-(cyclohexanecarbonyl)-5,8-dimethyl-7-(p-tolyl)-1,2,3,4-tetrahydroisoquinolin-6-yl)acetic Acid

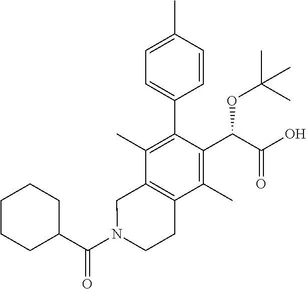 [SMILES: Cc1ccc(-c2c(C)c3c(c(C)c2[C@H](OC(C)(C)C)C(=O)O)CCN(C(=O)C2CCCCC2)C3)cc1]

The title compound was made in a manner similar to that described in example 5 from cyclohexanecarboxylic acid and isolated as a white solid after reverse phase HPLC.

¹H NMR (400 MHz, METHANOL-d₄) (mixture of rotamers) δ ppm 7.39-7.18 (m, 3H), 7.15-6.99 (m, 1H), 5.06 (s, 1H), 4.75-4.51 (m, 2H), 3.95-3.75 (m, 2H), 2.94-2.69 (m, 3H), 2.42 (s, 3H), 2.36-2.21 (m, 3H), 1.96-1.65 (m, 8H), 1.60-1.19 (m, 5H), 0.92 (s, 9H); LCMS (m/z) ES⁺=492.1 (M+1), ES⁻=490.1 (M−1).

### Example 48: (S)-2-(2-benzoyl-5,8-dimethyl-7-(p-tolyl)-1,2,3,4-tetrahydroisoquinolin-6-yl)-2-(tert-butoxy)acetic Acid

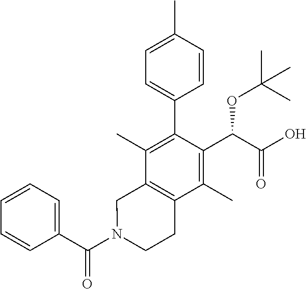 [SMILES: Cc1ccc(-c2c(C)c3c(c(C)c2[C@H](OC(C)(C)C)C(=O)O)CCN(C(=O)c2ccccc2)C3)cc1]

The title compound was made in a manner similar to that described in example 5 from benzoic acid and isolated as a white solid after reverse phase HPLC. ¹H NMR (400 MHz, METHANOL-d₄) (mixture of rotamers) δ ppm 7.62-7.39 (m, 5H), 7.36-7.17 (m, 3H), 7.15-6.93 (m, 1H), 5.14-4.99 (m, 1H), 4.83-4.46 (m, 2H), 4.10-3.62 (m, 2H), 2.99-2.73 (m, 2H), 2.52-2.36 (m, 3H), 2.30 (br. s., 3H), 2.02-1.50 (m, 3H), 0.93 (s, 9H); LCMS (m/z) ES⁺=486.1 (M+1), ES⁻=484.1 (M−1).

### Example 49: (2S)-2-(2-(bicyclo[2.2.1]heptan-2-yl)-5,8-dimethyl-7-(p-tolyl)-1,2,3,4-tetrahydroisoquinolin-6-yl)-2-(tert-butoxy)acetic Acid

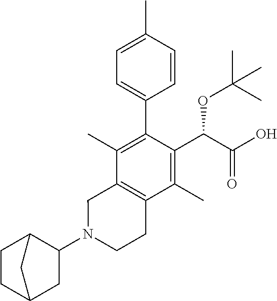 [SMILES: Cc1ccc(-c2c(C)c3c(c(C)c2[C@H](OC(C)(C)C)C(=O)O)CCN(C2CC4CCC2C4)C3)cc1]

The title compound was made in a manner similar to that described in example 1 step 11 from norcamphor, and hydrolyzed in a manner similar to that described in example 4 step 2. The product was isolated as a white solid after reverse phase HPLC as a TFA salt. ¹H NMR (400 MHz, METHANOL-d₄) δ ppm 7.36-7.19 (m, 3H), 7.14-6.98 (m, 1H), 5.07 (s, 1H), 4.56-4.18 (m, 2H), 3.93-3.80 (m, 1H), 3.77-3.57 (m, 1H), 3.48-3.26 (m, 1H), 3.18-3.04 (m, 2H), 2.88-2.75 (m, 1H), 2.49-2.39 (m, 4H), 2.38-2.15 (m, 4H), 1.95-1.83 (m, 3H), 1.75 (br. s., 2H), 1.68-1.41 (m, 4H), 1.41-1.23 (m, 1H), 1.04-0.80 (m, 9H); LCMS (m/z) ES⁺=476.2 (M+1), LCMS (m/z) ES⁻=474.1 (M−1).

### Example 50: (S)-2-(tert-butoxy)-2-(2-cycloheptyl-5,8-dimethyl-7-(p-tolyl)-1,2,3,4-tetrahydroisoquinolin-6-yl)acetic Acid

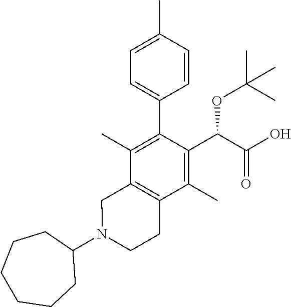 [SMILES: Cc1ccc(-c2c(C)c3c(c(C)c2[C@H](OC(C)(C)C)C(=O)O)CCN(C2CCCCCC2)C3)cc1]

The title compound was made in a manner similar to that described in example 49 from cycloheptanone, except the reductive amination was heated to 80° C. The final product was isolated as a white solid after reverse phase HPLC as a TFA salt. ¹H NMR (400 MHz, METHANOL-d₄) δ ppm 7.34-7.22 (m, 3H), 7.11-6.99 (m, 1H), 5.10-5.03 (m, 1H), 4.49-4.27 (m, 2H), 3.83-3.70 (m, 1H), 3.69-3.57 (m, 1H), 3.44-3.26 (m, 1H), 3.18-3.04 (m, 2H), 2.43 (s, 3H), 2.38-2.28 (m, 3H), 2.27-2.09 (m, 2H), 2.03-1.81 (m, 7H), 1.77-1.54 (m, 6H), 1.00-0.84 (m, 9H); LCMS (m/z) ES⁺=478.1 (M+1), ES⁻=476.0 (M−1).

### Example 51: (S)-2-(tert-butoxy)-2-(5,8-dimethyl-2-(spiro[2.5]octan-6-yl)-7-(p-tolyl)-1,2,3,4-tetrahydroisoquinolin-6-yl)acetic Acid

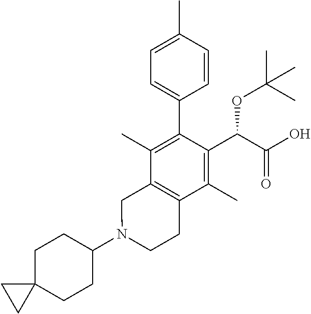 [SMILES: Cc1ccc(-c2c(C)c3c(c(C)c2[C@H](OC(C)(C)C)C(=O)O)CCN(C2CCC4(CC2)CC4)C3)cc1]

The title compound was made in a manner similar to that described in example 49 from spiro[2.5]octan-6-one and isolated as a white solid after reverse phase HPLC as a formic acid salt. ¹H NMR (400 MHz, METHANOL-d₄) δ ppm 8.35 (s, 1H), 7.40-7.19 (m, 3H), 7.06 (d, J=7.7 Hz, 1H), 5.03 (s, 1H), 4.40 (s, 2H), 3.69-3.55 (m, 2H), 3.49 (t, J=12.0 Hz, 1H), 3.11 (t, J=5.9 Hz, 2H), 2.42 (s, 3H), 2.34 (s, 3H), 2.19 (d, J=10.4 Hz, 2H), 2.07-1.75 (m, 7H), 1.14 (d, J=12.8 Hz, 2H), 0.91 (s, 9H), 0.49-0.29 (m, 4H); LCMS (m/z) ES⁺=490.6 (M+1).

### Example 52: (2S)-2-(tert-butoxy)-2-(2-(3,3-dimethylcyclohexyl)-5,8-dimethyl-7-(p-tolyl)-1,2,3,4-tetrahydroisoquinolin-6-yl)acetic Acid

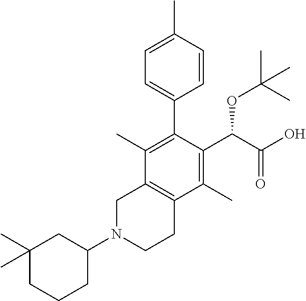 [SMILES: Cc1ccc(-c2c(C)c3c(c(C)c2[C@H](OC(C)(C)C)C(=O)O)CCN(C2CCCC(C)(C)C2)C3)cc1]

The title compound was made in a manner similar to that described in example 50 from 3,3-dimethylcyclohexanone and isolated as a white solid after reverse phase HPLC as a TFA salt (1:1 mixture of diastereomers). ¹H NMR (400 MHz, METHANOL-d₄) δ ppm 7.34-7.21 (m, 3H), 7.06 (t, J=8.7 Hz, 1H), 5.10-5.04 (m, 1H), 4.45-4.33 (m, 2H), 3.86 (br. s., 1H), 3.70-3.57 (m, 1H), 3.42-3.01 (m, 3H), 2.43 (s, 3H), 2.38-2.29 (m, 3H), 2.00-1.77 (m, 5H), 1.76-1.21 (m, 6H), 1.09 (s, 3H), 1.05 (s, 3H), 0.99-0.85 (m, 9H); LCMS (m/z) ES⁺=492.2 (M+1), ES⁻=490.1 (M−1).

### Example 53: (S)-2-(tert-butoxy)-2-(5,8-dimethyl-2-(2-(piperidin-1-yl)acetyl)-7-(p-tolyl)-1,2,3,4-tetrahydroisoquinolin-6-yl)acetic Acid

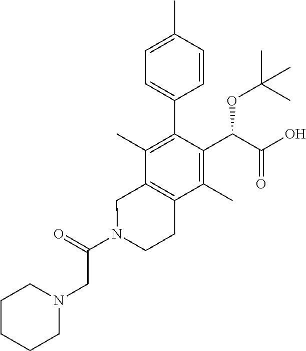 [SMILES: Cc1ccc(-c2c(C)c3c(c(C)c2[C@H](OC(C)(C)C)C(=O)O)CCN(C(=O)CN2CCCCC2)C3)cc1]

The title compound was made in a manner similar to that described in example 5 from 2-(piperidin-1-yl)acetic acid and isolated as a white solid after reverse phase HPLC as a formic acid salt. ¹H NMR (400 MHz, METHANOL-d₄) (mixture of rotamers) δ ppm 8.40 (br. s., 1H), 7.40-7.20 (m, 3H), 7.06 (d, J=7.3 Hz, 1H), 5.03 (s, 1H), 4.75-4.47 (m, 2H), 4.22 (s, 2H), 3.96-3.65 (m, 2H), 3.29-3.11 (m, 4H), 2.98-2.75 (m, 2H), 2.42 (s, 3H), 2.36-2.22 (m, 3H), 1.96-1.75 (m, 7H), 1.74-1.56 (m, 2H), 0.91 (s, 9H); LCMS (m/z) ES⁺=507.2 (M+1), ES⁻=505.1 (M−1).

### Example 54: (S)-2-(tert-butoxy)-2-(5,8-dimethyl-2-(2-oxo-2-(piperidin-1-yl)ethyl)-7-(p-tolyl)-1,2,3,4-tetrahydroisoquinolin-6-yl)acetic Acid

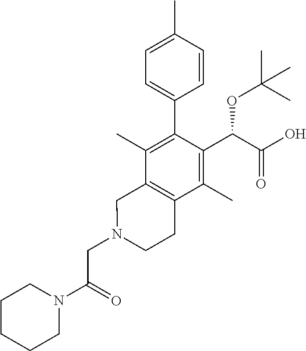 [SMILES: Cc1ccc(-c2c(C)c3c(c(C)c2[C@H](OC(C)(C)C)C(=O)O)CCN(CC(=O)N2CCCCC2)C3)cc1]

The title compound was made in a manner similar to that described in example 4 from 2-chloro-1-(piperidin-1-yl)ethanone and isolated as a white solid after reverse phase HPLC as a formic acid salt. ¹H NMR (400 MHz, METHANOL-d₄) δ ppm 8.37 (br. s., 1H), 7.39-7.20 (m, 3H), 7.06 (d, J=7.7 Hz, 1H), 5.04 (s, 1H), 4.23-4.00 (m, 4H), 3.68-3.55 (m, 2H), 3.53-3.42 (m, 2H), 3.40-3.24 (m, 2H), 3.09-2.96 (m, 2H), 2.42 (s, 3H), 2.32 (s, 3H), 1.84 (s, 3H), 1.79-1.52 (m, 6H), 0.92 (s, 9H); LCMS (m/z) ES⁺=507.1 (M+1), ES⁻=505.1 (M−1).

### Example 55: (S)-2-(2-benzyl-5,8-dimethyl-7-(p-tolyl)-1,2,3,4-tetrahydroisoquinolin-6-yl)-2-(tert-butoxy)acetic Acid

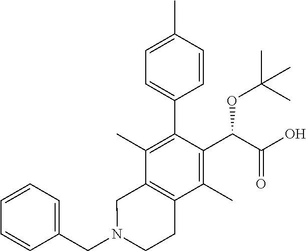 [SMILES: Cc1ccc(-c2c(C)c3c(c(C)c2[C@H](OC(C)(C)C)C(=O)O)CCN(Cc2ccccc2)C3)cc1]

### Step 1: (S)-methyl 2-(2-benzyl-5,8-dimethyl-7-(p-tolyl)-1,2,3,4-tetrahydroisoquinolin-6-yl)-2-(tert-butoxy)acetate

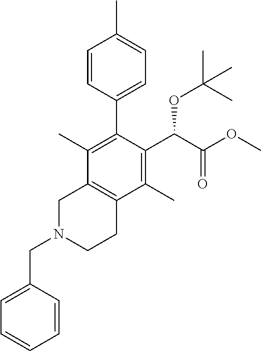 [SMILES: COC(=O)[C@@H](OC(C)(C)C)c1c(C)c2c(c(C)c1-c1ccc(C)cc1)CN(Cc1ccccc1)CC2]

A solution of (S)-methyl 2-(tert-butoxy)-2-(5,8-dimethyl-7-(p-tolyl)-1,2,3,4-tetrahydroisoquinolin-6-yl)acetate (10 mg, 0.025 mmol) in Methanol (0.5 mL) was treated with benzaldehyde (10 μl, 0.099 mmol), acetic acid (10 μl, 0.175 mmol), stirred at rt for 1 hour, and then heated to 85° C. for 18 hours. The reaction was treated with sodium triacetoxyborohydride (8.04 mg, 0.038 mmol) and stirred at rt for 4 hours. The mixture was diluted with aq. sat. NaHCO₃, extracted with EtOAc, washed with Brine, dried over Na₂SO₄, filtered, and concentrated. The resulting light yellow oil was carried on to the next step without further purification. LCMS (m/z) ES⁺=486.6 (M+1)

### Step 2: (S)-2-(2-benzyl-5,8-dimethyl-7-(p-tolyl)-1,2,3,4-tetrahydroisoquinolin-6-yl)-2-(tert-butoxy)acetic Acid

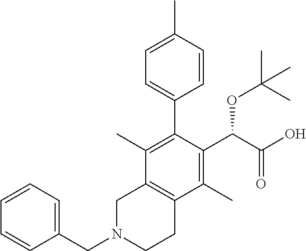 [SMILES: Cc1ccc(-c2c(C)c3c(c(C)c2[C@H](OC(C)(C)C)C(=O)O)CCN(Cc2ccccc2)C3)cc1]

The title compound was made in a manner similar to that described in example 4 step 2 and isolated as a white solid after reverse phase HPLC as a formic acid salt. ¹H NMR (400 MHz, METHANOL-d₄) δ ppm 7.56-7.31 (m, 6H), 7.24 (t, J=7.7 Hz, 2H), 7.02 (d, J=7.7 Hz, 1H), 4.99 (s, 1H), 4.21-4.00 (m, 2H), 3.96-3.78 (m, 2H), 3.23-3.07 (m, 2H), 3.01-2.81 (m, 2H), 2.41 (s, 3H), 2.30 (s, 3H), 1.75 (s, 3H), 0.89 (s, 9H); LCMS (m/z) ES⁺=472.4 (M+1), ES⁻=470.4 (M−1).

### Example 56: (S)-2-(tert-butoxy)-2-(2-(4,4-difluorocyclohexyl)-5,8-dimethyl-7-(p-tolyl)-1,2,3,4-tetrahydroisoquinolin-6-yl)acetic Acid

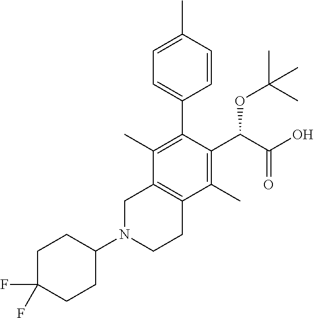 [SMILES: Cc1ccc(-c2c(C)c3c(c(C)c2[C@H](OC(C)(C)C)C(=O)O)CCN(C2CCC(F)(F)CC2)C3)cc1]

The title compound was made in a manner similar to that described in example 49 from 4,4-difluorocyclohexanone and isolated as a white solid after reverse phase HPLC as a TFA salt. ¹H NMR (400 MHz, METHANOL-d₄) δ ppm 7.40-7.17 (m, 3H), 7.06 (d, J=6.8 Hz, 1H), 5.07 (s, 1H), 4.45 (s, 2H), 3.89 (br. s., 1H), 3.73-3.55 (m, 1H), 3.50-3.35 (m, 1H), 3.14 (br. s., 2H), 2.43 (s, 3H), 2.38-2.18 (m, 7H), 2.14-1.83 (m, 7H), 0.92 (s, 9H);

LCMS (m/z) ES⁺=500.5 (M+1), ES⁻=498.4 (M−1).

### Example 57: (S)-2-(tert-butoxy)-2-(2-((3-fluorophenyl)sulfonyl)-5,8-dimethyl-7-(p-tolyl)-1,2,3,4-tetrahydroisoquinolin-6-yl)acetic Acid

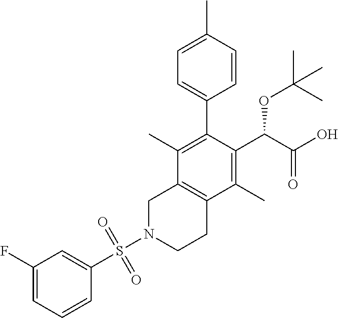 [SMILES: Cc1ccc(-c2c(C)c3c(c(C)c2[C@H](OC(C)(C)C)C(=O)O)CCN(S(=O)(=O)c2cccc(F)c2)C3)cc1]

### Step 1: (S)-methyl 2-(tert-butoxy)-2-(2-(3-fluorophenyl)sulfonyl)-5,8-dimethyl-7-(p-tolyl)-1,2,3,4-tetrahydroisoquinolin-6-yl)acetate

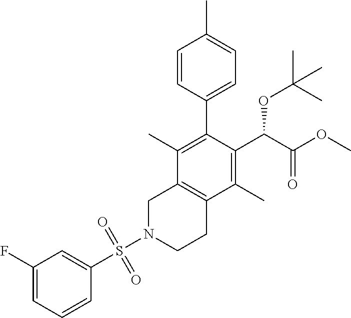 [SMILES: COC(=O)[C@@H](OC(C)(C)C)c1c(C)c2c(c(C)c1-c1ccc(C)cc1)CN(S(=O)(=O)c1cccc(F)c1)CC2]

An ice cold solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in Dichloromethane (DCM) (1 mL) was treated with pyridine (4.50 μl, 0.056 mmol), 3-fluorobenzene-1-sulfonyl chloride (49.2 mg, 0.253 mmol), and stirred at rt for 18 hours. The reaction was diluted with 1N HCl, extracted with DCM, washed with Brine, dried over Na2SO4, filtered, and concentrated. Purification with column chromatography (0-50% EtOAc/Hexane) afforded the title compound as white solid. ¹H NMR (400 MHz, CHLOROFORM-d) δ ppm 7.66 (d, J=7.9 Hz, 1H), 7.61-7.49 (m, 2H), 7.35-7.28 (m, 1H), 7.26-7.13 (m, 3H), 7.02 (d, J=7.7 Hz, 1H), 5.00 (s, 1H), 4.27-4.06 (m, 2H), 3.66 (s, 3H), 3.51-3.41 (m, 1H), 3.40-3.30 (m, 1H), 2.95-2.76 (m, 2H), 2.42 (s, 3H), 2.25 (s, 3H), 1.80 (s, 3H), 0.94 (s, 9H); LCMS (m/z) ES⁺=576.5 (M+23, weak).

### Step 2: (S)-2-(tert-butoxy)-2-(2-((3-fluorophenyl)sulfonyl)-5,8-dimethyl-7-(p-tolyl)-1,2,3,4-tetrahydroisoquinolin-6-yl)acetic Acid

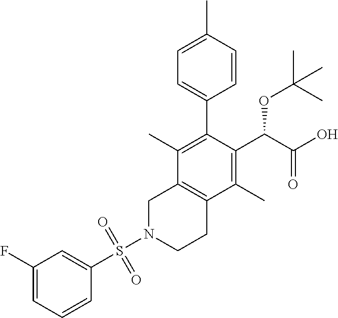 [SMILES: Cc1ccc(-c2c(C)c3c(c(C)c2[C@H](OC(C)(C)C)C(=O)O)CCN(S(=O)(=O)c2cccc(F)c2)C3)cc1]

The title compound was made in a manner similar to that described in example 4 step 2 and isolated as a white solid after reverse phase HPLC. ¹H NMR (400 MHz, METHANOL-d₄) δ ppm 7.76-7.55 (m, 3H), 7.47-7.35 (m, 1H), 7.32-7.20 (m, 3H), 7.05 (d, J=7.9 Hz, 1H), 5.03 (s, 1H), 4.33-4.10 (m, 2H), 3.58-3.45 (m, 1H), 3.45-3.37 (m, 1H), 2.81 (t, J=6.0 Hz, 2H), 2.41 (s, 3H), 2.21 (s, 3H), 1.82 (s, 3H), 0.90 (s, 9H); LCMS (m/z) ES⁻=538.3 (M−1).

### Example 58: (S)-2-(tert-butoxy)-2-(2-((cyclohexylmethyl)sulfonyl)-5,8-dimethyl-7-(p-tolyl)-1,2,3,4-tetrahydroisoquinolin-6-yl)acetic Acid

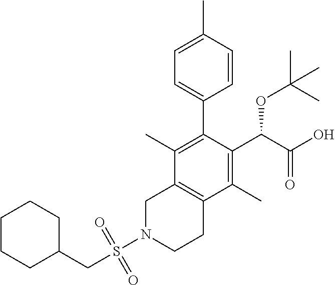 [SMILES: Cc1ccc(-c2c(C)c3c(c(C)c2[C@H](OC(C)(C)C)C(=O)O)CCN(S(=O)(=O)CC2CCCCC2)C3)cc1]

The title compound was made in a manner similar to that described in example 57 from cyclohexylmethanesulfonyl chloride, except it was heated to 45° C. after stirring at rt. The final product was isolated as a white solid after reverse phase HPLC. ¹H NMR (400 MHz, METHANOL-d₄) δ ppm 7.36-7.21 (m, 3H), 7.08 (d, J=7.1 Hz, 1H), 5.06 (s, 1H), 4.46-4.26 (m, 2H), 3.69-3.47 (m, 2H), 2.97 (d, J=5.9 Hz, 2H), 2.87 (t, J=5.9 Hz, 2H), 2.42 (s, 3H), 2.29 (s, 3H), 1.98-1.90 (m, 2H), 1.85 (s, 3H), 1.79-1.59 (m, 3H), 1.38-1.04 (m, 6H), 0.93 (s, 9H); LCMS (m/z) ES⁻=540.4 (M−1).

### Example 59: (2S)-2-(tert-butoxy)-2-(5,8-dimethyl-2-(3-phenylcyclohexyl)-7-(p-tolyl)-1,2,3,4-tetrahydroisoquinolin-6-yl)acetic Acid

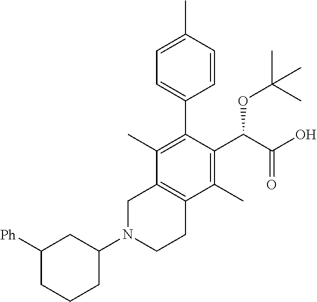 [SMILES: Cc1ccc(-c2c(C)c3c(c(C)c2[C@H](OC(C)(C)C)C(=O)O)CCN(C2CCCC(c4ccccc4)C2)C3)cc1]

The title compound was made in a manner similar to that described in example 49 from 3-phenylcyclohexanone and two diastereomeric mixtures were isolated as white solids after reverse phase HPLC as TFA salts. Diastereomeric mixture 1: ¹H NMR (400 MHz, METHANOL-d₄) δ ppm 7.46-7.15 (m, 8H), 7.14-6.97 (m, 1H), 5.07 (s, 1H), 4.43 (s, 2H), 3.97-3.85 (m, 1H), 3.63-3.51 (m, 1H), 3.50-3.37 (m, 1H), 3.37-3.23 (m, 1H), 3.12 (br. s., 2H), 2.63-2.39 (m, 4H), 2.38-2.24 (m, 4H), 2.21-1.98 (m, 3H), 1.97-1.62 (m, 6H), 1.00-0.84 (m, 9H); LCMS (m/z) ES⁺=540.5 (M+1), ES⁻=538.5 (M−1). Diastereomeric mixture 2: ¹H NMR (400 MHz, METHANOL-d₄) δ ppm 7.40-7.14 (m, 8H), 7.13-6.94 (m, 1H), 5.12-5.01 (m, 1H), 4.55-4.34 (m, 2H), 4.01-3.81 (m, 1H), 3.77-3.59 (m, 1H), 3.46-3.23 (m, 1H), 3.20-3.02 (m, 2H), 2.78 (t, J=11.4 Hz, 1H), 2.50-2.07 (m, 9H), 2.01-1.45 (m, 8H), 1.08-0.76 (m, 9H); LCMS (m/z) ES⁺=540.5 (M+1), ES⁻=538.5 (M−1).

### Example 60: (2S)-2-(2-(bicyclo[3.2.1]octan-2-yl)-5,8-dimethyl-7-(p-tolyl)-1,2,3,4-tetrahydroisoquinolin-6-yl)-2-(tert-butoxy)acetic Acid

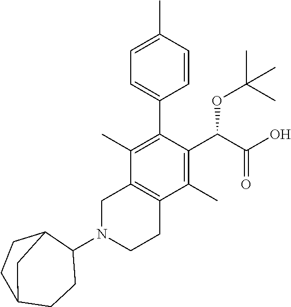 [SMILES: Cc1ccc(-c2c(C)c3c(c(C)c2[C@H](OC(C)(C)C)C(=O)O)CCN(C2CCC4CCC2C4)C3)cc1]

### Step 1: (2S)-methyl 2-(2-(bicyclo[3.2.1]octan-2-yl)-5,8-dimethyl-7-(p-tolyl)-1,2,3,4-tetrahydroisoquinolin-6-yl)-2-(tert-butoxy)acetate

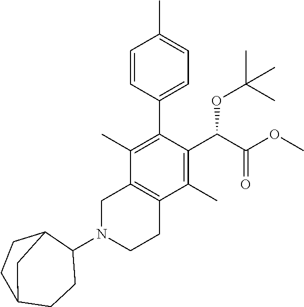 [SMILES: COC(=O)[C@@H](OC(C)(C)C)c1c(C)c2c(c(C)c1-c1ccc(C)cc1)CN(C1CCC3CCC1C3)CC2]

A solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in 1,2-Dichloroethane (DCE) (1 mL) was treated with bicyclo[3.2.1]octan-2-one (9.42 mg, 0.076 mmol) and stirred for 15 min at rt. The reaction was treated with sodium triacetoxyborohydride (16.08 mg, 0.076 mmol), stirred at rt for 18 hours, and then heated to 50 C for 3 hours. The reaction was treated with acetic acid (10 μl, 0.175 mmol), stirred at rt for 2 hours, heated to 50 C for 2 hours, and then cooled to rt over the weekend. The reaction was treated with titanium(IV) isopropoxide (100 μl, 0.341 mmol), sodium cyanoborohydride (4.77 mg, 0.076 mmol), and stirred at rt for 5 hours. The mixture was diluted with aq. sat. NaHCO₃, extracted with DCM, washed with Brine, dried over Na2SO4, filtered, and concentrated to give crude (2S)-methyl 2-(2-(bicyclo[3.2.1]octan-2-yl)-5,8-dimethyl-7-(p-tolyl)-1,2,3,4-tetrahydroisoquinolin-6-yl)-2-(tert-butoxy)acetate (16.1 mg, 0.032 mmol, 63.2% yield) as clear oil. LCMS (m/z) ES⁺=504.6 (M+1).

### Step 2: (2S)-2-(2-(bicyclo[3.2.1]octan-2-yl)-5,8-dimethyl-7-(p-tolyl)-1,2,3,4-tetrahydroisoquinolin-6-yl)-2-(tert-butoxy)acetic Acid

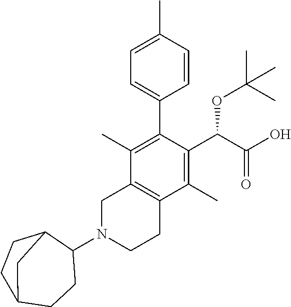 [SMILES: Cc1ccc(-c2c(C)c3c(c(C)c2[C@H](OC(C)(C)C)C(=O)O)CCN(C2CCC4CCC2C4)C3)cc1]

The title compound was made in a manner similar to that described in example 4 step 2. A 1:1 mixture of diastereomers was isolated as a white solid after reverse phase HPLC as TFA salt. ¹H NMR (400 MHz, METHANOL-d₄) δ ppm 7.35-7.21 (m, 3H), 7.13-7.00 (m, 1H), 5.07 (s, 1H), 4.64-4.22 (m, 2H), 4.09-3.94 (m, 1H), 3.55-3.38 (m, 1H), 3.37-3.19 (m, 1H), 3.18-3.02 (m, 2H), 2.92-2.73 (m, 1H), 2.43 (s, 3H), 2.39-2.14 (m, 5H), 2.09-1.94 (m, 1H), 1.90 (br. s., 3H), 1.87-1.43 (m, 8H), 1.01-0.84 (m, 9H); LCMS (m/z) ES⁺=490.5 (M+1), ES⁻=488.5 (M−1).

### Example 61: (S)-2-(tert-butoxy)-2-(2-cyclobutyl-5,8-dimethyl-7-(p-tolyl)-1,2,3,4-tetrahydroisoquinolin-6-yl)acetic Acid

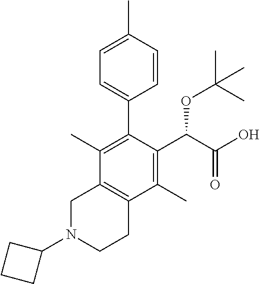 [SMILES: Cc1ccc(-c2c(C)c3c(c(C)c2[C@H](OC(C)(C)C)C(=O)O)CCN(C2CCC2)C3)cc1]

### Step 1: (S)-methyl 2-(tert-butoxy)-2-(2-cyclobutyl-5,8-dimethyl-7-(p-tolyl)-1,2,3,4-tetrahydroisoquinolin-6-yl)acetate

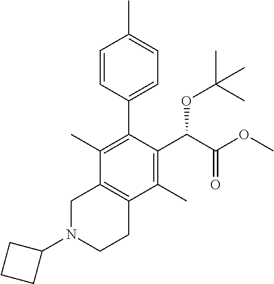 [SMILES: COC(=O)[C@@H](OC(C)(C)C)c1c(C)c2c(c(C)c1-c1ccc(C)cc1)CN(C1CCC1)CC2]

A solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in 1,2-Dichloroethane (DCE) (1 mL) was treated with cyclobutanone (17.72 mg, 0.253 mmol) and stirred for 15 min at rt. The reaction was treated with sodium triacetoxyborohydride (16.08 mg, 0.076 mmol) and stirred at rt for 18 hours. The reaction was heated to 50° C. for 3 hours. The reaction was treated with acetic acid (10 μl, 0.175 mmol), stirred at rt for 2 hours, and then at 50° C. for 1.5 hours. The mixture was treated with additional cyclobutanone (57 mg), sodium triacetoxyborohydride (16.08 mg, 0.076 mmol), stirred at 50° C. for 1 hour, and then cooled to rt over the weekend. The mixture was diluted with aq. sat. NaHCO₃, extracted with DCM, washed with Brine, dried over Na2SO4, filtered, and concentrated to give 26 mg crude product. Purification with column chromatography (0-100% EtOAc/Hexane) afforded mix (S)-methyl 2-(tert-butoxy)-2-(2-cyclobutyl-5,8-dimethyl-7-(p-tolyl)-1,2,3,4-tetrahydroisoquinolin-6-yl)acetate (9.2 mg, 0.020 mmol, 40.5% yield) as clear film. LCMS (m/z) ES⁺=450.6 (M+1).

### Step 2: (S)-2-(tert-butoxy)-2-(2-cyclobutyl-5,8-dimethyl-7-(p-tolyl)-1,2,3,4-tetrahydroisoquinolin-6-yl)acetic Acid

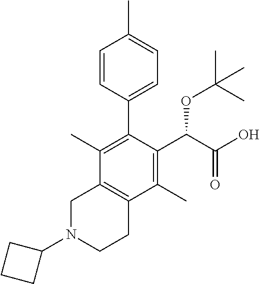 [SMILES: Cc1ccc(-c2c(C)c3c(c(C)c2[C@H](OC(C)(C)C)C(=O)O)CCN(C2CCC2)C3)cc1]

The title compound was made in a manner similar to that described in example 4 step 2 and isolated as a white solid after reverse phase HPLC as TFA salt. ¹H NMR (400 MHz, METHANOL-d₄) δ ppm 7.35-7.21 (m, 3H), 7.13-7.00 (m, 1H), 5.07 (br. s., 1H), 4.41 (d, J=15.4 Hz, 1H), 4.07 (d, J=15.4 Hz, 1H), 3.91 (quin, J=8.1 Hz, 1H), 3.82-3.66 (m, 1H), 3.26-2.98 (m, 3H), 2.57-2.25 (m, 10H), 2.03-1.84 (m, 5H), 0.92 (br. s., 9H); LCMS (m/z) ES⁺=436.4 (M+1), ES⁻=434.4 (M−1).

### Example 62: (S)-2-(tert-butoxy)-2-(5,8-dimethyl-2-(neopentylsulfonyl)-7-(p-tolyl)-1,2,3,4-tetrahydroisoquinolin-6-yl)acetic Acid

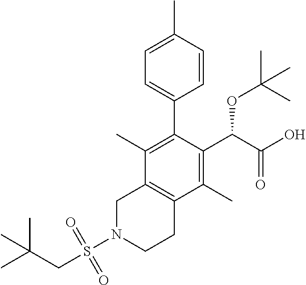 [SMILES: Cc1ccc(-c2c(C)c3c(c(C)c2[C@H](OC(C)(C)C)C(=O)O)CCN(S(=O)(=O)CC(C)(C)C)C3)cc1]

### Step 1: (S)-methyl 2-(tert-butoxy)-2-(5,8-dimethyl-2-(neopentylsulfonyl)-7-(p-tolyl)-1,2,3,4-tetrahydroisoquinolin-6-yl)acetate

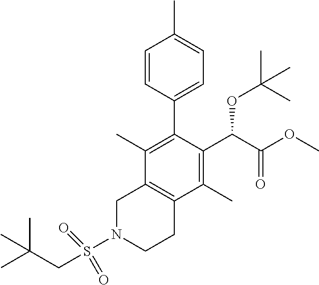 [SMILES: COC(=O)[C@@H](OC(C)(C)C)c1c(C)c2c(c(C)c1-c1ccc(C)cc1)CN(S(=O)(=O)CC(C)(C)C)CC2]

An ice cold solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in Dichloromethane (DCM) (1 mL) was treated with pyridine (4.50 μl, 0.056 mmol), 2,2-dimethylpropane-1-sulfonyl chloride (43.1 mg, 0.253 mmol), and stirred at rt for 18 hours. The reaction was heated to 50° C. for 2 hours, treated with additional pyridine (10 uL), 2,2-dimethylpropane-1-sulfonyl chloride (43.1 mg, 0.253 mmol), and stirred at 50° C. for 1 hour. The reaction was treated with DMAP (3.09 mg, 0.025 mmol), Et3N (0.014 mL, 0.101 mmol), and stirred at 50° C. for 1.5 hours. The mixture was cooled to rt, diluted with 1N HCl, extracted with DCM, washed with Brine, dried over Na2SO4, filtered, and concentrated. Purification with column chromatography (0-70% EtOAc/Hexane) afforded (S)-methyl 2-(tert-butoxy)-2-(5,8-dimethyl-2-(neopentylsulfonyl)-7-(p-tolyl)-1,2,3,4-tetrahydroisoquinolin-6-yl)acetate (6.9 mg, 0.013 mmol, 25.8% yield) as clear film. LCMS (m/z) ES⁺=530.6 (M+1, weak), 552.5 (M+23).

### Step 2: (S)-2-(tert-butoxy)-2-(5,8-dimethyl-2-(neopentylsulfonyl)-7-(p-tolyl)-1,2,3,4-tetrahydroisoquinolin-6-yl)acetic Acid

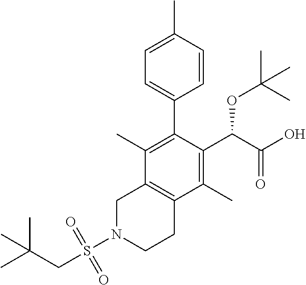 [SMILES: Cc1ccc(-c2c(C)c3c(c(C)c2[C@H](OC(C)(C)C)C(=O)O)CCN(S(=O)(=O)CC(C)(C)C)C3)cc1]

The title compound was made in a manner similar to that described in example 4 step 2 and isolated as a white solid after reverse phase HPLC. ¹H NMR (400 MHz, METHANOL-d₄) δ ppm 7.37-7.20 (m, 3H), 7.08 (d, J=7.7 Hz, 1H), 5.07 (s, 1H), 4.47-4.23 (m, 2H), 3.62-3.54 (m, 1H), 3.53-3.44 (m, 1H), 3.00 (s, 2H), 2.88 (t, J=5.8 Hz, 2H), 2.42 (s, 3H), 2.29 (s, 3H), 1.86 (s, 3H), 1.19 (s, 9H), 0.93 (s, 9H); LCMS (m/z) ES⁻=514.4 (M−1).

### Example 63: (S)-2-(tert-butoxy)-2-(2-(N-cyclohexylsulfamoyl)-5,8-dimethyl-7-(p-tolyl)-1,2,3,4-tetrahydroisoquinolin-6-yl)acetic Acid

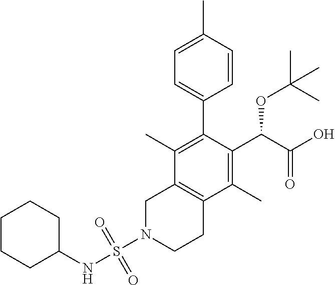 [SMILES: Cc1ccc(-c2c(C)c3c(c(C)c2[C@H](OC(C)(C)C)C(=O)O)CCN(S(=O)(=O)NC2CCCCC2)C3)cc1]

The title compound was made in a manner similar to that described in example 57 from cyclohexylsulfamoyl chloride, except it was heated to 50° C. The final product was isolated as a white solid after reverse phase HPLC. ¹H NMR (400 MHz, METHANOL-d₄) δ ppm 7.35-7.19 (m, 3H), 7.07 (d, J=7.5 Hz, 1H), 5.06 (s, 1H), 4.32-4.17 (m, 2H), 3.56-3.39 (m, 2H), 3.18-3.03 (m, 1H), 2.85 (t, J=5.8 Hz, 2H), 2.42 (s, 3H), 2.28 (s, 3H), 1.93 (br. s., 2H), 1.84 (s, 3H), 1.79-1.52 (m, 3H), 1.35-1.09 (m, 5H), 0.92 (s, 9H); LCMS (m/z) ES⁻=541.4 (M−1).

### Example 64: (S)-2-(tert-butoxy)-2-(2-((3-fluoro-2-methoxyphenyl)sulfonyl)-5,8-dimethyl-7-(p-tolyl)-1,2,3,4-tetrahydroisoquinolin-6-yl)acetic Acid

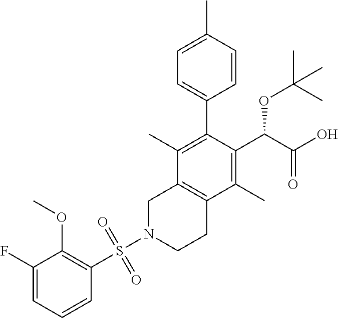 [SMILES: COc1c(F)cccc1S(=O)(=O)N1CCc2c(C)c([C@H](OC(C)(C)C)C(=O)O)c(-c3ccc(C)cc3)c(C)c2C1]

The title compound was made in a manner similar to that described in example 57 from 3-fluoro-2-methoxy-benzene-1-sulfonyl chloride, except it was heated to 45-55° C. The final product was isolated as a white solid after reverse phase HPLC. ¹H NMR (400 MHz, METHANOL-d₄) δ ppm 7.72 (d, J=7.7 Hz, 1H), 7.49-7.36 (m, 1H), 7.32-7.18 (m, 4H), 7.06 (d, J=7.7 Hz, 1H), 5.04 (s, 1H), 4.48-4.25 (m, 2H), 3.96 (s, 3H), 3.75-3.46 (m, 2H), 2.75 (t, J=5.9 Hz, 2H), 2.41 (s, 3H), 2.21 (s, 3H), 1.80 (s, 3H), 0.91 (s, 9H); LCMS (m/z) ES⁻=568.3 (M−1).

### Example 65: (S)-2-(tert-butoxy)-2-(2-(cyclopentylsulfonyl)-5,8-dimethyl-7-(p-tolyl)-1,2,3,4-tetrahydroisoquinolin-6-yl)acetic Acid

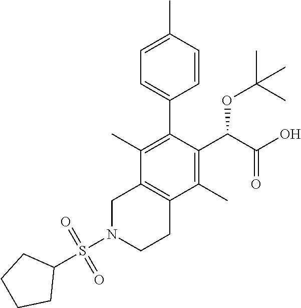 [SMILES: Cc1ccc(-c2c(C)c3c(c(C)c2[C@H](OC(C)(C)C)C(=O)O)CCN(S(=O)(=O)C2CCCC2)C3)cc1]

### Step 1: (S)-methyl 2-(tert-butoxy)-2-(2-(cyclopentylsulfonyl)-5,8-dimethyl-7-(p-tolyl)-1,2,3,4-tetrahydroisoquinolin-6-yl)acetate

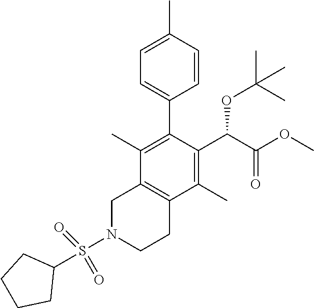 [SMILES: COC(=O)[C@@H](OC(C)(C)C)c1c(C)c2c(c(C)c1-c1ccc(C)cc1)CN(S(=O)(=O)C1CCCC1)CC2]

An ice cold solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in Dichloromethane (DCM) (1 mL) was treated with pyridine (4.50 μl, 0.056 mmol), cyclopentanesulfonyl chloride (42.6 mg, 0.253 mmol), and stirred at rt for 4.5 hours. The reaction was heated at 45° C. for 1 hour, treated with additional pyridine (10 uL), stirred at 55° C. for 18 hours, and cooled to rt over the weekend. The mixture was treated with DMAP (3.09 mg, 0.025 mmol), stirred at for 1.5 hours, treated with Et3N (0.014 mL, 0.101 mmol), and stirred at 50° C. for 2.5 hours. The reaction was cooled to rt, diluted with 1N HCl, extracted with DCM, washed with Brine, dried over Na₂SO₄, filtered, and concentrated. Purification with column chromatography (0-50% EtOAc/Hexane) afforded (S)-methyl 2-(tert-butoxy)-2-(2-(cyclopentylsulfonyl)-5,8-dimethyl-7-(p-tolyl)-1,2,3,4-tetrahydroisoquinolin-6-yl)acetate (9.7 mg, 0.018 mmol, 36.4% yield) as white solid. LCMS (m/z) ES⁺=550.5 (M+23, weak).

### Step 2: (S)-2-(tert-butoxy)-2-(2-(cyclopentylsulfonyl)-5,8-dimethyl-7-(p-tolyl)-1,2,3,4-tetrahydroisoquinolin-6-yl)acetic Acid

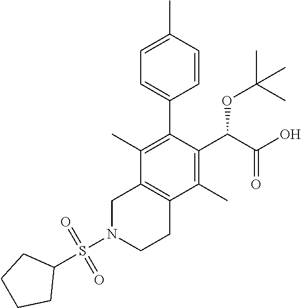 [SMILES: Cc1ccc(-c2c(C)c3c(c(C)c2[C@H](OC(C)(C)C)C(=O)O)CCN(S(=O)(=O)C2CCCC2)C3)cc1]

The title compound was made in a manner similar to that described in example 4 step 2 and isolated as a white solid after reverse phase HPLC. ¹H NMR (400 MHz, METHANOL-d₄) δ ppm 7.34-7.21 (m, 3H), 7.08 (d, J=7.3 Hz, 1H), 5.06 (s, 1H), 4.50-4.32 (m, 2H), 3.80-3.55 (m, 3H), 2.85 (t, J=5.9 Hz, 2H), 2.42 (s, 3H), 2.28 (s, 3H), 2.12-1.89 (m, 4H), 1.85 (s, 3H), 1.82-1.57 (m, 4H), 0.92 (s, 9H); LCMS (m/z) ES⁻=512.4 (M−1).

### Example 66: (S)-2-(tert-butoxy)-2-(2-(isopropylsulfonyl)-5,8-dimethyl-7-(p-tolyl)-1,2,3,4-tetrahydroisoquinolin-6-yl)acetic Acid

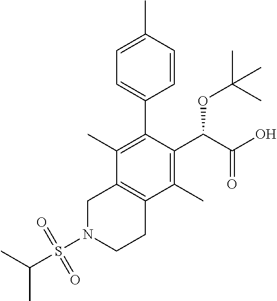 [SMILES: Cc1ccc(-c2c(C)c3c(c(C)c2[C@H](OC(C)(C)C)C(=O)O)CCN(S(=O)(=O)C(C)C)C3)cc1]

The title compound was made in a manner similar to that described in example 57 from propane-2-sulfonyl chloride, except it was heated to 45-55° C. The final product was isolated as a white solid after reverse phase HPLC. ¹H NMR (400 MHz, METHANOL-d₄) δ ppm 7.36-7.20 (m, 3H), 7.08 (d, J=7.1 Hz, 1H), 5.06 (s, 1H), 4.51-4.34 (m, 2H), 3.78-3.55 (m, 2H), 3.50-3.37 (m, 1H), 2.85 (t, J=5.8 Hz, 2H), 2.42 (s, 3H), 2.28 (s, 3H), 1.84 (s, 3H), 1.39-1.27 (m, 6H), 0.92 (s, 9H); LCMS (m/z) ES⁻=486.4 (M−1).

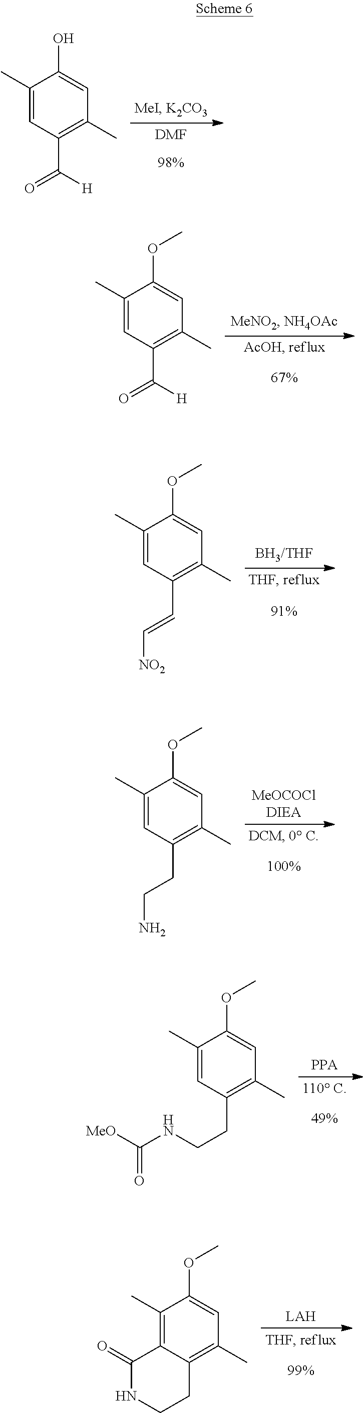

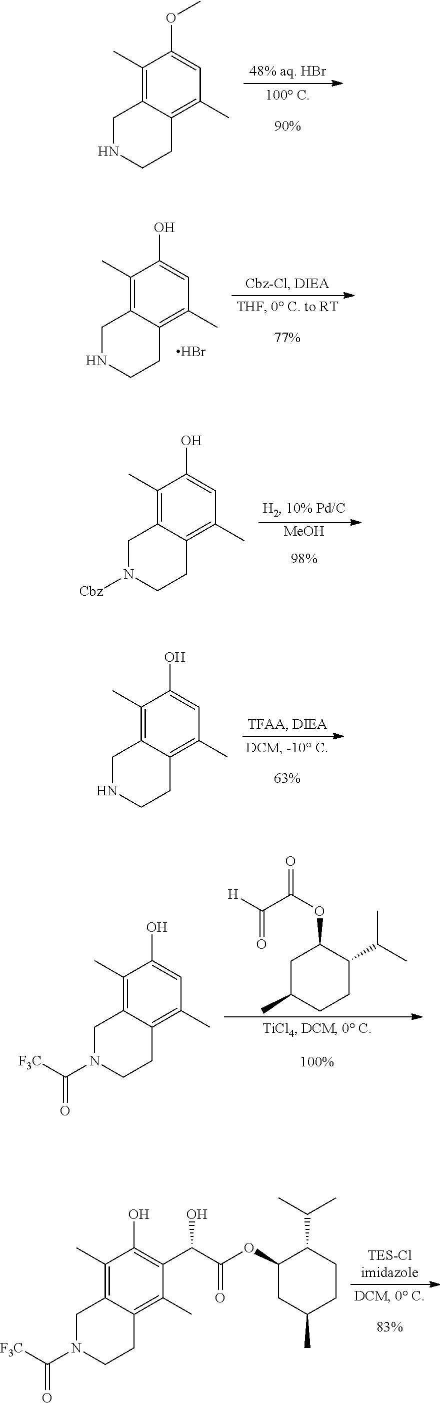

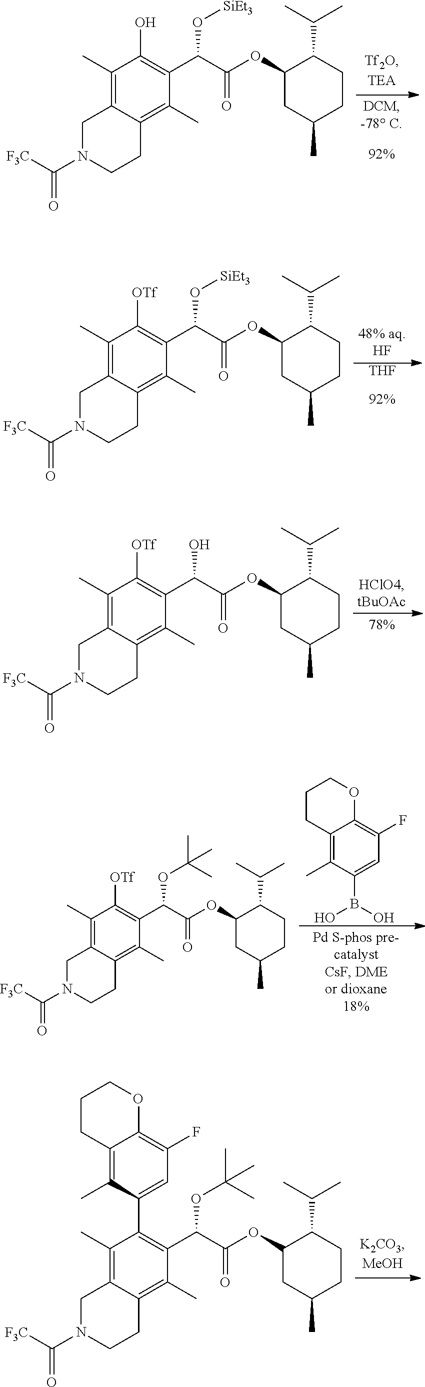

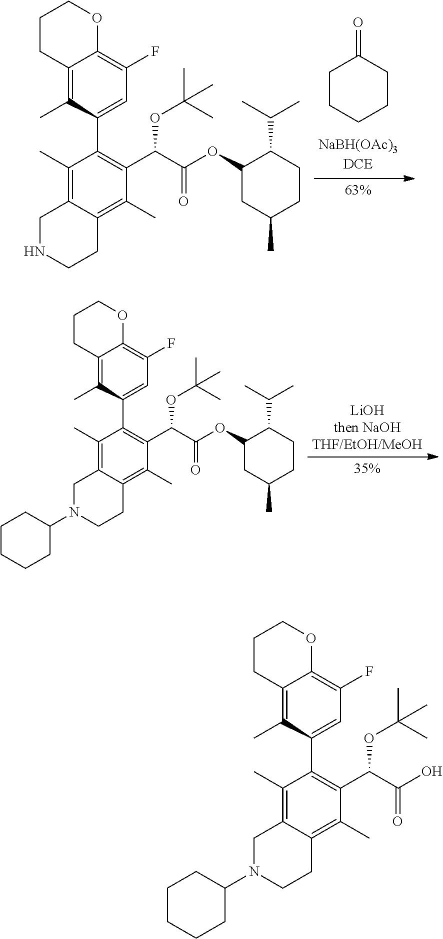

### Example 67: (2S)(M)-2-(tert-butoxy)-2-(2-cyclohexyl-7-(8-fluoro-5-methylchroman-6-yl)-5,8-dimethyl-1,2,3,4-tetrahydroisoquinolin-6-yl)acetic Acid

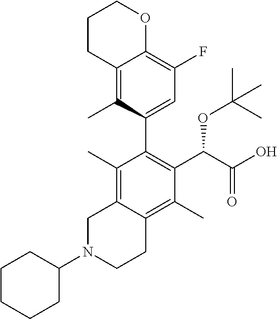

### Step 1: 4-Methoxy-2,5-dimethylbenzaldehyde

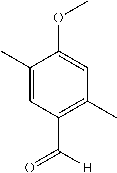

To a stirred solution of 4-hydroxy-2,5-dimethylbenzaldehyde (10.0 g, 66.6 mmol) in DMF (50 mL) was added methyl iodide (4.37 mL, 69.9 mmol) followed by K₂CO₃ (18.4 g, 133 mmol). The resulting mixture was stirred at RT. After 1.5 h TLC (silica gel, 75:25 hex/EtOAc) indicated complete conversion of the starting material to a new, higher R_{f} component. The mixture was partitioned between water and EtOAc and the phases separated. The aqueous solution was extracted once with EtOAc. The combined EtOAc solutions were washed with water (2×), brine (1×), dried over Na₂SO₄, and concentrated to dryness at reduced pressure to afford the title compound (10.7 g, 98%) as a pale yellow liquid. ¹H NMR (400 MHz, CDCl₃) δ 10.13 (s, 1H), 7.59 (s, 1H), 6.65 (s, 1H), 3.90 (s, 3H), 2.66 (s, 3H), 2.22 (s, 3H).

LCMS (ES+)(m/z): 165.21 (M+1).

### Step 2: (E)-1-Methoxy-2,5-dimethyl-4-(2-nitrovinyl)benzene

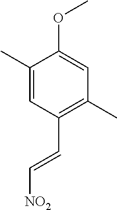

To a solution of 4-methoxy-2,5-dimethylbenzaldehyde (10.6 g, 64.6 mmol) in glacial AcOH (52 mL) was added nitromethane (10.4 mL, 194 mmol) followed by NH₄OAc (1.99 g, 25.8 mmol). The resulting solution was heated to reflux under nitrogen. After 18 h the solution was cooled to RT at which point a solid formed. The mixture was diluted with water (250 mL). The resulting suspension was stirred vigorously for 30 minutes. The solid was collected by filtration, washing twice with water. The solid was suction air dried for 1 h and then in vacuo for 2 h to give 12.0 g of an orange-brown solid. This material was recrystallized from hot hexane/EtOAc to give the title compound (8.89 g, 67%) as a yellow solid. ¹H NMR (400 MHz, CDCl₃) δ 8.26 (d, J=13.5 Hz, 1H), 7.49 (d, J=13.5 Hz, 1H), 7.32 (s, 1H), 6.69 (s, 1H), 3.88 (s, 3H), 2.47 (s, 3H), 2.19 (s, 3H).

### Step 3: 2-(4-Methoxy-2,5-dimethylphenyl)ethanamine

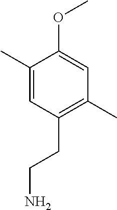

To 1M BH₃/THF (212 mL, 212 mmol) was added a solution of (E)-1-methoxy-2,5-dimethyl-4-(2-nitrovinyl)benzene (8.80 g, 42.5 mmol) in anhydrous THF (65 mL) by slow addition over 5 min. The resulting solution was heated to reflux with stirring under nitrogen. After 3 h the solution was cooled to RT and stirred overnight. The solution was cooled to 0° C. and treated with 6M aqueous HCl (142 mL, 849 mmol) by slow addition over 10 min. The mixture was diluted with water and stirred at RT for 30 min. The solution was extracted with ether (3×). The aqueous solution was cooled in an ice water bath and slowly treated with 10M aqueous NaOH (106 mL, 1062 mmol). The resulting cloudy solution was extracted with ether (3×). The combined ether extracts were washed with brine, dried over Na₂SO₄, and concentrated at reduced pressure to afford the title compound (6.94 g, 91%) as a yellow oil. ¹H NMR (400 MHz, CDCl₃) δ 6.91 (s, 1H), 6.64 (s, 1H), 3.81 (s, 3H), 2.85-2.94 (m, 2H), 2.63-2.74 (m, 2H), 2.30 (s, 3H), 2.18 (s, 3H), 1.24-1.61 (br s, 2H). LCMS (ES+)(m/z): 180.26 (M+1).

### Step 4: Methyl 4-methoxy-2,5-dimethyiphenethylcarbamate

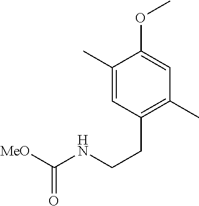 [SMILES: COC(=O)NCCc1cc(C)c(OC)cc1C]

A stirred solution of 2-(4-methoxy-2,5-dimethylphenyl)ethanamine (6.94 g, 38.78 mmol) and DIEA (10.1 mL, 58.1 mmol) in DCM (51 mL) at 0° C. was treated with methyl chloroformate (3.00 mL, 38.7 mmol) by dropwise addition. After 30 minutes LCMS indicated complete reaction. The solution was concentrated to dryness and the residue dissolved in EtOAc. The solution was washed with 10% aq. citric acid (2×), saturated aq. NaHCO₃ (2×), dried over Na₂SO₄, and concentrated to dryness at reduced pressure to give the title compound in quantitative yield. ¹H NMR (400 MHz, CDCl₃) δ 6.88 (s, 1H), 6.64 (s, 1H), 4.72 (br s, 1H), 3.81 (s, 3H), 3.68 (s, 3H), 3.35 (m, 2H), 2.74 (m, 2H), 2.31 (s, 3H), 2.17 (s, 3H). LCMS (ES+)(m/z): 238.32 (M+1).

### Step 5: 7-Methoxy-5,8-dimethyl-3,4-dihydroisoquinolin-1(2H)-one

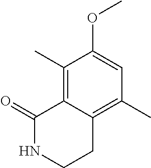

Polyphosphoric acid (30 g) was added to methyl 4-methoxy-2,5-dimethylphenethylcarbamate (9.10 g, 38.3 mmol) and the mixture heated to 100° C. with slow stirring. The reaction progress was monitored by LCMS. After 2.5 h the reaction temperature was increased to 110° C. After another 1.5 h LCMS indicated complete reaction. The mixture was cooled to about 60° C. and the residue was dissolved in 1:1 EtOAc/water. The resulting bi-phasic mixture stirred vigorously for 30 min, and then the phases separated. The aq. phase was extracted with two additional portions of EtOAc. The combined EtOAc solutions were washed with sat. aq. NaHCO₃ (2×), brine (1×), dried over Na₂SO₄ and concentrated to dryness at reduced pressure to give a grey solid. This material was triturated with hexane, filtered, and dried in vacuo to afford the title compound (3.87 g, 49%) as an off-white solid. ¹H NMR (400 MHz, CDCl₃) δ 6.82 (s, 1H), 6.30 (br s, 1H), 3.83 (s, 3H), 3.43 (m, 2H), 2.81 (m, 2H), 2.55 (s, 3H), 2.30 (s, 3H). LCMS (ES+)(m/z): 206.27 (M+1).

### Step 6: 7-Methoxy-5,8-dimethyl-1,2,3,4-tetrahydroisoquinoline

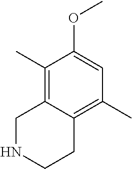

1M LAH/THF (56.4 mL, 56.4 mmol) was added to anhydrous THF (70 mL) under nitrogen. The solution was cooled to 0° C. and treated with a solution of 7-methoxy-5,8-dimethyl-3,4-dihydroisoquinolin-1(2H)-one (3.86 g, 18.8 mmol) in anhydrous THF (80 mL) by dropwise addition. The resulting solution was heated to reflux with stirring. After 2.5 h the cloudy solution was cooled to 0° C. and quenched by dropwise addition of water (2.1 mL), followed by 15% aqueous NaOH (2.1 mL), and then water (6.3 mL). After warming to RT and stirring for 1 h, celite was added and the mixture was filtered, washing with excess EtOAc. The filtrate was concentrated at reduced pressure to give the title compound (3.56 g, 99%) as a white solid. ¹H NMR (400 MHz, DMSO-d₆) δ 6.65 (s, 1H), 3.66-3.76 (m, 5H), 2.89 (t, J=5.9 Hz, 2H), 2.45 (t, J=5.8 Hz, 2H), 2.13 (s, 3H), 1.93 (s, 3H). LCMS (ES+)(m/z): 192.08 (M+1).

### Step 7: 5,8-Dimethyl-1,2,3,4-tetrahydroisoquinolin-7-ol hydrobromide

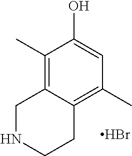

A suspension of 7-methoxy-5,8-dimethyl-1,2,3,4-tetrahydroisoquinoline (3.56 g, 18.6 mmol) in of 48% aq. HBr (50 mL) was heated to 100° C. with stirring under nitrogen. After 18 h the mixture was cooled to RT. The suspension was then cooled to 0° C., stirred for 1 hour, and the solid collected by vacuum filtration. The filter cake was washed with ice cold water (2×), ether (1×), and dried in vacuo to afford the title compound (4.34 g, 90%) as an off-white solid. ¹H NMR (400 MHz, DMSO-d₆) δ 9.20 (s, 1H), 9.03 (br s, 2H), 6.65 (s, 1H), 4.12 (br s, 2H), 3.25-3.41 (m, 2H), 2.73 (t, J=6.1 Hz, 2H), 2.10 (s, 3H), 1.96 (s, 3H). LCMS (ES+)(m/z): 178.24 (M+1).

### Step 8: Benzyl 7-hydroxy-5,8-dimethyl-3,4-dihydroisoquinoline-2(1H)-carboxylate

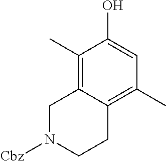 [SMILES: Cc1cc(O)c(C)c2c1CCN(C(=O)OCc1ccccc1)C2]

To a stirred suspension of 5,8-dimethyl-1,2,3,4-tetrahydroisoquinolin-7-ol hydrobromide (4.34 g, 16.8 mmol) and DIEA (4.40 mL, 25.2 mmol) in THF (80 mL) at 0° C. was added CbzCl (2.40 mL, 16.8 mmol) by dropwise addition. The reaction mixture was allowed to warm to RT. After 30 min LCMS indicated complete reaction. The suspension was partitioned between 0.5 M aq. HCl and EtOAc and the phases separated. The EtOAc solution was washed with water (2×), brine (1×), dried over Na₂SO₄ and concentrated at reduced pressure. The residue was subjected to flash chromatography (silica gel, 0-70% EtOAc/hexanes, gradient elution) to afford the title compound (4.02 g, 77%) as a white foam. ¹H NMR (400 MHz, DMSO-d₆) δ 8.98 (s, 1H), 7.26-7.44 (m, 5H), 6.55 (s, 1H), 5.13 (s, 2H), 4.36-4.51 (m, 2H), 3.60 (br s, 2H), 2.55 (t, J=5.9 Hz, 2H), 2.07 (s, 3H), 1.86-2.00 (m, 3H). LCMS (ES+)(m/z): 312.31 (M+1).

### Step 9: 5,8-Dimethyl-1,2,3,4-tetrahydroisoquinolin-7-ol

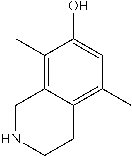

A solution of benzyl 7-hydroxy-5,8-dimethyl-3,4-dihydroisoquinoline-2(1H)-carboxylate (4.02 g, 12.9 mmol) in MeOH (150 mL) was subjected to hydrogenation at 40 psi in the presence of 10% Pd/C (0.40 g). After 2 hours LCMS indicated complete reaction and the product had precipitated. In order to remove the catalyst, the mixture was diluted with 100 mL of MeOH and 100 mL of DCM and heated to reflux under nitrogen until all of the solid product had dissolved. The catalyst was removed by filtration through celite and the filtrate concentrated to dryness at reduced pressure to give the title compound (2.24 g, 98%) as a pale yellow solid. ¹H NMR (400 MHz, DMSO-d₆) δ 8.78 (br s, 1H), 6.48 (s, 1H), 3.68 (s, 2H), 2.88 (t, J=5.8 Hz, 2H), 2.41 (t, J=5.7 Hz, 2H), 2.04 (s, 3H), 1.90 (s, 3H).

LCMS (ES+)(m/z): 178.23 (M+1).

### Step 10: 2,2,2-Trifluoro-1-(7-hydroxy-5,8-dimethyl-3,4-dihydroisoquinolin-2(1H)-yl)ethanone

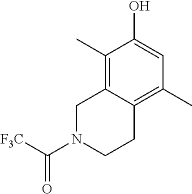 [SMILES: Cc1cc(O)c(C)c2c1CCN(C(=O)C(F)(F)F)C2]

To a stirred suspension of 5,8-dimethyl-1,2,3,4-tetrahydroisoquinolin-7-ol (2.24 g, 12.6 mmol) in anhydrous DCM (65 mL) at −10° C. was added DIEA (2.65 mL, 15.2 mmol). This was followed by dropwise addition of a solution of trifluoroacetic anhydride (1.80 mL, 12.6 mmol) in DCM (8 mL) over 5 min. After stirring at −10° C. for 1 h, LCMS indicated complete reaction. The solution was diluted with DCM, washed with 1N aq. HCl (2×), water (1×), dried over Na₂SO₄, and concentrated to dryness at reduced pressure. The crude product was recrystallized from hot hexane/EtOAc to afford the title compound (2.18 g, 63%) as a white solid. ¹H NMR (400 MHz, DMSO-d₆, mixture of rotamers) δ 9.04-9.18 (m, 1H), 6.50-6.64 (m, 1H), 4.60 (s, 2H), 3.71-3.83 (m, 2H), 2.60-2.72 (m, 2H), 1.90-2.16 (m, 6H). LCMS (ES+)(m/z): 274.26 (M+1).

### Step 11: (S)-(1R,2S,5R)-2-Isopropyl-5-methylcyclohexyl 2-hydroxy-2-(7-hydroxy-5,8-dimethyl-2-(2,22-trifluoroacetyl)-1,2,3,4-tetrahydroisoquinolin-6-yl)acetate

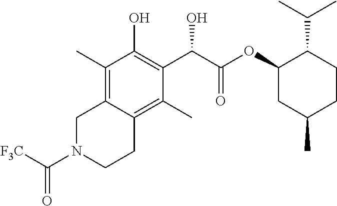 [SMILES: Cc1c(O)c([C@H](O)C(=O)O[C@@H]2C[C@H](C)CC[C@H]2C(C)C)c(C)c2c1CN(C(=O)C(F)(F)F)CC2]

To a stirred solution of 2,2,2-trifluoro-1-(7-hydroxy-5,8-dimethyl-3,4-dihydroisoquinolin-2(1H)-yl)ethanone (1.98 g, 7.25 mmol) in anhydrous DCM (65 mL) at 0° C. was added 1M TiCl₄/DCM (8.70 mL, 8.70 mmol) over a 5 min period. A dark red-brown solution was produced which was stirred at 0° C. After 10 min a solution of (1R,2S,5R)-2-isopropyl-5-methylcyclohexyl 2-oxoacetate (2.46 g, 11.6 mmol) in DCM (20 mL) was added dropwise. The resulting mixture was stirred at 0° C. After 1.5 hours LCMS indicated complete conversion to the desired product. The mixture was partitioned between DCM and 1N aq. HCl and the phases separated. The aqueous phase was extracted with DCM (2×). The combined DCM solutions were dried over Na₂SO₄ and concentrated at reduced pressure. The residue was subjected to flash chromatography (silica gel 0-100% EtOAc/hexanes, gradient elution) to afford the title compound in quantitative yield as a light tan foam. ¹H NMR (400 MHz, DMSO-d₆, mixture of rotamers) δ 8.62-8.76 (m, 1H), 5.56-6.66 (m, 2H), 4.50-4.68 (m, 3H), 3.66-3.88 (m, 2H), 2.63-2.82 (m, 2H), 0.57-2.23 (m, 24H). LCMS (ES+)(m/z): 486.48 (M+1).

### Step 12: (S)-(1R,2S,5R)-2-Isopropyl-5-methylcyclohexyl 2-(7-hydroxy-5,8-dimethyl-2-(2,2,2-trifluoroacetyl)-1,2,3,4-tetrahydroisoquinolin-6-yl)-2-((triethylsilyl)oxy)acetate

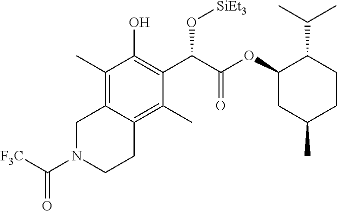

To a stirred solution of (S)-(1R,2S,5R)-2-isopropyl-5-methylcyclohexyl 2-hydroxy-2-(7-hydroxy-5,8-dimethyl-2-(2,2,2-trifluoroacetyl)-1,2,3,4-tetrahydroisoquinolin-6-yl)acetate (3.50 g, 7.21 mmol) and imidazole (0.74 g, 10.8 mmol) in anhydrous DCM (48 mL) at 0° C. was added a solution of TES-Cl (1.10 mL, 6.49 mmol) in DCM (5 mL) by dropwise addition. This produced a white suspension that was stirred at 0° C. After 30 min LCMS indicated about 50% conversion. The solution was treated with additional imidazole (0.25 g, 0.50 equiv.) and TES-Cl (0.363 mL, 0.30 equiv.) in 2 mL DCM, and stirring at 0° C. continued. After 30 additional min, another 0.363 mL of TES-Cl was added. After another 30 min, the mixture was partitioned between 1N aq. HCl and DCM and the phases separated. The aqueous phase was extracted with one additional portion of DCM. The combined DCM solutions were washed with 1N HCl (1×), brine (1×), dried over Na₂SO₄, and concentrated at reduced pressure. The crude residue was subjected to flash chromatography (silica gel, 0-70% EtOAc/hexanes, gradient elution) to give the title compound (3.59 g, 83%) as a tacky white foam. ¹H NMR (400 MHz, DMSO-d₆, mixture of rotamers) δ 8.37-8.51 (m, 1H), 5.96 (br s, 1H), 4.52-4.71 (m, 3H), 3.71-3.89 (m, 2H), 2.61-2.82 (m, 2H), 2.00-2.16 (m, 6H), 1.74-1.95 (m, 2H), 1.61 (m, 2H), 1.24-1.52 (m, 2H), 0.45-1.14 (m, 27H). LCMS (ES+)(m/z): 622.67 (M+Na).

### Step 13: (S)-(1R,2S,5R)-2-Isopropyl-5-methylcyclohexyl 2-(5,8-dimethyl-2-(2,2,2-trifluoroacetyl)-7-(((trifluoromethyl)sulfonyl)oxy)-1,2,3,4-tetrahydroisoquinolin-6-yl)-2-((triethylsilyl)oxy)acetate

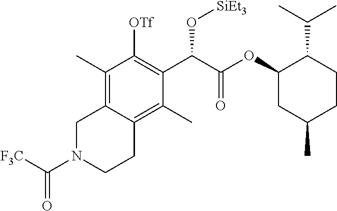

To a stirred solution of (S)-(1R,2S,5R)-2-isopropyl-5-methylcyclohexyl 2-(7-hydroxy-5,8-dimethyl-2-(2,2,2-trifluoroacetyl)-1,2,3,4-tetrahydroisoquinolin-6-yl)-2-((triethylsilyl)oxy)acetate (3.55 g, 5.92 mmol) and TEA (2.06 mL, 14.8 mmol) in anhydrous DCM (60 mL) at −78° C. was added 1M triflic anhydride/DCM (7.40 mL, 7.40 mmol) by dropwise addition. After 1 hour LCMS indicated complete reaction. The solution was partitioned between 1N aqueous HCl and DCM and the phases separated. The aqueous solution was extracted with one additional portion of DCM. The combined DCM solutions were washed with 1N HCl (1×), brine (1×), dried over Na₂SO₄, and concentrated at reduced pressure. The crude material was subjected to flash chromatography (silica gel, 0-15% EtOAc/hexanes, gradient elution) to give the title compound (4.00 g, 92%) as a white foam. ¹H NMR (400 MHz, DMSO-d₆, mixture of rotamers) δ 5.58-5.81 (m, 1H), 4.58-4.82 (m, 3H), 3.71-3.97 (m, 2H), 2.73-2.96 (m, 2H), 2.15-2.24 (m, 7H), 1.91-2.00 (m, 1H), 1.76-1.83 (m, 1H), 1.57-1.66 (m, 2H), 1.26-1.36 (m, 1H), 0.78-0.92 (m, 18H), 0.71 (d, J=7.0 Hz, 3H), 0.47-0.63 (m, 6H). LCMS (ES+)(m/z): 754.64 (M+Na).

### Step 14: (S)-(1R,2S,5R)-2-Isopropyl-5-methylcyclohexyl 2-(5,8-dimethyl-2-(2,2,2-trifluoroacetyl)-7-(((trifluoromethyl)sulfonyl)oxy)-1,2,3,4-tetrahydroisoquinolin-6-yl)-2-hydroxyacetate

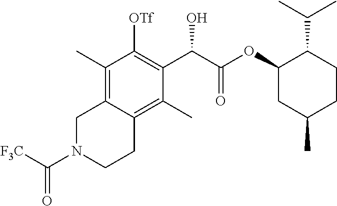 [SMILES: Cc1c2c(c(C)c([C@H](O)C(=O)O[C@@H]3C[C@H](C)CC[C@H]3C(C)C)c1OS(=O)(=O)C(F)(F)F)CCN(C(=O)C(F)(F)F)C2]

To a stirred solution of (S)-(1R,2S,5R)-2-isopropyl-5-methylcyclohexyl 2-(5,8-dimethyl-2-(2,2,2-trifluoroacetyl)-7-(((trifluoromethyl)sulfonyl)oxy)-1,2,3,4-tetrahydroisoquinolin-6-yl)-2-((triethylsilyl)oxy)acetate (4.00 g, 5.47 mmol) in THF (50 mL) in a teflon vessel was added 48% aq. HF. The resulting solution was stirred at RT. After 18 h LCMS indicated complete reaction. The solution was diluted with EtOAc, washed with sat. aq. NaHCO₃ (3×), dried over Na₂SO₄, and concentrated to dryness. The residue was subjected to flash chromatography (silica gel, 0-100% EtOAc/hexanes, gradient elution) to afford the title compound (3.12 g, 92%) as a white foam. ¹H NMR (400 MHz, DMSO-d₆) δ 6.36-6.45 (m, 1H), 5.46 (d, J=4.9 Hz, 1H), 4.56-4.81 (m, 3H), 3.69-3.97 (m, 2H), 2.71-2.93 (m, 2H), 2.12-2.26 (m, 6H), 1.71-1.93 (m, 2H), 1.54-1.65 (m, 2H), 1.34-1.50 (m, 1H), 1.19-1.31 (m, 1H), 0.61-1.09 (m, 12H). LCMS (ES+)(m/z): 635.56 (M+18).

### Step 15: (S)-(1R,2S,5R)-2-isopropyl-5-methylcyclohexyl 2-(tert-butoxy)-2-(5,8-dimethyl-2-(2,2,2-trifluoroacetyl)-7-(((trifluoromethyl)sulfonyl)oxy)-1,2,3,4-tetrahydroisoquinolin-6-yl)acetate

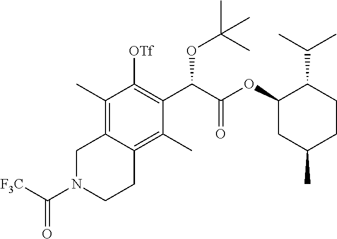 [SMILES: Cc1c2c(c(C)c([C@H](OC(C)(C)C)C(=O)O[C@@H]3C[C@H](C)CC[C@H]3C(C)C)c1OS(=O)(=O)C(F)(F)F)CCN(C(=O)C(F)(F)F)C2]

To a stirred solution of (S)-(1R,2S,5R)-2-isopropyl-5-methylcyclohexyl 2-(5,8-dimethyl-2-(2,2,2-trifluoroacetyl)-7-(((trifluoromethyl)sulfonyl)oxy)-1,2,3,4-tetrahydroisoquinolin-6-yl)-2-hydroxyacetate (0.250 g, 0.41 mmol) in 20 mL of t-BuOAc was added perchloric acid (49 uL, 0.81 mmol). After 50 minutes the solution was poured into rapidly stirred saturated aqueous NaHCO₃. After stirring for several minutes the phases were separated. The aqueous solution was extracted once with EtOAc. The combined organic solutions were washed with saturated aqueous NaHCO₃ (1×), dried over sodium sulfate, and concentrated to dryness at reduced pressure. The residue was subjected to flash chromatography (silica gel, 0-50% EtOAc/hexanes) to afford the title compound as a white foam (0.214 g, 78%). ¹H NMR (400 MHz, CHLOROFORM-d) δ=5.60-5.50 (m, 1H), 4.89-4.51 (m, 3H), 4.16-3.66 (m, 2H), 2.98-2.68 (m, 2H), 2.33-2.19 (m, 6H), 2.12-1.83 (m, 2H), 1.66 (d, J=11.9 Hz, 2H), 1.52-1.30 (m, 2H), 1.26-1.13 (m, 10H), 1.11-0.97 (m, 1H), 0.88 (t, J=6.0 Hz, 7H), 0.76 (d, J=6.8 Hz, 3H); LC/MS (m/z) ES⁺=696 (M+23).

### Step 16: (S)-(1S,2S,5R)-2-isopropyl-5-methylcyclohexyl 2-(tert-butoxy)-2-((R)-7-(8-fluoro-5-methylchroman-6-yl)-5,8-dimethyl-2-(2,2,2-trifluoroacetyl)-1,2,3,4-tetrahydroisoquinolin-6-yl)acetate

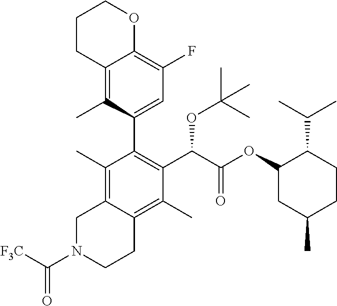

A degassed mixture of (S)-(1S,2S,5R)-2-isopropyl-5-methylcyclohexyl 2-(tert-butoxy)-2-(5,8-dimethyl-2-(2,2,2-trifluoroacetyl)-7-(((trifluoromethyl)sulfonyl)oxy)-1,2,3,4-tetrahydroisoquinolin-6-yl)acetate (50.0 mg, 0.074 mmol), (8-fluoro-5-methylchroman-6-yl)boronic acid (39.0 mg, 0.186 mmol) and CsF (64.0 mg, 0.421 mmol) in 1,2-dimethoxyethane (1.0 mL) was treated with (2-dicyclohexylphosphino-2′,6′-dimethoxy-1,1′-biphenyl)[2-(2-aminoethylphenyl)]palladium(II) chloride-methyl-t-butyl ether adduct (16.94 mg, 0.022 mmol), further degassed for several minutes and then the mixture was irradiated in the microwave at 130° C. for 40 minutes. The mixture was degassed while adding additional CsF (80 mg), (8-fluoro-5-methylchroman-6-yl)boronic acid (39.0 mg, 0.186 mmol) and finally Pd S-phos precatalyst (16.94 mg, 0.022 mmol). The mixture was irradiated in the microwave at 130° C. for 30 minutes. LCMS indicated complete consumption of starting material. The crude reaction mixture was saved in order to be processed with additional batches. Two additional experiments were similarly performed on 31 mg and 84 mg of triflate starting material using either 1,2-dimethoxyethane or 1,4-dioxane as solvent. Upon complete of the reactions, the crude reaction mixtures were combined, diluted with EtOAc, washed with water and then brine, dried over sodium sulfate, filtered and concentrated under reduced pressure. The residue was purified on silica gel (12 g column, 0-15% hexanes/EtOAc gradient elution) to afford the title compound as a pale yellow residue (31 mg, 18%). 1H NMR indicated a 9:1 mixture of diastereomers. LC/MS (m/z) ES⁺=712 (M+23).

### Step 17: (S)-(1S,2S,5R)-2-isopropyl-5-methylcyclohexyl 2-(tert-butoxy)-2-((R)-7-(8-fluoro-5-methylchroman-6-yl)-5,8-dimethyl-1,2,3,4-tetrahydroisoquinolin-6-yl)acetate

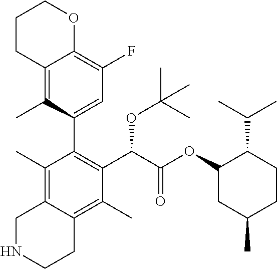

A solution of (S)-(1S,2S,5R)-2-isopropyl-5-methylcyclohexyl 2-(tert-butoxy)-2-((R)-7-(8-fluoro-5-methylchroman-6-yl)-5,8-dimethyl-2-(2,2,2-trifluoroacetyl)-1,2,3,4-tetrahydroisoquinolin-6-yl)acetate (31.0 mg, 0.045 mmol) in methanol (0.8 mL) was treated with potassium carbonate (3.60 mg, 0.026 mmol) and the mixture was allowed to stir at ambient temperature for 7 hours. The mixture was concentrated under reduced pressure. Water was added and the mixture was extracted with EtOAc. The combined extracts were dried over sodium sulfate, filtered and concentrated under reduced pressure to afford a pale yellow residue (28 mg) that was used in the next step without purification. LC/MS (m/z) ES⁺=594 (M+1)

### Step 18: (S)-(1S,2S,5R)-2-isopropyl-5-methylcyclohexyl 2-(tert-butoxy)-2-((R)-2-cyclohexyl-7-(8-fluoro-5-methylchroman-6-yl)-5,8-dimethyl-1,2,3,4-tetrahydroisoquinolin-6-yl)acetate

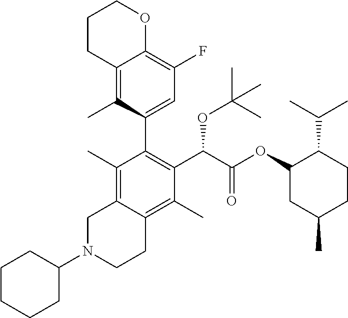

A mixture of the crude (S)-(1S,2S,5R)-2-isopropyl-5-methylcyclohexyl 2-(tert-butoxy)-2-((R)-7-(8-fluoro-5-methylchroman-6-yl)-5,8-dimethyl-1,2,3,4-tetrahydroisoquinolin-6-yl)acetate (28 mg, 0.046 mmol) in 1,2-dichloroethane (0.8 mL) was treated with cyclohexanone (7.45 μl, 0.072 mmol), followed by sodium triacetoxyborohydride (19.05 mg, 0.090 mmol) and the mixture was allowed to stir at ambient temperature overnight. The mixture was diluted with EtOAc, washed with saturated sodium bicarbonate, followed by brine, dried over sodium sulfate, filtered and then concentrated under reduced pressure. The residue was purified by reverse phase HPLC to afford a pale yellow residue (19 mg, 63%). LC/MS (m/z) ES⁺=676 (M+1)

### Step 19: (S)-2-(tert-butoxy)-2-((R)-2-cyclohexyl-7-(8-fluoro-5-methylchroman-6-yl)-5,8-dimethyl-1,2,3,4-tetrahydroisoquinolin-6-yl)acetic Acid

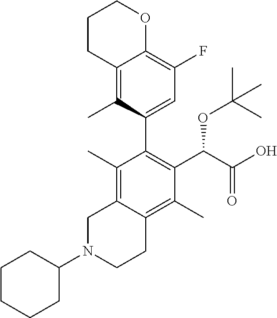

A solution of (S)-(1S,2S,5R)-2-isopropyl-5-methylcyclohexyl 2-(tert-butoxy)-2-((R)-2-cyclohexyl-7-(8-fluoro-5-methylchroman-6-yl)-5,8-dimethyl-1,2,3,4-tetrahydroisoquinolin-6-yl)acetate (19 mg, 0.028 mmol) in ethanol (0.5 mL) and tetrahydrofuran (0.5 mL) was treated with 2M LiOH (0.5 mL). The mixture was heated at 80° C. for one hour after which time LCMS indicated no reaction had occurred. The mixture was transferred to a microwave vial and irradiated in the microwave at 100-140° C. in 10-30 minute intervals over the course of four hours. Intermittently, the reaction mixture was treated with 50% NaOH (230 uL, added in three portions) and MeOH (600 uL, added in two portions). The mixture was concentrated under reduced pressure. Water was added and the mixture was adjusted to ˜pH 7 with 1N HCl. The resulting mixture was extracted with EtOAc. The combined extracts were dried over sodium sulfate, filtered and concentrated. The residue was purified by reverse phase HPLC to afford the title compound as a white solid (5.3 mg, 35%). ¹H NMR (400 MHz, CHLOROFORM-d) δ=6.63 (d, J=11.5 Hz, 1H), 4.85-4.13 (m, 3H), 3.85-3.75 (m, 1H), 3.50 (s, 1H), 2.84-2.53 (m, 4H), 2.34 (s, 3H), 2.19-1.94 (m, 4H), 1.90-1.78 (m, 2H), 1.74-1.47 (m, 6H), 1.44-0.99 (m, 18H); LC/MS (m/z) ES⁺=538 (M+1).

### Example 68: (S)-2-(tert-butoxy)-2-(2-(cycloheptylsulfonyl)-5,8-dimethyl-7-(p-tolyl)-1,2,3,4-tetrahydroisoquinolin-6-yl)acetic Acid

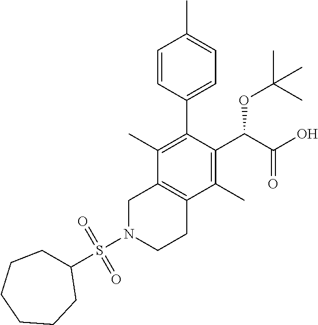 [SMILES: Cc1ccc(-c2c(C)c3c(c(C)c2[C@H](OC(C)(C)C)C(=O)O)CCN(S(=O)(=O)C2CCCCCC2)C3)cc1]

The title compound was made in a manner similar to that described in example 57 from cycloheptanesulfonyl chloride, except it was heated to 50° C. The final product was isolated as a white solid after reverse phase HPLC. ¹H NMR (400 MHz, METHANOL-d₄) δ ppm 7.38-7.20 (m, 3H), 7.08 (d, J=7.3 Hz, 1H), 5.06 (s, 1H), 4.53-4.32 (m, 2H), 3.76-3.54 (m, 2H), 3.42-3.22 (m, 1H), 2.93-2.74 (m, 2H), 2.42 (s, 3H), 2.28 (s, 3H), 2.23-2.08 (m, 2H), 1.94-1.73 (m, 7H), 1.69-1.45 (m, 6H), 0.93 (s, 9H); LCMS (m/z) ES⁻=540.4 (M−1).

### Example 69: (S)-2-(tert-butoxy)-2-(2-cyclooctyl-5,8-dimethyl-7-(p-tolyl)-1,2,3,4-tetrahydroisoquinolin-6-yl)acetic Acid

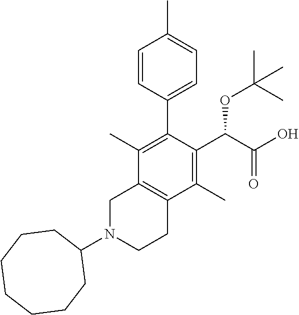 [SMILES: Cc1ccc(-c2c(C)c3c(c(C)c2[C@H](OC(C)(C)C)C(=O)O)CCN(C2CCCCCCC2)C3)cc1]

### Step 1: (S)-methyl 2-(tert-butoxy)-2-(2-cyclooctyl-5,8-dimethyl-7-(p-tolyl)-1,2,3,4-tetrahydroisoquinolin-6-yl)acetate

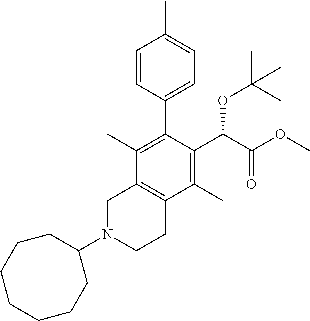 [SMILES: COC(=O)[C@@H](OC(C)(C)C)c1c(C)c2c(c(C)c1-c1ccc(C)cc1)CN(C1CCCCCCC1)CC2]

A solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in 1,2-Dichloroethane (DCE) (1 mL) was treated with cyclooctanone (9.57 mg, 0.076 mmol) and stirred for 15 min at rt. The reaction was treated with sodium triacetoxyborohydride (16.08 mg, 0.076 mmol) and stirred at rt for 18 hours. The reaction was treated with additional cyclooctanone (9.57 mg, 0.076 mmol), sodium triacetoxyborohydride (16.08 mg, 0.076 mmol) and stirred at rt for 1.5 hours. The mixture was treated with acetic acid (5.79 μl, 0.101 mmol), stirred at rt for 1.5 hours, and then heated to 50° C. for 2.5 hours. The reaction was cooled to rt, diluted with aq. sat. NaHCO3, extracted with DCM, washed with Brine, dried over Na2SO4, filtered, and concentrated. Purification with column chromatography (0-100% EtOAc/Hexane) afforded title compound (8 mg, 0.016 mmol, 31.3% yield) as clear film. LCMS (m/z) ES⁺=506.6 (M+1).

### Step 2: (S)-2-(tert-butoxy)-2-(2-cyclooctyl-5,8-dimethyl-7-(p-tolyl)-1,2,3,4-tetrahydroisoquinolin-6-yl)acetic Acid

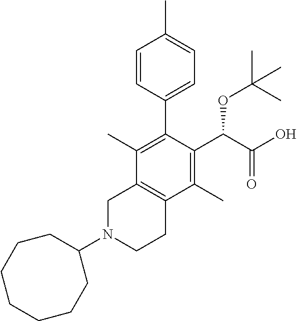 [SMILES: Cc1ccc(-c2c(C)c3c(c(C)c2[C@H](OC(C)(C)C)C(=O)O)CCN(C2CCCCCCC2)C3)cc1]

The title compound was made in a manner similar to that described in example 4 step 2 and isolated as a white solid after reverse phase HPLC as a TFA salt. ¹H NMR (400 MHz, METHANOL-d₄) δ ppm 7.37-7.21 (m, 3H), 7.12-6.97 (m, 1H), 5.11-5.01 (m, 1H), 4.49-4.27 (m, 2H), 3.83-3.65 (m, 2H), 3.46-3.34 (m, 1H), 3.18-3.01 (m, 2H), 2.43 (s, 3H), 2.38-2.25 (m, 3H), 2.19-1.85 (m, 9H), 1.79-1.47 (m, 8H), 1.00-0.81 (m, 9H); LCMS (m/z) ES⁺=492.5 (M+1), ES⁻=490.4 (M−1).

### Example 70: (2S)-2-(tert-butoxy)-2-(2-(1-cyclohexylethyl)-5,8-dimethyl-7-(p-tolyl)-1,2,3,4-tetrahydroisoquinolin-6-yl)acetic Acid

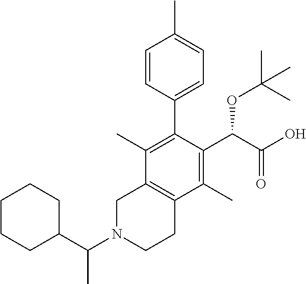 [SMILES: Cc1ccc(-c2c(C)c3c(c(C)c2[C@H](OC(C)(C)C)C(=O)O)CCN(C(C)C2CCCCC2)C3)cc1]

### Step 1: (2S)-methyl 2-(tert-butoxy)-2-(2-(1-cyclohexylethyl)-5,8-dimethyl-7-(p-tolyl)-1,2,3,4-tetrahydroisoquinolin-6-yl)acetate

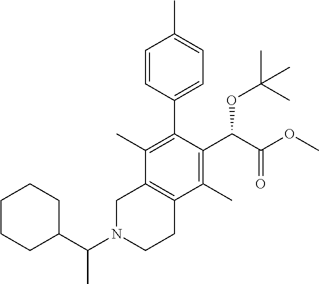 [SMILES: COC(=O)[C@@H](OC(C)(C)C)c1c(C)c2c(c(C)c1-c1ccc(C)cc1)CN(C(C)C1CCCCC1)CC2]

A solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in 1,2-Dichloroethane (DCE) (1 mL) was treated with 1-cyclohexylethanone (9.57 mg, 0.076 mmol) and stirred for 15 min at rt. The reaction was treated with sodium triacetoxyborohydride (16.08 mg, 0.076 mmol) and stirred at rt for 18 hours. The reaction was treated with additional 1-cyclohexylethanone (9.57 mg, 0.076 mmol), acetic acid (5.79 μl, 0.101 mmol), sodium triacetoxyborohydride (16.08 mg, 0.076 mmol), stirred at rt for 1.5 hours, and then at 50° C. for 1.5 hours. The reaction was treated with titanium(IV) isopropoxide (0.059 mL, 0.202 mmol), sodium cyanoborohydride (6.36 mg, 0.101 mmol), and stirred at 50° C. for 5.5 hours. The reaction was cooled to rt overnight, diluted with aq. sat. NaHCO₃, extracted with DCM, washed with Brine, dried over Na2SO4, filtered, and concentrated. Purification with column chromatography (0-50% EtOAc/Hexane) afforded title compound (5.5 mg, 10.88 μmol, 21.51% yield) as clear film. LCMS (m/z) ES⁺=506.5 (M+1).

### Step 2: (2S)-2-(tert-butoxy)-2-(2-(1-cyclohexylethyl)-5,8-dimethyl-7-(p-tolyl)-1,2,3,4-tetrahydroisoquinolin-6-yl)acetic Acid

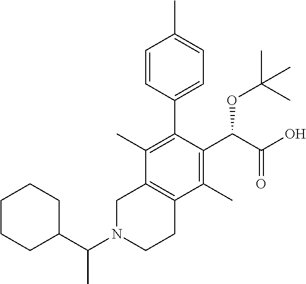 [SMILES: Cc1ccc(-c2c(C)c3c(c(C)c2[C@H](OC(C)(C)C)C(=O)O)CCN(C(C)C2CCCCC2)C3)cc1]

The title compound was made in a manner similar to that described in example 4 step 2 and isolated as a white solid after reverse phase HPLC as a TFA salt. ¹H NMR (400 MHz, METHANOL-d₄) (mixture of diastereomers) δ ppm 7.37-7.21 (m, 3H), 7.13-6.98 (m, 1H), 5.12-5.02 (m, 1H), 4.58-4.27 (m, 2H), 3.94-3.76 (m, 1H), 3.47-3.23 (m, 2H), 3.18-3.04 (m, 2H), 2.43 (s, 3H), 2.38-2.28 (m, 3H), 2.00-1.13 (m, 17H), 1.00-0.82 (m, 9H); LCMS (m/z) ES⁺=492.5 (M+1), ES⁻=490.5 (M−1).

### Example 71 and 72: (2S)-2-(tert-butoxy)-2-(5,8-dimethyl-2-((3R)-3-methylcyclohexyl)-7-(p-tolyl)-1,2,3,4-tetrahydroisoquinolin-6-yl)acetic Acid

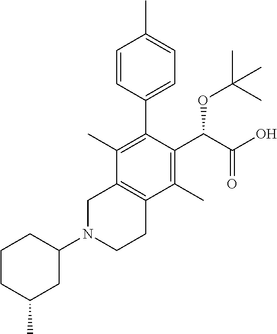 [SMILES: Cc1ccc(-c2c(C)c3c(c(C)c2[C@H](OC(C)(C)C)C(=O)O)CCN(C2CCC[C@@H](C)C2)C3)cc1]

The title compound was made in a manner similar to that described in example 49 from (R)-3-methylcyclohexanone. Isomer 1 and mixture of both isomers were isolated as white solids after reverse phase HPLC as TFA salt.

Isomer 1: ¹H NMR (400 MHz, METHANOL-d₄) δ ppm 7.36-7.20 (m, 3H), 7.13-6.97 (m, 1H), 5.13-5.01 (m, 1H), 4.40 (br. s., 2H), 3.96-3.79 (m, 1H), 3.75-3.58 (m, 1H), 3.41-3.21 (m, 1H), 3.11 (br. s., 2H), 2.43 (s, 3H), 2.37-2.04 (m, 5H), 2.03-1.82 (m, 5H), 1.82-1.42 (m, 5H), 1.11 (d, J=7.1 Hz, 3H), 1.00-0.80 (m, 9H); LCMS (m/z) ES⁺=478.4 (M+1), ES⁻=476.4 (M−1).

1:1 mixture of isomers: ¹H NMR (400 MHz, METHANOL-d₄) δ ppm 7.39-7.20 (m, 3H), 7.13-6.97 (m, 1H), 5.06 (br. s., 1H), 4.40 (br. s., 2H), 3.96-3.77 (m, 1H), 3.74-3.43 (m, 1H), 3.38-3.24 (m, 1H), 3.19-2.97 (m, 2H), 2.43 (s, 3H), 2.37-2.08 (m, 5H), 2.06-1.83 (m, 5H), 1.82-1.44 (m, 5H), 1.16-1.03 (m, 3H), 0.99-0.83 (m, 9H); LCMS (m/z) ES⁺=478.5 (M+1), ES⁻=476.3 (M−1).

### Example 73: (2S)-2-(tert-butoxy)-2-(5,8-dimethyl-2-((3R)-3-methylcyclopentyl)-7-(p-tolyl)-1,2,3,4-tetrahydroisoquinolin-6-yl)acetic Acid

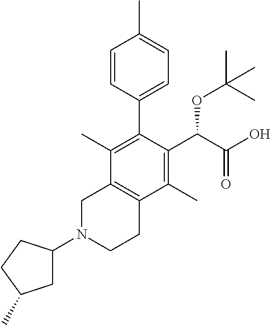 [SMILES: Cc1ccc(-c2c(C)c3c(c(C)c2[C@H](OC(C)(C)C)C(=O)O)CCN(C2CC[C@@H](C)C2)C3)cc1]

The title compound was made in a manner similar to that described in example 49 from (R)-3-methylcyclopentanone and isolated as a white solid after reverse phase HPLC as TFA salt. ¹H NMR (400 MHz, METHANOL-d₄) (mixture of diastereomers) δ ppm 7.36-7.20 (m, 3H), 7.13-6.97 (m, 1H), 5.10-5.01 (m, 1H), 4.57-4.17 (m, 2H), 3.96-3.73 (m, 2H), 3.44-3.24 (m, 1H), 3.19-3.00 (m, 2H), 2.53-2.21 (m, 8H), 2.20-1.75 (m, 6H), 1.52-1.27 (m, 2H), 1.18-1.05 (m, 3H), 1.01-0.80 (m, 9H); LCMS (m/z) ES⁺=464.4 (M+1).

### Example 74: (2S)-2-(2-(bicyclo[3.2.1]octan-3-yl)-5,8-dimethyl-7-(p-tolyl)-1,2,3,4-tetrahydroisoquinolin-6-yl)-2-(tert-butoxy)acetic Acid

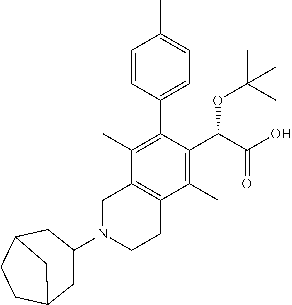 [SMILES: Cc1ccc(-c2c(C)c3c(c(C)c2[C@H](OC(C)(C)C)C(=O)O)CCN(C2CC4CCC(C4)C2)C3)cc1]

The title compound was made in a manner similar to that described in example 70 from bicyclo[3.2.1]octan-3-one and isolated as a white solid after reverse phase HPLC as TFA salt. ¹H NMR (400 MHz, METHANOL-d₄) δ ppm 7.38-7.20 (m, 3H), 7.15-6.97 (m, 1H), 5.06 (br. s., 1H), 4.53-4.20 (m, 2H), 4.04-3.81 (m, 1H), 3.74-3.55 (m, 1H), 3.42-3.18 (m, 1H), 3.17-2.95 (m, 2H), 2.57-2.39 (m, 5H), 2.33 (br. s., 3H), 2.24-2.02 (m, 2H), 1.90 (s, 3H), 1.86-1.46 (m, 8H), 1.02-0.79 (m, 9H); LCMS (m/z) ES⁺=490.4 (M+1), ES⁻=488.4 (M−1).

### Example 75: (S)-2-(tert-butoxy)-2-(2-(cycloheptanecarbonyl)-5,8-dimethyl-7-(p-tolyl)-1,2,3,4-tetrahydroisoquinolin-6-yl)acetic Acid

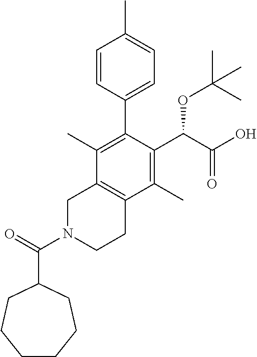 [SMILES: Cc1ccc(-c2c(C)c3c(c(C)c2[C@H](OC(C)(C)C)C(=O)O)CCN(C(=O)C2CCCCCC2)C3)cc1]

The title compound was made in a manner similar to that described in example 5 from cycloheptanecarboxylic acid and isolated as a white solid after reverse phase HPLC.

¹H NMR (400 MHz, METHANOL-d₄) δppm 7.37-7.19 (m, 3H), 7.13-7.01 (m, 1H), 5.06 (s, 1H), 4.73-4.51 (m, 2H), 3.92-3.75 (m, 2H), 3.06-2.65 (m, 3H), 2.42 (s, 3H), 2.34-2.20 (m, 3H), 1.95-1.42 (m, 15H), 0.92 (s, 9H); LCMS (m/z) ES⁺=506.5 (M+1), ES⁻=504.5 (M−1).

### Example 76: (S)-2-(tert-butoxy)-2-(5,8-dimethyl-2-(1-methylcyclohexanecarbonyl)-7-(p-tolyl)-1,2,3,4-tetrahydroisoquinolin-6-yl)acetic Acid

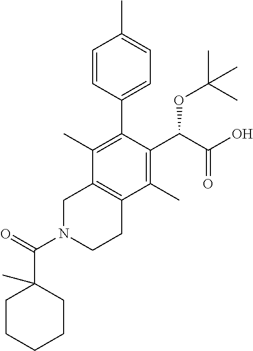 [SMILES: Cc1ccc(-c2c(C)c3c(c(C)c2[C@H](OC(C)(C)C)C(=O)O)CCN(C(=O)C2(C)CCCCC2)C3)cc1]

### Step 1: (S)-methyl 2-(tert-butoxy)-2-(5,8-dimethyl-2-(1-methylcyclohexanecarbonyl)-7-(p-tolyl)-1,2,3,4-tetrahydroisoquinolin-6-yl)acetate

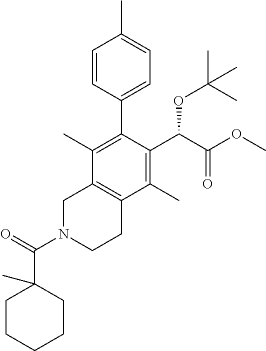

A solution of (S)-methyl 2-(tert-butoxy)-2-(5,8-dimethyl-7-(p-tolyl)-1,2,3,4-tetrahydroisoquinolin-6-yl)acetate (20 mg, 0.051 mmol) in N,N-Dimethylformamide (DMF) (1 mL) was treated with 1-methylcyclohexanecarboxylic acid (10.79 mg, 0.076 mmol), DIEA (0.018 mL, 0.101 mmol), HATU (23.07 mg, 0.061 mmol), and stirred at rt for 2 hours. The reaction was combined with another batch of crude material (0.051 mmol scale reaction), diluted with water, extracted with EtOAc, washed with water, Brine, dried over Na₂SO₄, filtered, and concentrated to give crude (S)-methyl 2-(tert-butoxy)-2-(5,8-dimethyl-2-(1-methylcyclohexanecarbonyl)-7-(p-tolyl)-1,2,3,4-tetrahydroisoquinolin-6-yl)acetate (72 mg, 0.139 mmol, 182% yield) as yellow oil. LCMS (m/z) ES⁺=520.5 (M+1). The residue was used without further purification.

### Step 2: (S)-2-(tert-butoxy)-2-(5,8-dimethyl-2-(1-methylcyclohexanecarbonyl)-7-(p-tolyl)-1,2,3,4-tetrahydroisoquinolin-6-yl)acetic Acid

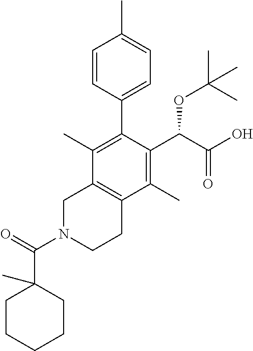 [SMILES: Cc1ccc(-c2c(C)c3c(c(C)c2[C@H](OC(C)(C)C)C(=O)O)CCN(C(=O)C2(C)CCCCC2)C3)cc1]

The title compound was made in a manner similar to that described in example 4 step 2 and isolated as a white solid after reverse phase HPLC. ¹H NMR (400 MHz, METHANOL-d₄) δ ppm 7.37-7.20 (m, 3H), 7.07 (d, J=7.1 Hz, 1H), 5.06 (s, 1H), 4.77-4.56 (m, 2H), 4.09-3.75 (m, 2H), 2.81 (t, J=5.7 Hz, 2H), 2.42 (s, 3H), 2.28 (s, 3H), 2.20-2.04 (m, 2H), 1.88 (s, 3H), 1.68-1.23 (m, 11H), 0.92 (s, 9H); LCMS (m/z) ES⁺=506.5 (M+1), ES⁻=504.5 (M−1).

### Example 77: (2S)-2-(tert-butoxy)-2-(5,8-dimethyl-2-(1,2,3,4-tetrahydronaphthalen-2-yl)-7-(p-tolyl)-1,2,3,4-tetrahydroisoquinolin-6-yl)acetic Acid

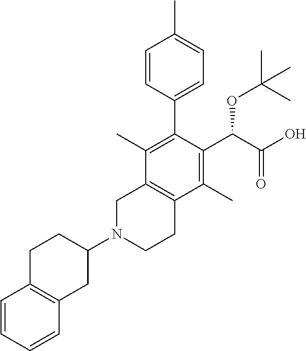 [SMILES: Cc1ccc(-c2c(C)c3c(c(C)c2[C@H](OC(C)(C)C)C(=O)O)CCN(C2CCc4ccccc4C2)C3)cc1]

The title compound was made in a manner similar to that described in example 49 from beta-tetralone and isolated as a white solid after reverse phase HPLC as a TFA salt.

¹H NMR (400 MHz, METHANOL-d₄) δ ppm 7.37-7.25 (m, 3H), 7.24-7.13 (m, 4H), 7.07 (br. s., 1H), 5.08 (s, 1H), 4.60-4.44 (m, 2H), 4.06-3.81 (m, 2H), 3.57-3.41 (m, 1H), 3.40-3.24 (m, 2H), 3.20-2.93 (m, 4H), 2.57-2.39 (m, 4H), 2.35 (s, 3H), 2.19-1.97 (m, 1H), 1.92 (s, 3H), 0.93 (br. s., 9H); LCMS (m/z) ES⁺=512.5 (M+1), ES⁻=510.4 (M−1).

### Example 78: (2S)-2-(tert-butoxy)-2-(5,8-dimethyl-2-(1,2,3,4-tetrahydronaphthalene-2-carbonyl)-7-(p-tolyl)-1,2,3,4-tetrahydroisoquinolin-6-yl)acetic Acid

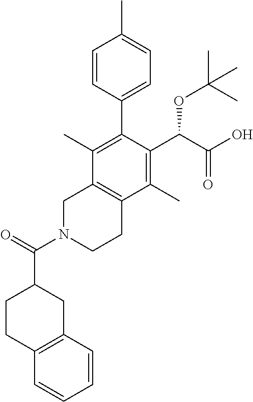 [SMILES: Cc1ccc(-c2c(C)c3c(c(C)c2[C@H](OC(C)(C)C)C(=O)O)CCN(C(=O)C2CCc4ccccc4C2)C3)cc1]

The title compound was made in a manner similar to that described in example 5 from 1,2,3,4-tetrahydronaphthalene-2-carboxylic acid and isolated as a white solid after reverse phase HPLC. ¹H NMR (400 MHz, METHANOL-d₄) δ ppm 7.37-7.21 (m, 3H), 7.15-6.92 (m, 5H), 5.07 (s, 1H), 4.78-4.57 (m, 2H), 4.01-3.79 (m, 2H), 3.28-3.15 (m, 1H), 3.08-2.75 (m, 6H), 2.42 (br. s., 3H), 2.30 (s, 3H), 2.15-1.78 (m, 5H), 0.93 (s, 9H); LCMS (m/z) ES⁺=540.5 (M+1), ES⁻=538.5 (M−1).

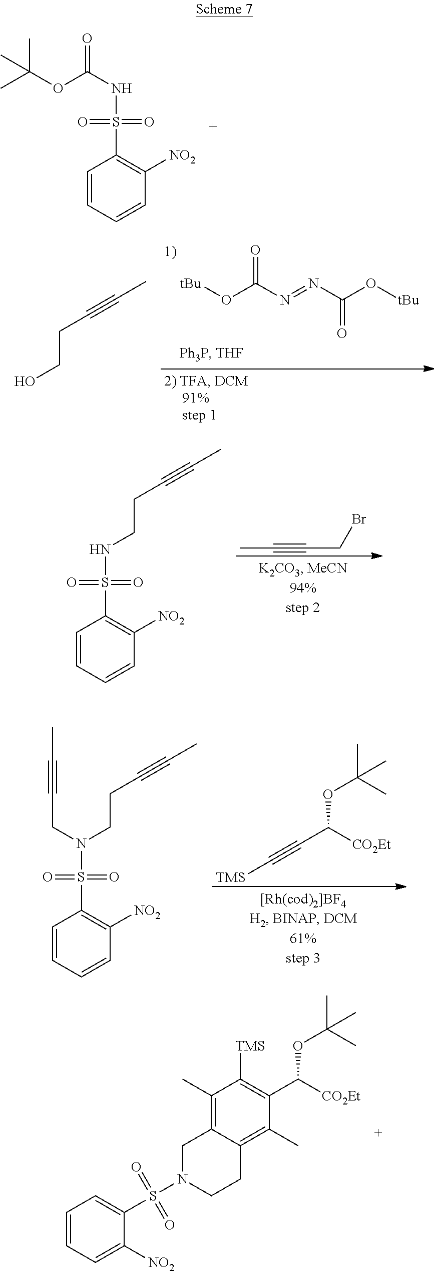

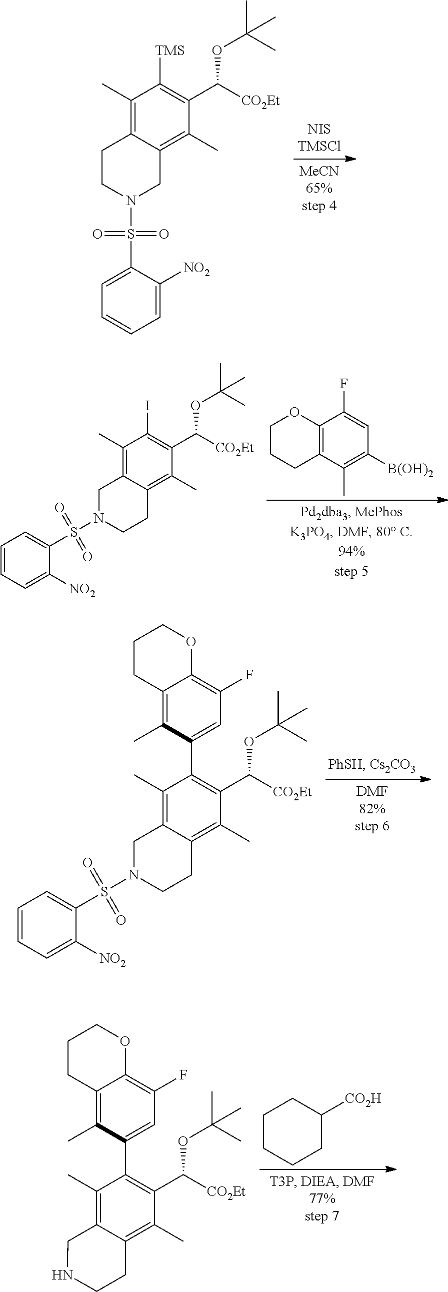

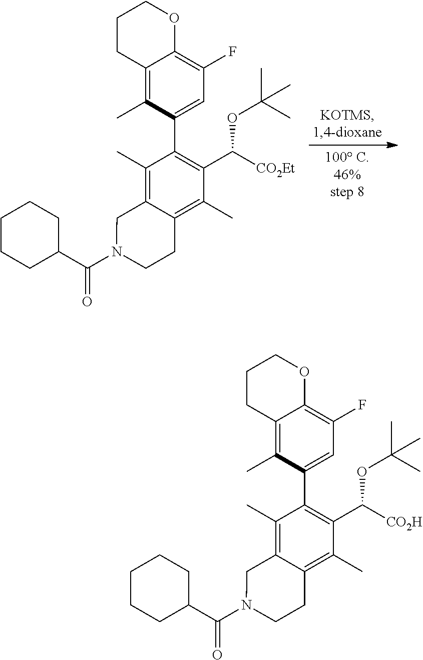

### Example 79: (S)-2-(tert-Butoxy)-2-((R)-2-(cyclohexanecarbonyl)-7-(8-fluoro-5-methylchroman-6-yl)-5,8-dimethyl-1,2,3,4-tetrahydroisoquinolin-6-yl)acetic Acid

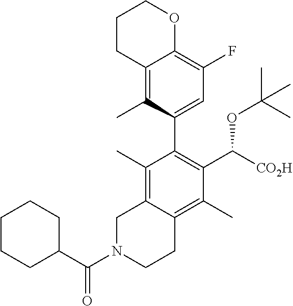

### Step 1: 2-Nitro-N-(pent-3-yn-1-yl)benzenesulfonamide

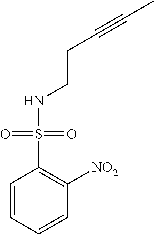 [SMILES: CC#CCCNS(=O)(=O)c1ccccc1[N+](=O)[O-]]

To a stirred suspension of tert-butyl (2-nitrophenyl)sulfonylcarbamate (21.0 g, 69.5 mmol), triphenylphosphine (21.9 g, 83.0 mmol), and pent-3-yn-1-ol (6.43 g, 76.0 mmol) in THF (107 mL) at 0° C. was slowly added di-tert-butylazodicarboxylate (19.2 g, 83.0 mmol) in small portions over 15 minutes. The yellow color slowly dissipated following each addition until the last addition, at which point the color persisted. The yellow solution was stirred at 0° C. for 15 minutes and then warmed to RT. Monitoring the reaction by LCMS for another 3 h indicated that the reaction had proceeded to about 90% and then stalled. The solution was cooled back to 0° C., treated with an additional 2.73 g (0.15 equiv) of triphenylphosphine, 0.86 g (0.15 equiv) of pent-3-yn-1-ol followed by 2.40 g (0.15 equiv) of di-tert-butyl diazene-1,2-dicarboxylate. After stirring at 0° C. for 10 minutes the solution was warmed to RT. After another 2 h the solution was concentrated to a syrup by rotary evaporation. Ether was added (about 300 mL) and the solution stirred which resulted in crystallization of the triphenylphosphine oxide by-product. After stirring for 20 min the solid was removed by filtration, washing twice with a small amount of ether. The filtrate was allowed to stand at RT overnight during which time additional triphenylphosphine oxide crystallized. The solid was filtered off and the filtrate concentrated at reduced pressure. The residue was redissolved in DCM and again concentrated to afford tert-butyl (2-nitrophenyl)sulfonyl(pent-3-yn-1-yl)carbamate as a pale yellow syrup (61.8 g). This material was dissolved in DCM (150 mL), the solution treated with TFA (150 mL), and stirred at RT. Vigorous gas evolution was observed. After 1.5 h the solution was concentrated at reduced pressure. The residue was redissolved in 250 mL of DCM and again concentrated to afford a yellow oil. This material was dissolved in EtOAc (300 mL). The solution was washed with water (2×150 mL), brine (1×150 mL), dried over Na₂SO₄, and concentrated at reduced pressure to give a pale yellow oil (51.2 g). A solid began to crystallize in this material after sitting for a brief time at RT. The mixture was triturated with addition of 1:1 ether/hexane (150 mL), which induced further crystallization. The suspension was stirred at 0° C. for 1 hour. The solid was collected by filtration, washing twice with cold 1:1 ether/hexane, and dried in vacuo to give the title compound (16.9 g, 91%) as a white solid. ¹H NMR (400 MHz, CDCl₃) δ 8.12-8.19 (m, 1H), 7.88-7.94 (m, 1H), 7.73-7.80 (m, 2H), 5.73 (br s, 1H), 3.22 (q, J=6.4 Hz, 2H), 2.32-2.40 (m, 2H), 1.73 (m, 3H). LCMS (ES+)(m/z): 269.15 (M+1).

### Step 2: N-(But-2-yn-1-yl)-2-nitro-N-(pent-3-yn-1-yl)benzenesulfonamide

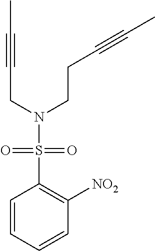 [SMILES: CC#CCCN(CC#CC)S(=O)(=O)c1ccccc1[N+](=O)[O-]]

To a stirred solution of 2-nitro-N-(pent-3-yn-1-yl)benzenesulfonamide (16.8 g, 62.6 mmol) and 1-bromobut-2-yne (5.48 mL, 62.6 mmol) in MeCN was added K₂CO₃ (17.3 g, 125 mmol). The resulting mixture was stirred at RT. After 5 h the mixture was treated with an additional portion of 1-bromobut-2-yne (1.10 mL, 12.5 mmol). After another 2 h the mixture was filtered to remove solids, washing with MeCN (3×). The filtrate was concentrated at reduced pressure and the residue dissolved in EtOAc (250 mL). The solution was washed with dilute brine (1×), sat. brine (1×), dried over Na₂SO₄, and concentrated at reduced pressure to afford a yellow syrup (22.0 g). This material was dissolved in ether (50 mL) during which time a white solid began to crystallize. The mixture was stirred and treated with hexane (75 mL) over 15 minutes. The resulting suspension was stirred at RT for 3 days. The solid was collected by vacuum filtration, washing with 3:2 hexane/ether. Drying in vacuo afforded the title compound (18.8 g, 94%) as an off-white solid. ¹H NMR (400 MHz, CDCl₃) δ 8.04-8.11 (m, 1H), 7.61-7.74 (m, 3H), 4.21 (d, J=2.0 Hz, 2H), 3.53 (t, J=7.3 Hz, 2H), 2.46 (m, 2H), 1.74 (m, 3H), 1.70 (s, 3H).

LCMS (ES+)(m/z): 321.19 (M+1).

### Step 3: (S)-Ethyl 2-(tert-butoxy)-2-(5,8-dimethyl-2-((2-nitrophenyl)sulfonyl)-7-(trimethylsilyl)-1,2,3,4-tetrahydroisoquinolin-6-yl)acetate and (S)-ethyl 2-(tert-butoxy)-2-(5,8-dimethyl-2-((2-nitrophenyl)sulfonyl)-6-(trimethylsilyl)-1,2,3,4-tetrahydroisoquinolin-7-yl)acetate

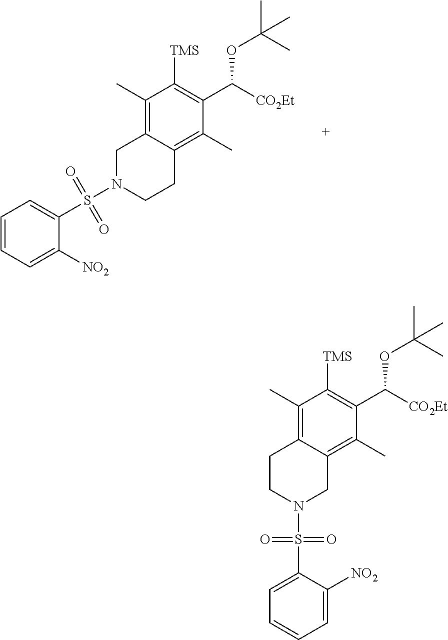 [SMILES: CCOC(=O)[C@@H](OC(C)(C)C)c1c(C)c2c(c(C)c1[Si](C)(C)C)CN(S(=O)(=O)c1ccccc1[N+](=O)[O-])CC2 ; CCOC(=O)[C@@H](OC(C)(C)C)c1c(C)c2c(c(C)c1[Si](C)(C)C)CCN(S(=O)(=O)c1ccccc1[N+](=O)[O-])C2]

A suspension of [Rh(cod)₂]BF₄ (0.950 g, 2.34 mmol) and BINAP (1.46 g, 2.34 mmol) in anhydrous DCM (75 mL) was sparged with H₂ for 5 minutes and stirred under 1 atm (balloon) of H₂. After 1 hour the solution was concentrated to dryness. The residue was dissolved in DCM (50 mL) and the solution treated with a solution of (S)-ethyl 2-(tert-butoxy)-4-(trimethylsilyl)but-3-ynoate (3.00 g, 11.7 mmol) in DCM (50 mL). The resulting solution was heated to 40° C. under a nitrogen atmosphere and was treated with a solution of N-(but-2-yn-1-yl)-2-nitro-N-(pent-3-yn-1-yl)benzenesulfonamide (7.50 g, 23.4 mmol) in 75 mL of DCM via addition funnel over a 2 h period. The solution was then stirred at 40° C. for an additional 1 h, and cooled to RT. The solution was concentrated to dryness at reduced pressure and the residue subjected to flash chromatography (silica gel, 0-50% EtOAc/hexanes, gradient elution) to afford the titles compounds (4.12 g, 61%) as a white foam as a mixture of regioisomers (as determined by LCMS) that was carried to the next step without separation. LCMS (ES+)(m/z): 577.46 (M+1).

### Step 4: (S)-Ethyl 2-(tert-butoxy)-2-(7-iodo-5,8-dimethyl-2-((2-nitrophenyl)sulfonyl)-1,2,3,4-tetrahydroisoquinolin-6-yl)acetate

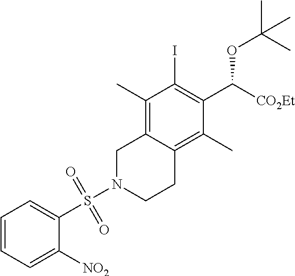 [SMILES: CCOC(=O)[C@@H](OC(C)(C)C)c1c(C)c2c(c(C)c1I)CN(S(=O)(=O)c1ccccc1[N+](=O)[O-])CC2]

To a stirred solution of (S)-ethyl 2-(tert-butoxy)-2-(5,8-dimethyl-2-((2-nitrophenyl)sulfonyl)-7-(trimethylsilyl)-1,2,3,4-tetrahydroisoquinolin-6-yl)acetate and (S)-ethyl 2-(tert-butoxy)-2-(5,8-dimethyl-2-((2-nitrophenyl)sulfonyl)-6-(trimethylsilyl)-1,2,3,4-tetrahydroisoquinolin-7-yl)acetate (4.12 g, 7.14 mmol) in MeCN (71 mL) was added N-iodosuccinimide (1.93 g, 8.57 mmol) followed by TMS-Cl (91 μL, 0.714 mmol). After stirring at RT for 40 min, the solution was partitioned between EtOAc and aqueous NaHCO₃, and the phases separated. The aqueous phase was extracted with one additional portion of EtOAc. The combined EtOAc solutions were washed with aqueous sodium thiosulfate (1×), sat. aqueous NaHCO₃ (2×), sat. brine (1×), dried over Na₂SO₄, and concentrated at reduced pressure to give a pale yellow foam (4.47 g). The desired regioisomer (lower Rf) was isolated by flash chromatography (silica gel, 0-100% (5:4:1 hex/DCM/EtOAc)/hexane, gradient elution). This afforded the title compound (2.91 g, 65%) as a white solid. ¹H NMR (400 MHz, CDCl₃) δ 8.03-8.09 (m, 1H), 7.61-7.77 (m, 3H), 5.92 (s, 1H), 4.42 (s, 2H), 4.04-4.26 (m, 2H), 3.52-3.66 (m, 2H), 2.74-2.85 (m, 2H), 2.46 (s, 3H), 2.26 (s, 3H), 1.22 (s, 12H). LCMS (ES+)(m/z): 631.28 (M+1).

### Step 5: (S)-Ethyl 2-(tert-butoxy)-2-((R)-7-(8-fluoro-5-methylchroman-6-yl)-5,8-dimethyl-2-((2-nitrophenyl)sulfonyl)-1,2,3,4-tetrahydroisoquinolin-6-yl)acetate

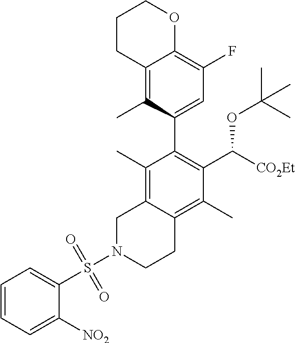

A mixture of (S)-ethyl 2-(tert-butoxy)-2-(7-iodo-5,8-dimethyl-2-((2-nitrophenyl)sulfonyl)-1,2,3,4-tetrahydroisoquinolin-6-yl)acetate (0.500 g, 0.793 mmol), (8-fluoro-5-methylchroman-6-yl)boronic acid (0.333 g, 1.59 mmol), K₃PO₄ (0.505 g, 2.38 mmol), and MePhos (58 mg, 0.159 mmol) in DMF (8 mL) in a sealed tube was sparged with nitrogen for 5 min and treated with Pd(dba)₂ (91 mg, 0.159 mmol). The vessel was capped and the mixture heated to 80° C. with stirring. After 2 hours the mixture was cooled to RT. The mixture was diluted with EtOAc and the solids removed by filtration through celite, washing with excess EtOAc. The filtrate was washed with dilute brine (2×), sat. brine (1×), dried over Na₂SO₄, and concentrated at reduced pressure. The residue was purified by flash chromatography (silica gel, 0-40% EtOAc/hexanes, gradient elution) to give the title compound (0.50 g, 94%) as a tan foam. ¹H NMR (400 MHz, CDCl₃) δ 8.02-8.09 (m, 1H), 7.68-7.76 (m, 2H), 7.61-7.67 (m, 1H), 6.63 (d, J=11.2 Hz, 1H), 4.94 (s, 1H), 3.98-4.50 (m, 6H), 3.68-3.77 (m, 1H), 3.48-3.59 (m, 1H), 2.88 (t, J=5.68 Hz, 2H), 2.72 (t, J=6.4 Hz, 2H), 2.34 (s, 3H), 2.10-2.19 (m, 2H), 1.78 (s, 3H), 1.76 (s, 3H), 1.14 (t, J=7.1 Hz, 3H), 1.09 (s, 9H). LCMS (ES+)(m/z): 669.56 (M+1).

### Step 6: (S)-Ethyl 2-(tert-butoxy)-2-((R)-7-(8-fluoro-5-methylchroman-6-yl)-5,8-dimethyl-1,2,3,4-tetrahydroisoquinolin-6-yl)acetate

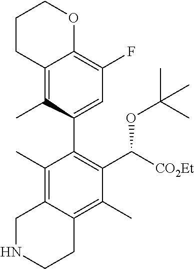

To a stirred solution of (S)-ethyl 2-(tert-butoxy)-2-((R)-7-(8-fluoro-5-methylchroman-6-yl)-5,8-dimethyl-2-((2-nitrophenyl)sulfonyl)-1,2,3,4-tetrahydroisoquinolin-6-yl)acetate (0.876 g, 1.31 mmol) in DMF (8 mL) was added Cs₂CO₃ (1.71 g, 5.24 mmol) followed by thiophenol (0.54 mL, 5.24 mmol). After 1 h the mixture was partitioned between water and EtOAc and the phases separated. The aqueous phase was extracted with one additional portion of EtOAc. The combined EtOAc solutions were washed with dilute brine (2×), sat. brine (1×), dried over Na₂SO₄ and concentrated at reduced pressure. The resulting yellow oil was subjected to flash chromatography (silica gel, 0-10% 2M NH₃ in MeOH/DCM, gradient elution) to give the title compound (0.520 g, 82%) as a white foam. ¹H NMR (400 MHz, CDCl₃) δ 6.67 (d, J=11.4 Hz, 1H), 4.94 (s, 1H), 4.28 (t, J=5.0 Hz, 2H), 3.98-4.12 (m, 2H), 3.89-3.95 (m, 2H), 3.05-3.26 (m, 2H), 2.62-2.77 (m, 4H), 2.34 (s, 3H), 2.08-2.21 (m, 2H), 1.80 (s, 3H), 1.69 (s, 3H), 1.14 (t, J=7.1 Hz, 3H), 1.09 (s, 9H). LCMS (ES+)(m/z): 484.49 (M+1).

### Step 7: (S)-Ethyl 2-(tert-butoxy)-2-((R)-2-(cyclohexanecarbonyl)-7-(8-fluoro-5-methylchroman-6-yl)-5,8-dimethyl-1,2,3,4-tetrahydroisoquinolin-6-yl)acetate

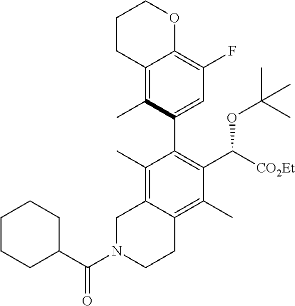

To a stirred solution of (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate (50 mg, 0.103 mmol), cyclohexanecarboxylic acid (20 mg, 0.155 mmol), and DIEA (54 μL, 0.310 mmol) in anhydrous DMF (4 mL) at 0° C. was added 50% T3P/EtOAc (0.13 mL, 0.207 mmol) by dropwise addition. After 2 h the solution was diluted with EtOAc. The resulting solution was washed with 10% aq. citric acid/brine (2×), sat. aq. NaHCO₃ (2×), dried over Na₂SO₄ and concentrated to dryness at reduced pressure. Purification by flash chromatography (silica gel, 0-100% EtOAc/hexanes, gradient elution) gave the title compound (47 mg, 77%) as a white foam. ¹H NMR (400 MHz, CDCl₃, mixture of rotamers) δ 6.60-6.71 (m, 1H), 4.96 (s, 1H), 3.62-4.85 (m, 8H), 2.52-2.91 (m, 5H), 2.32-2.41 (m, 3H), 2.10-2.20 (m, 2H), 1.49-1.90 (m, 13H), 1.04-1.40 (m, 15H). LCMS (ES+)(m/z): 594.59 (M+1).

### Step 8: (S)-2-(tert-Butoxy)-2-((R)-2-(cyclohexanecarbonyl)-7-(8-fluoro-5-methylchroman-6-yl)-5,8-dimethyl-1,2,3,4-tetrahydroisoquinolin-6-yl)acetic Acid

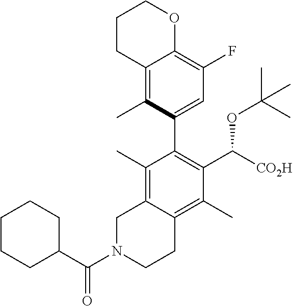

To a stirred solution of (S)-ethyl 2-(tert-butoxy)-2-((R)-2-(cyclohexanecarbonyl)-7-(8-fluoro-5-methylchroman-6-yl)-5,8-dimethyl-1,2,3,4-tetrahydroisoquinolin-6-yl)acetate (45 mg, 0.076 mmol) in 1,4-dioxane (3 mL) was added KOTMS (49 mg, 0.379 mmol) and the resulting mixture was heated to 90° C. Reaction progress was monitored by LCMS. After 30 min the temperature was increased to 100° C. After 1 hour LCMS indicated little or no conversion. The mixture was treated with an additional 30 mg of KOTMS and stirring at 100° C. continued. After another 1.5 h the mixture was cooled to RT, partitioned between DCM and 0.5M aqueous HCl, and the phases separated. The aqueous phase was extracted with DCM (2×). The combined DCM extracts were combined and concentrated at reduced pressure. The residue was subjected to RP-HPLC purification (C18, 10-100% MeCN/H₂O with 0.1% formic acid) to afford the title compound (19.5 mg, 46%) as a white solid. ¹H NMR (400 MHz, CDCl₃, mixture of rotamers) δ 6.62-6.77 (m, 1H), 5.09 (br s, 1H), 4.47-4.70 (m, 2H), 4.22-4.32 (m, 2H), 3.68-3.93 (m, 2H), 2.50-2.93 (m, 5H), 2.26 (br s, 3H), 2.07-2.18 (m, 2H), 1.46-1.93 (m, 14H), 1.22-1.41 (m, 2H) 1.12 (s, 9H).

LCMS (ES+)(m/z): 588.55 (M+Na).

### Example 80: (S)-2-(tert-Butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetic Acid

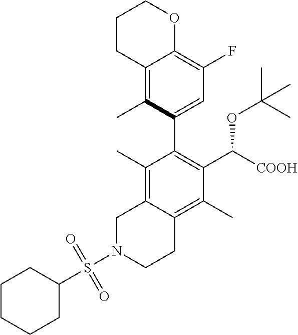

### Step 1: (S)-Ethyl 2-(tert-butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetate

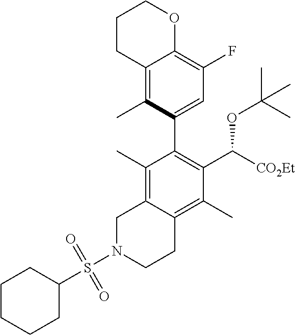

To a stirred solution of (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate (50 mg, 0.103 mmol) and DIEA (54 μL, 0.310 mmol) in anhydrous DCM (4 mL) at 0° C. was added a solution of cyclohexanesulfonyl chloride (28.3 mg, 0.155 mmol) in DCM by dropwise addition. After 2 hours the solution was diluted with EtOAc. The resulting solution was washed with 10% aq. citric acid/brine (2×), sat. aq. NaHCO₃ (2×), dried over Na₂SO₄ and concentrated to dryness at reduced pressure. Purification by flash chromatography (silica gel, 0-50% EtOAc/hexanes, gradient elution) afforded the title compound (34 mg, 52%) as a white foam. ¹H NMR (400 MHz, CDCl₃) δ 6.65 (d, J=11.4 Hz, 1H), 4.96 (s, 1H), 4.22-4.49 (m, 4H), 3.96-4.15 (m, 2H), 3.51-3.74 (m, 2H), 2.95-3.07 (m, 1H), 2.84 (t, J=5.6 Hz, 2H), 2.72 (t, J=6.3 Hz, 2H), 2.36 (s, 3H), 2.08-2.22 (m, 4H), 1.85-1.94 (m, 2H), 1.50-1.66 (m, 10H), 1.04-1.38 (m, 14H). LCMS (ES+)(m/z): 630.69 (M+1).

### Step 2: (S)-2-(tert-Butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetic Acid

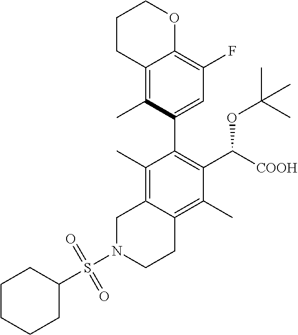

The title compound was prepared in 72% yield from (S)-ethyl 2-(tert-butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetate according to example 79, step 8. ¹H NMR (400 MHz, CDCl₃) δ 6.69 (d, J=11.2 Hz, 1H), 5.10 (br s, 1H), 4.19-4.51 (m, 5H), 3.57-3.72 (m, 2H), 2.95-3.08 (m, 1H), 2.81-2.91 (m, 2H), 2.60-2.78 (m, 2H), 2.07-2.34 (m, 6H), 1.48-1.98 (m, 12H), 1.06-1.41 (m, 11H). LCMS (ES+)(m/z): 602.50 (M+1).

### Example 81: (S)-2-(tert-Butoxy)-2-((R)-2-(4,4-dimethylcyclohexyl)-7-(8-fluoro-5-methylchroman-6-yl)-5,8-dimethyl-1,2,3,4-tetrahydroisoquinolin-6-yl)acetic Acid

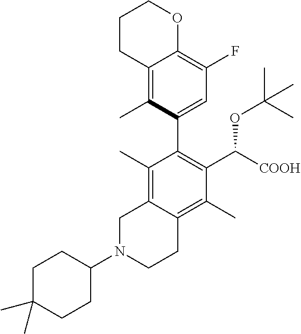

### Step 1: (S)-Ethyl 2-(tert-butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetate

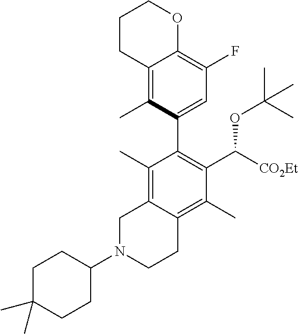

To a stirred solution of (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate (50 mg, 0.103 mmol) in DCE (4 mL) was added a solution of 4,4-dimethylcyclohexanone (26 mg, 0.207 mmol) in DCE (1 mL). After 15 min the solution was treated with NaBH(OAc)₃ (66 mg, 0.310 mmol) and the resulting mixture stirred at RT. After 18 h the solution was diluted with 10% aqueous Na₂CO₃ followed by DCM. The bi-phasic mixture was stirred vigorously for 20 min and the phases separated. The aqueous solution was extracted with DCM (2×). The combined DCM solutions were dried over Na₂SO₄ and concentrated at reduced pressure. The residue was subjected to flash chromatography (silica gel, 0-10% 2M NH₃ in MeOH/DCM) to afford the title compound (55 mg, 90%) as a white foam. ¹H NMR (400 MHz, CDCl₃) δ 6.66 (d, J=11.4 Hz, 1H), 4.92 (s, 1H), 4.28 (t, J=5.0 Hz, 2H), 3.92-4.09 (m, 2H), 3.77 (d, J=15.2 Hz, 1H), 3.59 (d, J=15.2 Hz, 1H), 2.98-3.09 (m, 1H), 2.58-2.88 (m, 5H), 2.31-2.49 (m, 4H), 2.07-2.21 (m, 2H), 1.43-1.91 (m, 12H), 1.21-1.34 (m, 2H), 1.05-1.17 (m, 12H), 0.90-0.98 (m, 6H). LCMS (ES+)(m/z): 594.64 (M+1).

### Step 2: (S)-2-(tert-Butoxy)-2-((R)-2-(4,4-dimethylcyclohexyl)-7-(8-fluoro-5-methylchroman-6-yl)-5,8-dimethyl-1,2,3,4-tetrahydroisoquinolin-6-yl)acetic Acid

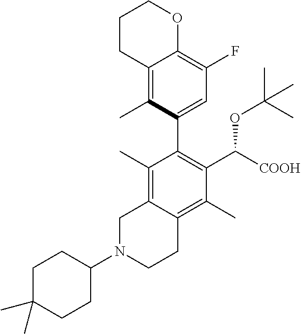

The title compound was prepared as the formate salt in 31% yield from (S)-ethyl 2-(tert-butoxy)-2-((R)-2-(cyclohexylsulfonyl)-7-(8-fluoro-5-methylchroman-6-yl)-5,8-dimethyl-1,2,3,4-tetrahydroisoquinolin-6-yl)acetate according to example 79, step 8. ¹H NMR (400 MHz, CDCl₃) δ 8.33 (s, 1H, formate proton), 6.63 (d, J=11.4 Hz, 1H), 4.79 (br s, 1H), 4.21-4.32 (m, 2H), 4.00 (d, J=15.0 Hz, 1H), 3.65 (d, J=14.5 Hz, 1H), 3.48-3.57 (m, 1H), 2.55-3.13 (m, 5H), 2.33 (s, 3H), 2.07-2.18 (m, 2H), 1.84-2.00 (m, 2H), 1.44-1.78 (m, 9H), 1.19-1.35 (m, 2H), 1.06-1.14 (2, 9H), 0.85-0.98 (m, 6H). LCMS (ES+)(m/z): 566.57 (M+1).

### Example 82: (S)-2-(tert-Butoxy)-2-((R)-2-(3-fluoro-2-methoxybenzoyl)-7-(8-fluoro-5-methylchroman-6-yl)-5,8-dimethyl-1,2,3,4-tetrahydroisoquinolin-6-yl)acetic Acid

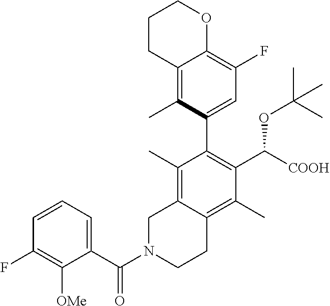

### Step 1: (S)-Ethyl 2-(tert-butoxy)-2-((R)-2-(3-fluoro-2-methoxybenzoyl)-7-(8-fluoro-5-methylchroman-6-yl)-5,8-dimethyl-1,2,3,4-tetrahydroisoquinolin-6-yl)acetate

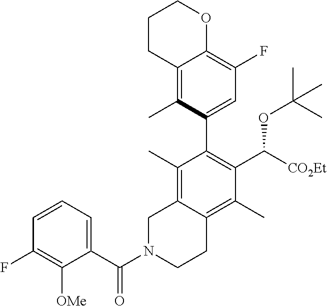

The title compound was prepared in 81% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and 3-fluoro-2-methoxybenzoic acid according to example 79, step 7. ¹H NMR (400 MHz, CDCl₃, mixture of rotamers) δ 6.97-7.22 (m, 3H), 6.54-6.74 (m, 1H), 4.56-5.12 (m, 2H), 3.39-4.41 (m, 9H), 2.59-2.98 (m, 4H), 2.28-2.44 (m, 3H), 2.07-2.22 (m, 2H), 1.76-1.91 (m, 4H), 1.44-1.63 (m, 3H), 1.02-1.33 (m, 12H). LCMS (ES+)(m/z): 636.56 (M+1).

### Step 2: (S)-2-(tert-Butoxy)-2-((R)-2-(3-fluoro-2-methoxybenzoyl)-7-(8-fluoro-5-methylchroman-6-yl)-5,8-dimethyl-1,2,3,4-tetrahydroisoquinolin-6-yl)acetic Acid

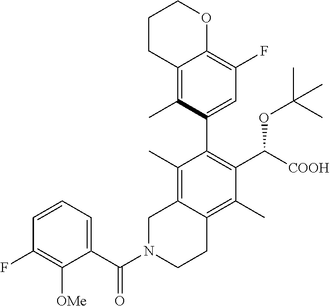

The title compound was prepared in 31% yield from (S)-ethyl 2-(tert-butoxy)-2-((R)-2-(3-fluoro-2-methoxybenzoyl)-7-(8-fluoro-5-methylchroman-6-yl)-5,8-dimethyl-1,2,3,4-tetrahydroisoquinolin-6-yl)acetate according to example 79, step 8. ¹H NMR (400 MHz, CDCl₃, mixture of rotamers) δ 6.96-7.23 (m, 3H), 6.59-6.77 (m, 1H), 4.65-5.20 (m, 2H), 3.44-4.44 (m, 8H), 2.58-2.98 (m, 4H), 2.05-2.36 (m, 5H), 1.76-1.95 (m, 6H), 1.05-1.18 (m, 9H). LCMS (ES+)(m/z): 608.54 (M+1).

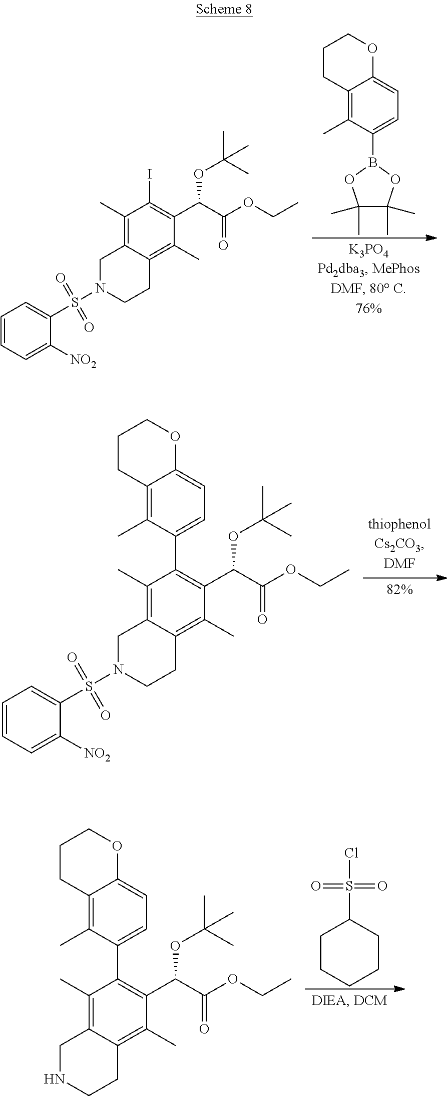

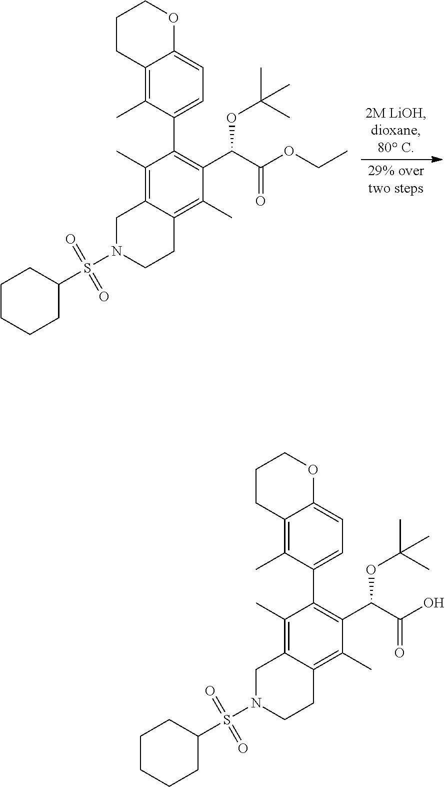 [SMILES: CCOC(=O)[C@@H](OC(C)(C)C)c1c(C)c2c(c(C)c1-c1ccc3c(c1C)CCCO3)CN(S(=O)(=O)C1CCCCC1)CC2 ; Cc1c(-c2c(C)c3c(c(C)c2[C@H](OC(C)(C)C)C(=O)O)CCN(S(=O)(=O)C2CCCCC2)C3)ccc2c1CCCO2]

### Example 83: (2S)(M)-2-(tert-butoxy)-2-(-2-(cyclohexylsulfonyl)-5,8-dimethyl-7-(5-methylchroman-6-yl)-1,2,3,4-tetrahydroisoquinolin-6-yl)acetic Acid

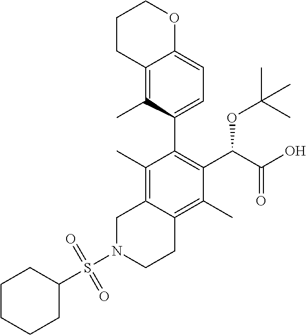

### Step 1: (S)-ethyl 2-(tert-butoxy)-2-((R)-5,8-dimethyl-7-(5-methylchroman-6-yl)-2-((2-nitrophenyl)sulfonyl)-1,2,3,4-tetrahydroisoquinolin-6-yl)acetate

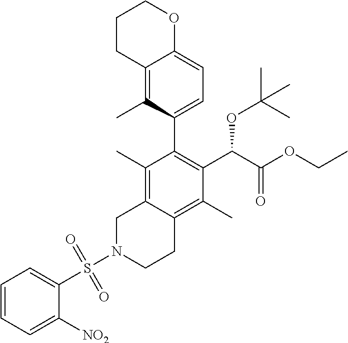

A degassed mixture of (S)-ethyl 2-(tert-butoxy)-2-(7-iodo-5,8-dimethyl-2-((2-nitrophenyl)sulfonyl)-1,2,3,4-tetrahydroisoquinolin-6-yl)acetate (150 mg, 0.238 mmol) in N,N-dimethylformamide (2.5 mL) was degassed while adding 4,4,5,5-tetramethyl-2-(5-methylchroman-6-yl)-1,3,2-dioxaborolane (130 mg, 0.476 mmol), K₃PO₄ (152 mg, 0.714 mmol), MePhos (17.34 mg, 0.048 mmol) and finally Pd₂dba₃ (43.6 mg, 0.048 mmol). The vial containing the mixture was capped and then immersed into an 80° C. oil bath and stirred for 150 minutes. The mixture was cooled to ambient temperature, diluted with EtOAc, washed with water, then brine, dried over sodium sulfate, filtered and concentrated under reduced pressure. The residue was purified on silica gel (0-30% hexanes/EtOAc) to afford the title compound as an off-white colored foam (0.118 g, 76%). 1H NMR indicated a 9:1 mixture of diastereomers. LC/MS (m/z) ES⁺=673 (M+23).

### Step 2: (S)-ethyl 2-(tert-butoxy)-2-((R)-5,8-dimethyl-7-(5-methylchroman-6-yl)-1,2,3,4-tetrahydroisoquinolin-6-yl)acetate

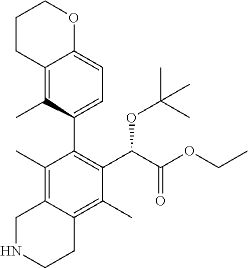

A mixture of (S)-ethyl 2-(tert-butoxy)-2-((R)-5,8-dimethyl-7-(5-methylchroman-6-yl)-2-((2-nitrophenyl)sulfonyl)-1,2,3,4-tetrahydroisoquinolin-6-yl)acetate (118 mg, 0.181 mmol) and cesium carbonate (236 mg, 0.725 mmol) in N,N-dimethylformamide (4.0 mL) was treated with thiophenol (0.075 mL, 0.725 mmol) and the mixture was stirred at ambient temperature until LCMS indicated the reaction to be complete (10 minutes). Water was added and the mixture was extracted with EtOAc. The extracts were washed with brine, dried over sodium sulfate, filtered and concentrated. The residue was purified on silica gel (12 g column, 0-10% DCM/MeOH) to afford a white foam (69 mg, 82%). LC/MS (m/z) ES⁺=466 (M+1).

### Step 3: (S)-ethyl 2-(tert-butoxy)-2-((R)-2-(cyclohexylsulfonyl)-5,8-dimethyl-7-(5-methylchroman-6-yl)-1,2,3,4-tetrahydroisoquinolin-6-yl)acetate

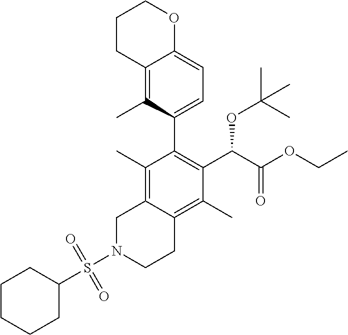

A solution (S)-ethyl 2-(tert-butoxy)-2-((R)-5,8-dimethyl-7-(5-methylchroman-6-yl)-1,2,3,4-tetrahydroisoquinolin-6-yl)acetate (40.0 mg, 0.086 mmol) in dichloromethane (0.8 mL) was treated with DIEA (0.045 mL, 0.258 mmol), followed by cyclohexanesulfonyl chloride (0.020 mL, 0.137 mmol) and the mixture was allowed to stir at ambient temperature overnight. The mixture was diluted with dichloromethane, washed with saturated sodium bicarbonate, followed by brine. The organic phase was then dried over sodium sulfate, filtered and concentrated under reduced pressure to afford a pale brown residue (75 mg) that was used in the next step without further purification. LC/MS (m/z) ES⁺=634 (M+23).

### Step 4: (2S)(M)-2-(tert-butoxy)-2-(-2-(cyclohexylsulfonyl)-5,8-dimethyl-7-(5-methylchroman-6-yl)-1,2,3,4-tetrahydroisoquinolin-6-yl)acetic Acid

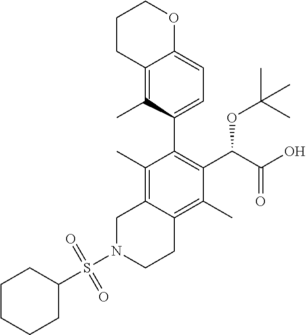

A solution of the crude (S)-ethyl 2-(tert-butoxy)-2-((R)-2-(cyclohexylsulfonyl)-5,8-dimethyl-7-(5-methylchroman-6-yl)-1,2,3,4-tetrahydroisoquinolin-6-yl)acetate (75 mg) in 1,4-dioxane (1.0 mL) was treated with potassium trimethylsilanolate (49.0 mg, 0.344 mmol) and the flask containing the mixture was immersed into a 90° C. oil bath and then stirred for 20 minutes. LCMS indicated no reaction had occurred. Additional potassium trimethylsilanolate (49.0 mg, 0.344 mmol) was added and stirring at 90° C. was continued for 25 minutes. The temperature was increased to 100° C. and stirred for 70 minutes. Additional potassium trimethylsilanolate (49.0 mg, 0.344 mmol) was added and stirring at 100° C. was continued for 50 minutes. LCMS indicated that no desired product was formed. The mixture was cooled to ambient temperature. 1N HCl was added and the mixture was extracted with EtOAc. The extracts were washed with brine, dried over sodium sulfate, filtered and concentrated. The residue was purified by reverse phase HPLC to afford a colorless residue. The residue was dissolved in 1,4-dioxane (0.8 mL) and then treated with 2M LiOH (0.180 mL) and the mixture was stirred in a sealed vial at 65° C. overnight. LCMS indicated ˜30% conversion to the desired product. Stirring was continued at 65° C. overnight. LCMS indicated ˜40% conversion to the desired product. Additional 2M LiOH (0.180 mL) and dioxane (0.5 mL) were added and the temperature was increased to 80° C. The mixture was stirred at 80° C. for 4 days and then concentrated under reduced pressure. 1N HCl was added to the resulting residue and then extracted with EtOAc. The extracts were washed with brine, dried over sodium sulfate, filtered and concentrated. The residue was purified by reverse phase HPLC to afford the title compound as a white solid (14 mg, 29% over 2 steps). ¹H NMR (400 MHz, CHLOROFORM-d) δ=6.88-6.66 (m, 2H), 5.15 (m, 1H), 4.53-4.27 (m, 2H), 4.19 (t, J=4.9 Hz, 2H), 3.78-3.52 (m, 2H), 3.12-2.57 (m, 5H), 2.37-2.00 (m, 7H), 1.96-1.82 (m, 5H), 1.67-1.50 (m, 5H), 1.37-1.19 (m, 4H), 1.15-0.95 (m, 9H); LC/MS (m/z) ES⁺=584 (M+1).

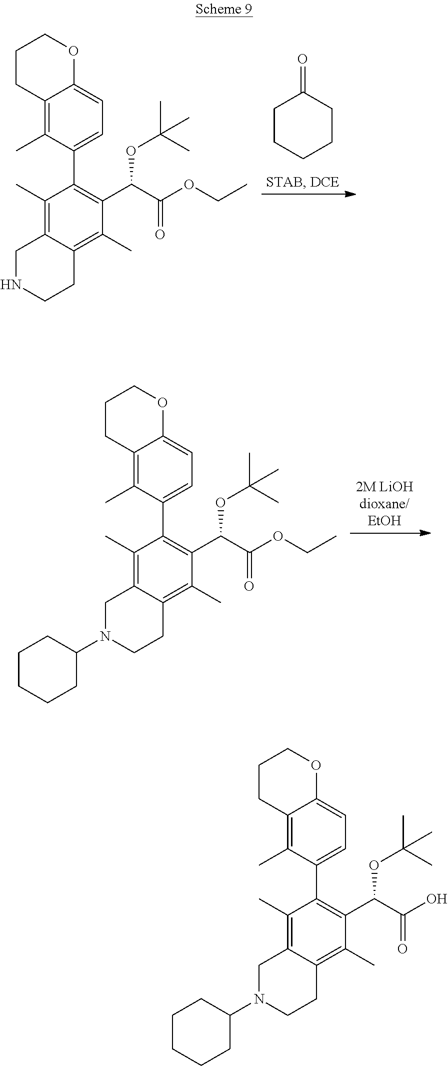 [SMILES: CCOC(=O)[C@@H](OC(C)(C)C)c1c(C)c2c(c(C)c1-c1ccc3c(c1C)CCCO3)CNCC2 ; O=C1CCCCC1 ; CCOC(=O)[C@@H](OC(C)(C)C)c1c(C)c2c(c(C)c1-c1ccc3c(c1C)CCCO3)CN(C1CCCCC1)CC2 ; Cc1c(-c2c(C)c3c(c(C)c2[C@H](OC(C)(C)C)C(=O)O)CCN(C2CCCCC2)C3)ccc2c1CCCO2]

### Example 84: (S)(M)-2-(tert-butoxy)-2-(2-cyclohexyl-5,8-dimethyl-7-(5-methylchroman-6-yl)-1,2,3,4-tetrahydroisoquinolin-6-yl)acetic Acid

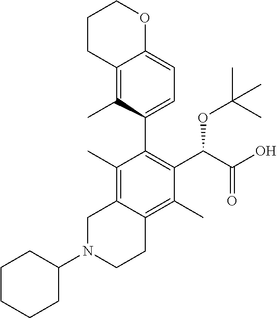

### Step 1: (S)-ethyl 2-(tert-butoxy)-2-((R)-2-cyclohexyl-5,8-dimethyl-7-(5-methylchroman-6-yl)-1,2,3,4-tetrahydroisoquinolin-6-yl)acetate

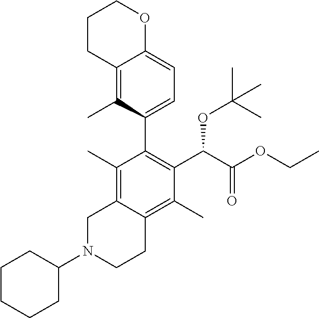

A solution of (2S)-ethyl 2-(tert-butoxy)-2-(5,8-dimethyl-7-(5-methylchroman-6-yl)-1,2,3,4-tetrahydroisoquinolin-6-yl)acetate (34.0 mg, 0.073 mmol) in 1,2-dichloroethane (0.8 mL) was treated with cyclohexanone (10.59 μl, 0.102 mmol), followed by sodium triacetoxyborohydride (31.0 mg, 0.146 mmol) and the mixture was allowed to stir at ambient temperature overnight. The mixture was diluted with EtOAc, washed with saturated sodium bicarbonate, followed by brine, dried over sodium sulfate, filtered and concentrated under reduced pressure to a colorless residue (48 mg) which was used in the next step without further purification.

### Step 2: (S)(M)-2-(tert-butoxy)-2-(2-cyclohexyl-5,8-dimethyl-7-(5-methylchroman-6-yl)-1,2,3,4-tetrahydroisoquinolin-6-yl)acetic Acid

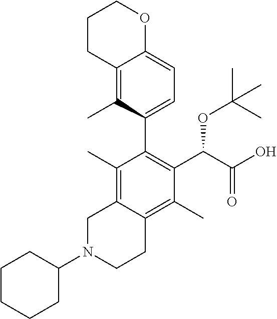

A solution of the crude (S)-ethyl 2-(tert-butoxy)-2-((R)-2-cyclohexyl-5,8-dimethyl-7-(5-methylchroman-6-yl)-1,2,3,4-tetrahydroisoquinolin-6-yl)acetate (48 mg) in 1,4-dioxane (1.0 mL) was treated with potassium trimethylsilanolate (41.6 mg, 0.292 mmol) and the flask containing the mixture was immersed into a 90° C. oil bath and allowed to stir for 25 minutes. LCMS indicated little to no reaction. Additional potassium trimethylsilanolate (41.6 mg, 0.292 mmol) was added and stirring at 90° C. was continued for 20 minutes. The temperature was increased to 100° C. and stirred for 70 minutes. Additional potassium trimethylsilanolate (41.6 mg, 0.292 mmol) was added and stirring at 100° C. was continued for 50 minutes. LCMS indicated that little to no desired product was formed. The mixture was cooled to ambient temperature. 1N HCl was added and the mixture was extracted with EtOAc. The extracts were washed with brine, dried over sodium sulfate, filtered and concentrated under reduced pressure. The residue was purified by reverse phase HPLC to afford a colorless residue. A solution of the purified product in 1,4-dioxane (0.6 mL) and ethanol (0.2 mL) was treated with 2M LiOH (0.18 mL, 0.360 mmol) and then stirred in a sealed vial at 90° C. overnight. LCMS indicated >80% conversion to the desired product. Stirring at 90° C. was continued overnight. The mixture was concentrated. 1N HCl was added to the resulting residue and then extracted with EtOAc. The extracts were washed with brine, dried over sodium sulfate, filtered and concentrated. The residue was purified by reverse phase HPLC to afford the title compound as a pale yellow solid (6 mg). ¹H NMR (400 MHz, CHLOROFORM-d) δ=6.78 (d, J=8.2 Hz, 1H), 6.65 (d, J=8.2 Hz, 1H), 4.79 (br. s., 1H), 4.25-4.12 (m, 2H), 3.83 (d, J=14.7 Hz, 1H), 3.59-3.33 (m, 1H), 3.13-2.97 (m, 1H), 2.85-2.56 (m, 5H), 2.39-2.27 (m, 3H), 2.18-1.97 (m, 4H), 1.91-1.65 (m, 4H), 1.63-1.50 (m, 5H), 1.47-1.23 (m, 5H), 1.20-1.04 (m, 10H); LC/MS (m/z) ES⁺=520 (M+1).

### Example 85: (S)-2-(tert-Butoxy)-2-((R)-2-(cyclohexylmethyl)-7-(8-fluoro-5-methylchroman-6-yl)-5,8-dimethyl-1,2,3,4-tetrahydroisoquinolin-6-yl)acetic Acid

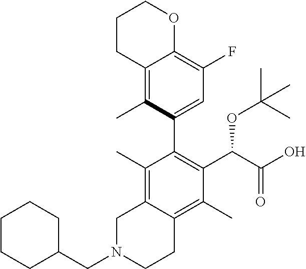

### Step 1: (S)-Ethyl 2-(tert-butoxy)-2-((R)-2-(cyclohexylmethyl)-7-(8-fluoro-5-methylchroman-6-yl)-5,8-dimethyl-1,2,3,4-tetrahydroisoquinolin-6-yl)acetate

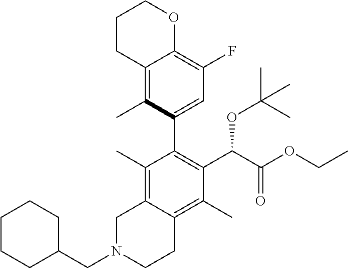

The title compound was prepared in 88% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and cyclohexanecarbaldehyde according to example 81, step 1. ¹H NMR (400 MHz, CDCl₃) δ 6.66 (d, J=11.5 Hz, 1H), 4.92 (s, 1H), 4.28 (t, J=4.9 Hz, 2H), 3.92-4.12 (m, 2H), 3.57 (d, J=15.4 Hz, 1H), 3.45 (d, J=6.4 Hz, 1H), 3.36 (d, J=15.4 Hz, 1H), 2.62-2.90 (m, 4H), 2.47-2.59 (m, 1H), 2.28-2.40 (m, 5H), 2.06-2.21 (m, 2H), 1.51-1.95 (m, 13H), 0.84-1.39 (m, 16H). LCMS (ES+)(m/z): 580.61 (M+1).

### Step 2: (S)-2-(tert-Butoxy)-2-((R)-2-(cyclohexylmethyl)-7-(8-fluoro-5-methylchroman-6-yl)-5,8-dimethyl-1,2,3,4-tetrahydroisoquinolin-6-yl)acetic Acid

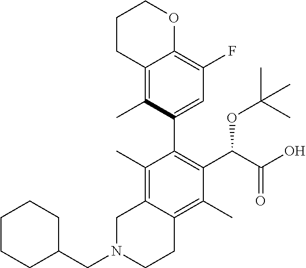

To a stirred solution of (S)-ethyl 2-(tert-butoxy)-2-((R)-2-(cyclohexylmethyl)-7-(8-fluoro-5-methylchroman-6-yl)-5,8-dimethyl-1,2,3,4-tetrahydroisoquinolin-6-yl)acetate (52 mg, 0.090 mmol) in 2:1 dioxane/EtOH (3 mL) in a glass pressure vessel was added 2M aq. LiOH (0.89 mL, 1.79 mmol). The reaction vessel was sealed and the mixture heated to 80° C. After 1 h, LCMS indicated only a few percent conversion and an appreciable amount of solid remained suspended in the reaction mixture. The mixture was treated with 0.5 mL of water which dissolved most of the solid. The resulting mixture was heated at 85° C. for 18 h, at which point LCMS indicated complete reaction. The mixture was cooled to RT, diluted with DCM, and poured into rapidly stirred 1M aq. HCl (25 mL). The pH was adjusted to about 6.0 by addition of 1M aq. NaOH, and the phases separated. The aqueous phase was extracted with DCM (2×). The combined DCM solutions were dried over Na₂SO₄ and concentrated to dryness at reduced pressure. The residue was subjected to RP-HPLC purification (C18, 10-100% MeCN/H₂O with 0.1% formic acid) to afford the title compound (28.4 mg, 53%) as the formate salt as a white solid. ¹H NMR (400 MHz, CDCl₃) δ 8.33 (s, 1H, formate proton), 6.63 (d, J=11.4 Hz, 1H), 4.85 (br s, 1H), 4.26 (t, J=4.8 Hz, 2H), 3.99 (d, J=15.6 Hz, 1H), 3.38-3.58 (m, 3H), 2.57-3.11 (m, 6H), 2.30-2.36 (s, 3H), 2.07-2.17 (m, 2H), 1.54-1.93 (m, 13H), 0.89-1.36 (m, 13H).

LCMS (ES+)(m/z): 552.48 (M+1).

### Example 86: (S)-2-(tert-butoxy)-2-(7-(4-chlorophenyl)-2-(cyclohexylsulfonyl)-5,8-dimethyl-1,2,3,4-tetrahydroisoquinolin-6-yl)acetic Acid

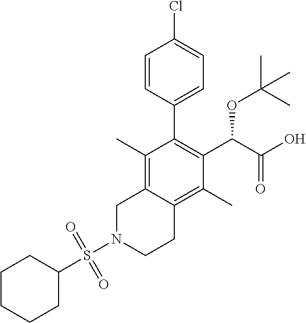 [SMILES: Cc1c2c(c(C)c([C@H](OC(C)(C)C)C(=O)O)c1-c1ccc(Cl)cc1)CCN(S(=O)(=O)C1CCCCC1)C2]

The title compound was prepared in a manner similar to that described in example 79 steps 1-6 and example 83 steps 3-4 from (4-chlorophenyl)boronic acid and was isolated as a white solid after reverse phase HPLC. ¹H NMR (400 MHz, METHANOL-d₄) δ=7.52-7.37 (m, 1H), 7.24-7.16 (m, 1H), 4.97 (s, 1H), 4.51-4.34 (m, 2H), 3.73-3.54 (m, 2H), 3.24-3.13 (m, 1H), 2.89-2.80 (m, 2H), 2.30 (s, 3H), 2.13 (d, J=11.5 Hz, 2H), 1.94-1.81 (m, 5H), 1.75-1.66 (m, 1H), 1.59-1.46 (m, 2H), 1.43-1.14 (m, 3H), 0.95 (s, 9H); LC/MS (m/z) ES⁺=571 (M+23).

### Example 87: (S)-2-(tert-butoxy)-2-(7-(4-chlorophenyl)-2-cyclohexyl-5,8-dimethyl-1,2,3,4-tetrahydroisoquinolin-6-yl)acetic Acid

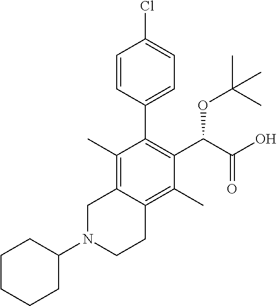 [SMILES: Cc1c2c(c(C)c([C@H](OC(C)(C)C)C(=O)O)c1-c1ccc(Cl)cc1)CCN(C1CCCCC1)C2]

The title compound was prepared in a manner similar to that described in example 84 and was isolated as a tan solid after reverse phase HPLC. ¹H NMR (400 MHz, METHANOL-d₄) δ=7.66-7.60 (m, 1H), 7.47-7.40 (m, 2H), 7.17-7.12 (m, 1H), 4.80 (s, 1H), 4.26 (s, 2H), 3.48 (br. s., 2H), 3.15-2.91 (m, 2H), 2.40-2.30 (m, 3H), 2.24-2.13 (m, 2H), 2.04-1.94 (m, 2H), 1.90-1.81 (m, 3H), 1.80-1.70 (m, 1H), 1.68-1.55 (m, 2H), 1.52-1.37 (m, 2H), 1.35-1.20 (m, 2H), 0.89 (s, 9H); LC/MS (m/z) ES⁺=484 (M+1).

### Example 88: (S)(M)-2-(tert-butoxy)-2-(7-(8-chloro-5-methylchroman-6-yl)-2-(cyclohexylsulfonyl)-5,8-dimethyl-1,2,3,4-tetrahydroisoquinolin-6-yl)acetic Acid

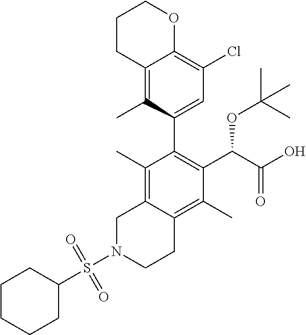

The title compound was prepared in a manner similar to that described in example 79 steps 1-6 and example 83 steps 3-4 from 2-(8-chloro-5-methylchroman-6-yl)-4,4,5,5-tetramethyl-1,3,2-dioxaborolane and was isolated as a white solid after reverse phase HPLC. ¹H NMR (400 MHz, METHANOL-d₄) δ=6.86 (s, 1H), 4.97 (s, 1H), 4.42 (s, 2H), 4.26 (t, J=5.0 Hz, 2H), 3.65 (t, J=5.9 Hz, 2H), 3.23-3.12 (m, 1H), 2.84 (t, J=5.7 Hz, 2H), 2.74 (t, J=6.4 Hz, 2H), 2.35 (s, 3H), 2.17-2.05 (m, 4H), 1.92-1.81 (m, 5H), 1.76 (s, 3H), 1.74-1.66 (m, 1H), 1.59-1.45 (m, 2H), 1.42-1.17 (m, 3H), 1.08 (s, 9H); LC/MS (m/z) ES⁺=641 (M+23).

### Example 89: (S)(M)-2-(tert-butoxy)-2-(7-(8-chloro-5-methylchroman-6-yl)-2-cyclohexyl-5,8-dimethyl-1,2,3,4-tetrahydroisoquinolin-6-yl)acetic Acid

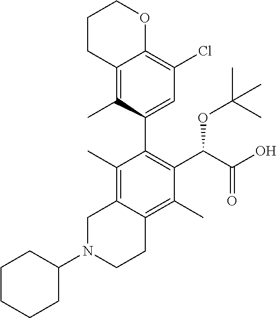

The title compound was prepared in a manner similar to that described in example 84 and was isolated as a tan solid after reverse phase HPLC. ¹H NMR (400 MHz, METHANOL-d₄) δ=6.82 (s, 1H), 4.79 (s, 1H), 4.24 (t, J=5.0 Hz, 2H), 4.20-4.08 (m, 2H), 3.55-3.44 (m, 1H), 3.34-3.16 (m, 2H), 3.15-2.88 (m, 2H), 2.83-2.62 (m, 2H), 2.40 (s, 3H), 2.24-2.03 (m, 4H), 2.00-1.90 (m, 2H), 1.83-1.68 (m, 7H), 1.66-1.52 (m, 2H), 1.49-1.34 (m, 2H), 1.32-1.21 (m, 1H), 1.06 (s, 9H); LC/MS (m/z) ES⁺=554 (M+1).

### Example 90: (S)-2-((R)-2-Benzyl-7-(8-fluoro-5-methylchroman-6-yl)-5,8-dimethyl-1,2,3,4-tetrahydroisoquinolin-6-yl)-2-(tert-butoxy)acetic Acid

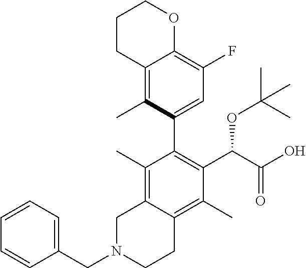

### Step 1: (2S)-Ethyl 2-(2-benzyl-7-(8-fluoro-5-methylchroman-6-yl)-5,8-dimethyl-1,2,3,4-tetrahydroisoquinolin-6-yl)-2-(tert-butoxy)acetate

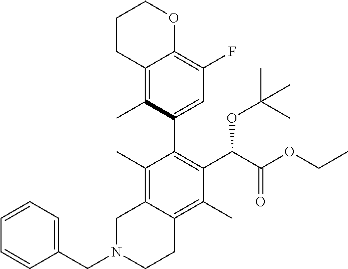

The title compound was prepared in 89% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and benzaldehyde according to example 81, step 1. ¹H NMR (400 MHz, CDCl₃) δ 7.24-7.46 (m, 5H), 6.65 (d, J=11.4 Hz, 1H), 4.93 (s, 1H), 4.28 (t, J=5.1 Hz, 2H), 3.92-4.12 (m, 2H), 3.41-3.82 (m, 4H), 2.52-2.93 (m, 6H), 2.33 (s, 3H), 2.08-2.19 (m, 2H), 1.79 (s, 3H), 1.67 (s, 3H), 1.05-1.18 (m, 12H). LCMS (ES+)(m/z): 574.51 (M+1).

### Step 2: (S)-2-((R)-2-Benzyl-7-(8-fluoro-5-methylchroman-6-yl)-5,8-dimethyl-1,2,3,4-tetrahydroisoquinolin-6-yl)-2-(tert-butoxy)acetic Acid

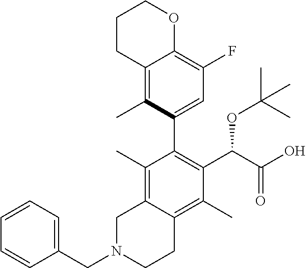

The title compound was prepared in 54% yield from (2S)-ethyl 2-(2-benzyl-7-(8-fluoro-5-methylchroman-6-yl)-5,8-dimethyl-1,2,3,4-tetrahydroisoquinolin-6-yl)-2-(tert-butoxy)acetate according to example 85, step 2. ¹H NMR (400 MHz, CDCl₃) δ 7.22-7.45 (m, 5H), 6.61 (d, J=11.4 Hz, 1H), 4.83 (br s, 1H), 4.19-4.36 (m, 2H), 3.97 (d, J=12.6 Hz, 1H), 3.67-3.83 (m, 2H), 3.26-3.48 (m, 1H), 2.41-3.00 (m, 6H), 2.29 (s, 3H), 2.03-2.19 (m, 2H), 1.49 (s, 3H), 1.36 (br s, 3H), 1.07-1.19 (m, 9H). LCMS (ES⁺)(m/z): 546.49 (M+1).

### Example 91: (S)-2-((R)-2-Benzoyl-7-(8-fluoro-5-methylchroman-6-yl)-5,8-dimethyl-1,2,3,4-tetrahydroisoquinolin-6-yl)-2-(tert-butoxy)acetic Acid

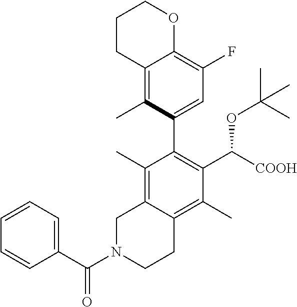

### Step 1: (S)-Ethyl 2-((R)-2-benzoyl-7-(8-fluoro-5-methylchroman-6-yl)-5,8-dimethyl-1,2,3,4-tetrahydroisoquinolin-6-yl)-2-(tert-butoxy)acetate

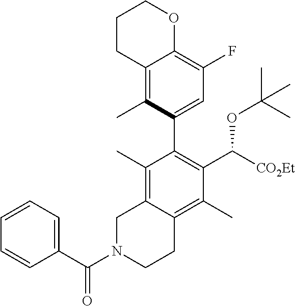

The title compound was prepared in 92% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and benzoyl chloride according to example 80, step 1. ¹H NMR (400 MHz, CDCl₃, mixture of rotamers) δ 7.36-7.56 (m, 5H), 6.58-6.75 (m, 1H), 4.58-5.03 (m, 2H), 4.23-4.34 (m, 2H), 3.96-4.18 (m, 2H), 3.49-3.84 (m, 1H), 2.61-2.96 (m, 5H), 2.38 (s, 3H), 2.08-2.21 (m, 2H), 1.41-1.96 (m, 7H), 1.05-1.21 (m, 12H). LCMS (ES+)(m/z): 588.53 (M+1).

### Step 2: (S)-2-((R)-2-Benzoyl-7-(8-fluoro-5-methylchroman-6-yl)-5,8-dimethyl-1,2,3,4-tetrahydroisoquinolin-6-yl)-2-(tert-butoxy)acetic Acid

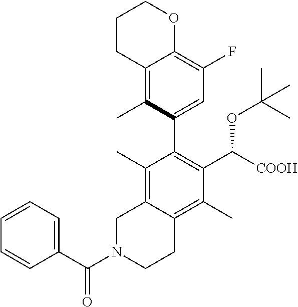

The title compound was prepared in 41% yield from (S)-ethyl 2-((R)-2-benzoyl-7-(8-fluoro-5-methylchroman-6-yl)-5,8-dimethyl-1,2,3,4-tetrahydroisoquinolin-6-yl)-2-(tert-butoxy)acetate according to example 79, step 8. ¹H NMR (400 MHz, CDCl₃, mixture of rotamers) δ 7.39-7.54 (m, 5H), 6.63-6.76 (m 1H), 5.10 (br s, 1H), 4.80 (br s, 1H), 4.22-4.32 (m, 3H), 3.68 (br s, 1H), 3.49 (br s, 1H), 2.58-2.97 (m, 4H), 2.27 (br s, 3H), 2.07-2.18 (m, 2H) 1.86 (br s, 6H), 1.13 (s, 9H). LCMS (ES+)(m/z): 560.44 (M+1).

### Example 92: (S)-2-(tert-Butoxy)-2-((R)-2-(3-fluoro-4-methoxybenzoyl)-7-(8-fluoro-5-methylchroman-6-yl)-5,8-dimethyl-1,2,3,4-tetrahydroisoquinolin-6-yl)acetic Acid

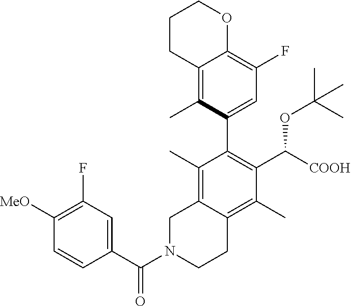

### Step 1: (S)-Ethyl 2-(tert-butoxy)-2-((R)-2-(3-fluoro-4-methoxybenzoyl)-7-(8-fluoro-5-methylchroman-6-yl)-5,8-dimethyl-1,2,3,4-tetrahydroisoquinolin-6-yl)acetate

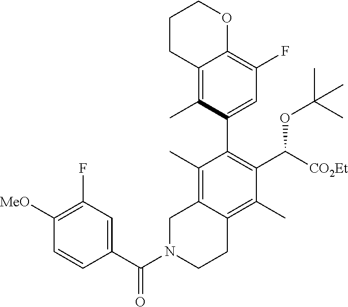

The title compound was prepared in quantitative yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and 3-fluoro-4-methoxybenzoic acid according to example 79, step 7. ¹H NMR (400 MHz, CDCl₃, mixture of rotamers) δ 7.20-7.32 (m, 2H), 7.01 (t, J=8.0 Hz, 1H), 6.65 (d, J=11.2 Hz, 1H), 4.97 (br s, 1H), 4.51-5.01 (m, 3H), 4.28 (t, J=4.7 Hz, 2H), 3.51-4.18 (m, 6H), 2.61-2.97 (m, 4H), 2.38 (s, 3H), 2.09-2.21 (m, 2H), 1.52-1.94 (m, 6H), 1.03-1.21 (m, 12H). LCMS (ES+)(m/z): 636.48 (M+1).

### Step 2: (S)-2-(tert-Butoxy)-2-((R)-2-(3-fluoro-4-methoxybenzoyl)-7-(8-fluoro-5-methylchroman-6-yl)-5,8-dimethyl-1,2,3,4-tetrahydroisoquinolin-6-yl)acetic Acid

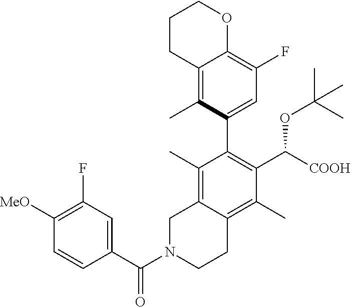

The title compound was prepared in 24% yield from (S)-ethyl 2-(tert-butoxy)-2-((R)-2-(3-fluoro-4-methoxybenzoyl)-7-(8-fluoro-5-methylchroman-6-yl)-5,8-dimethyl-1,2,3,4-tetrahydroisoquinolin-6-yl)acetate according to example 79, step 8. ¹H NMR (400 MHz, CDCl₃, mixture of rotamers) δ 7.22-7.33 (m, 2H), 7.02 (t, J=8.2 Hz, 1H), 6.69 (d, J=10.8 Hz, 1H), 4.67-5.18 (m, 1H), 4.27 (t, J=4.9 Hz, 2H), 3.58-4.05 (m, 8H), 1.72-2.96 (m, 14H), 1.13 (s, 9H). LCMS (ES+)(m/z): 608.51 (M+1).

### Example 93: (S)-2-(tert-Butoxy)-2-((R)-7-(8-fluoro-5-methylchroman-6-yl)-2-(3-isopropoxybenzoyl)-5,8-dimethyl-1,2,3,4-tetrahydroisoquinolin-6-yl)acetic Acid

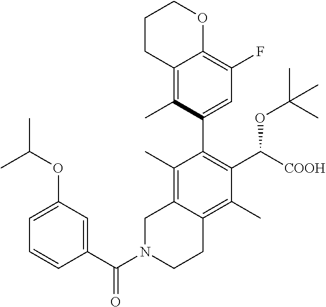

### Step 1: (S)-Ethyl 2-(tert-butoxy)-2-((R)-7-(8-fluoro-5-methylchroman-6-yl)-2-(3-isopropoxybenzoyl)-5,8-dimethyl-1,2,3,4-tetrahydroisoquinolin-6-yl)acetate

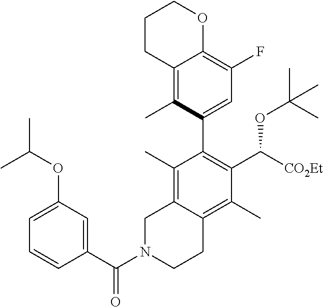

The title compound was prepared in 98% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and 3-isopropoxybenzoic acid according to example 79, step 7. ¹H NMR (400 MHz, CDCl₃, mixture of rotamers) δ 7.26-7.40 (m, 1H), 6.90-7.07 (m, 3H), 6.58-6.74 (m, 1H), 3.52-5.04 (m, 10H), 2.59-3.00 (m, 4H), 2.37 (s, 3H), 2.09-2.21 (m, 2H), 1.68-1.93 (m, 6H) 1.01-1.44 (m, 18H). LCMS (ES+)(m/z): 646.57 (M+1).

### Step 2: (S)-2-(tert-Butoxy)-2-((R)-7-(8-fluoro-5-methylchroman-6-yl)-2-(3-isopropoxybenzoyl)-5,8-dimethyl-1,2,3,4-tetrahydroisoquinolin-6-yl)acetic Acid

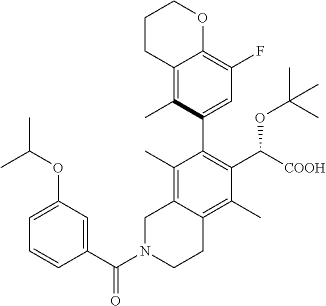

The title compound was prepared in 24% yield from (S)-ethyl 2-(tert-butoxy)-2-((R)-7-(8-fluoro-5-methylchroman-6-yl)-2-(3-isopropoxybenzoyl)-5,8-dimethyl-1,2,3,4-tetrahydroisoquinolin-6-yl)acetate according to example 79, step 8. ¹H NMR (400 MHz, CDCl₃, mixture of rotamers) δ 7.25-7.40 (m, 1H), 6.91-7.07 (m, 3H), 6.63-6.76 (m, 1H), 3.60-5.18 (m, 8H), 2.58-2.98 (m, 4H), 2.27 (br s, 3H), 2.06-2.18 (m, 2H) 0.97-1.95 (m, 21H). LCMS (ES+)(m/z): 618.55 (M+1).

### Example 94: (S)-2-(tert-Butoxy)-2-((R)-7-(8-fluoro-5-methylchroman-6-yl)-5,8-dimethyl-2-(4-(trifluoromethyl)benzoyl)-1,2,3,4-tetrahydroisoquinolin-6-yl)acetic Acid

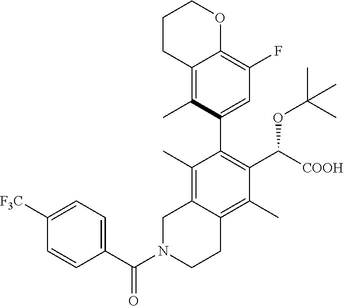

### Step 1: (S)-Ethyl 2-(tert-butoxy)-2-((R)-7-(8-fluoro-5-methylchroman-6-yl)-5,8-dimethyl-2-(4-(trifluoromethyl)benzoyl)-1,2,3,4-tetrahydroisoquinolin-6-yl)acetate

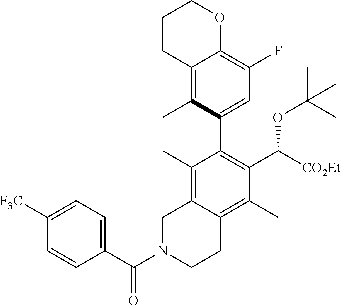

The title compound was prepared in 98% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and 4-(trifluoromethyl)benzoic acid according to example 79, step 7. ¹H NMR (400 MHz, CDCl₃, mixture of rotamers) δ 7.54-7.84 (m, 4H), 6.59-6.76 (m, 1H), 3.90-5.19 (m, 9H), 3.49-3.79 (m, 1H), 2.59-3.02 (m, 4H), 0.89-2.46 (m, 22H). LCMS (ES+)(m/z): 656.50 (M+1).

### Step 2: (S)-2-(tert-Butoxy)-2-((R)-7-(8-fluoro-5-methylchroman-6-yl)-5,8-dimethyl-2-(4-(trifluoromethyl)benzoyl)-1,2,3,4-tetrahydroisoquinolin-6-yl)acetic Acid

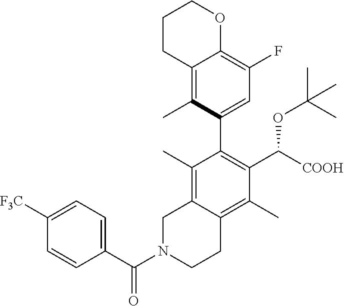

The title compound was prepared in 11% yield from (S)-ethyl 2-(tert-butoxy)-2-((R)-7-(8-fluoro-5-methylchroman-6-yl)-5,8-dimethyl-2-(4-(trifluoromethyl)benzoyl)-1,2,3,4-tetrahydroisoquinolin-6-yl)acetate according to example 79, step 8. ¹H NMR (400 MHz, CDCl₃, mixture of rotamers) δ 7.53-7.81 (m, 4H), 6.60-6.77 (m, 1H), 3.53-5.18 (m, 7H), 2.59-2.99 (m, 5H), 2.04-2.34 (m, 5H), 1.87 (br s, 6H), 1.13 (s, 9H). LCMS (ES⁺)(m/z): 628.50 (M+1).

### Example 95: (S)(M)-2-(tert-butoxy)-2-(2-(cyclohexylsulfonyl)-5,8-dimethyl-7-(5-methyl-3,4-dihydro-2H-benzo[b][1,4]oxazin-6-yl)-1,2,3,4-tetrahydroisoquinolin-6-yl)acetic Acid

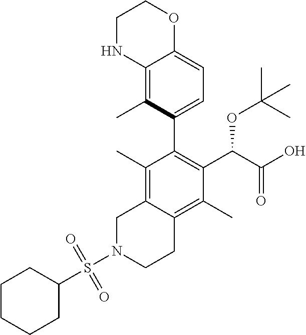

The title compound was prepared in a manner similar to that described in example 79 steps 1-6 and example 83 steps 3-4 from 5-methyl-6-(4,4,5,5-tetramethyl-1,3,2-dioxaborolan-2-yl)-3,4-dihydro-2H-benzo[b][1,4]oxazine and was isolated as a white solid after reverse phase HPLC. ¹H NMR (400 MHz, METHANOL-d₄) δ=6.63 (d, J=8.1 Hz, 1H), 6.34 (d, J=8.2 Hz, 1H), 5.08 (s, 1H), 4.42 (s, 2H), 4.22 (t, J=4.1 Hz, 2H), 3.65 (t, J=5.9 Hz, 2H), 3.46 (d, J=3.8 Hz, 2H), 3.22-3.11 (m, 1H), 2.84 (t, J=5.8 Hz, 2H), 2.33 (s, 3H), 2.12 (d, J=11.4 Hz, 2H), 1.86 (d, J=12.8 Hz, 2H), 1.78-1.65 (m, 7H), 1.52 (dq, J=2.7, 12.3 Hz, 2H), 1.41-1.15 (m, 4H), 1.07 (s, 9H); LC/MS (m/z) ES⁺=585 (M+1).

### Example 96: (S)-2-(tert-Butoxy)-2-((R)-7-(8-fluoro-5-methylchroman-6-yl)-5,8-dimethyl-2-(6-methylnicotinoyl)-1,2,3,4-tetrahydroisoquinolin-6-yl)acetic Acid

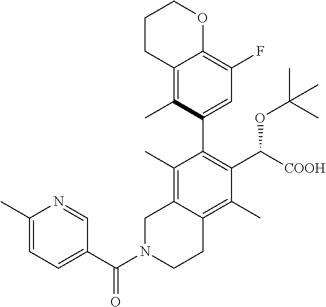

### Step 1: (S)-Ethyl 2-(tert-butoxy)-2-((R)-7-(8-fluoro-5-methylchroman-6-yl)-5,8-dimethyl-2-(6-methylnicotinoyl)-1,2,3,4-tetrahydroisoquinolin-6-yl)acetate

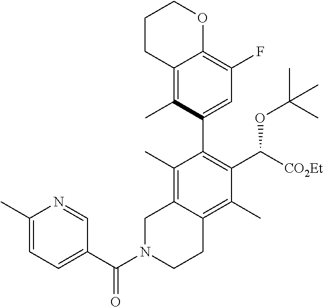

The title compound was prepared in 98% yield from (S)-ethyl 2-(tert-butoxy)-2-((R)-7-(8-fluoro-5-methylchroman-6-yl)-5,8-dimethyl-1,2,3,4-tetrahydroisoquinolin-6-yl)acetate and 6-methylnicotinic acid according to example 79, step 7. ¹H NMR (400 MHz, CDCl₃, mixture of rotamers) δ 8.65 (s, 1H), 7.73 (d, J=7.9 Hz, 1H), 7.17-7.32 (m, 1H), 6.56-6.73 (m, 1H), 3.49-5.05 (m, 9H), 2.53-2.98 (m, 7H), 2.37 (s, 3H), 2.09-2.21 (m, 2H), 1.47-1.96 (m, 6H), 1.02-1.22 (m, 12H). LCMS (ES+)(m/z): 603.50 (M+1).

### Step 2: (S)-2-(tert-Butoxy)-2-((R)-7-(8-fluoro-5-methylchroman-6-yl)-5,8-dimethyl-2-(6-methylnicotinoyl)-1,2,3,4-tetrahydroisoquinolin-6-yl)acetic Acid

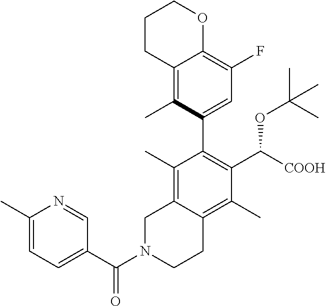

The title compound was prepared in 20% yield from (S)-ethyl 2-(tert-butoxy)-2-((R)-7-(8-fluoro-5-methylchroman-6-yl)-5,8-dimethyl-2-(6-methylnicotinoyl)-1,2,3,4-tetrahydroisoquinolin-6-yl)acetate according to example 79, step 8. ¹H NMR (400 MHz, CDCl₃, mixture of rotamers) δ 8.66 (s, 1H), 7.76 (d, J=7.5 Hz, 1H), 7.20-7.32 (m, 1H), 6.59-6.79 (m, 1H), 3.57-5.19 (m, 7H), 2.52-2.99 (m, 7H), 2.29 (s, 3H), 2.05-2.19 (m, 2H), 1.85 (br s, 6H), 1.13 (s, 9H). LCMS (ES+)(m/z): 575.45 (M+1).

### Example 97: (S)-2-(tert-butoxy)-2-((R)-7-(8-fluoro-5-methylchroman-6-yl)-2-(4-methoxy-3-methylbenzoyl)-5,8-dimethyl-1,2,3,4-tetrahydroisoquinolin-6-yl)acetic Acid

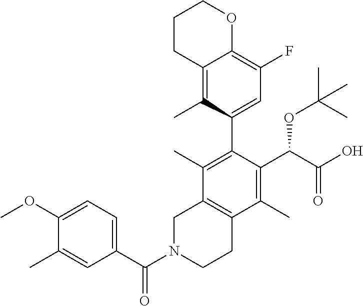

The title compound was made in a manner similar to that described in example 5 from 4-methoxy-3-methylbenzoic acid and isolated as a white solid after reverse phase HPLC. ¹H NMR (400 MHz, METHANOL-d₄) δ ppm 7.42-7.18 (m, 2H), 7.00 (d, J=7.5 Hz, 1H), 6.60 (d, J=10.4 Hz, 1H), 5.00 (br. s., 1H), 4.79-4.51 (m, 2H), 4.23 (br. s., 2H), 4.06-3.64 (m, 5H), 2.87 (br. s., 2H), 2.73 (br. s., 2H), 2.37 (s, 3H), 2.23 (br. s., 3H), 2.12 (br. s., 2H), 1.95-1.46 (m, 6H), 1.09 (s, 9H); LCMS (m/z) ES⁺=604.4 (M+1), ES⁻=602.4 (M−1).

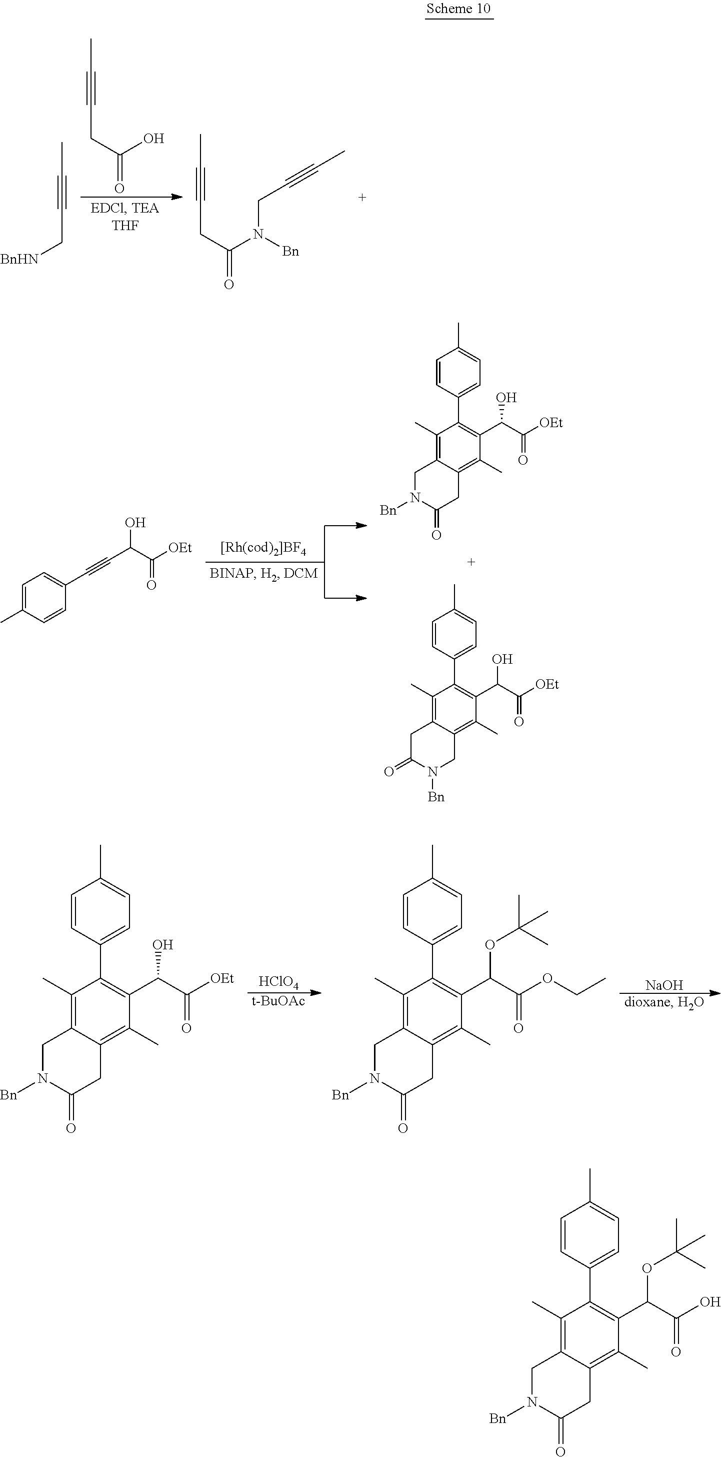

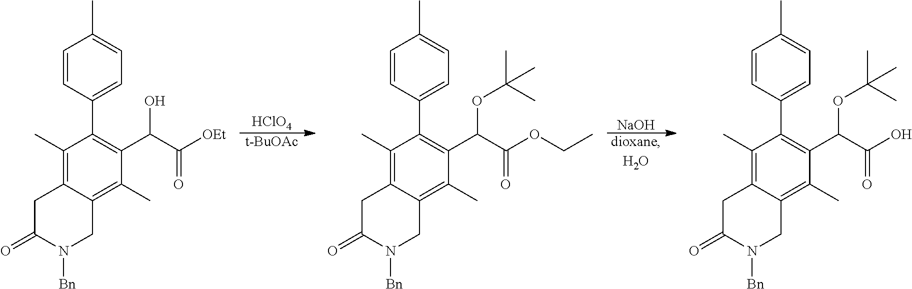

### Example 98: 2-(2-Benzyl-5,8-dimethyl-3-oxo-7-(p-tolyl)-1,2,3,4-tetrahydroisoquinolin-6-yl)-2-(tert-butoxy)acetic Acid

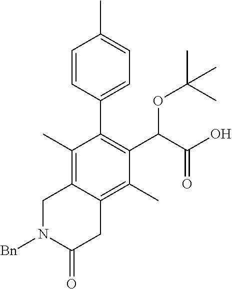 [SMILES: Cc1ccc(-c2c(C)c3c(c(C)c2C(OC(C)(C)C)C(=O)O)CC(=O)N(Cc2ccccc2)C3)cc1]

### Step 1: N-benzyl-N-(but-2-yn-1-yl)pent-3-ynamide

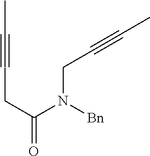 [SMILES: CC#CCC(=O)N(CC#CC)Cc1ccccc1]

To a mixture of N-benzylbut-2-yn-1-amine (1.43 g, 8.99 mmol), pent-3-ynoic acid (800 mg, 8.16 mmol) in THF (15 mL) was added EDCl (2.33 g, 12.24 mmol) and TEA (1.65 g, 16.32 mmol). After stirred at r.t. for 1.5 hr, the resulting mixture was partitioned between EtOAc and H₂O. The organic layer was washed with brine, dried over Na₂SO₄, filtered and concentrated to give the crude product which was purified by flash chromatography (silica gel, 0-10% EtOAc in PE) to afford the title compound (1.1 g, 56% yield) as a colorless oil. LC-MS (ESI): m/z (M+1)=240.42.

### Step 2: Ethyl (S)-2-(2-benzyl-5,8-dimethyl-3-oxo-7-(p-tolyl)-1,2,3,4-tetrahydroisoquino lin-6-yl)-2-h

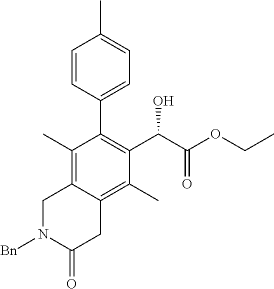 [SMILES: CCOC(=O)[C@@H](O)c1c(C)c2c(c(C)c1-c1ccc(C)cc1)CN(Cc1ccccc1)C(=O)C2]

A mixture of Rh(cod)₂BF₄ (10 mg, 0.023 mmol) and BINAP (15 mg, 0.023 mmol) in DCM (1 mL) was stirred at r.t. under H₂ atmosphere for 5 hr. After concentration, the resulting mixture was dissolved in DCM (1 mL) and ethyl 2-hydroxy-4-(p-tolyl) but-3-ynoate (50 mg, 0.229 mmol) was introduced. The reaction mixture was heated up to 40° C. in a seal tube and N-benzyl-N-(but-2-yn-1-yl)pent-3-ynamide (109 mg, 0.458 mmol) was added as a DCM solution (1 mL) over 30 min. After stirred at 40° C. for 30 min, the resulting mixture was concentrated to give the crude product which was purified by flash chromatography (silica gel, 0-30% EtOAc in PE) to afford the title compound (16 mg, 15% yield). LC-MS (ESI): m/z (M+1)=458.09.

### Step 3: Ethyl 2-(2-benzyl-5,8-dimethyl-3-oxo-7-(p-tolyl)-1,2,3,4-tetrahydroisoquinolin-6-yl)-2-(tert-butoxy)acetate

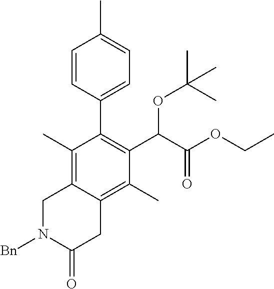 [SMILES: CCOC(=O)C(OC(C)(C)C)c1c(C)c2c(c(C)c1-c1ccc(C)cc1)CN(Cc1ccccc1)C(=O)C2]

To a solution of ethyl (S)-2-(2-benzyl-5,8-dimethyl-3-oxo-7-(p-tolyl)-1,2,3,4-tetrahydro isoquinolin-6-yl)-2-hydroxyacetate (16 mg, 0.035 mmol) in t-BuOAc (1.75 mL) was added HClO₄ (5 mg, 0.035 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (9 mg, 50% yield). LC-MS (ESI): m/z (M+1)=514.12.

### Step 4: 2-(2-Benzyl-5,8-dimethyl-3-oxo-7-(p-tolyl)-1,2,3,4-tetrahydroisoquinolin-6-yl)-2-(tert-butoxy)acetic Acid

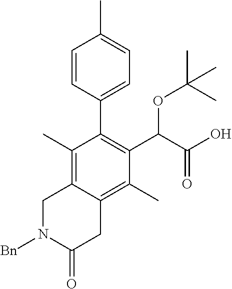 [SMILES: Cc1ccc(-c2c(C)c3c(c(C)c2C(OC(C)(C)C)C(=O)O)CC(=O)N(Cc2ccccc2)C3)cc1]

A mixture of ethyl 2-(2-benzyl-5,8-dimethyl-3-oxo-7-(p-tolyl)-1,2,3,4-tetrahydroisoquinolin-6-yl)-2-(tert-butoxy)acetate (9 mg, 0.018 mmol) in dioxane (1.0 mL) and H₂O (0.2 mL) was added LiOH (7 mg, 0.18 mmol). After stirred at 80° C. for 4 hr, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (2.3 mg, 27% yield). LC-MS (ESI): m/z (M+1)=486.11. ¹H NMR (400 MHz, CDCl₃) δ 9.68 (br, 1H), 7.38-7.27 (m, 5H), 7.26-7.18 (m, 3H), 7.01 (d, J=8.4 Hz, 1H), 5.14 (s, 1H), 4.88-4.73 (m, 2H), 4.35 (s, 2H), 3.69 (q, J=20.3 Hz, 2H), 2.40 (s, 3H), 2.25 (s, 3H), 1.76 (s, 3H), 0.97 (s, 9H).

### Example 99: 2-(2-Benzyl-5,8-dimethyl-3-oxo-6-(p-tolyl)-1,2,3,4-tetrahydroisoquinolin-7-yl)-2-(tert-butoxy)acetic Acid

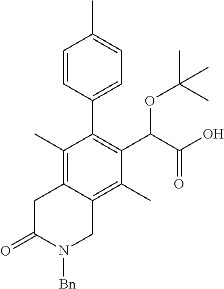 [SMILES: Cc1ccc(-c2c(C)c3c(c(C)c2C(OC(C)(C)C)C(=O)O)CN(Cc2ccccc2)C(=O)C3)cc1]

### Step 1: Ethyl (S)-2-(2-benzyl-5,8-dimethyl-3-oxo-6-(p-tolyl)-1,2,3,4-tetrahydroisoquino lin-7-yl)-2-hydroxyacetate

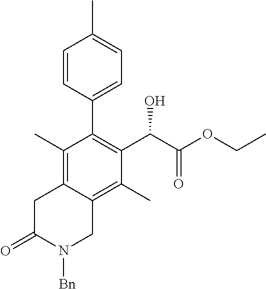 [SMILES: CCOC(=O)[C@@H](O)c1c(C)c2c(c(C)c1-c1ccc(C)cc1)CC(=O)N(Cc1ccccc1)C2]

A mixture of Rh(cod)₂BF₄ (10 mg, 0.023 mmol) and BINAP (15 mg, 0.023 mmol) in DCM (1 mL) was stirred at r.t. under H₂ atmosphere for 5 hr. After concentration, the resulting mixture was dissolved in DCM (1 mL) and ethyl 2-hydroxy-4-(p-tolyl) but-3-ynoate (50 mg, 0.229 mmol) was introduced. The reaction mixture was heated up to 40° C. in a seal tube and N-benzyl-N-(but-2-yn-1-yl)pent-3-ynamide (109 mg, 0.458 mmol) was added as a DCM solution (1 mL) over 30 min. After stirred at 40° C. for 30 min, the resulting mixture was concentrated to give the crude product which was purified by flash chromatography (silica gel, 0-30% EtOAc in PE) to afford the title compound (20 mg, 19% yield). LC-MS (ESI): m/z (M+1)=458.09.

### Step 2: Ethyl 2-(2-benzyl-5,8-dimethyl-3-oxo-6-(p-tolyl)-1,2,3,4-tetrahydroisoquinolin 2-(tert-butoxy)acetate

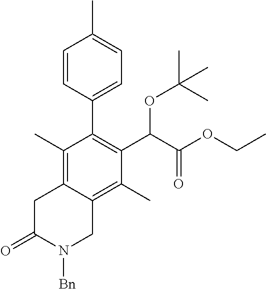 [SMILES: CCOC(=O)C(OC(C)(C)C)c1c(C)c2c(c(C)c1-c1ccc(C)cc1)CC(=O)N(Cc1ccccc1)C2]

To a solution of ethyl (S)-2-(2-benzyl-5,8-dimethyl-3-oxo-6-(p-tolyl)-1,2,3,4-tetrahydro isoquinolin-7-yl)-2-hydroxyacetate (20 mg, 0.043 mmol) in t-BuOAc (2 mL) was added HClO₄ (6.2 mg, 0.043 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (17 mg, 77% yield). LC-MS (ESI): m/z (M+1)=514.09.

### Step 3: 2-(2-Benzyl-5,8-dimethyl-3-oxo-6-(p-tolyl)-1,2,3,4-tetrahydroisoquinolin-7-yl)-2-(tert-butoxy)acetic Acid

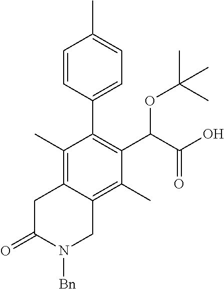 [SMILES: Cc1ccc(-c2c(C)c3c(c(C)c2C(OC(C)(C)C)C(=O)O)CN(Cc2ccccc2)C(=O)C3)cc1]

A mixture of ethyl 2-(2-benzyl-5,8-dimethyl-3-oxo-6-(p-tolyl)-1,2,3,4-tetrahydroisoquinolin-7-yl)-2-(tert-butoxy)acetate (17 mg, 0.033 mmol) in dioxane (1.0 mL) and H₂O (0.2 mL) was added LiOH (15.8 mg, 0.662 mmol). After stirred at 80° C. for 4 hr, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (6.8 mg, 42% yield). LC-MS (ESI): m/z (M+1)=486.39. ¹H NMR (400 MHz, CDCl₃) δ 9.68 (br, 1H), 7.37-7.28 (m, 5H), 7.26-7.20 (m, 3H), 7.05 (d, J=7.3 Hz, 1H), 5.15 (s, 1H), 4.82 (dd, J=54.3, 14.8 Hz, 2H), 4.38 (s, 2H), 3.76-3.58 (m, 2H), 2.41 (s, 3H), 2.09 (s, 3H), 1.91 (s, 3H), 0.95 (s, 9H).

### Example 100: (S)-2-(2-Benzyl-5,8-dimethyl-3-oxo-6-(p-tolyl)-1,2,3,4-tetrahydroisoquinolin-7-yl)-2-(tert-butoxy)acetic Acid

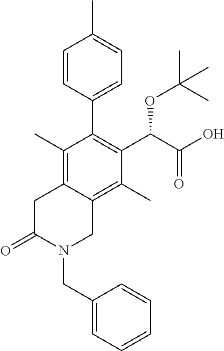 [SMILES: Cc1ccc(-c2c(C)c3c(c(C)c2[C@H](OC(C)(C)C)C(=O)O)CN(Cc2ccccc2)C(=O)C3)cc1]

### Step 1: (S)-ethyl 2-(2-benzyl-5,8-dimethyl-3-oxo-6-(trimethylsilyl)-1,2,3,4-tetrahydroisoquinolin-7-yl)-2-hydroxyacetate

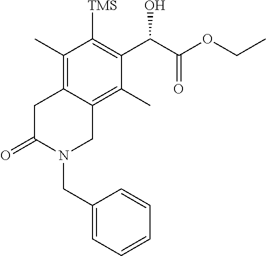 [SMILES: CCOC(=O)[C@@H](O)c1c(C)c2c(c(C)c1[Si](C)(C)C)CC(=O)N(Cc1ccccc1)C2]

A solution of BINAP (62.2 mg, 0.100 mmol) and bis(1,5-cyclooctadiene)rhodium(I)tetrafluoroborate (40.5 mg, 0.100 mmol) in dichloromethane (5 mL) was stirred for 5 minutes at ambient temperature. The mixture was purged with hydrogen gas for one minute and then stirred under a balloon of hydrogen for one hour. The mixture was concentrated. A solution of (S)-ethyl 2-hydroxy-4-(trimethylsilyl)but-3-ynoate (100 mg, 0.499 mmol) in dichloromethane (3 mL) was added. The mixture was heated to 40° C. and a solution of N-benzyl-N-(but-2-yn-1-yl)pent-3-ynamide (358 mg, 1.498 mmol) in dichloromethane (3 mL) was slowly added to the mixture over a period of 2 hours. The mixture was allowed to stir at 40° C. for 18 hours then let sit over the weekend at ambient temperature. LCMS/TLC indicated a mixture of regioisomers was formed. The mixture was concentrated and then purified on silica gel (0-50% hexanes/EtOAc gradient elution, earlier eluting regioisomer) to afford the title compound as a pale yellow residue (84 mg, 38%). ¹H NMR (400 MHz, CHLOROFORM-d) δ=7.38-7.22 (m, 5H), 5.55 (s, 1H), 4.86-4.71 (m, 2H), 4.37-4.23 (m, 3H), 4.20-4.09 (m, 1H), 3.64 (s, 2H), 3.31 (br. s., 1H), 2.37 (s, 3H), 1.99 (s, 3H), 1.19 (t, J=7.1 Hz, 3H), 0.46 (s, 9H); LC/MS (m/z) ES⁺=440 (M+1).

### Step 2: (S)-Ethyl 2-(2-benzyl-6-iodo-5,8-dimethyl-3-oxo-1,2,3,4-tetrahydroisoquinolin-7-yl)-2-hydroxyacetate

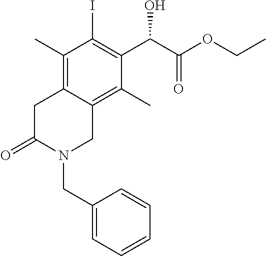 [SMILES: CCOC(=O)[C@@H](O)c1c(C)c2c(c(C)c1I)CC(=O)N(Cc1ccccc1)C2]

An ice cold solution of (S)-ethyl 2-(2-benzyl-5,8-dimethyl-3-oxo-6-(trimethylsilyl)-1,2,3,4-tetrahydroisoquinolin-7-yl)-2-hydroxyacetate (84 mg, 0.191 mmol) in dichloromethane (1.0 mL) was treated slowly with ICl (1M solution in dichloromethane) (0.210 mL, 0.210 mmol) and the mixture was stirred at ice bath temperature for 10 minutes. The mixture was quenched with saturated sodium thiosulfate and then extracted with dichloromethane. The extracts were washed with saturated sodium bicarbonate, then brine, dried over sodium sulfate, filtered and concentrated under reduced pressure to afford a yellow residue (102 mg, quantitative). LC/MS (m/z) ES⁺=494 (M+1).

### Step 3: (S)-Ethyl 2-(2-benzyl-6-iodo-5,8-dimethyl-3-oxo-1,2,3,4-tetrahydroisoquinolin-7-yl)-2-(tert-butoxy)acetate

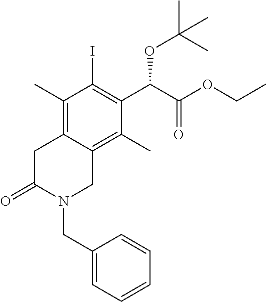 [SMILES: CCOC(=O)[C@@H](OC(C)(C)C)c1c(C)c2c(c(C)c1I)CC(=O)N(Cc1ccccc1)C2]

A suspension of (S)-ethyl 2-(2-benzyl-6-iodo-5,8-dimethyl-3-oxo-1,2,3,4-tetrahydroisoquinolin-7-yl)-2-hydroxyacetate in t-Butyl acetate (3.0 mL) was treated with perchloric acid (0.046 mL, 0.764 mmol) and the pale yellow solution was allowed to stir at ambient temperature for 45 minutes. The mixture was partitioned between EtOAc and 1N NaOH. The organic phase was washed with brine, dried over sodium sulfate, filtered and concentrated under reduced pressure. The residue was purified on silica gel (12 g column, 0-50% hexanes/EtOAc) to afford a pale yellow foam (57 mg, 50%). ¹H NMR (400 MHz, CHLOROFORM-d) δ=7.38-7.28 (m, 5H), 5.92 (s, 1H), 4.79 (s, 2H), 4.30 (s, 2H), 4.23-4.04 (m, 2H), 3.72 (s, 2H), 2.52 (s, 3H), 2.16 (s, 3H), 1.24-1.15 (m, 12H); LC/MS (m/z) ES⁺=550 (M+1).

### Step 4: (S)-Ethyl 2-(2-benzyl-5,8-dimethyl-3-oxo-6-(p-tolyl)-1,2,3,4-tetrahydroisoquinolin-7-yl)-2-(tert-butoxy)acetate

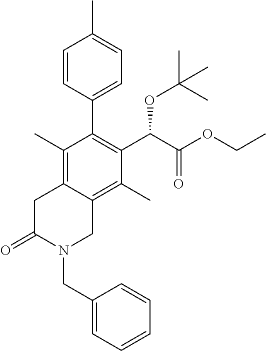 [SMILES: CCOC(=O)[C@@H](OC(C)(C)C)c1c(C)c2c(c(C)c1-c1ccc(C)cc1)CC(=O)N(Cc1ccccc1)C2]

A mixture of (S)-ethyl 2-(2-benzyl-6-iodo-5,8-dimethyl-3-oxo-1,2,3,4-tetrahydroisoquinolin-7-yl)-2-(tert-butoxy)acetate (20.50 mg, 0.037 mmol) in N,N-dimethylformamide (1.0 mL) was degassed while adding p-tolylboronic acid (10.15 mg, 0.075 mmol), K₃PO₄ (23.76 mg, 0.112 mmol), MePhos (2.72 mg, 7.46 μmol) and finally Pd₂dba₃ (6.83 mg, 7.46 μmol). The mixture was degassed for several minutes and then stirred at 80° C. overnight. The mixture was cooled to ambient temperature, diluted with EtOAc, washed with water, then brine, dried over sodium sulfate, filtered and concentrated under reduced pressure. The residue was purified by reverse phase HPLC to afford a pale yellow residue (7.4 mg, 39%). ¹H NMR (400 MHz, CHLOROFORM-d) δ=7.39-7.28 (m, 5H), 7.25-7.16 (m, 3H), 7.06-7.01 (m, 1H), 4.98 (s, 1H), 4.82 (s, 2H), 4.39 (s, 2H), 4.22-3.98 (m, 2H), 3.67 (s, 2H), 2.43 (s, 3H), 2.19 (s, 3H), 1.88 (s, 3H), 1.21 (t, J=7.1 Hz, 3H), 0.94 (s, 9H); LC/MS (m/z) ES⁺=514 (M+1).

### Step 5: (S)-2-(2-Benzyl-5,8-dimethyl-3-oxo-6-(p-tolyl)-1,2,3,4-tetrahydroisoquinolin-7-yl)-2-(tert-butoxy)acetic Acid

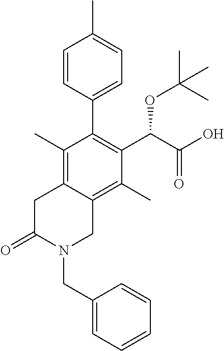 [SMILES: Cc1ccc(-c2c(C)c3c(c(C)c2[C@H](OC(C)(C)C)C(=O)O)CN(Cc2ccccc2)C(=O)C3)cc1]

A mixture of (S)-ethyl 2-(2-benzyl-5,8-dimethyl-3-oxo-6-(p-tolyl)-1,2,3,4-tetrahydroisoquinolin-7-yl)-2-(tert-butoxy)acetate (7.4 mg, 0.014 mmol) in tetrahydrofuran (0.6 mL) and ethanol (0.3 mL) was treated with 2M LiOH (0.3 mL, 0.600 mmol). The mixture was heated to 70° C. and stirred for 8 hours. The mixture was then allowed to stir at ambient temperature overnight. The mixture was concentrated, diluted with 1N HCl then extracted with EtOAc. The combined extracts were washed with brine, dried over sodium sulfate, filtered and concentrated under reduced pressure to a pale yellow residue. The residue was purified by reverse phase HPLC to afford the title compound as a white solid (3.2 mg, 47%). ¹H NMR (400 MHz, CHLOROFORM-d) δ=7.40-7.20 (m, 8H), 7.10-7.01 (m, 1H), 5.16 (s, 1H), 4.93-4.85 (m, 1H), 4.79-4.71 (m, 1H), 4.39 (s, 2H), 3.78-3.56 (m, 2H), 2.42 (s, 3H), 2.09 (s, 3H), 1.92 (s, 3H), 0.96 (s, 9H); LC/MS (m/z) ES⁺=486 (M+1).

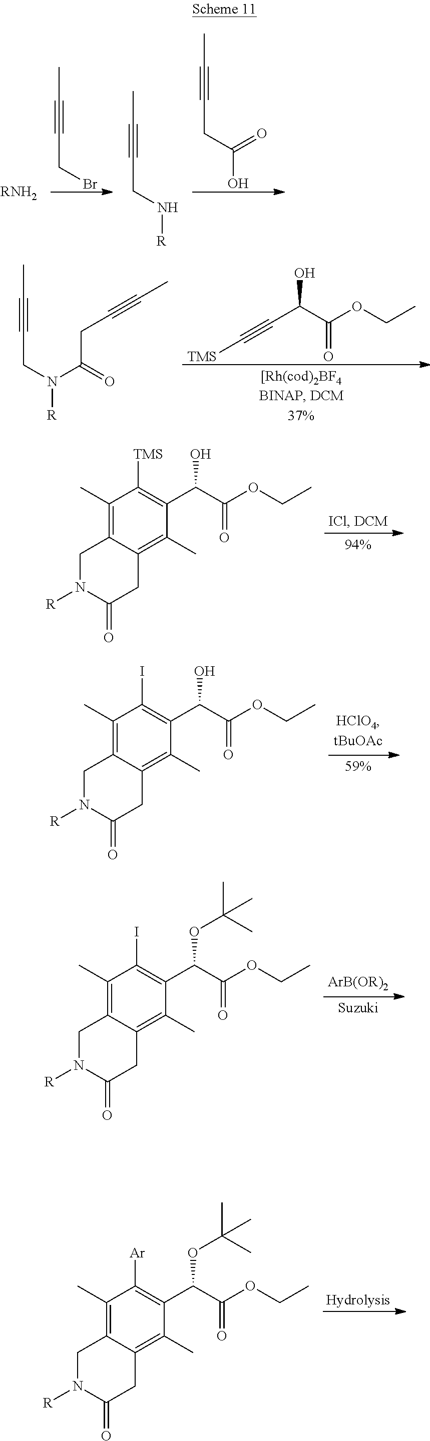

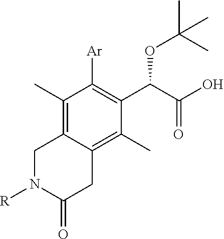

### Example 101: (S)-2-(2-Benzyl-5,8-dimethyl-3-oxo-7-(p-tolyl)-1,2,3,4-tetrahydroisoquinolin-6-yl)-2-(tert-butoxy)acetic Acid

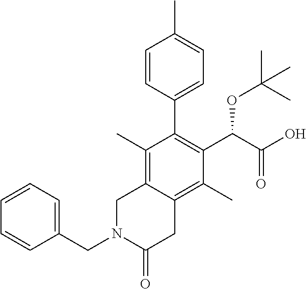 [SMILES: Cc1ccc(-c2c(C)c3c(c(C)c2[C@H](OC(C)(C)C)C(=O)O)CC(=O)N(Cc2ccccc2)C3)cc1]

### Step 1: (S)-Ethyl 2-(2-benzyl-5,8-dimethyl-3-oxo-7-(trimethylsilyl)-1,2,3,4-tetrahydroisoquinolin-6-yl)-2-hydroxyacetate

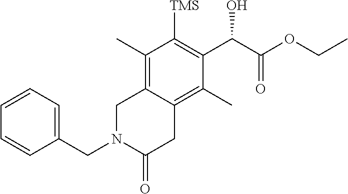 [SMILES: CCOC(=O)[C@@H](O)c1c(C)c2c(c(C)c1[Si](C)(C)C)CN(Cc1ccccc1)C(=O)C2]

A solution of BINAP (62.2 mg, 0.100 mmol) and bis(1,5-cyclooctadiene)rhodium(I)tetrafluoroborate (40.5 mg, 0.100 mmol) in dichloromethane (5 mL) was stirred for 5 minutes at ambient temperature. The mixture was purged with hydrogen gas for one minute and then stirred under a balloon of hydrogen for one hour. The mixture was concentrated. A solution of (S)-ethyl 2-hydroxy-4-(trimethylsilyl)but-3-ynoate (100 mg, 0.499 mmol) in dichloromethane (3 mL) was added. The mixture was heated to 40° C. and a solution of N-benzyl-N-(but-2-yn-1-yl)pent-3-ynamide (358 mg, 1.498 mmol) in dichloromethane (3 mL) was slowly added to the mixture over a period of 2 hours. The mixture was allowed to stir at 40° C. for 18 hours then let sit over the weekend at ambient temperature. LCMS/TLC indicated a mixture of regioisomers was formed. The mixture was concentrated and then purified on silica gel (0-50% hexanes/EtOAc gradient elution, later eluting regioisomer) to afford the title compound as a pale yellow residue (81 mg, 37%). ¹H NMR (400 MHz, CHLOROFORM-d) δ=7.38-7.27 (m, 5H), 5.54 (s, 1H), 4.80 (s, 2H), 4.40-4.26 (m, 3H), 4.24-4.09 (m, 1H), 3.63 (s, 2H), 3.35 (s, 1H), 2.21 (s, 3H), 2.14 (s, 3H), 1.22 (t, J=7.1 Hz, 3H), 0.44 (s, 9H); LC/MS (m/z) ES⁺=440 (M+1).

### Step 2: (S)-Ethyl 2-(2-benzyl-7-iodo-5,8-dimethyl-3-oxo-1,2,3,4-tetrahydroisoquinolin-6-yl)-2-hydroxyacetate

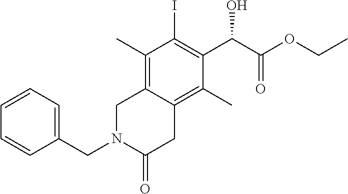 [SMILES: CCOC(=O)[C@@H](O)c1c(C)c2c(c(C)c1I)CN(Cc1ccccc1)C(=O)C2]

An ice cold solution of (S)-ethyl 2-(2-benzyl-5,8-dimethyl-3-oxo-7-(trimethylsilyl)-1,2,3,4-tetrahydroisoquinolin-6-yl)-2-hydroxyacetate (81 mg, 0.184 mmol) in dichloromethane (1.0 mL) was treated slowly with ICl (1M solution in dichloromethane) (0.202 mL, 0.202 mmol) and the mixture was stirred at ice bath temperature for 10 minutes. The mixture was quenched with saturated sodium thiosulfate then extracted with dichloromethane. The extracts were washed with saturated sodium bicarbonate, then brine, dried over sodium sulfate, filtered and concentrated under reduced pressure to a pale yellow solid (85 mg, 94%). LC/MS (m/z) ES⁺=494 (M+1).

### Step 3: (S)-Ethyl 2-(2-benzyl-7-iodo-5,8-dimethyl-3-oxo-1,2,3,4-tetrahydroisoquinolin-6-yl)-2-(tert-butoxy)acetate

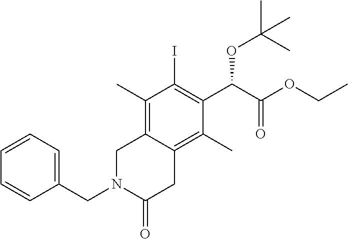 [SMILES: CCOC(=O)[C@@H](OC(C)(C)C)c1c(C)c2c(c(C)c1I)CN(Cc1ccccc1)C(=O)C2]

A suspension of (S)-ethyl 2-(2-benzyl-7-iodo-5,8-dimethyl-3-oxo-1,2,3,4-tetrahydroisoquinolin-6-yl)-2-hydroxyacetate (85 mg, 0.176 mmol) in t-Butyl acetate (3.0 mL) was treated with perchloric acid (0.041 mL, 0.688 mmol) to give a mixture that turned into an orange colored solution over several minutes. The mixture was allowed to stir at ambient temperature for 45 minutes. The mixture was partitioned between EtOAc and 1N NaOH and the phases were separated. The organic phase was washed with brine, dried over sodium sulfate, filtered and concentrated under reduced pressure. The residue was purified on silica gel (12 g column, 0-50% hexanes/EtOAc) to afford a pale yellow residue (56 mg, 59%). ¹H NMR (400 MHz, CHLOROFORM-d) δ=7.39-7.28 (m, 5H), 5.90 (s, 1H), 4.79 (s, 2H), 4.39 (s, 2H), 4.26-4.07 (m, 2H), 3.75-3.52 (m, 2H), 2.35 (s, 3H), 2.31 (s, 3H), 1.25-1.17 (m, 12H); LC/MS (m/z) ES⁺=550 (M+1).

### Step 4: (S)-Ethyl 2-(2-benzyl-5,8-dimethyl-3-oxo-7-(p-tolyl)-1,2,3,4-tetrahydroisoquinolin-6-yl)-2-(tert-butoxy)acetate

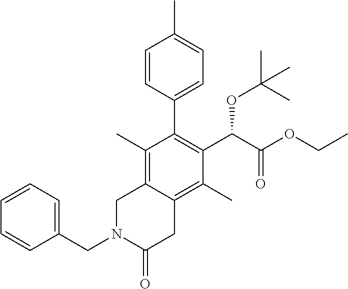 [SMILES: CCOC(=O)[C@@H](OC(C)(C)C)c1c(C)c2c(c(C)c1-c1ccc(C)cc1)CN(Cc1ccccc1)C(=O)C2]

A mixture of (S)-ethyl 2-(2-benzyl-7-iodo-5,8-dimethyl-3-oxo-1,2,3,4-tetrahydroisoquinolin-6-yl)-2-(tert-butoxy)acetate (25.5 mg, 0.046 mmol) in N,N-dimethylformamide (1.0 mL) was degassed while adding p-tolylboronic acid (12.62 mg, 0.093 mmol), K₃PO₄ (29.6 mg, 0.139 mmol), MePhos (3.38 mg, 9.28 μmol) and finally Pd₂dba₃ (8.50 mg, 9.28 μmol). The mixture was degassed for several minutes and then stirred at 80° C. overnight. The mixture was cooled to ambient temperature, diluted with EtOAc, washed with water, then brine, dried over sodium sulfate, filtered and concentrated under reduced pressure. The residue was purified by reverse phase HPLC to afford a pale yellow residue (11.8 mg, 50%). ¹H NMR (400 MHz, CHLOROFORM-d) δ=7.38-7.15 (m, 8H), 7.05-6.98 (m, 1H), 4.97 (s, 1H), 4.81 (s, 2H), 4.41-4.33 (m, 2H), 4.25-4.04 (m, 2H), 3.78-3.61 (m, 2H), 2.42 (s, 3H), 2.33 (s, 3H), 1.74 (s, 3H), 1.23 (t, J=7.1 Hz, 3H), 0.98-0.90 (m, 9H); LC/MS (m/z) ES⁺=514 (M+1).

### Step 5: (S)-2-(2-Benzyl-5,8-dimethyl-3-oxo-7-(p-tolyl)-1,2,3,4-tetrahydroisoquinolin-6-yl)-2-(tert-butoxy)acetic Acid

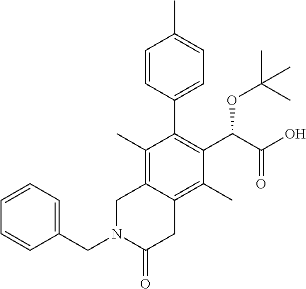 [SMILES: Cc1ccc(-c2c(C)c3c(c(C)c2[C@H](OC(C)(C)C)C(=O)O)CC(=O)N(Cc2ccccc2)C3)cc1]

A mixture of (S)-ethyl 2-(2-benzyl-5,8-dimethyl-3-oxo-7-(p-tolyl)-1,2,3,4-tetrahydroisoquinolin-6-yl)-2-(tert-butoxy)acetate (11.8 mg, 0.023 mmol) in ethanol (0.5 mL) and tetrahydrofuran (0.5 mL) was treated with 2M LiOH (0.3 mL, 0.046 mmol). The mixture was heated to 70° C. and stirred for 10 hours, then allowed to stir at ambient temperature overnight. The mixture was concentrated, diluted with 1N HCl and then extracted with EtOAc. The combined extracts were washed with brine, dried over sodium sulfate, filtered and concentrated under reduced pressure to a pale yellow residue. The residue was purified by reverse phase HPLC to afford a white solid (3.1 mg, 28%). ¹H NMR (400 MHz, CHLOROFORM-d) δ=7.40-7.18 (m, 8H), 7.07-6.98 (m, 1H), 5.15 (s, 1H), 4.88-4.75 (m, 2H), 4.36 (s, 2H), 3.81-3.59 (m, 2H), 2.41 (s, 3H), 2.26 (s, 3H), 1.77 (s, 3H), 0.98 (s, 9H); LC/MS (m/z) ES⁺=486 (M+1).

### Example 102: (S)(M)-2-(2-benzyl-7-(8-fluoro-5-methylchroman-6-yl)-5,8-dimethyl-3-oxo-1,2,3,4-tetrahydroisoquinolin-6-yl)-2-(tert-butoxy)acetic Acid

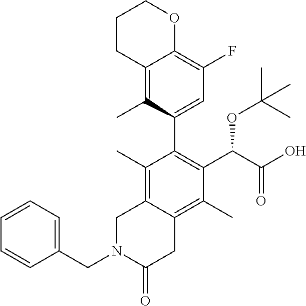

The title compound was prepared in a manner similar to that described in example 101 from (8-fluoro-5-methylchroman-6-yl)boronic acid and was isolated as a white solid after reverse phase HPLC. ¹H NMR (400 MHz, CHLOROFORM-d) δ=7.39-7.26 (m, 5H), 6.66 (d, J=11.4 Hz, 1H), 5.08 (br. s., 1H), 4.90-4.83 (m, 1H), 4.81-4.74 (m, 1H), 4.37 (s, 2H), 4.26 (t, J=5.0 Hz, 2H), 3.73 (s, 2H), 2.76-2.58 (m, 2H), 2.29 (s, 3H), 2.17-2.07 (m, 2H), 1.82 (s, 3H), 1.66 (s, 3H), 1.12 (s, 9H); LC/MS (m/z) ES⁺=560 (M+1).

### Example 103: (S)-2-(tert-Butoxy)-2-((R)-7-(8-fluoro-5-methylchroman-6-yl)-2-(3-fluorophenyl)-5,8-dimethyl-3-oxo-1,2,3,4-tetrahydroisoquinolin-6-yl)acetic Acid

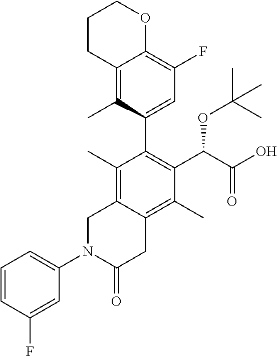

### Step 1: N-(but-2-yn-1-yl)-3-fluoroaniline

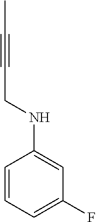

At 0° C., to a solution of 3-fluoroaniline (3.0 g, 27.0 mmol) and K₂CO₃ (3.73 g, 27.0 mmol) in DMF (50 mL) was added 1-bromobut-2-yne (3.95 g, 29.7 mmol). After stirred at r.t. overnight, the resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (2.0 g, 45% yield) as a colorless oil. LC-MS (ESI): m/z (M+1)=164.86.

### Step 2: N-(But-2-yn-1-yl)-N-(3-fluorophenyl)pent-3-ynamide

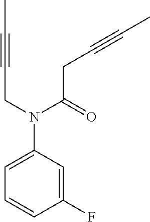 [SMILES: CC#CCC(=O)N(CC#CC)c1cccc(F)c1]

To a mixture of N-(but-2-yn-1-yl)-3-fluoroaniline (2.0 g, 12.4 mmol), pent-3-ynoic acid (1.82 g, 18.6 mmol) in DMF (20 mL) was added T₃P (50% solution in EtOAc, 19.7 g, 31.0 mmol) and DIPEA (5.6 g, 43.4 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (2.0 g, 66% yield) as a yellow oil. LC-MS (ESI): m/z (M+1)=244.40.

### Step 3: Ethyl (S)-2-(2-(3-fluorophenyl)-5,8-dimethyl-3-oxo-7-(trimethylsilyl)-1,2,3,4-tetrahydroisoquinolin-6-yl)-2-hydroxyacetate

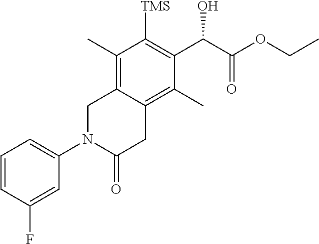 [SMILES: CCOC(=O)[C@@H](O)c1c(C)c2c(c(C)c1[Si](C)(C)C)CN(c1cccc(F)c1)C(=O)C2]

A mixture of Rh(cod)₂BF₄ (171 mg, 0.42 mmol) and BINAP (261 mg, 0.42 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (823 mg, 4.2 mmol) was introduced. The reaction mixture was heated up to 80° C. in a seal tube and N-(but-2-yn-1-yl)-N-(3-fluorophenyl)pent-3-ynamide (500 mg, 2.1 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (250 mg, 27% yield). LC-MS (ESI): m/z (M+1)=444.32.

### Step 4: Ethyl (S)-2-(2-(3-fluorophenyl)-7-iodo-5,8-dimethyl-3-oxo-1,2,3,4-tetrahydro isoquinolin-6-yl)-2-hydroxyacetate

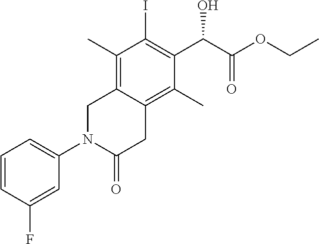 [SMILES: CCOC(=O)[C@@H](O)c1c(C)c2c(c(C)c1I)CN(c1cccc(F)c1)C(=O)C2]

At 0° C., to a solution of ethyl (S)-2-(2-(3-fluorophenyl)-5,8-dimethyl-3-oxo-7-(trimethyl silyl)-1,2,3,4-tetrahydroisoquinolin-6-yl)-2-hydroxyacetate (100 mg, 0.23 mmol) in DCM (2 mL) was added ICl (2.5M solution in DCM, 0.1 mL, 0.25 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 5: Ethyl (S)-2-(tert-butoxy)-2-(2-(3-fluorophenyl)-7-iodo-5,8-dimethyl-3-oxo-1,2,3,4-tetrahydroisoquinolin-6-yl)acetate

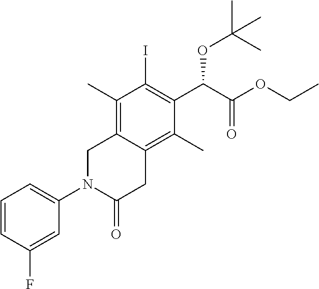 [SMILES: CCOC(=O)[C@@H](OC(C)(C)C)c1c(C)c2c(c(C)c1I)CN(c1cccc(F)c1)C(=O)C2]

To a solution of ethyl (S)-2-(2-(3-fluorophenyl)-7-iodo-5,8-dimethyl-3-oxo-1,2,3,4-tetrahydroisoquinolin-6-yl)-2-hydroxyacetate (crude product from the previous step, 0.23 mmol) in t-BuOAc (2 mL) was added HClO₄ (20 mg, 0.2 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (90 mg, 71% yield over two steps). LC-MS (ESI): m/z (M+1)=554.53.

### Step 6: Ethyl (2S)-2-(tert-butoxy)-2-(7-(8-fluoro-5-methylchroman-6-yl)-2-(3-fluoro phenyl)-5,8-dimethyl-3-oxo-1,2,3,4-tetrahydroisoquinolin-6-yl)acetate

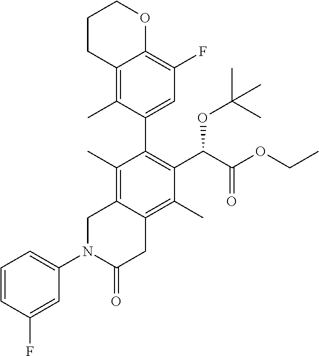 [SMILES: CCOC(=O)[C@@H](OC(C)(C)C)c1c(C)c2c(c(C)c1-c1cc(F)c3c(c1C)CCCO3)CN(c1cccc(F)c1)C(=O)C2]

A mixture of ethyl (S)-2-(tert-butoxy)-2-(2-(3-fluorophenyl)-7-iodo-5,8-dimethyl-3-oxo-1,2,3,4-tetrahydroisoquinolin-6-yl)acetate (90 mg, 0.16 mmol), (8-fluoro-5-methyl chroman-6-yl) boronic acid (136 mg, 0.65 mmol), Pd(Pt-Bu₃)₂ (16 mg, 0.032 mmol) and Cs₂CO₃ (211 mg, 0.65 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (70 mg, 73% yield). LC-MS (ESI): m/z (M+1)=592.62.

### Step 7: (S)-2-(tert-Butoxy)-2-((R)-7-(8-fluoro-5-methylchroman-6-yl)-2-(3-fluorophenyl)-5,8-dimethyl-3-oxo-1,2,3,4-tetrahydroisoquinolin-6-yl)acetic Acid

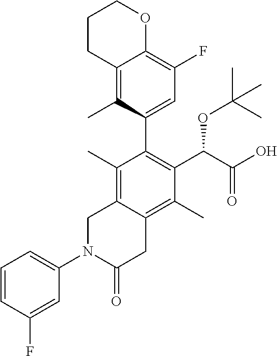

A mixture of ethyl (2S)-2-(tert-butoxy)-2-(7-(8-fluoro-5-methylchroman-6-yl)-2-(3-fluorophenyl)-5,8-dimethyl-3-oxo-1,2,3,4-tetrahydroisoquinolin-6-yl)acetate (70 mg, 0.12 mmol) in EtOH (1 mL) and dioxane (2 mL) was added LiOH (2.5N, 0.47 mL, 1.2 mmol). After stirred at r.t. for 1 hr, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to give the product as a mixture diastereomers which was further separated by chiral SFC (Superchiral S—OZ column with CO₂/EtOH=70/30 (v/v)) to afford the title compound (20 mg, 30% yield). LC-MS (ESI): m/z (M+1)=564.27. ¹H NMR (400 MHz, CDCl₃) δ 9.69 (br, 1H), 7.47-7.36 (m, 1H), 7.22-7.09 (m, 2H), 7.02 (td, J=8.5, 2.0 Hz, 1H), 6.70 (d, J=11.3 Hz, 1H), 5.11 (s, 1H), 4.88-4.74 (m, 2H), 4.33-4.21 (m, 2H), 3.88-3.73 (m, 2H), 2.77-2.62 (m, 2H), 2.34 (s, 3H), 2.17-2.06 (m, 2H), 1.87 (s, 3H), 1.79 (s, 3H), 1.14 (s, 9H).

### Example 104: (S)-2-(tert-butoxy)-2-((R)-2-cyclohexyl-7-(8-fluoro-5-methylchroman-6-yl)-5,8-dimethyl-3-oxo-1,2,3,4-tetrahydroisoquinolin-6-yl)acetic Acid

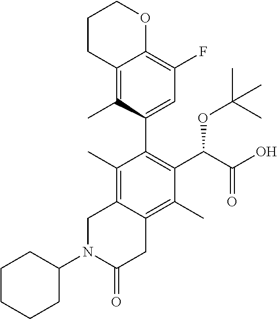

### Step 1: (S)-Ethyl 2-(2-cyclohexyl-5,8-dimethyl-3-oxo-7-(trimethylsilyl)-1,2,3,4-tetrahydroisoquinolin-6-yl)-2-hydroxyacetate

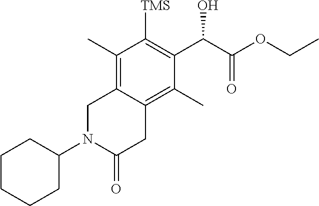 [SMILES: CCOC(=O)[C@@H](O)c1c(C)c2c(c(C)c1[Si](C)(C)C)CN(C1CCCCC1)C(=O)C2]

The title compound was prepared in 30% yield from (S)-ethyl 2-hydroxy-4-(trimethylsilyl)but-3-ynoate and N-(but-2-yn-1-yl)-N-cyclohexylpent-3-ynamide according to example 101, step 1. ¹H NMR (400 MHz, CHLOROFORM-d) δ 5.56 (s, 1H), 4.52-4.67 (m, 1H), 4.10-4.41 (m, 4H), 3.53 (s, 2H), 3.39 (s, 1H), 2.38 (s, 3H), 2.13 (s, 3H), 1.09-1.95 (m, 13H), 0.48 (s, 9H).

### Step 2: (S)-ethyl 2-(tert-butoxy)-2-(2-cyclohexyl-7-iodo-5,8-dimethyl-3-oxo-1,2,3,4-tetrahydroisoquinolin-6-yl)acetate

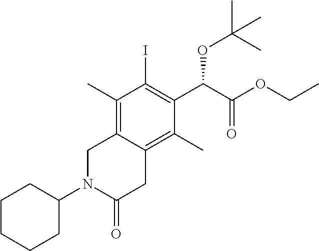 [SMILES: CCOC(=O)[C@@H](OC(C)(C)C)c1c(C)c2c(c(C)c1I)CN(C1CCCCC1)C(=O)C2]

The title compound was made in a manner similar to that described in example 101 step 2-3 in 45% yield as yellow foam. ¹H NMR (400 MHz, CHLOROFORM-d) δ ppm 5.91 (s, 1H), 4.66-4.52 (m, 1H), 4.43-4.30 (m, 2H), 4.24-4.06 (m, 2H), 3.66-3.37 (m, 2H), 2.53 (s, 3H), 2.30 (s, 3H), 1.92-1.79 (m, 2H), 1.79-1.63 (m, 4H), 1.57-1.36 (m, 4H), 1.31-1.07 (m, 12H); LCMS (m/z) ES⁺=542.3 (M+1).

### Step 3: (2S)-ethyl 2-(tert-butoxy)-2-(2-cyclohexyl-7-(8-fluoro-5-methylchroman-6-yl)-5,8-dimethyl-3-oxo-1,2,3,4-tetrahydroisoquinolin-6-yl)acetate

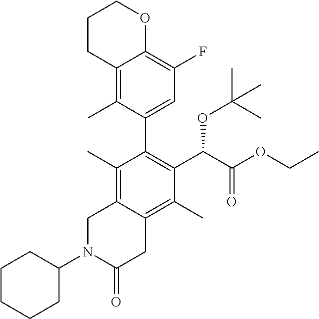 [SMILES: CCOC(=O)[C@@H](OC(C)(C)C)c1c(C)c2c(c(C)c1-c1cc(F)c3c(c1C)CCCO3)CN(C1CCCCC1)C(=O)C2]

A solution of (S)-ethyl 2-(tert-butoxy)-2-(2-cyclohexyl-7-iodo-5,8-dimethyl-3-oxo-1,2,3,4-tetrahydroisoquinolin-6-yl)acetate (46 mg, 0.085 mmol) in 1,4-Dioxane (1.5 mL) was degassed with N₂ while adding (8-fluoro-5-methylchroman-6-yl)boronic acid (71.4 mg, 0.340 mmol), Cs2CO₃ (83 mg, 0.255 mmol), and bis(tri-t-butylphosphine)palladium(O) (8.68 mg, 0.017 mmol). The reaction was stirred in a preheated 90° C. oil bath for 10 min. The mixture was cooled to rt, diluted with EtOAc, washed with Brine, dried over Na2SO4, filtered, and concentrated. Purification with column chromatography (0-80% EtOAc/Hexane) afforded ˜3:1 mixture of (M)(S): (P)(S) atropisomers of (2S)-ethyl 2-(tert-butoxy)-2-(2-cyclohexyl-7-(8-fluoro-5-methylchroman-6-yl)-5,8-dimethyl-3-oxo-1,2,3,4-tetrahydroisoquinolin-6-yl)acetate (39.6 mg, 0.068 mmol, 80% yield) as yellow film. LCMS (m/z) ES⁺=580.5 (M+1).

### Step 4: (S)-2-(tert-butoxy)-2-((R)-2-cyclohexyl-7-(8-fluoro-5-methylchroman-6-yl)-5,8-dimethyl-3-oxo-1,2,3,4-tetrahydroisoquinolin-6-yl)acetic Acid

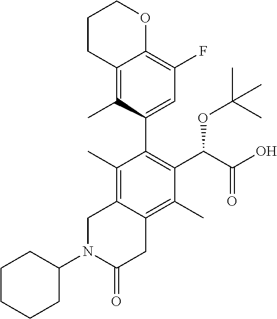

A solution of (2S)-ethyl 2-(tert-butoxy)-2-(2-cyclohexyl-7-(8-fluoro-5-methylchroman-6-yl)-5,8-dimethyl-3-oxo-1,2,3,4-tetrahydroisoquinolin-6-yl)acetate (67 mg, 0.116 mmol) in 1,4-Dioxane (1.5 mL) and Ethanol (0.5 mL) was treated with 2M LiOH (0.5 mL, 1.000 mmol) and stirred at 100° C. for 12 hours. The reaction was cooled to rt and concentrated. The residue was diluted with EtOAc, washed with 1N HCl, Brine, dried over Na2SO4, filtered, and concentrated. Purification with reverse phase HPLC (30-100% MeCN-0.1% formic acid/H2O-0.1% formic acid) afforded the title compound ((M)(S) atropisomer) as white solid. NMR indicated approx. 5% (P)(S) atropisomer. ¹H NMR (400 MHz, METHANOL-d₄) δ ppm 6.61 (d, J=11.4 Hz, 1H), 4.99 (s, 1H), 4.56-4.37 (m, 3H), 4.23 (t, J=5.0 Hz, 2H), 3.60 (s, 2H), 2.73 (t, J=6.4 Hz, 2H), 2.39 (s, 3H), 2.19-2.06 (m, 2H), 1.95-1.84 (m, 5H), 1.83-1.77 (m, 3H), 1.76-1.58 (m, 5H), 1.53-1.36 (m, 2H), 1.31-1.19 (m, 1H), 1.12-0.94 (m, 9H); LCMS (m/z) ES⁺=552.4 (M+1), ES⁻=550.3 (M−1).

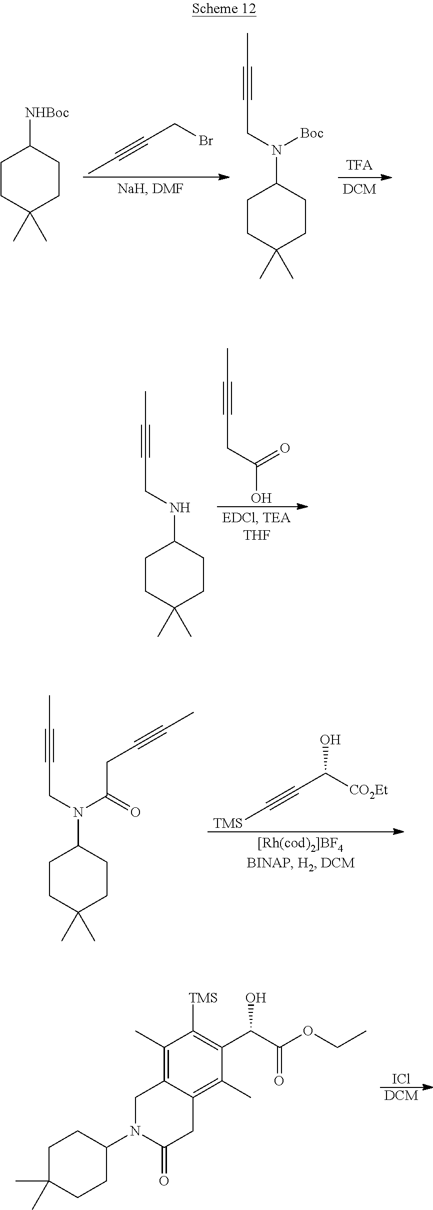

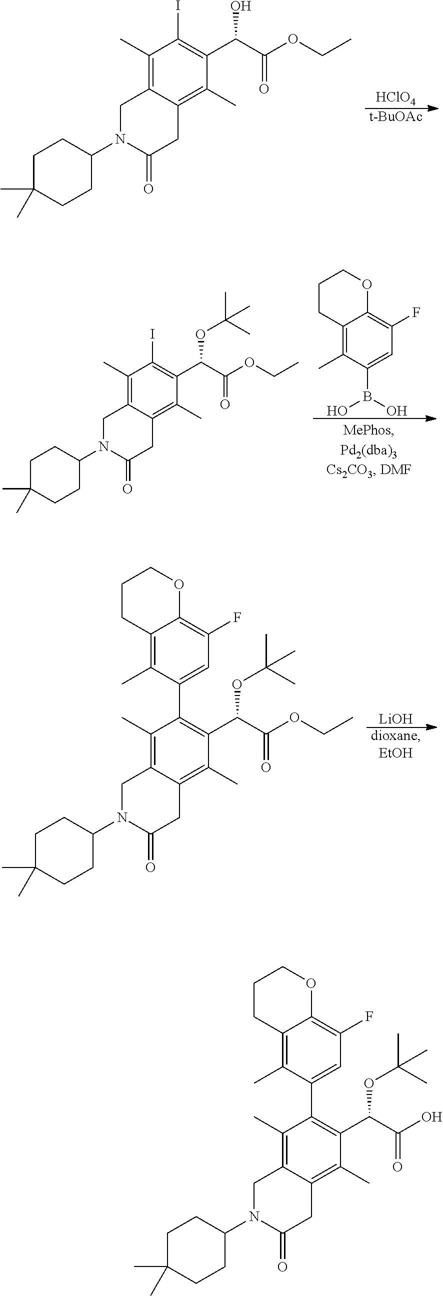

### Example 105: (S)-2-(tert-butoxy)-2-((R)-2-(4,4-dimethylcyclohexyl)-7-(8-fluoro-5-methylchroman-6-yl)-5,8-dimethyl-3-oxo-1,2,3,4-tetrahydroisoquinolin-6-yl)acetic Acid

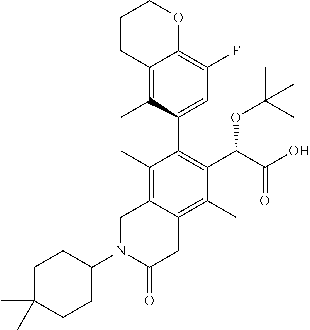

### Step 1: tert-Butyl but-2-yn-1-yl(4,4-dimethylcyclohexyl)carbamate

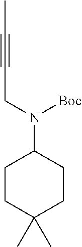 [SMILES: CC#CCN(C(=O)OC(C)(C)C)C1CCC(C)(C)CC1]

At 0° C., to a solution of tert-butyl (4,4-dimethylcyclohexyl)carbamate (2.21 g, 9.7 mmol) in DMF (20 mL) was added NaH (60%, 467 mg, 11.67 mmol). The resulting mixture was stirred at r.t. for 30 min before the introduction of 1-bromobut-2-yne (1.42 g, 10.7 mmol). The reaction mixture was stirred at r.t. for 2 hr and quenched with sat. NH₄Cl aq. solution and extracted with EtOAc. The organic layer was washed with brine, dried over Na₂SO₄, filtered and concentrated to give the crude product which was purified by flash chromatography (silica gel, 0-20% EtOAc in PE) to afford the title compound (1.0 g, 37% yield). LC-MS (ESI): m/z (M+1)=280.56.

### Step 2: N-(but-2-yn-1-yl)-4,4-dimethylcyclohexan-1-amine

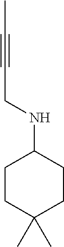

A mixture of tert-butyl but-2-yn-1-yl(4,4-dimethylcyclohexyl)carbamate (1.0 g, 3.58 mmol) in TEA (2 mL) and DCM (10 mL) was stirred at r.t. for 3 hr. The resulting mixture was concentrated and diluted with DCM and washed with sat. NaHCO₃ aq. solution. The organic layer was washed with brine, dried over Na₂SO₄, filtered and concentrated to afford the title compound (586 mg, 92% yield). LC-MS (ESI): m/z (M+1)=180.58.

### Step 3: N-(but-2-yn-1-yl)-N-(4,4-dimethylcyclohexyl)pent-3-ynamide

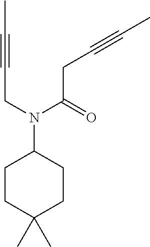 [SMILES: CC#CCC(=O)N(CC#CC)C1CCC(C)(C)CC1]

To a mixture of N-(but-2-yn-1-yl)-4,4-dimethylcyclohexan-1-amine (586 mg, 3.27 mmol), pent-3-ynoic acid (480 mg, 4.9 mmol) in THF (10 mL) was added EDCl (936 mg, 4.9 mmol) and TEA (660 mg, 6.53 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (600 mg, 71% yield). LC-MS (ESI): m/z (M+1)=260.40.

### Step 4: Ethyl (S)-2-(2-(4,4-dimethylcyclohexyl)-5,8-dimethyl-3-oxo-7-(trimethylsilyl)-1,2,3,4-tetrahydroisoquinolin-6-yl)-2-hydroxyacetate

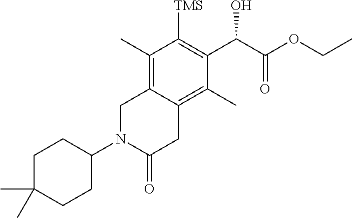 [SMILES: CCOC(=O)[C@@H](O)c1c(C)c2c(c(C)c1[Si](C)(C)C)CN(C1CCC(C)(C)CC1)C(=O)C2]

A mixture of Rh(cod)₂BF₄ (130 mg, 0.32 mmol) and BINAP (199 mg, 0.32 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (648 mg, 3.2 mmol) was introduced. The reaction mixture was heated up to 80° C. in a seal tube and N-(but-2-yn-1-yl)-N-(4,4-dimethylcyclohexyl) pent-3-ynamide (420 mg, 1.6 mmol) was added as a DCE solution (5 mL) over 3 hr. After stirred at 80° C. for 30 min, the resulting mixture was concentrated to give the crude product which was purified by flash chromatography (silica gel, 0-30% EtOAc in PE) to afford the title compound (290 mg, 39% yield). LC-MS (ESI): m/z (M+1)=460.16.

### Step 5: Ethyl (S)-2-(2-(4,4-dimethylcyclohexyl)-7-iodo-5,8-dimethyl-3-oxo-1,2,3,4-tetrahydroisoquinolin-6-yl)-2-hydroxyacetate

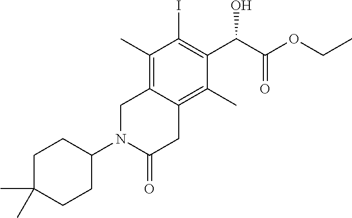 [SMILES: CCOC(=O)[C@@H](O)c1c(C)c2c(c(C)c1I)CN(C1CCC(C)(C)CC1)C(=O)C2]

At 0° C., to a solution of ethyl (S)-2-(2-(4,4-dimethylcyclohexyl)-5,8-dimethyl-3-oxo-7-(trimethylsilyl)-1,2,3,4-tetrahydroisoquinolin-6-yl)-2-hydroxyacetate (290 mg, 0.63 mmol) in DCM (2 mL) was added ICl (2.5M solution in DCM, 0.27 mL, 0.68 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 6: Ethyl (S)-2-(tert-butoxy)-2-(2-(4,4-dimethylcyclohexyl)-7-iodo-5,8-dimethyl-3-oxo-1,2,3,4-tetrahydroisoquinolin-6-yl)acetate

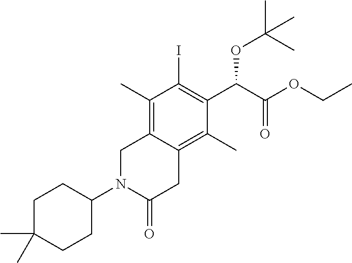 [SMILES: CCOC(=O)[C@@H](OC(C)(C)C)c1c(C)c2c(c(C)c1I)CN(C1CCC(C)(C)CC1)C(=O)C2]

To a solution of ethyl (S)-2-(2-(4,4-dimethylcyclohexyl)-7-iodo-5,8-dimethyl-3-oxo-1,2,3,4-tetrahydroisoquinolin-6-yl)-2-hydroxyacetate (crude product from the previous step, 0.63 mmol) in t-BuOAc (4 mL) was added HClO₄ (63 mg, 0.62 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (200 mg, 56% yield over two steps). LC-MS (ESI): m/z (M+1)=570.40.

### Step 7: Ethyl (2S)-2-(tert-butoxy)-2-(2-(4,4-dimethylcyclohexyl)-7-(8-fluoro-5-methyl chroman-6-yl)-5,8-dimethyl-3-oxo-1,2,3,4-tetrahydroisoquinolin-6-yl)acetate

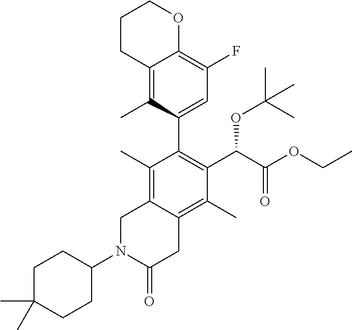

A mixture of ethyl (S)-2-(tert-butoxy)-2-(2-(4,4-dimethylcyclohexyl)-7-iodo-5,8-dimethy l-3-oxo-1,2,3,4-tetrahydroisoquinolin-6-yl)acetate (50 mg, 0.08 mmol), (8-fluoro-5-methylchroman-6-yl) boronic acid (73 mg, 0.35 mmol), MePhos (7 mg, 0.018 mmol) Pd₂(dba)₃ (16 mg, 0.018 mmol) and Cs₂CO₃ (58 mg, 0.18 mmol) in DMF (2 mL) was stirred at 90° C. under N₂ atmosphere overnight. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (41 mg, 85% yield). LC-MS (ESI): m/z (M+1)=608.90.

### Step 8: (S)-2-(tert-butoxy)-2-((R)-2-(4,4-dimethylcyclohexyl)-7-(8-fluoro-5-methylchroman-6-yl)-5,8-dimethyl-3-oxo-1,2,3,4-tetrahydroisoquinolin-6-yl)acetic Acid

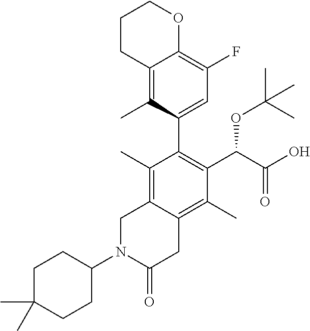

A mixture of ethyl (2S)-2-(tert-butoxy)-2-(2-(4,4-dimethylcyclohexyl)-7-(8-fluoro-5-methylchroman-6-yl)-5,8-dimethyl-3-oxo-1,2,3,4-tetrahydroisoquinolin-6-yl)acetate (41 mg, 0.067 mmol) in EtOH (0.5 mL) and dioxane (1 mL) was added LiOH (2.5N, 0.27 mL, 0.67 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (15 mg, 38% yield). LC-MS (ESI): m/z (M+1)=580.56. ¹H NMR (400 MHz, CDCl₃) δ 9.65 (br, 1H), 6.70 (d, J=11.3 Hz, 1H), 5.08 (s, 1H), 4.57-4.49 (m, 1H), 4.42-4.24 (m, 4H), 3.67-3.56 (m, 2H), 2.76-2.62 (m, 2H), 2.28 (s, 3H), 2.17-2.08 (m, 2H), 1.84 (d, J=11.3 Hz, 6H), 1.78-1.68 (m, 2H), 1.59-1.47 (m, 6H), 1.11 (s, 9H), 0.96 (d, J=11.0 Hz, 6H).

### Example 106: (S)-2-(tert-butoxy)-2-((R)-2-(cyclohexylmethyl)-7-(8-fluoro-5-methylchroman-6-yl)-5,8-dimethyl-3-oxo-1,2,3,4-tetrahydroisoquinolin-6-yl)acetic Acid

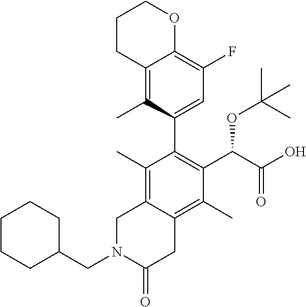

### Step 1: N-(Cyclohexylmethyl)but-2-yn-1-amine

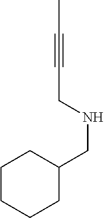

At 0° C., 1-bromobut-2-yne (1.48 g, 11.1 mmol) was slowed added to cyclohexyl methanamine (10 g, 88.3 mmol). After stirred at 0° C. for 30 min, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (1.5 g, 82% yield). LC-MS (ESI): m/z (M+1)=166.50.

### Step 2: N-(But-2-yn-1-yl)-N-(cyclohexylmethyl)pent-3-ynamide

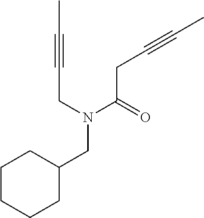 [SMILES: CC#CCC(=O)N(CC#CC)CC1CCCCC1]

To a mixture of N-(cyclohexylmethyl)but-2-yn-1-amine (1.5 g, 9.1 mmol), pent-3-ynoic acid (1.3 g, 13.6 mmol) in THF (20 mL) was added EDCl (2.6 g, 13.6 mmol) and TEA (1.8 g, 17.8 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (1.3 g, 59% yield) as a yellow oil. LC-MS (ESI): m/z (M+1)=246.44.

### Step 3: Ethyl (S)-2-(2-(cyclohexylmethyl)-5,8-dimethyl-3-oxo-7-(trimethylsilyl)-1,2,3,4-tetrahydroisoquinolin-6-yl)-2-hydroxyacetate

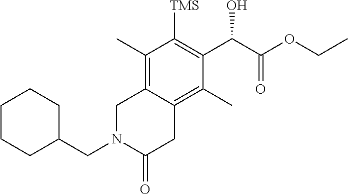 [SMILES: CCOC(=O)[C@@H](O)c1c(C)c2c(c(C)c1[Si](C)(C)C)CN(CC1CCCCC1)C(=O)C2]

A mixture of Rh(cod)₂BF₄ (150 mg, 0.37 mmol) and BINAP (230 mg, 0.0.37 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (734 mg, 3.7 mmol) was introduced. The reaction mixture was heated up to 80° C. in a seal tube and N-(but-2-yn-1-yl)-N-(cyclohexylmethyl)pent-3-ynamide (450 mg, 1.8 mmol) was added as a DCE solution (5 mL) over 3 hr. After stirred at 80° C. for 30 min, the resulting mixture was concentrated to give the crude product which was purified by flash chromatography (silica gel, 0-40% EtOAc in PE) to afford the title compound (150 mg, 19% yield). LC-MS (ESI): m/z (M+1)=446.41.

### Step 4: Ethyl (S)-2-(2-(cyclohexylmethyl)-7-iodo-5,8-dimethyl-3-oxo-1,2,3,4-tetrahydroisoquinolin-6-yl)-2-hydroxyacetate

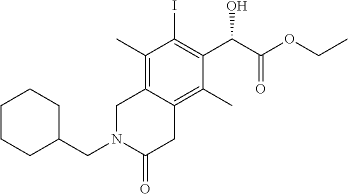 [SMILES: CCOC(=O)[C@@H](O)c1c(C)c2c(c(C)c1I)CN(CC1CCCCC1)C(=O)C2]

At 0° C., to a solution of ethyl (S)-2-(2-(cyclohexylmethyl)-5,8-dimethyl-3-oxo-7-(trimethylsilyl)-1,2,3,4-tetrahydroisoquinolin-6-yl)-2-hydroxyacetate (150 mg, 0.34 mmol) in DCM (2 mL) was added ICl (2.5M solution in DCM, 0.15 mL, 0.37 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=500.32.

### Step 5: Ethyl (S)-2-(tert-butoxy)-2-(2-(cyclohexylmethyl)-7-iodo-5,8-dimethyl-3-oxo-1,2,3,4-tetrahydroisoquinolin-6-yl)acetate

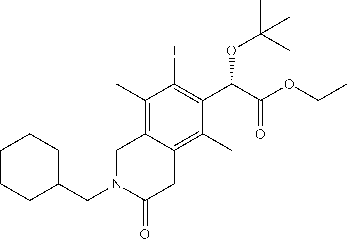 [SMILES: CCOC(=O)[C@@H](OC(C)(C)C)c1c(C)c2c(c(C)c1I)CN(CC1CCCCC1)C(=O)C2]

To a solution of ethyl (S)-2-(2-(cyclohexylmethyl)-7-iodo-5,8-dimethyl-3-oxo-1,2,3,4-tetrahydroisoquinolin-6-yl)-2-hydroxyacetate (crude product from the previous step, 0.34 mmol) in t-BuOAc (2 mL) was added HClO₄ (34 mg, 0.34 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (68 mg, 36% yield over two steps). LC-MS (ESI): m/z (M+1)=556.71.

### Step 6: (S)-ethyl 2-(tert-butoxy)-2-((R)-2-(cyclohexylmethyl)-7-(8-fluoro-5-methylchroman-6-yl)-5,8-dimethyl-3-oxo-1,2,3,4-tetrahydroisoquinolin-6-yl)acetate

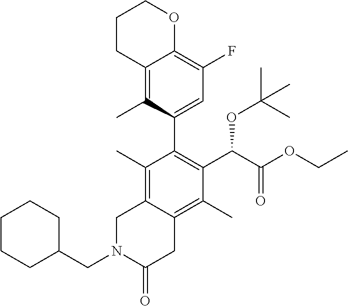

A mixture of ethyl (S)-2-(tert-butoxy)-2-(2-(cyclohexylmethyl)-7-iodo-5,8-dimethyl-3-oxo-1,2,3,4-tetrahydroisoquinolin-6-yl)acetate (68 mg, 0.12 mmol), (8-fluoro-5-methyl chroman-6-yl) boronic acid (101 mg, 0.48 mmol), Pd(Pt-Bu₃)₂ (12 mg, 0.024 mmol) and Cs₂CO₃ (156 mg, 0.48 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (15 mg, 21% yield). LC-MS (ESI): m/z (M+1)=594.72.

### Step 7: (S)-2-(tert-butoxy)-2-((R)-2-(cyclohexylmethyl)-7-(8-fluoro-5-methylchroman-6-yl)-5,8-dimethyl-3-oxo-1,2,3,4-tetrahydroisoquinolin-6-yl)acetic Acid

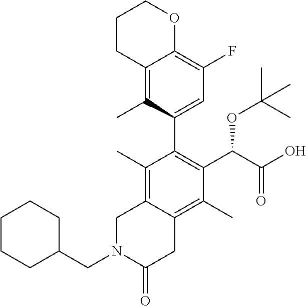

A mixture of ethyl (2S)-2-(tert-butoxy)-2-(2-(cyclohexylmethyl)-7-(8-fluoro-5-methyl chroman-6-yl)-5,8-dimethyl-3-oxo-1,2,3,4-tetrahydroisoquinolin-6-yl)acetate (15 mg, 0.025 mmol) in EtOH (0.5 mL) and dioxane (1 mL) was added LiOH (2.5N, 0.1 mL, 0.25 mmol). After stirred at 100° c. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (9 mg, 63% yield). LC-MS (ESI): m/z (M+1)=566.90. ¹H NMR (400 MHz, CDCl₃) δ 9.53 (br, 1H), 6.70 (d, J=11.2 Hz, 1H), 5.08 (s, 1H), 4.42 (s, 2H), 4.32-4.22 (m, 2H), 3.62 (s, 2H), 3.47-3.35 (m, 2H), 2.74-2.62 (m, 2H), 2.37-2.21 (m, 3H), 2.12 (dd, J=6.1, 3.8 Hz, 2H), 1.89-1.56 (m, 13H), 1.25-0.99 (m, 13H).

### Example 107: (S)-2-(tert-butoxy)-2-((R)-2-(3-fluoro-2-methoxyphenyl)-7-(8-fluoro-5-methylchroman-6-yl)-5,8-dimethyl-3-oxo-1,2,3,4-tetrahydroisoquinolin-6-yl)acetic Acid

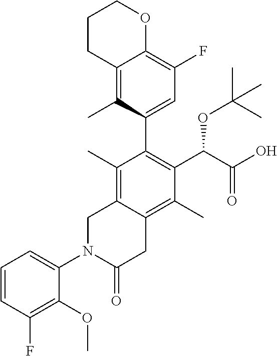

### Step 1: N-(but-2-yn-1-yl)-3-fluoro-2-methoxyaniline

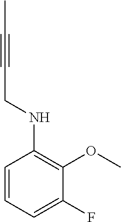

A mixture of 3-fluoro-2-methoxyaniline (1.0 g, 7.1 mmol) and K₂CO₃ (1.04 g, 7.8 mmol) in DMF (15 mL) was added 1-bromobut-2-yne (981 mg, 7.1 mmol) was stirred at r.t. overnight. The resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (552 mg, 40% yield) as a yellow oil. LC-MS (ESI): m/z (M+1)=193.91.

### Step 2: N-(but-2-yn-1-yl)-N-(3-fluoro-2-methoxyphenyl)pent-3-ynamide

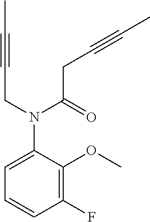 [SMILES: CC#CCC(=O)N(CC#CC)c1cccc(F)c1OC]

To a mixture of N-(but-2-yn-1-yl)-3-fluoro-2-methoxyaniline (552 mg, 2.8 mmol), pent-3-ynoic acid (421 mg, 4.3 mmol) in DMF (5 mL) was added T₃P (50% solution in EtOAc, 4.5 g, 7.1 mmol) and DIPEA (1.3 g, 10 mmol). After stirred at r.t. for 30 min, the resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (450 mg, 59% yield) as a yellow oil. LC-MS (ESI): m/z (M+1)=274.18.

### Step 3: ethyl (S)-2-(2-(3-fluoro-2-methoxyphenyl)-5,8-dimethyl-3-oxo-7-(trimethyl silyl)-1,2,3,4-tetrahydroisoquinolin-6-yl)-2-hydroxyacetate

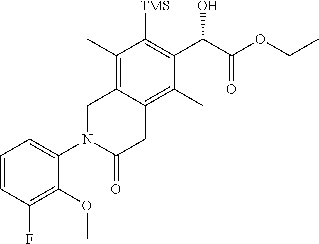 [SMILES: CCOC(=O)[C@@H](O)c1c(C)c2c(c(C)c1[Si](C)(C)C)CN(c1cccc(F)c1OC)C(=O)C2]

A mixture of Rh(cod)₂BF₄ (134 mg, 0.33 mmol) and BINAP (205 mg, 0.33 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (662 mg, 3.3 mmol) was introduced. The reaction mixture was heated up to 80° C. in a seal tube and N-(but-2-yn-1-yl)-N-(3-fluoro-2-methoxphenyl) pent-3-ynamide (450 mg, 1.65 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (130 mg, 17% yield). LC-MS (ESI): m/z (M+1)=474.42.

### Step 4: ethyl (S)-2-(2-(3-fluoro-2-methoxyphenyl)-7-iodo-5,8-dimethyl-3-oxo-1,2,3,4-tetrahydroisoquinolin-6-yl)-2-hydroxyacetate

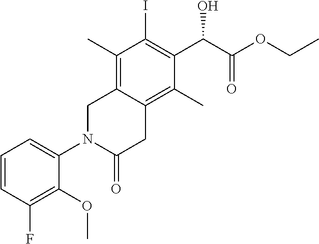 [SMILES: CCOC(=O)[C@@H](O)c1c(C)c2c(c(C)c1I)CN(c1cccc(F)c1OC)C(=O)C2]

At 0° C., to a solution of ethyl (S)-2-(2-(3-fluoro-2-methoxyphenyl)-5,8-dimethyl-3-oxo-7-(trimethylsilyl)-1,2,3,4-tetrahydroisoquinolin-6-yl)-2-hydroxyacetate (130 mg, 0.27 mmol) in DCM (2 mL) was added ICl (2.5M solution in DCM, 0.11 mL, 0.27 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=528.24.

### Step 5: ethyl (S)-2-(tert-butoxy)-2-(2-(3-fluoro-2-methoxyphenyl)-7-iodo-5,8-dimethyl-3-oxo-1,2,3,4-tetrahydroisoquinolin-6-yl)acetate

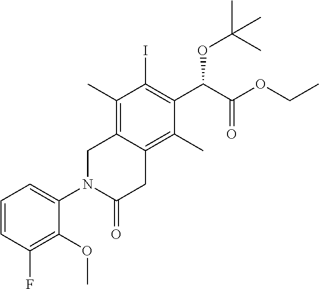 [SMILES: CCOC(=O)[C@@H](OC(C)(C)C)c1c(C)c2c(c(C)c1I)CN(c1cccc(F)c1OC)C(=O)C2]

To a solution of ethyl (S)-2-(2-(3-fluoro-2-methoxphenyl)-7-iodo-5,8-dimethyl-3-oxo-1,2,3,4-tetrahydroisoquinolin-6-yl)-2-hydroxyacetate (crude product from the previous step, 0.27 mmol) in t-BuOAc (2 mL) was added HClO₄ (28 mg, 0.28 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (100 mg, 63% yield over two steps). LC-MS (ESI): m/z (M+1)=584.41.

### Step 6: (S)-ethyl 2-(tert-butoxy)-2-((R)-2-(3-fluoro-2-methoxyphenyl)-7-(8-fluoro-5-methylchroman-6-yl)-5,8-dimethyl-3-oxo-1,2,3,4-tetrahydroisoquinolin-6-yl)acetate

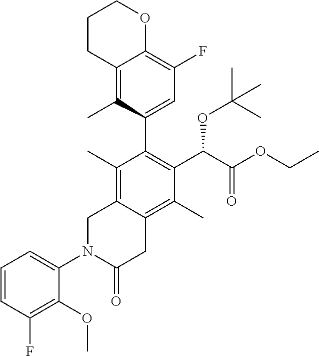

A mixture of ethyl (S)-2-(tert-butoxy)-2-(2-(3-fluoro-2-methoxyphenyl)-7-iodo-5,8-dimethyl-3-oxo-1,2,3,4-tetrahydroisoquinolin-6-yl)acetate (50 mg, 0.085 mmol), (8-fluoro-5-methyl chroman-6-yl) boronic acid (71 mg, 0.34 mmol), Pd(Pt-Bu₃)₂ (8 mg, 0.017 mmol) and Cs₂CO₃ (110 mg, 0.34 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (35 mg, 66% yield). LC-MS (ESI): m/z (M+1)=622.84.

### Step 7: (S)-2-(tert-butoxy)-2-((R)-2-(3-fluoro-2-methoxyphenyl)-7-(8-fluoro-5-methylchroman-6-yl)-5,8-dimethyl-3-oxo-1,2,3,4-tetrahydroisoquinolin-6-yl)acetic Acid

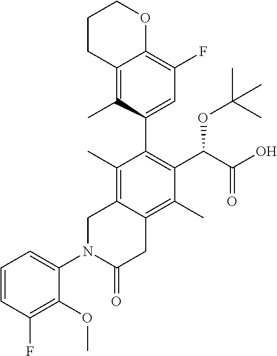

A mixture of ethyl (2S)-2-(tert-butoxy)-2-(2-(3-fluoro-2-methoxyphenyl)-7-(8-fluoro-5-methylchroman-6-yl)-5,8-dimethyl-3-oxo-1,2,3,4-tetrahydroisoquinolin-6-yl)acetate (35 mg, 0.056 mmol) in EtOH (0.5 mL) and dioxane (1 mL) was added LiOH (2.5N, 0.23 mL, 0.56 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (13 mg, 39% yield). LC-MS (ESI): m/z (M+1)=594.66. ¹H NMR (400 MHz, CDCl₃) δ 9.60 (br, 1H), 7.18-6.98 (m, 3H), 6.70 (d, J=11.5 Hz, 1H), 5.12 (s, 1H), 4.81-4.60 (m, 2H), 4.36-4.17 (m, 2H), 3.92 (d, J=1.7 Hz, 3H), 3.81 (s, 2H), 2.77-2.62 (m, 2H), 2.33 (s, 3H), 2.17-2.07 (m, 2H), 1.87 (s, 3H), 1.75 (s, 3H), 1.14 (s, 9H).

### Example 108: (S)-2-(tert-butoxy)-2-((R)-7-(8-fluoro-5-methylchroman-6-yl)-5,8-dimethyl-3-oxo-2-(tetrahydro-2H-pyran-4-yl)-1,2,3,4-tetrahydroisoquinolin-6-yl)acetic Acid

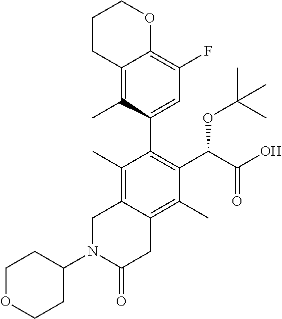

### Step 1: tert-butyl but-2-yn-1-yl(tetrahydro-2H-pyran-4-yl)carbamate

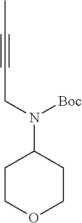 [SMILES: CC#CCN(C(=O)OC(C)(C)C)C1CCOCC1]

At 0° C., to a solution of tert-butyl (tetrahydro-2H-pyran-4-yl)carbamate (1 g, 5.0 mmol) in THF (10 mL) was added NaH (60%, 240 mg, 6.0 mmol). The reaction mixture was stirred at 0° C. for 30 min before the addition of 1-bromobut-2-yne (990 mg, 7.5 mmol). After stirred at r.t. overnight, the resulting mixture was quenched with sat. NH₄Cl aq. solution and extracted with EtOAc. The organic layer was washed with brine, dried over Na₂SO₄, filtered and concentrated to give the crude product which was purified by flash chromatography (silica gel, 0-20% EtOAc in PE) to afford the title compound (1.0 g, 79% yield). LC-MS (ESI): m/z (M+1)=254.33.

### Step 2: N-(but-2-yn-1-yl)tetrahydro-2H-pyran-4-amine

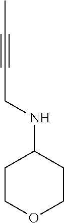

A mixture of tert-butyl but-2-yn-1-yl(tetrahydro-2H-pyran-4-yl)carbamate (1.0 g, 3.16 mmol) in TEA (10 mL) and DCM (20 mL) was stirred at r.t. for 2 hr. The resulting mixture was concentrated under reduced pressure and the residue was dissolved in DCM, washed with sat. NaHCO₃ aq. solution and brine, dried over Na₂SO₄, filtered and concentrated to afford the title compound (660 mg, 99% yield) which was used in the next step without further purification. LC-MS (ESI): m/z (M+1)=154.41.

### Step 3: N-(but-2-yn-1-yl)-N-(tetrahydro-2H-pyran-4-yl)pent-3-ynamide

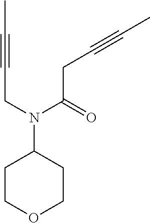 [SMILES: CC#CCC(=O)N(CC#CC)C1CCOCC1]

To a mixture of N-(but-2-yn-1-yl)tetrahydro-2H-pyran-4-amine (600 mg, 3.9 mmol), pent-3-ynoic acid (578 mg, 5.9 mmol) in THF (20 mL) was added EDCl (1.1 g, 5.9 mmol) and TEA (789 mg, 7.8 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (636 mg, 70% yield). LC-MS (ESI): m/z (M+1)=234.47.

### Step 4: ethyl (S)-2-(5,8-dimethyl-3-oxo-2-(tetrahydro-2H-pyran-4-yl)-7-(trimethylsilyl)-1,2,3,4-tetrahydroisoquinolin-6-yl)-2-hydroxyacetate

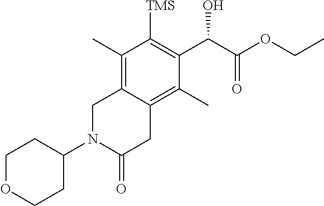 [SMILES: CCOC(=O)[C@@H](O)c1c(C)c2c(c(C)c1[Si](C)(C)C)CN(C1CCOCC1)C(=O)C2]

A mixture of Rh(cod)₂BF₄ (219 mg, 0.54 mmol) and BINAP (336 mg, 0.54 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (1.1 g, 5.4 mmol) was introduced. The reaction mixture was heated up to 80° C. in a seal tube and N-(but-2-yn-1-yl)-N-(tetrahydro-2H-pyran-4-yl)pent-3-ynamide (636 mg, 2.7 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (640 mg, 55% yield). LC-MS (ESI): m/z (M+1)=434.94.

### Step 5: ethyl (S)-2-hydroxy-2-(7-iodo-5,8-dimethyl-3-oxo-2-(tetrahydro-2H-pyran-4-yl)-1,2,3,4-tetrahydroisoquinolin-6-yl)acetate

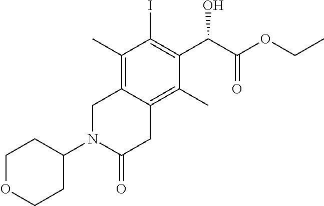 [SMILES: CCOC(=O)[C@@H](O)c1c(C)c2c(c(C)c1I)CN(C1CCOCC1)C(=O)C2]

At 0° C., to a solution of ethyl (S)-2-(5,8-dimethyl-3-oxo-2-(tetrahydro-2H-pyran-4-yl)-7-(trimethylsilyl)-1,2,3,4-tetrahydroisoquinolin-6-yl)-2-hydroxyacetate (64 mg, 0.15 mmol) in DCM (1 mL) was added ICl (2.5M solution in DCM, 0.06 mL, 0.15 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 6: ethyl (S)-2-(tert-butoxy)-2-(7-iodo-5,8-dimethyl-3-oxo-2-(tetrahydro-2H-pyran-1,2,3,4-tetrahydroisoquinolin-6-yl)acetate

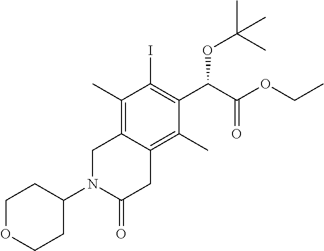 [SMILES: CCOC(=O)[C@@H](OC(C)(C)C)c1c(C)c2c(c(C)c1I)CN(C1CCOCC1)C(=O)C2]

To a solution of ethyl (S)-2-hydroxy-2-(7-iodo-5,8-dimethyl-3-oxo-2-(tetrahydro-2H-pyran-4-yl)-1,2,3,4-tetrahydroisoquinolin-6-yl)acetate (crude product from the previous step, 0.15 mmol) in t-BuOAc (2 mL) was added HClO₄ (15 mg, 0.15 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (50 mg, 61% yield over two steps). LC-MS (ESI): m/z (M+1)=544.28.

### Step 7: (S)-ethyl 2-(tert-butoxy)-2-((R)-7-(8-fluoro-5-methylchroman-6-yl)-5,8-dimeth yl-3-oxo-2-(tetrahydro-2H-pyran-4-yl)-1,2,3,4-tetrahydroisoquinolin-6-yl)acetate

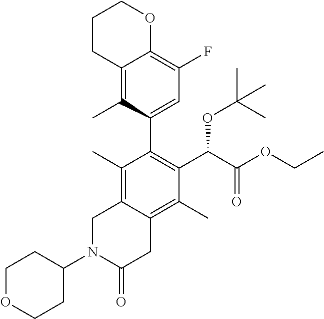

A mixture of ethyl (S)-2-(tert-butoxy)-2-(7-iodo-5,8-dimethyl-3-oxo-2-(tetrahydro-2H-pyran-4-yl)-1,2,3,4-tetrahydroisoquinolin-6-yl)acetate (50 mg, 0.09 mmol), (8-fluoro-5-methylchroman-6-yl) boronic acid (38 mg, 0.18 mmol), Pd(Pt-Bu₃)₂ (9 mg, 0.018 mmol) and Cs₂CO₃ (60 mg, 0.18 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (35 mg, 67% yield). LC-MS (ESI): m/z (M+1)=582.53.

### Step 8: (S)-2-(tert-butoxy)-2-((R)-7-(8-fluoro-5-methylchroman-6-yl)-5,8-dimethyl-3-oxo-2-(tetrahydro-2H-pyran-4-yl)-1,2,3,4-tetrahydroisoquinolin-6-yl)acetic Acid

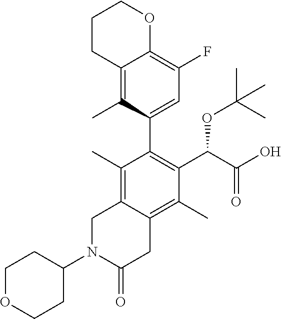

A mixture of ethyl (2S)-2-(tert-butoxy)-2-(7-(8-fluoro-5-methylchroman-6-yl)-5,8-di methyl-3-oxo-2-(tetrahydro-2H-pyran-4-yl)-1,2,3,4-tetrahydroisoquinolin-6-yl)acetate (35 mg, 0.06 mmol) in EtOH (0.5 mL) and dioxane (1 mL) was added LiOH (2.5N, 0.24 mL, 0.6 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (16 mg, 48% yield). LC-MS (ESI): m/z (M+1)=554.48. ¹H NMR (400 MHz, CDCl₃) δ 9.62 (br, 1H), 6.69 (d, J=11.1 Hz, 1H), 5.09 (s, 1H), 4.97-4.89 (m, 1H), 4.41-4.24 (m, 4H), 4.07 (d, J=11.6 Hz, 2H), 3.70-3.52 (m, 4H), 2.76-2.63 (m, 2H), 2.29 (s, 3H), 2.18-2.07 (m, 2H), 2.00-1.77 (m, 8H), 1.71-1.64 (m, 2H), 1.12 (s, 9H).

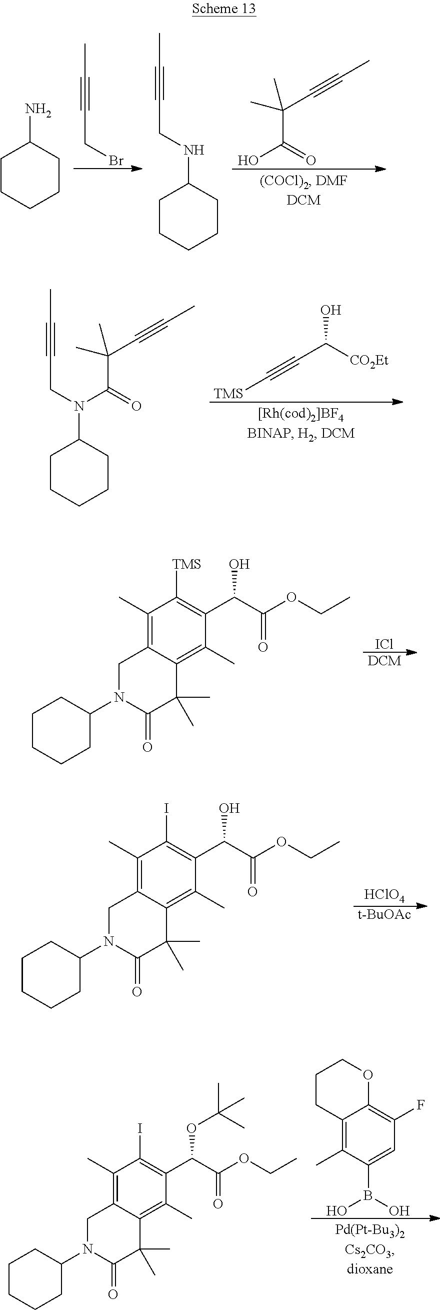

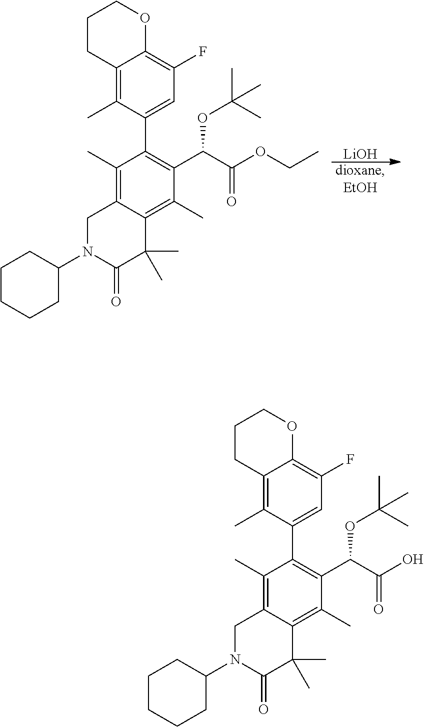

### Example 109: ((S)-2-(tert-butoxy)-2-((R)-2-cyclohexyl-7-(8-fluoro-5-methylchroman-6-yl)-4,4,5,8-tetramethyl-3-oxo-1,2,3,4-tetrahydroisoquinolin-6-yl)acetic Acid

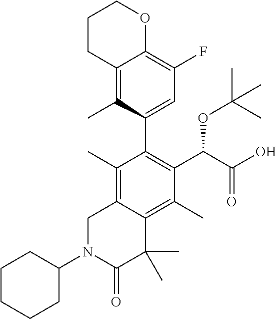

### Step 1: N-(but-2-yn-1-yl)cyclohexanamine

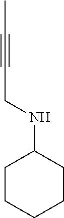

At 0° C., 1-bromobut-2-yne (1 g, 7.5 mmol) was slowed added to cyclohexanamine (5 g, 50.5 mmol). After stirred at 0° C. for 30 min, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (1.5 g, 80% yield). LC-MS (ESI): m/z (M+1)=152.32.

### Step 2: N-(but-2-yn-1-yl)-N-cyclohexyl-2,2-dimethylpent-3-ynamide

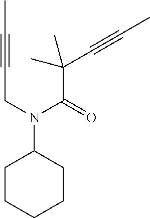 [SMILES: CC#CCN(C(=O)C(C)(C)C#CC)C1CCCCC1]

At 0° C., to a solution of 2,2-dimethylpent-3-ynoic acid (275 mg, 2.18 mmol) in DCM (5 mL) was added (COCl)₂ (369 mg, 2.91 mmol) and one drop of DMF. After stirred at 0° C. for 2 hr, the resulting mixture was concentrated and the residue was dissolved in DCM and introduced to a solution of N-(but-2-yn-1-yl)cyclohexanamine (220 mg, 1.46 mmol) in DCM (5 mL). The reaction mixture was stirred at r.t. for 2 hr and concentrated under reduced pressure to give the crude product which was purified by flash chromatography (silica gel, 0-20% EtOAc in PE) to afford the title compound (225 mg, 60% yield) as a yellow oil. LC-MS (ESI): m/z (M+1)=260.32.

### Step 3: ethyl (S)-2-(2-cyclohexyl-4,4,5,8-tetramethyl-3-oxo-7-(trimethylsilyl)-1,2,3,4-tetrahydroisoquinolin-6-yl)-2-hydroxyacetate

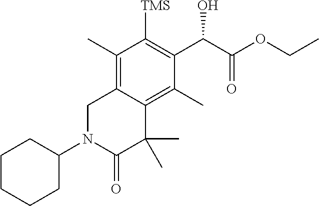 [SMILES: CCOC(=O)[C@@H](O)c1c(C)c2c(c(C)c1[Si](C)(C)C)CN(C1CCCCC1)C(=O)C2(C)C]

A mixture of Rh(cod)₂BF₄ (36 mg, 0.09 mmol) and BINAP (56 mg, 0.09 mmol) in DCM (2 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (184 mg, 0.92 mmol) was introduced. The reaction mixture was heated up to 80° C. in a seal tube and N-(but-2-yn-1-yl)-N-cyclohexyl-2,2-dimethyl pent-3-ynamide (120 mg, 0.46 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (53 mg, 25% yield). LC-MS (ESI): m/z (M+1)=460.54.

### Step 4: ethyl (S)-2-(2-cyclohexyl-7-iodo-4,4,5,8-tetramethyl-3-oxo-1,2,3,4-tetrahydro isoquinolin-6-yl)-2-hydroxyacetate

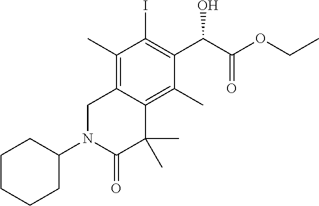 [SMILES: CCOC(=O)[C@@H](O)c1c(C)c2c(c(C)c1I)CN(C1CCCCC1)C(=O)C2(C)C]

At 0° C., to a solution of ethyl (S)-2-(2-cyclohexyl-4,4,5,8-tetramethyl-3-oxo-7-(trimethyl silyl)-1,2,3,4-tetrahydroisoquinolin-6-yl)-2-hydroxyacetate (53 mg, 0.12 mmol) in DCM (2 mL) was added ICl (2.5M solution in DCM, 0.05 mL, 0.12 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=514.30.

### Step 5: ethyl (S)-2-(tert-butoxy)-2-(2-cyclohexyl-7-iodo-4,4,5,8-tetramethyl-3-oxo-1,2,3,4-tetrahydroisoquinolin-6-yl)acetate

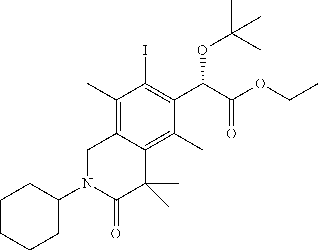 [SMILES: CCOC(=O)[C@@H](OC(C)(C)C)c1c(C)c2c(c(C)c1I)CN(C1CCCCC1)C(=O)C2(C)C]

To a solution of ethyl (S)-2-(2-cyclohexyl-7-iodo-4,4,5,8-tetramethyl-3-oxo-1,2,3,4-tetrahydroisoquinolin-6-yl)-2-hydroxyacetate (crude product from the previous step, 0.12 mmol) in t-BuOAc (2 mL) was added HClO₄ (12 mg, 0.12 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (21 mg, 31% yield over two steps). LC-MS (ESI): m/z (M+1)=570.43.

### Step 6: (S)-ethyl 2-(tert-butoxy)-2-((R)-2-cyclohexyl-7-(8-fluoro-5-methylchroman-6-yl)-4,4,5,8-tetramethyl-3-oxo-1,2,3,4-tetrahydroisoquinolin-6-yl)acetate

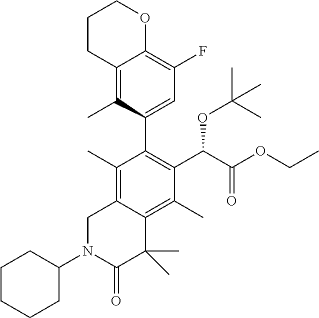

A mixture of ethyl (S)-2-(tert-butoxy)-2-(2-cyclohexyl-7-iodo-4,4,5,8-tetramethyl-3-oxo-1,2,3,4-tetrahydroisoquinolin-6-yl)acetate (21 mg, 0.037 mmol), (8-fluoro-5-methylchroman-6-yl) boronic acid (31 mg, 0.147 mmol), Pd(Pt-Bu₃)₂ (4 mg, 0.007 mmol) and Cs₂CO₃ (36 mg, 0.111 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (20 mg, 89% yield). LC-MS (ESI): m/z (M+1)=608.71.

### Step 7: (S)-2-(tert-butoxy)-2-((R)-2-cyclohexyl-7-(8-fluoro-5-methylchroman-6-yl)-4,4,5,8-tetramethyl-3-oxo-1,2,3,4-tetrahydroisoquinolin-6-yl)acetic acid

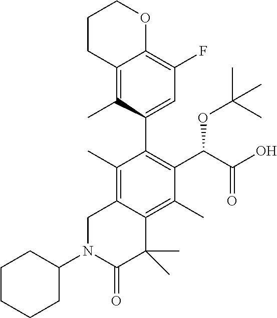

A mixture of ethyl (2S)-2-(tert-butoxy)-2-(2-cyclohexyl-7-(8-fluoro-5-methylchroman-6-yl)-4,4,5,8-tetramethyl-3-oxo-1,2,3,4-tetrahydroisoquinolin-6-yl)acetate (20 mg, 0.033 mmol) in EtOH (0.5 mL) and dioxane (1 mL) was added LiOH (2.5N, 0.15 mL, 0.37 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (9 mg, 47% yield). LC-MS (ESI): m/z (M+1)=580.45. ¹H NMR (400 MHz, CDCl₃) δ 9.57 (br, 1H), 6.67 (d, J=10.9 Hz, 1H), 5.15 (s, 1H), 4.65-4.55 (m, 1H), 4.39-4.21 (m, 4H), 2.75-2.62 (m, 2H), 2.49 (s, 3H), 2.17-2.08 (m, 2H), 1.81-1.47 (m, 22H), 1.11 (s, 9H).

### Example 110: ((S)-2-(tert-butoxy)-2-((R)-7-(8-fluoro-5-methylchroman-6-yl)-2-isobutyl-5,8-dimethyl-3-oxo-1,2,3,4-tetrahydroisoquinolin-6-yl)acetic Acid

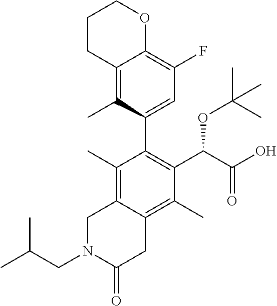

### Step 1: N-isobutylbut-2-yn-1-amine

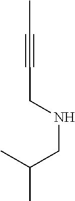

At 0° C., 1-bromobut-2-yne (1.82 g, 13.7 mmol) was slowed added to 2-methylpropan-1-amine (5 g, 68.4 mmol). After stirred at 0° C. for 10 min, the resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (850 mg, 50% yield) as a yellow oil. LC-MS (ESI): m/z (M+1)=126.24.

### Step 2: N-(but-2-yn-1-yl)-N-isobutylpent-3-ynamide

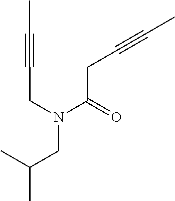

To a mixture of N-isobutylbut-2-yn-1-amine (436 mg, 3.5 mmol), pent-3-ynoic acid (513 mg, 5.2 mmol) in THF (10 mL) was added EDCl (1.33 g, 6.97 mmol) and TEA (2 mL, 13.9 mmol). After stirred at r.t. for 1 hr, the resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (365 mg, 51% yield) as a yellow oil. LC-MS (ESI): m/z (M+1)=206.32.

### Step 3: ethyl (S)-2-hydroxy-2-(2-isobutyl-5,8-dimethyl-3-oxo-7-(trimethylsilyl)-1,2,3,4-tetrahydroisoquinolin-6-yl)acetate

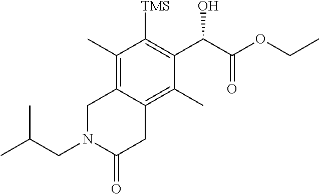 [SMILES: CCOC(=O)[C@@H](O)c1c(C)c2c(c(C)c1[Si](C)(C)C)CN(CC(C)C)C(=O)C2]

A mixture of Rh(cod)₂BF₄ (146 mg, 0.36 mmol) and BINAP (224 mg, 0.36 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (712 mg, 3.56 mmol) was introduced. The reaction mixture was heated up to 80° C. in a seal tube and N-(but-2-yn-1-yl)-N-isobutylpent-3-ynamide (365 mg, 1.78 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (120 mg, 17% yield). LC-MS (ESI): m/z (M+1)=406.56.

### Step 4: ethyl (S)-2-hydroxy-2-(7-iodo-2-isobutyl-5,8-dimethyl-3-oxo-1,2,3,4-tetrahydroisoquinolin-6-yl)acetate

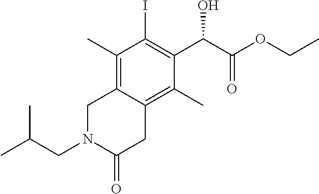 [SMILES: CCOC(=O)[C@@H](O)c1c(C)c2c(c(C)c1I)CN(CC(C)C)C(=O)C2]

At 0° C., to a solution of ethyl (S)-2-hydroxy-2-(2-isobutyl-5,8-dimethyl-3-oxo-7-(trimethylsilyl)-1,2,3,4-tetrahydroisoquinolin-6-yl)acetate (120 mg, 0.296 mmol) in DCM (2 mL) was added ICl (2.5M solution in DCM, 0.12 mL, 0.296 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 5: ethyl (S)-2-(tert-butoxy)-2-(7-iodo-2-isobutyl-5,8-dimethyl-3-oxo-1,2,3,4-tetrahydroisoquinolin-6-yl)acetate

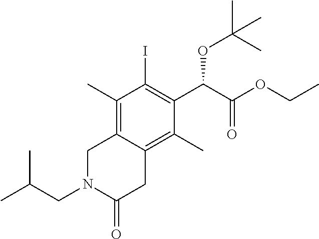 [SMILES: CCOC(=O)[C@@H](OC(C)(C)C)c1c(C)c2c(c(C)c1I)CN(CC(C)C)C(=O)C2]

To a solution of ethyl (S)-2-hydroxy-2-(7-iodo-2-isobutyl-5,8-dimethyl-3-oxo-1,2,3,4-tetrahydroisoquinolin-6-yl)acetate (crude product from the previous step, 0.296 mmol) in t-BuOAc (2 mL) was added HClO₄ (30 mg, 0.30 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (130 mg, 85% yield over two steps). LC-MS (ESI): m/z (M+1)=516.43.

### Step 6: (S)-ethyl 2-(tert-butoxy)-2-((R)-7-(8-fluoro-5-methylchroman-6-yl)-2-isobutyl-5,8-dimethyl-3-oxo-1,2,3,4-tetrahydroisoquinolin-6-yl)acetate

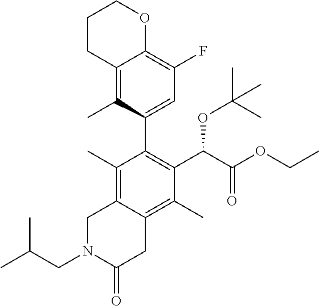

A mixture of ethyl (S)-2-(tert-butoxy)-2-(7-iodo-2-isobutyl-5,8-dimethyl-3-oxo-1,2,3,4-tetrahydroisoquinolin-6-yl)acetate (50 mg, 0.097 mmol), (8-fluoro-5-methylchroman-6-yl) boronic acid (81 mg, 0.388 mmol), Pd(Pt-Bu₃)₂ (10 mg, 0.0194 mmol) and Cs₂CO₃ (95 mg, 0.291 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (42 mg, 78% yield). LC-MS (ESI): m/z (M+1)=554.83.

### Step 7: ((S)-2-(tert-butoxy)-2-((R)-7-(8-fluoro-5-methylchroman-6-yl)-2-isobutyl-5,8-dimethyl-3-oxo-1,2,3,4-tetrahydroisoquinolin-6-yl)acetic Acid

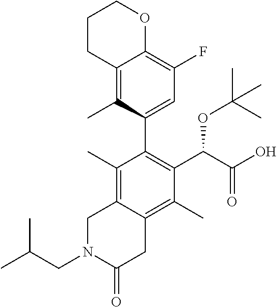

A mixture of ethyl (2S)-2-(tert-butoxy)-2-(7-(8-fluoro-5-methylchroman-6-yl)-2-iso butyl-5,8-dimethyl-3-oxo-1,2,3,4-tetrahydroisoquinolin-6-yl)acetate (42 mg, 0.076 mmol) in EtOH (0.5 mL) and dioxane (1 mL) was added LiOH (2.5N, 0.3 mL, 0.75 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (22 mg, 55% yield). LC-MS (ESI): m/z (M+1)=526.25. ¹H NMR (400 MHz, CDCl₃) δ 9.57 (br, 1H), 6.70 (d, J=11.2 Hz, 1H), 5.09 (s, 1H), 4.42 (s, 2H), 4.32-4.21 (m, 2H), 3.63 (d, J=5.0 Hz, 2H), 3.48-3.32 (m, 2H), 2.76-2.62 (m, 2H), 2.28 (s, 3H), 2.18-2.03 (m, 3H), 1.83 (d, J=24.3 Hz, 6H), 1.12 (s, 9H), 0.95 (d, J=6.6 Hz, 6H).

### Example 111: (2S)-2-(tert-butoxy)-2-((7R)-2-(3,3-dimethylcyclohexyl)-7-(8-fluoro-5-methylchroman-6-yl)-5,8-dimethyl-3-oxo-1,2,3,4-tetrahydroisoquinolin-6-yl)acetic Acid

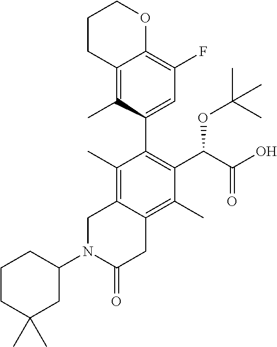

### Step 1: N-(but-2-yn-1-yl)-3,3-dimethylcyclohexan-1-amine

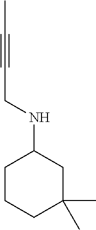

At 0° C., 1-bromobut-2-yne (1.04 g, 7.85 mmol) was slowed added to 3,3-dimethylcyclohexan-1-amine (5 g, 39.4 mmol). After stirred at 0° C. for 10 min, the resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (1.03 g, 73% yield) as a yellow oil. LC-MS (ESI): m/z (M+1)=180.31.

### Step 2: N-(but-2-yn-1-yl)-N-(3,3-dimethylcyclohexyl)pent-3-ynamide

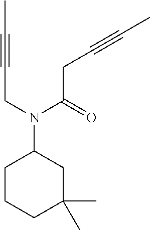 [SMILES: CC#CCC(=O)N(CC#CC)C1CCCC(C)(C)C1]

To a mixture of N-(but-2-yn-1-yl)-3,3-dimethylcyclohexan-1-amine (460 mg, 2.57 mmol), pent-3-ynoic acid (377 mg, 3.85 mmol) in THF (10 mL) was added EDCl (984 mg, 5.13 mmol) and TEA (1.04 g, 10.26 mmol). After stirred at r.t. for 1 hr, the resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (480 mg, 61% yield) as a yellow oil. LC-MS (ESI): m/z (M+1)=260.41.

### Step 3: ethyl (2S)-2-(2-(3,3-dimethylcyclohexyl)-5,8-dimethyl-3-oxo-7-(trimethylsilyl)-1,2,3,4-tetrahydroisoquinolin-6-yl)-2-hydroxyacetate

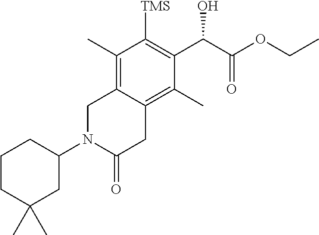 [SMILES: CCOC(=O)[C@@H](O)c1c(C)c2c(c(C)c1[Si](C)(C)C)CN(C1CCCC(C)(C)C1)C(=O)C2]

A mixture of Rh(cod)₂BF₄ (128 mg, 0.315 mmol) and BINAP (196 mg, 0.315 mmol) in DCM (5 mL) was stirred at r.t. under H₂ atmosphere for 6 hr. After concentration, the resulting mixture was dissolved in DCE (5 mL) and ethyl (S)-2-hydroxy-4-(trimethyl silyl)but-3-ynoate (630 mg, 3.15 mmol) was introduced. The reaction mixture was heated up to 80° C. in a seal tube and N-(but-2-yn-1-yl)-N-(3,3-dimethylcyclohexyl) pent-3-ynamide (408 mg, 1.57 mmol) was added as a DCE solution (5 mL) over 3 hr. After stirred at 80° C. for 30 min, the resulting mixture was concentrated to give the crude product which was purified by flash chromatography (silica gel, 0-50% EtOAc in PE) to afford the title compound (270 mg, 20% yield). LC-MS (ESI): m/z (M+1)=460.85.

### Step 4: ethyl (2S)-2-(2-(3,3-dimethylcyclohexyl)-7-iodo-5,8-dimethyl-3-oxo-1,2,3,4-tetrahydroisoquinolin-6-yl)-2-hydroxyacetate

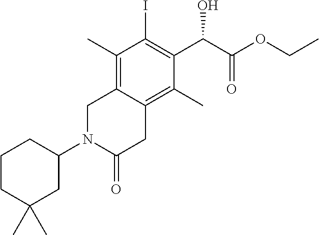 [SMILES: CCOC(=O)[C@@H](O)c1c(C)c2c(c(C)c1I)CN(C1CCCC(C)(C)C1)C(=O)C2]

At 0° C., to a solution of ethyl (2S)-2-(2-(3,3-dimethylcyclohexyl)-5,8-dimethyl-3-oxo-7-(trimethylsilyl)-1,2,3,4-tetrahydroisoquinolin-6-yl)-2-hydroxyacetate (270 mg, 0.587 mmol) in DCM (10 mL) was added ICl (2.5M solution in DCM, 0.23 mL, 0.58 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=514.60.

### Step 5: ethyl (2S)-2-(tert-butoxy)-2-(2-(3,3-dimethylcyclohexyl)-7-iodo-5,8-dimethyl-3-oxo-1,2,3,4-tetrahydroisoquinolin-6-yl)acetate

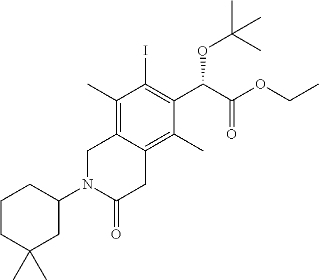 [SMILES: CCOC(=O)[C@@H](OC(C)(C)C)c1c(C)c2c(c(C)c1I)CN(C1CCCC(C)(C)C1)C(=O)C2]

To a solution of ethyl (2S)-2-(2-(3,3-dimethylcyclohexyl)-7-iodo-5,8-dimethyl-3-oxo-1,2,3,4-tetrahydroisoquinolin-6-yl)-2-hydroxyacetate (crude product from the previous step, 0.587 mmol) in t-BuOAc (5 mL) was added HClO₄ (60 mg, 0.60 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (100 mg, 30% yield over two steps). LC-MS (ESI): m/z (M+1)=570.62.

### Step 6: (2S)-ethyl 2-(tert-butoxy)-2-((7R)-2-(3,3-dimethylcyclohexyl)-7-(8-fluoro-5-methylchroman-6-yl)-5,8-dimethyl-3-oxo-1,2,3,4-tetrahydroisoquinolin-6-yl)acetate

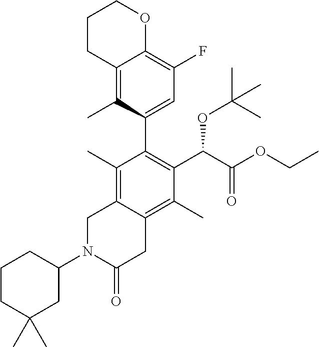

A mixture of ethyl (2S)-2-(tert-butoxy)-2-(2-(3,3-dimethylcyclohexyl)-7-iodo-5,8-dimethyl-3-oxo-1,2,3,4-tetrahydroisoquinolin-6-yl)acetate (50 mg, 0.088 mmol), (8-fluoro-5-methylchroman-6-yl) boronic acid (74 mg, 0.35 mmol), Pd(Pt-Bu₃)₂ (9 mg, 0.0176 mmol) and Cs₂CO₃ (86 mg, 0.263 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (50 mg, 94% yield). LC-MS (ESI): m/z (M+1)=608.84.

### Step 7: (2S)-2-(tert-butoxy)-2-((7R)-2-(3,3-dimethylcyclohexyl)-7-(8-fluoro-5-methylchroman-6-yl)-5,8-dimethyl-3-oxo-1,2,3,4-tetrahydroisoquinolin-6-yl)acetic Acid

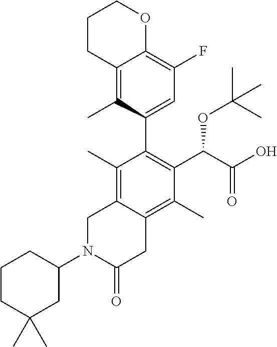

A mixture of ethyl (2S)-2-(tert-butoxy)-2-(2-(3,3-dimethylcyclohexyl)-7-(8-fluoro-5-methylchroman-6-yl)-5,8-dimethyl-3-oxo-1,2,3,4-tetrahydroisoquinolin-6-yl)acetate (50 mg, 0.0823 mmol) in EtOH (0.5 mL) and dioxane (1 mL) was added LiOH (2.5N, 0.33 mL, 0.823 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (15.6 mg, 33% yield). LC-MS (ESI): m/z (M+1)=580.46. ¹H NMR (400 MHz, CDCl₃) δ 9.57 (br, 1H), 6.69 (d, J=11.2 Hz, 1H), 5.07 (s, 1H), 4.89-4.78 (m, 1H), 4.44-4.17 (m, 4H), 3.69-3.48 (m, 2H), 2.78-2.62 (m, 2H), 2.28 (s, 3H), 2.17-2.08 (m, 2H), 1.84 (d, J=13.5 Hz, 6H), 1.66-1.37 (m, 8H), 1.24-0.86 (m, 15H).

### Example 112: (S)-2-(tert-butoxy)-2-((R)-7-(8-fluoro-5-methylchroman-6-yl)-5,8-dimethyl-2-(1-methylpiperidin-4-yl)-3-oxo-1,2,3,4-tetrahydroisoquinolin-6-yl)acetic Acid

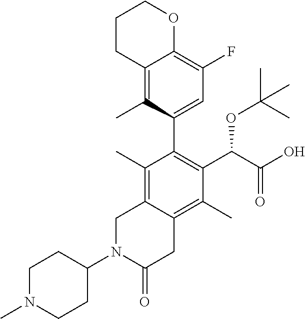

### Step 1: tert-butyl 4-(but-2-yn-1-ylamino)piperidine-1-carboxylate

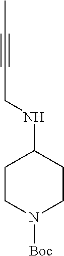 [SMILES: CC#CCNC1CCN(C(=O)OC(C)(C)C)CC1]

At 0° C., 1-bromobut-2-yne (1.0 g, 7.52 mmol) was slowed added to tert-butyl 4-aminopiperidine-1-carboxylate (5 g, 25.0 mmol). After stirred at 0° C. for 30 min, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (1.01 g, 35% yield) as a white solid. LC-MS (ESI): m/z (M+1)=253.20.

### Step 2: tert-butyl 4-(N-(but-2-yn-1-yl)pent-3-ynamido)piperidine-1-carboxylate

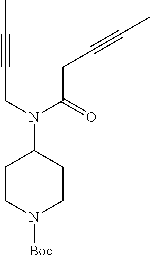 [SMILES: CC#CCC(=O)N(CC#CC)C1CCN(C(=O)OC(C)(C)C)CC1]

To a mixture of tert-butyl 4-(but-2-yn-1-ylamino)piperidine-1-carboxylate (500 mg, 1.98 mmol), pent-3-ynoic acid (291 mg, 2.97 mmol) in THF (10 mL) was added EDCl (597 mg, 2.97 mmol) and TEA (400 mg, 3.96 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (500 mg, 75% yield) as a white solid. LC-MS (ESI): m/z (M+1)=333.26.

### Step 3: tert-butyl (S)-4-(6-(2-ethoxy-1-hydroxy-2-oxoethyl)-5,8-dimethyl-3-oxo-7-(trimethylsilyl)-3,4-dihydroisoquinolin-2(1H)-yl)piperidine-1-carboxylate

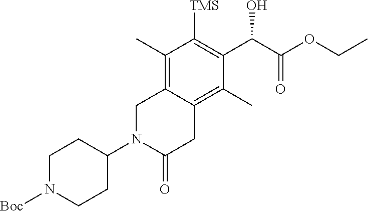 [SMILES: CCOC(=O)[C@@H](O)c1c(C)c2c(c(C)c1[Si](C)(C)C)CN(C1CCN(C(=O)OC(C)(C)C)CC1)C(=O)C2]

A mixture of Rh(cod)₂BF₄ (146 mg, 0.36 mmol) and BINAP (224 mg, 0.36 mmol) in DCM (10 mL) was stirred at r.t. under H₂ atmosphere for 6 hr. After concentration, the resulting mixture was dissolved in DCE (10 mL) and ethyl (S)-2-hydroxy-4-(trimethyl silyl)but-3-ynoate (720 mg, 3.6 mmol) was introduced. The reaction mixture was heated up to 80° C. in a seal tube and tert-butyl 4-(N-(but-2-yn-1-yl)pent-3-ynamido) piperidine-1-carboxylate (500 mg, 1.8 mmol) was added as a DCE solution (10 mL) over 3 hr. After stirred at 80° C. for 30 min, the resulting mixture was concentrated to give the crude product which was purified by flash chromatography (silica gel, 0-50% EtOAc in PE) to afford the title compound (200 mg, 25% yield). LC-MS (ESI): m/z (M+1)=533.41.

### Step 4: tert-butyl (S)-4-(6-(2-ethoxy-1-hydroxy-2-oxoethyl)-7-iodo-5,8-dimethyl-3-oxo-3,4-dihydroisoquinolin-2(1H)-yl)piperidine-1-carboxylate

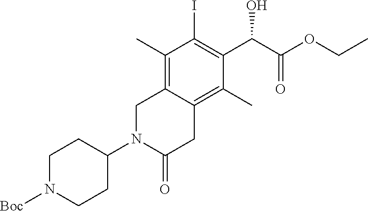 [SMILES: CCOC(=O)[C@@H](O)c1c(C)c2c(c(C)c1I)CN(C1CCN(C(=O)OC(C)(C)C)CC1)C(=O)C2]

At 0° C., to a solution of tert-butyl (S)-4-(6-(2-ethoxy-1-hydroxy-2-oxoethyl)-5,8-di methyl-3-oxo-7-(trimethylsilyl)-3,4-dihydroisoquinolin-2(1H)-yl)piperidine-1-carboxylate (200 mg, 0.38 mmol) in DCM (2 mL) was added ICl (2.5M solution in DCM, 0.15 mL, 0.38 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=587.22.

### Step 5: ethyl (S)-2-hydroxy-2-(7-iodo-5,8-dimethyl-3-oxo-2-(piperidin-4-yl)-1,2,3,4-tetrahydroisoquinolin-6-yl)acetate

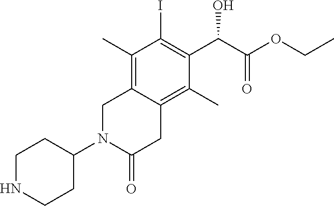 [SMILES: CCOC(=O)[C@@H](O)c1c(C)c2c(c(C)c1I)CN(C1CCNCC1)C(=O)C2]

To a solution of tert-butyl (S)-4-(6-(2-ethoxy-1-hydroxy-2-oxoethyl)-7-iodo-5,8-di methyl-3-oxo-3,4-dihydroisoquinolin-2(1H)-yl)piperidine-1-carboxylate (crude product from the previous step, 0.38 mmol) in DCM (2 mL) was added 4N HCl in dioxane (2 mL). After stirred at r.t. for 1 hr, the resulting mixture was concentrated to give the title compound as a crude product (HCl salt) which was used in the next step without further purification. LC-MS (ESI): m/z (M+1)=487.25.

### Step 6: ethyl (S)-2-(tert-butoxy)-2-(7-iodo-5,8-dimethyl-3-oxo-2-(piperidin-4-yl)-1,2,3,4-tetrahydroisoquinolin-6-yl)acetate

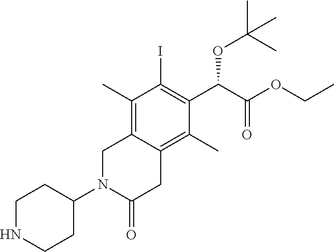 [SMILES: CCOC(=O)[C@@H](OC(C)(C)C)c1c(C)c2c(c(C)c1I)CN(C1CCNCC1)C(=O)C2]

To a solution of ethyl (S)-2-hydroxy-2-(7-iodo-5,8-dimethyl-3-oxo-2-(piperidin-4-yl)-1,2,3,4-tetrahydroisoquinolin-6-yl)acetate (crude product from the previous step, 0.38 mmol) in t-BuOAc (2 mL) was added HClO₄ (38 mg, 0.38 mmol). After stirred at r.t. for 3 hr, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-10% MeOH in DCM) to afford the title compound (44 mg, 21% yield over three steps). LC-MS (ESI): m/z (M+1)=543.62.

### Step 7: ethyl (S)-2-(tert-butoxy)-2-(7-iodo-5,8-dimethyl-2-(1-methylpiperidin-4-yl)-3-oxo-1,2,3,4-tetrahydroisoquinolin-6-yl)acetate

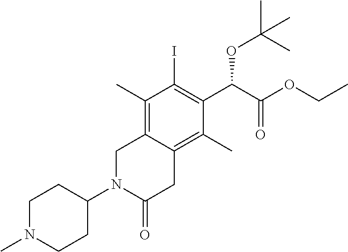 [SMILES: CCOC(=O)[C@@H](OC(C)(C)C)c1c(C)c2c(c(C)c1I)CN(C1CCN(C)CC1)C(=O)C2]

A mixture of ethyl (S)-2-(tert-butoxy)-2-(7-iodo-5,8-dimethyl-3-oxo-2-(piperidin-4-yl)-1,2,3,4-tetrahydroisoquinolin-6-yl)acetate (44 mg, 0.08 mol), paraformaldehyde (3.6 mg, 0.12 mmol) and NaBH(OAc)₃ (37 mg, 0.176 mmol) in DCE (2 mL) was stirred at r.t. for 48 hr. The resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-10% MeOH in DCM) to afford the title compound (32 mg, 72% yield). LC-MS (ESI): m/z (M+1)=557.41.

### Step 8: (S)-ethyl 2-(tert-butoxy)-2-((R)-7-(8-fluoro-5-methylchroman-6-yl)-5,8-dimethyl-2-(1-methylpiperidin-4-yl)-3-oxo-1,2,3,4-tetrahydroisoquinolin-6-yl)acetate

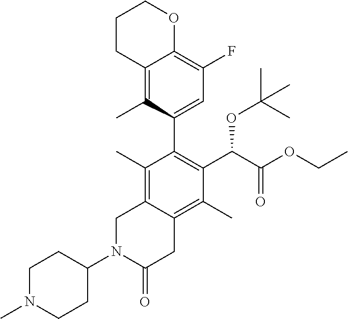

A mixture of ethyl (S)-2-(tert-butoxy)-2-(7-iodo-5,8-dimethyl-2-(1-methylpiperidin-4-yl)-3-oxo-1,2,3,4-tetrahydroisoquinolin-6-yl)acetate (32 mg, 0.057 mmol), (8-fluoro-5-methylchroman-6-yl) boronic acid (48 mg, 0.23 mmol), Pd(Pt-Bu₃)₂ (5.9 mg, 0.01 mmol) and Cs₂CO₃ (55 mg, 0.17 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-10% MeOH in DCM) to afford the title compound (27 mg, 79% yield). LC-MS (ESI): m/z (M+1)=595.22.

### Step 9: (S)-2-(tert-butoxy)-2-((R)-7-(8-fluoro-5-methylchroman-6-yl)-5,8-dimethyl-2-(1-methylpiperidin-4-yl)-3-oxo-1,2,3,4-tetrahydroisoquinolin-6-yl)acetic Acid

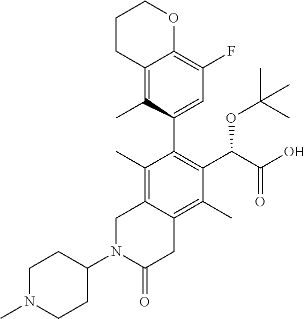

A mixture of ethyl (2S)-2-(tert-butoxy)-2-(7-(8-fluoro-5-methylchroman-6-yl)-5,8-dimethyl-2-(1-methylpiperidin-4-yl)-3-oxo-1,2,3,4-tetrahydroisoquinolin-6-yl)acetate (27 mg, 0.045 mmol) in EtOH (0.5 mL) and dioxane (1 mL) was added LiOH (2.5N, 0.18 mL, 0.45 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (6 mg, 23% yield). LC-MS (ESI): m/z (M+1)=567.41. ¹H NMR (400 MHz, CDCl₃) δ 6.63 (d, J=11.3 Hz, 1H), 4.93 (s, 1H), 4.59-4.52 (m, 1H), 4.33-4.15 (m, 4H), 3.79-3.63 (m, 2H), 3.38-3.12 (m, 4H), 2.74-2.35 (m, 10H), 2.17-2.10 (m, 2H), 1.97 (s, 3H), 1.76-1.67 (m, 5H), 1.08 (s, 9H) (the acid proton was not observed).

### Example 113: ((S)-2-(tert-butoxy)-2-((R)-7-(8-fluoro-5-methylchroman-6-yl)-5,8-dimethyl-3-oxo-2-((tetrahydro-2H-pyran-4-yl)methyl)-1,2,3,4-tetrahydroisoquinolin-6-yl)acetic Acid

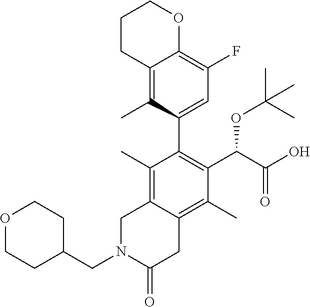

### Step 1: N-((tetrahydro-2H-pyran-4-yl)methyl)but-2-yn-1-amine

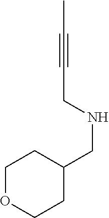

At 0° C., 1-bromobut-2-yne (1.0 g, 7.52 mmol) was slowed added to (tetrahydro-2H-pyran-4-yl)methanamine (5 g, 43.5 mmol). After stirred at 0° C. for 30 min, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (1.1 g, 50% yield) as a colorless oil. LC-MS (ESI): m/z (M+1)=168.12.

### Step 2: N-(but-2-yn-1-yl)-N-((tetrahydro-2H-pyran-4-yl)methyl)pent-3-ynamide

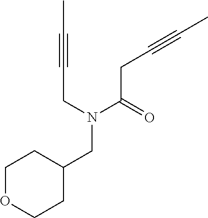 [SMILES: CC#CCC(=O)N(CC#CC)CC1CCOCC1]

To a mixture of N-((tetrahydro-2H-pyran-4-yl)methyl)but-2-yn-1-amine (500 mg, 3.0 mmol), pent-3-ynoic acid (441 mg, 4.5 mmol) in THF (10 mL) was added EDCl (860 mg, 4.5 mmol) and TEA (606 mg, 6.0 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-10% MeOH in DCM) to afford the title compound (620 mg, 85% yield) as a colorless oil. LC-MS (ESI): m/z (M+1)=248.49.

### Step 3: ethyl (S)-2-(5,8-dimethyl-3-oxo-2-((tetrahydro-2H-pyran-4-yl)methyl)-7-(trimethylsilyl)-1,2,3,4-tetrahydroisoquinolin-6-yl)-2-hydroxyacetate

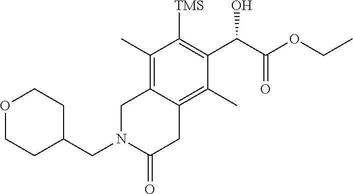 [SMILES: CCOC(=O)[C@@H](O)c1c(C)c2c(c(C)c1[Si](C)(C)C)CN(CC1CCOCC1)C(=O)C2]

A mixture of Rh(cod)₂BF₄ (130 mg, 0.32 mmol) and BINAP (200 mg, 0.32 mmol) in DCM (10 mL) was stirred at r.t. under H₂ atmosphere for 6 hr. After concentration, the resulting mixture was dissolved in DCE (10 mL) and ethyl (S)-2-hydroxy-4-(trimethyl silyl)but-3-ynoate (648 mg, 3.2 mmol) was introduced. The reaction mixture was heated up to 80° C. in a seal tube and N-(but-2-yn-1-yl)-N-((tetrahydro-2H-pyran-4-yl) methyl)pent-3-ynamide (400 mg, 1.6 mmol) was added as a DCE solution (10 mL) over 3 hr. After stirred at 80° C. for 30 min, the resulting mixture was concentrated to give the crude product which was purified by flash chromatography (silica gel, 0-50% EtOAc in PE) to afford the title compound (200 mg, 27% yield). LC-MS (ESI): m/z (M+1)=448.12.

### Step 4: ethyl (S)-2-hydroxy-2-(7-iodo-5,8-dimethyl-3-oxo-2-((tetrahydro-2H-pyran-4-yl) methyl)-1,2,3,4-tetrahydroisoquinolin-6-yl)acetate

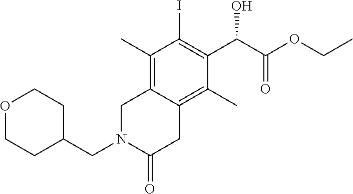 [SMILES: CCOC(=O)[C@@H](O)c1c(C)c2c(c(C)c1I)CN(CC1CCOCC1)C(=O)C2]

At 0° C., to a solution of ethyl (S)-2-(5,8-dimethyl-3-oxo-2-((tetrahydro-2H-pyran-4-yl) methyl)-7-(trimethylsilyl)-1,2,3,4-tetrahydroisoquinolin-6-yl)-2-hydroxyacetate (40 mg, 0.09 mmol) in DCM (1 mL) was added ICl (2.5M solution in DCM, 0.03 mL, 0.09 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification. LC-MS (ESI): m/z (M+1)=502.24.

### Step 5: ethyl (S)-2-(tert-butoxy)-2-(7-iodo-5,8-dimethyl-3-oxo-2-((tetrahydro-2H-pyran-4-yl)methyl)-1,2,3,4-tetrahydroisoquinolin-6-yl)acetate

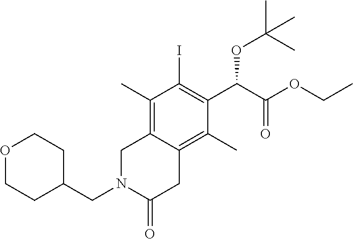 [SMILES: CCOC(=O)[C@@H](OC(C)(C)C)c1c(C)c2c(c(C)c1I)CN(CC1CCOCC1)C(=O)C2]

To a solution of ethyl (S)-2-hydroxy-2-(7-iodo-5,8-dimethyl-3-oxo-2-((tetrahydro-2H-pyran-4-yl)methyl)-1,2,3,4-tetrahydroisoquinolin-6-yl)acetate (crude product from the previous step, 0.09 mmol) in t-BuOAc (1 mL) was added HClO₄ (9 mg, 0.09 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-100% EtOAc in PE) to afford the title compound (30 mg, 60% yield over two steps). LC-MS (ESI): m/z (M+1)=558.25.

### Step 6: (S)-ethyl 2-(tert-butoxy)-2-((R)-7-(8-fluoro-5-methylchroman-6-yl)-5,8-dimethyl-3-oxo-2-((tetrahydro-2H-pyran-4-yl)methyl)-1,2,3,4-tetrahydroisoquinolin-6-yl)acetate

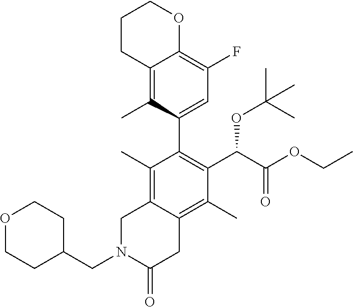

A mixture of ethyl (S)-2-(tert-butoxy)-2-(7-iodo-5,8-dimethyl-3-oxo-2-((tetrahydro-2H-pyran-4-yl)methyl)-1,2,3,4-tetrahydroisoquinolin-6-yl)acetate (30 mg, 0.054 mmol), (8-fluoro-5-methylchroman-6-yl) boronic acid (45 mg, 0.22 mmol), Pd(Pt-Bu₃)₂ (5.6 mg, 0.011 mmol) and Cs₂CO₃ (53 mg, 0.16 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (25 mg, 91% yield). LC-MS (ESI): m/z (M+1)=596.40.

### Step 7: ((S)-2-(tert-butoxy)-2-((R)-7-(8-fluoro-5-methylchroman-6-yl)-5,8-dimethyl-3-oxo-2-((tetrahydro-2H-pyran-4-yl)methyl)-1,2,3,4-tetrahydroisoquinolin-6-yl)acetic Acid

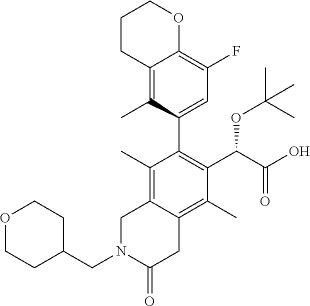

A mixture of ethyl (2S)-2-(tert-butoxy)-2-(7-(8-fluoro-5-methylchroman-6-yl)-5,8-di methyl-3-oxo-2-((tetrahydro-2H-pyran-4-yl)methyl)-1,2,3,4-tetrahydroisoquinolin-6-yl)acetate (27 mg, 0.045 mmol) in EtOH (0.5 mL) and dioxane (1 mL) was added LiOH (2.5N, 0.18 mL, 0.45 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (12 mg, 47% yield). LC-MS (ESI): m/z (M+1)=568.32. ¹H NMR (400 MHz, CDCl₃) δ 9.56 (br, 1H), 6.69 (d, J=11.3 Hz, 1H), 5.07 (s, 1H), 4.45 (s, 2H), 4.27 (dd, J=10.2, 4.4 Hz, 2H), 3.98 (d, J=11.3 Hz, 2H), 3.62 (s, 2H), 3.53-3.33 (m, 4H), 2.75-2.60 (m, 2H), 2.29 (s, 3H), 2.15-2.02 (m, 3H), 1.87-1.76 (m, 6H), 1.63-1.57 (m, 2H), 1.47-1.37 (m, 2H), 1.12 (s, 9H).

### Example 114: (S)-2-(tert-butoxy)-2-((R)-2-(4,4-difluorocyclohexyl)-7-(8-fluoro-5-methylchroman-6-yl)-5,8-dimethyl-3-oxo-1,2,3,4-tetrahydroisoquinolin-6-yl)acetic Acid

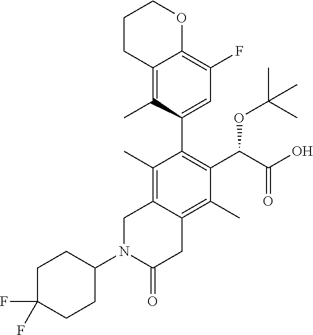

### Step 1: tert-butyl but-2-yn-1-yl(4,4-difluorocyclohexyl)carbamate

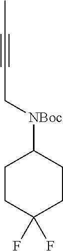 [SMILES: CC#CCN(C(=O)OC(C)(C)C)C1CCC(F)(F)CC1]

At 0° C., to a solution tert-butyl (4,4-difluorocyclohexyl)carbamate (1.0 g, 4.25 mmol) in DMF (10 mL) was added NaH (60%, 255 mg, 6.38 mmol). The reaction mixture was stirred at r.t. for 30 min before the introduction of 1-bromobut-2-yne (622 mg, 4.67 mmol). After stirred at r.t. overnight, the resulting mixture was quenched with sat. NH4Cl aq. solution and extracted with EtOAc. The organic layer was washed with brine, dried over Na₂SO₄, filtered and concentrated to give the crude product which was purified by flash chromatography (silica gel, 0-20% EtOAc in PE) to afford the title compound (1.0 g, 81% yield). LC-MS (ESI): m/z (M+1)=288.24.

### Step 2: N-(but-2-yn-1-yl)-4,4-difluorocyclohexan-1-amine

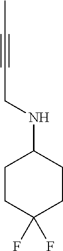

To a solution of tert-butyl but-2-yn-1-yl(4,4-difluorocyclohexyl)carbamate (500 mg, 1.74 mmol) in DCM (5 mL) was added 4N HCl in dioxane (5 mL). After stirred at r.t. for 1 hr, the resulting mixture was concentrated to afford the title compound (crude, as HCl salt) which was used in the next step without further purification. LC-MS (ESI): m/z (M+1)=188.65.

### Step 3: N-(but-2-yn-1-yl)-N-(4,4-difluorocyclohexyl)pent-3-ynamide

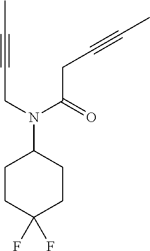 [SMILES: CC#CCC(=O)N(CC#CC)C1CCC(F)(F)CC1]

To a mixture of N-(but-2-yn-1-yl)-4,4-difluorocyclohexan-1-amine (250 mg, 1.34 mmol), pent-3-ynoic acid (196 mg, 2.0 mmol) in THF (10 mL) was added EDCl (382 mg, 2.0 mmol) and TEA (270 mg, 2.68 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (360 mg, 78% yield) as a colorless oil. LC-MS (ESI): m/z (M+1)=268.32.

### Step 4: ethyl (S)-2-(2-(4,4-difluorocyclohexyl)-5,8-dimethyl-3-oxo-7-(trimethylsilyl)-1,2,3,4-tetrahydroisoquinolin-6-yl)-2-hydroxyacetate

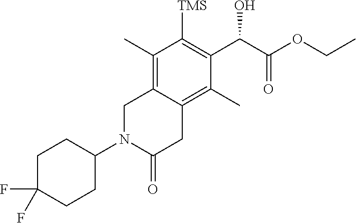 [SMILES: CCOC(=O)[C@@H](O)c1c(C)c2c(c(C)c1[Si](C)(C)C)CN(C1CCC(F)(F)CC1)C(=O)C2]

A mixture of Rh(cod)₂BF₄ (85 mg, 0.21 mmol) and BINAP (131 mg, 0.21 mmol) in DCM (5 mL) was stirred at r.t. under H₂ atmosphere for 6 hr. After concentration, the resulting mixture was dissolved in DCE (10 mL) and ethyl (S)-2-hydroxy-4-(trimethyl silyl)but-3-ynoate (420 mg, 2.1 mmol) was introduced. The reaction mixture was heated up to 80° C. in a seal tube and N-(but-2-yn-1-yl)-N-(4,4-difluorocyclohexyl) pent-3-ynamide (280 mg, 1.05 mmol) was added as a DCE solution (10 mL) over 3 hr. After stirred at 80° C. for 30 min, the resulting mixture was concentrated to give the crude product which was purified by flash chromatography (silica gel, 0-50% EtOAc in PE) to afford the title compound (210 mg, 24% yield). LC-MS (ESI): m/z (M+1)=468.35.

### Step 5: ethyl (S)-2-(2-(4,4-difluorocyclohexyl)-7-iodo-5,8-dimethyl-3-oxo-1,2,3,4-tetrahydroisoquinolin-6-yl)-2-hydroxyacetate

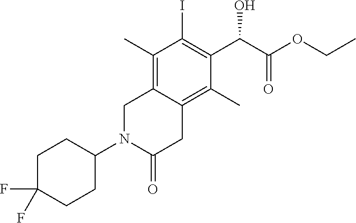 [SMILES: CCOC(=O)[C@@H](O)c1c(C)c2c(c(C)c1I)CN(C1CCC(F)(F)CC1)C(=O)C2]

At 0° C., to a solution of ethyl (S)-2-(2-(4,4-difluorocyclohexyl)-5,8-dimethyl-3-oxo-7-(tri methylsilyl)-1,2,3,4-tetrahydroisoquinolin-6-yl)-2-hydroxyacetate (120 mg, 0.26 mmol) in DCM (2 mL) was added ICl (2.5M solution in DCM, 0.1 mL, 0.25 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 6: ethyl (S)-2-(tert-butoxy)-2-(2-(4,4-difluorocyclohexyl)-7-iodo-5,8-dimethyl-3-oxo-1,2,3,4-tetrahydroisoquinolin-6-yl)acetate

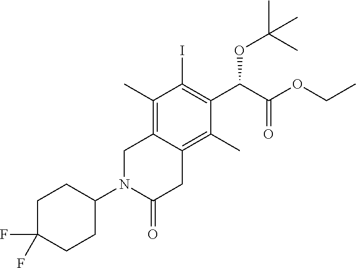 [SMILES: CCOC(=O)[C@@H](OC(C)(C)C)c1c(C)c2c(c(C)c1I)CN(C1CCC(F)(F)CC1)C(=O)C2]

To a solution of ethyl (S)-2-(2-(4,4-difluorocyclohexyl)-7-iodo-5,8-dimethyl-3-oxo-1,2,3,4-tetrahydroisoquinolin-6-yl)-2-hydroxyacetate (crude product from the previous step, 0.26 mmol) in t-BuOAc (2 mL) was added HClO₄ (26 mg, 0.26 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (80 mg, 53% yield over two steps).

LC-MS (ESI): m/z (M+1)=578.18.

### Step 7: (S)-ethyl 2-(tert-butoxy)-2-((R)-2-(4,4-difluorocyclohexyl)-7-(8-fluoro-5-methylchroman-6-yl)-5,8-dimethyl-3-oxo-1,2,3,4-tetrahydroisoquinolin-6-yl)acetate

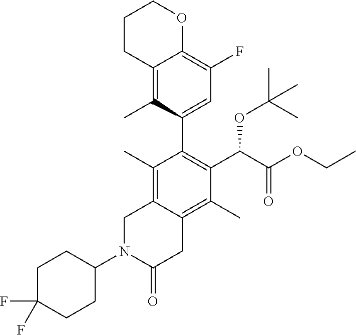

A mixture of ethyl (S)-2-(tert-butoxy)-2-(2-(4,4-difluorocyclohexyl)-7-iodo-5,8-dimethyl-3-oxo-1,2,3,4-tetrahydroisoquinolin-6-yl)acetate (40 mg, 0.069 mmol), (8-fluoro-5-methylchroman-6-yl) boronic acid (23 mg, 0.11 mmol), Pd₂(dba)₃ (13 mg, 0.014 mmol), MePhos (5.1 mg, 0.014) and K₃PO₄ (44 mg, 0.208 mmol) in DMF (1 mL) was stirred at 80° C. under N₂ atmosphere overnight. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (20 mg, 48% yield). LC-MS (ESI): m/z (M+1)=616.28.

### Step 8: (S)-2-(tert-butoxy)-2-((R)-2-(4,4-difluorocyclohexyl)-7-(8-fluoro-5-methylchroman-6-yn-5,8-dimethyl-3-oxo-1,2,3,4-tetrahydroisoquinolin-6-yl)acetic Acid

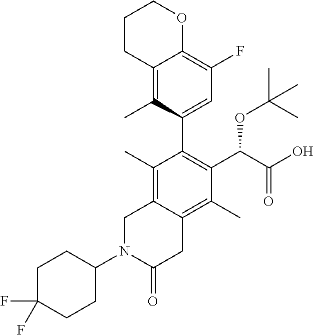

A mixture of ethyl (2S)-2-(tert-butoxy)-2-(2-(4,4-difluorocyclohexyl)-7-(8-fluoro-5-methylchroman-6-yl)-5,8-dimethyl-3-oxo-1,2,3,4-tetrahydroisoquinolin-6-yl)acetate (20 mg, 0.0322 mmol) in EtOH (0.25 mL) and dioxane (0.5 mL) was added LiOH (2.5N, 0.13 mL, 0.32 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (9.6 mg, 51% yield). LC-MS (ESI): m/z (M+1)=588.51. ¹H NMR (400 MHz, CDCl₃) δ 9.54 (br, 1H), 6.68 (d, J=11.3 Hz, 1H), 5.08 (s, 1H), 4.78 (t, J=11.4 Hz, 1H), 4.40-4.18 (m, 4H), 3.71-3.54 (m, 2H), 2.77-2.61 (m, 2H), 2.36-2.07 (m, 7H), 2.04-1.70 (m, 12H), 1.12 (s, 9H).

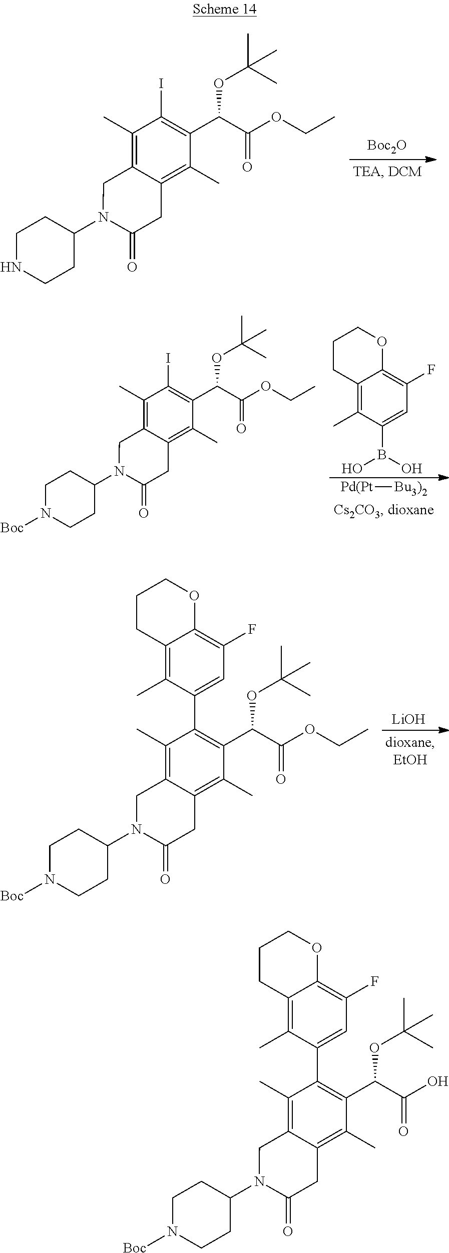

### Example 115: ((S)-2-(tert-butoxy)-2-((R)-2-(1-(tert-butoxycarbonyl)piperidin-4-yl)-7-(8-fluoro-5-methylchroman-6-yl)-5,8-dimethyl-3-oxo-1,2,3,4-tetrahydroisoquinolin-6-yl)acetic Acid

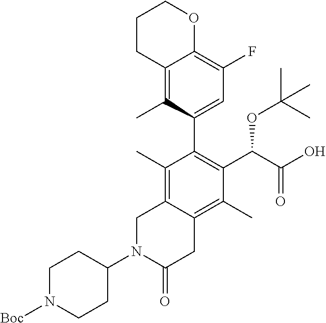

### Step 1: tert-butyl (S)-4-(6-(1-(tert-butoxy)-2-ethoxy-2-oxoethyl)-7-iodo-5,8-dimethyl-3-oxo-3,4-dihydroisoquinolin-2(1H)-yl)piperidine-1-carboxylate

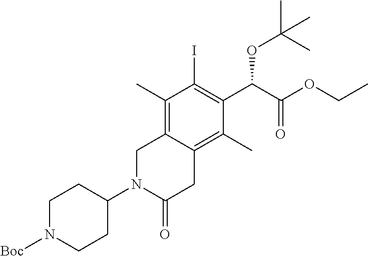 [SMILES: CCOC(=O)[C@@H](OC(C)(C)C)c1c(C)c2c(c(C)c1I)CN(C1CCN(C(=O)OC(C)(C)C)CC1)C(=O)C2]

A mixture of ethyl (S)-2-(tert-butoxy)-2-(7-iodo-5,8-dimethyl-3-oxo-2-(piperidin-4-yl)-1,2,3,4-tetrahydroisoquinolin-6-yl)acetate (20 mg, 0.04 mmol), Boc₂O (8 mg, 0.06 mmol) and TEA (8 mg, 0.08 mmol) in DCM (2 mL) was stirred at r.t. overnight. The resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (15 mg, 60% yield). LC-MS (ESI): m/z (M+1)=643.33.

### Step 2: tert-butyl 4-((R)-6-((S)-1-(tert-butoxy)-2-ethoxy-2-oxoethyl)-7-(8-fluoro-5-methylchroman-6-yl)-5,8-dimethyl-3-oxo-3,4-dihydroisoquinolin-2(1H)-yl)piperidine-1-carboxylate

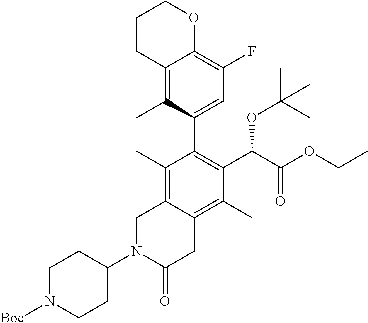

A mixture of tert-butyl (S)-4-(6-(1-(tert-butoxy)-2-ethoxy-2-oxoethyl)-7-iodo-5,8-di methyl-3-oxo-3,4-dihydroisoquinolin-2(1H)-yl)piperidine-1-carboxylate (15 mg, 0.023 mmol), (8-fluoro-5-methylchroman-6-yl) boronic acid (20 mg, 0.092 mmol), Pd(Pt-Bu₃)₂ (2.4 mg, 0.0046 mmol) and Cs₂CO₃ (22.5 mg, 0.069 mmol) in dioxane (1 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (10 mg, 63% yield). LC-MS (ESI): m/z (M+1)=681.40.

### Step 3: ((S)-2-(tert-butoxy)-2-((R)-2-(1-(tert-butoxycarbonyl)piperidin-4-yl)-7-(8-fluoro-5-methylchroman-6-yl)-5,8-dimethyl-3-oxo-1,2,3,4-tetrahydroisoquinolin-6-yl)acetic Acid

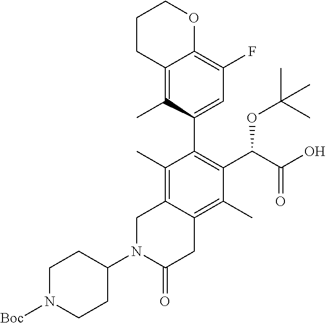

A mixture of tert-butyl 4-(6-((S)-1-(tert-butoxy)-2-ethoxy-2-oxoethyl)-7-(8-fluoro-5-methylchroman-6-yl)-5,8-dimethyl-3-oxo-3,4-dihydroisoquinolin-2(1H)-yl)piperidine-1-carboxylate (10 mg, 0.015 mmol) in EtOH (0.25 mL) and dioxane (0.5 mL) was added LiOH (2.5N, 0.06 mL, 0.15 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (5.6 mg, 59% yield). LC-MS (ESI): m/z (M+1)=653.43. ¹H NMR (400 MHz, CDCl₃) δ 9.68 (br, 1H), 6.69 (d, J=11.3 Hz, 1H), 5.08 (s, 1H), 4.86-4.73 (m, 1H), 4.56-3.98 (m, 6H), 3.64 (s, 2H), 2.94-2.77 (m, 2H), 2.76-2.57 (m, 2H), 2.29 (s, 3H), 2.18-2.06 (m, 2H), 1.91-1.59 (m, 10H), 1.47 (s, 9H), 1.12 (s, 9H).

### Example 116: (2S)-2-(tert-butoxy)-2-((7R)-2-(2,2-dimethylcyclohexyl)-7-(8-fluoro-5-methylchroman-6-yl)-5,8-dimethyl-3-oxo-1,2,3,4-tetrahydroisoquinolin-6-yl)acetic Acid

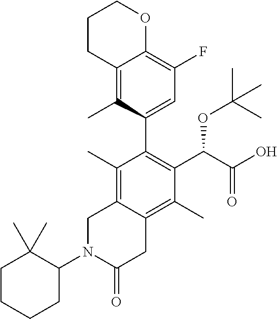

### Step 1: N-(but-2-yn-1-yl)-2,2-dimethylcyclohexan-1-amine

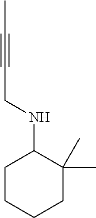

At 0° C., to a solution tert-butyl (2,2-dimethylcyclohexyl)carbamate (2.0 g, 8.8 mmol) in DMF (20 mL) was added NaH (60%, 528 mg, 13.2 mmol). The reaction mixture was stirred at r.t. for 30 min before the introduction of 1-bromobut-2-yne (1.29 g, 9.7 mmol). After stirred at r.t. overnight, the resulting mixture was quenched with sat. NH4Cl aq. solution and extracted with EtOAc. The organic layer was washed with brine, dried over Na₂SO₄, filtered and concentrated to give the crude product which was purified by flash chromatography (silica gel, 0-20% EtOAc in PE) to afford the title compound (2.0 g, 81% yield). LC-MS (ESI): m/z (M+1)=280.34. To a solution of tert-butyl but-2-yn-1-yl(2,2-dimethylcyclohexyl)carbamate (500 mg, 1.79 mmol) in DCM (10 mL) was added 4N HCl in dioxane (5 mL). After stirred at r.t. for 1 hr, the resulting mixture was concentrated to afford the title compound (crude, as HCl salt) which was used in the next step without further purification. LC-MS (ESI): m/z (M+1)=180.40.

### Step 2: N-(but-2-yn-1-yl)-N-(2,2-dimethylcyclohexyl)pent-3-ynamide

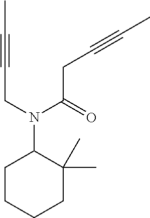 [SMILES: CC#CCC(=O)N(CC#CC)C1CCCCC1(C)C]

To a mixture of N-(but-2-yn-1-yl)-2,2-dimethylcyclohexan-1-amine (500 mg, 2.8 mmol), pent-3-ynoic acid (411 mg, 4.2 mmol) in THF (10 mL) was added EDCl (802 mg, 4.2 mmol) and TEA (566 mg, 5.6 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-50% EtOAc in PE) to afford the title compound (250 mg, 34% yield) as a colorless oil. LC-MS (ESI): m/z (M+1)=260.36.

### Step 3: ethyl (2S)-2-(2-(2,2-dimethylcyclohexyl)-5,8-dimethyl-3-oxo-7-(trimethylsilyl)-1,2,3,4-tetrahydroisoquinolin-6-yl)-2-hydroxyacetate

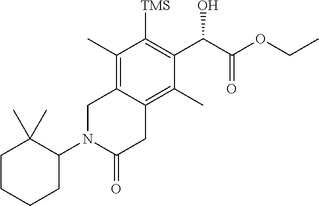 [SMILES: CCOC(=O)[C@@H](O)c1c(C)c2c(c(C)c1[Si](C)(C)C)CN(C1CCCCC1(C)C)C(=O)C2]

A mixture of Rh(cod)₂BF₄ (77 mg, 0.19 mmol) and BINAP (118 mg, 0.19 mmol) in DCM (5 mL) was stirred at r.t. under H₂ atmosphere for 6 hr. After concentration, the resulting mixture was dissolved in DCE (10 mL) and ethyl (S)-2-hydroxy-4-(trimethyl silyl)but-3-ynoate (386 mg, 1.93 mmol) was introduced. The reaction mixture was heated up to 80° C. in a seal tube and N-(but-2-yn-1-yl)-N-(2,2-dimethylcyclohexyl) pent-3-ynamide (250 mg, 0.96 mmol) was added as a DCE solution (10 mL) over 3 hr. After stirred at 80° C. for 30 min, the resulting mixture was concentrated to give the crude product which was purified by flash chromatography (silica gel, 0-30% EtOAc in PE) to afford the title compound (170 mg, 39% yield). LC-MS (ESI): m/z (M+1)=460.42.

### Step 4: ethyl (2S)-2-(2-(2,2-dimethylcyclohexyl)-7-iodo-5,8-dimethyl-3-oxo-1,2,3,4-tetrahydroisoquinolin-6-yl)-2-hydroxyacetate

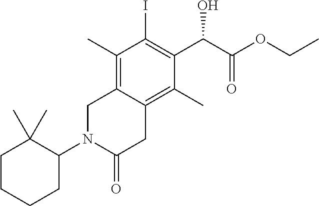 [SMILES: CCOC(=O)[C@@H](O)c1c(C)c2c(c(C)c1I)CN(C1CCCCC1(C)C)C(=O)C2]

At 0° C., to a solution of ethyl (2S)-2-(2-(2,2-dimethylcyclohexyl)-5,8-dimethyl-3-oxo-7-(trimethylsilyl)-1,2,3,4-tetrahydroisoquinolin-6-yl)-2-hydroxyacetate (170 mg, 0.37 mmol) in DCM (4 mL) was added ICl (2.5M solution in DCM, 0.15 mL, 0.37 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 6: ethyl (2S)-2-(tert-butoxy)-2-(2-(2,2-dimethylcyclohexyl)-7-iodo-5,8-dimethyl-3-oxo-1,2,3,4-tetrahydroisoquinolin-6-yl)acetate

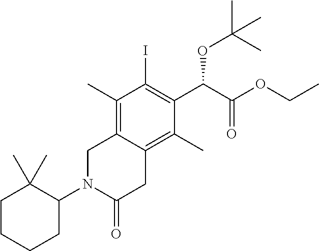 [SMILES: CCOC(=O)[C@@H](OC(C)(C)C)c1c(C)c2c(c(C)c1I)CN(C1CCCCC1(C)C)C(=O)C2]

To a solution of ethyl (2S)-2-(2-(2,2-dimethylcyclohexyl)-7-iodo-5,8-dimethyl-3-oxo-1,2,3,4-tetrahydroisoquinolin-6-yl)-2-hydroxyacetate (crude product from the previous step, 0.37 mmol) in t-BuOAc (2 mL) was added HClO₄ (37 mg, 0.37 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (20 mg, 10% yield over two steps).

LC-MS (ESI): m/z (M+1)=570.37.

### Step 7: (2S)-ethyl 2-(tert-butoxy)-2-((7R)-2-(2,2-dimethylcyclohexyl)-7-(8-fluoro-5-methylchroman-6-yl)-5,8-dimethyl-3-oxo-1,2,3,4-tetrahydroisoquinolin-6-yl)acetate

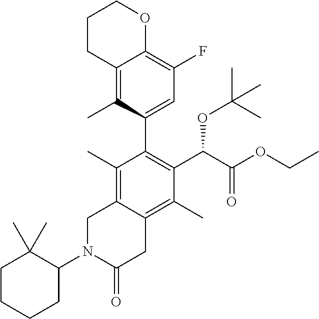

A mixture of ethyl (2S)-2-(tert-butoxy)-2-(2-(2,2-dimethylcyclohexyl)-7-iodo-5,8-di methyl-3-oxo-1,2,3,4-tetrahydroisoquinolin-6-yl)acetate (20 mg, 0.035 mmol), (8-fluoro-5-methylchroman-6-yl) boronic acid (30 mg, 0.141 mmol), Pd(Pt-Bu₃)₂ (3.6 mg, 0.007 mmol) and Cs₂CO₃ (36 mg, 0.11 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-40% EtOAc in PE) to afford the title compound (18 mg, 86% yield). LC-MS (ESI): m/z (M+1)=608.90.

### Step 8: (2S)-2-(tert-butoxy)-2-((7R)-2-(2,2-dimethylcyclohexyl)-7-(8-fluoro-5-methylchroman-6-yl)-5,8-dimethyl-3-oxo-1,2,3,4-tetrahydroisoquinolin-6-yl)acetic acid

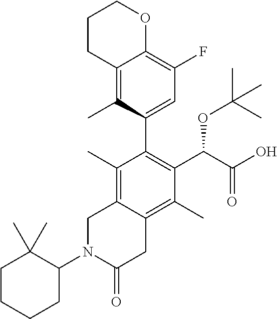

A mixture of ethyl (2S)-2-(tert-butoxy)-2-(2-(2,2-dimethylcyclohexyl)-7-(8-fluoro-5-methylchroman-6-yl)-5,8-dimethyl-3-oxo-1,2,3,4-tetrahydroisoquinolin-6-yl)acetate (18 mg, 0.029 mmol) in EtOH (0.5 mL) and dioxane (1.0 mL) was added LiOH (2.5N, 0.12 mL, 0.3 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (8 mg, 47% yield). LC-MS (ESI): m/z (M+1)=580.45. ¹H NMR (400 MHz, CDCl₃) δ 9.54 (br, 1H), 6.69 (d, J=11.3 Hz, 1H), 5.09 (s, 1H), 4.57-4.46 (m, 1H), 4.44-4.15 (m, 4H), 3.74-3.51 (m, 2H), 2.78-2.58 (m, 2H), 2.32 (s, 3H), 2.20-2.06 (m, 2H), 1.87-1.41 (m, 14H), 1.26-0.78 (m, 15H).

### Example 117: ((2S)-2-(tert-butoxy)-2-((7R)-2-(3,3-difluorocyclohexyl)-7-(8-fluoro-5-methylchroman-6-yl)-5,8-dimethyl-3-oxo-1,2,3,4-tetrahydroisoquinolin-6-yl)acetic acid

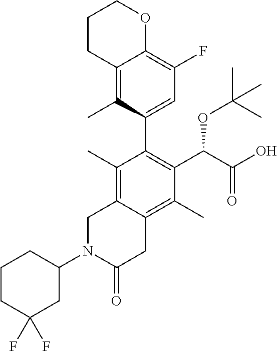

### Step 1: tert-butyl (3,3-difluorocyclohexyl)carbamate

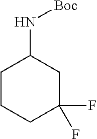 [SMILES: CC(C)(C)OC(=O)NC1CCCC(F)(F)C1]

A mixture of 3,3-difluorocyclohexan-1-amine (1.3 g, 9.6 mmol), Boc₂O (4.2 g, 19.2 mmol) and TEA (1.95 g, 19.2 mmol) in THF (20 mL) was stirred at r.t. overnight. The resulting mixture was partitioned between EtOAc and H₂O. The organic layer was washed with brine, dried over Na₂SO₄, filtered and concentrated to give the crude product which was purified by flash chromatography (silica gel, 0-20% EtOAc in PE) to afford the title compound (1.4 g, 62% yield) as a yellow solid. LC-MS (ESI): m/z (M+1)=236.24.

### Step 2: tert-butyl but-2-yn-1-yl(3,3-difluorocyclohexyl)carbamate

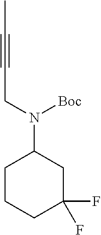 [SMILES: CC#CCN(C(=O)OC(C)(C)C)C1CCCC(F)(F)C1]

At 0° C., to a solution tert-butyl (3,3-difluorocyclohexyl)carbamate (1.3 g, 5.5 mmol) in DMF (20 mL) was added NaH (60%, 332 mg, 8.29 mmol). The reaction mixture was stirred at r.t. for 30 min before the introduction of 1-bromobut-2-yne (808 mg, 6.08 mmol). After stirred at r.t. overnight, the resulting mixture was quenched with sat. NH4Cl aq. solution and extracted with EtOAc. The organic layer was washed with brine, dried over Na₂SO₄, filtered and concentrated to give the crude product which was purified by flash chromatography (silica gel, 0-20% EtOAc in PE) to afford the title compound (1.5 g, 94% yield). LC-MS (ESI): m/z (M+1)=288.31.

### Step 3: N-(but-2-yn-1-yl)-3,3-difluorocyclohexan-1-amine

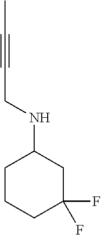

A mixture of tert-butyl but-2-yn-1-yl(3,3-difluorocyclohexyl)carbamate (1.5 g, 5.22 mmol) in 4N HCl in dioxane (15 mL) and DCM (20 mL) was stirred at r.t. for 2 hr. The resulting mixture was concentrated under reduced pressure and the residue was diluted with DCM and washed with sat. NaHCO₃ aq. solution and brine, dried over Na₂SO₄, filtered and concentrated to afford the title compound (936 mg, 96% yield) which was used in the next step without further purification. LC-MS (ESI): m/z (M+1)=188.50.

### Step 4: N-(but-2-yn-1-yl)-N-(3,3-difluorocyclohexyl)pent-3-ynamide

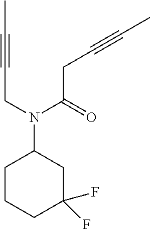 [SMILES: CC#CCC(=O)N(CC#CC)C1CCCC(F)(F)C1]

To a mixture of N-(but-2-yn-1-yl)-3,3-difluorocyclohexan-1-amine (500 mg, 2.67 mmol), pent-3-ynoic acid (393 mg, 4.0 mmol) in THF (10 mL) was added EDCl (764 mg, 4.0 mmol) and TEA (536 mg, 5.3 mmol). After stirred at r.t. for 3 hr, the resulting mixture was partitioned between EtOAc and H₂O. The organic layer was washed with brine, dried over Na₂SO₄, filtered and concentrated to give the crude product which was purified by flash chromatography (silica gel, 0-30% EtOAc in PE) to afford the title compound (500 mg, 70% yield) as a colorless oil. LC-MS (ESI): m/z (M+1)=268.56.

### Step 5: ethyl (2S)-2-(2-(3,3-difluorocyclohexyl)-5,8-dimethyl-3-oxo-7-(trimethylsilyl)-1,2,3,4-tetrahydroisoquinolin-6-yl)-2-hydroxyacetate

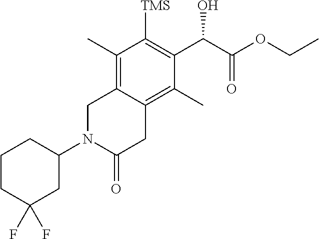 [SMILES: CCOC(=O)[C@@H](O)c1c(C)c2c(c(C)c1[Si](C)(C)C)CN(C1CCCC(F)(F)C1)C(=O)C2]

A mixture of Rh(cod)₂BF₄ (150 mg, 0.37 mmol) and BINAP (230 mg, 0.37 mmol) in DCM (10 mL) was stirred at r.t. under H₂ atmosphere for 6 hr. After concentration, the resulting mixture was dissolved in DCE (10 mL) and ethyl (S)-2-hydroxy-4-(trimethyl silyl)but-3-ynoate (748 mg, 3.74 mmol) was introduced. The reaction mixture was heated up to 80° C. in a seal tube and N-(but-2-yn-1-yl)-N-(3,3-difluorocyclohexyl) pent-3-ynamide (500 mg, 1.87 mmol) was added as a DCE solution (10 mL) over 3 hr. After stirred at 80° C. for 30 min, the resulting mixture was concentrated to give the crude product which was purified by flash chromatography (silica gel, 0-50% EtOAc in PE) to afford the title compound (300 mg, 34% yield). LC-MS (ESI): m/z (M+1)=468.32.

### Step 6: ethyl (2S)-2-(2-(3,3-difluorocyclohexyl)-7-iodo-5,8-dimethyl-3-oxo-1,2,3,4-tetrahydroisoquinolin-6-yl)-2-hydroxyacetate

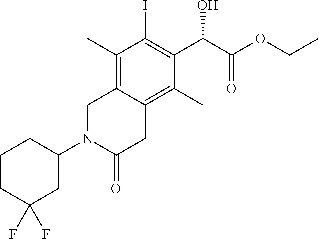 [SMILES: CCOC(=O)[C@@H](O)c1c(C)c2c(c(C)c1I)CN(C1CCCC(F)(F)C1)C(=O)C2]

At 0° C., to a solution of ethyl (2S)-2-(2-(3,3-difluorocyclohexyl)-5,8-dimethyl-3-oxo-7-(trimethylsilyl)-1,2,3,4-tetrahydroisoquinolin-6-yl)-2-hydroxyacetate (100 mg, 0.21 mmol) in DCM (2 mL) was added ICl (2.5M solution in DCM, 0.13 mL, 0.32 mmol). After stirred at 0° C. for 10 min, the resulting mixture was quenched with sat. Na₂S₂O₃ aq. solution and extracted with DCM. The organic layer was washed with brine, dried over Na₂SO₄, filtered and concentrated to give the title compound as a crude product which was used in the next step without further purification.

### Step 7: ethyl (2S)-2-(tert-butoxy)-2-(2-(3,3-difluorocyclohexyl)-7-iodo-5,8-dimethyl-3-oxo-1,2,3,4-tetrahydroisoquinolin-6-yl)acetate

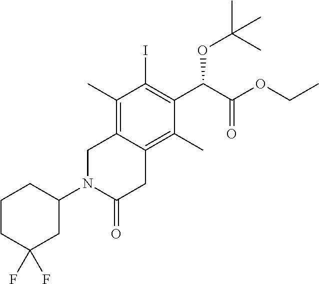 [SMILES: CCOC(=O)[C@@H](OC(C)(C)C)c1c(C)c2c(c(C)c1I)CN(C1CCCC(F)(F)C1)C(=O)C2]

To a solution of ethyl (2S)-2-(2-(3,3-difluorocyclohexyl)-7-iodo-5,8-dimethyl-3-oxo-1,2,3,4-tetrahydroisoquinolin-6-yl)-2-hydroxyacetate (crude product from the previous step, 0.21 mmol) in t-BuOAc (2 mL) was added HClO₄ (32 mg, 0.32 mmol). After stirred at r.t. for 30 min, the resulting mixture was quenched with sat. NaHCO₃ aq. solution and extracted with EtOAc. The organic layer was washed with brine, dried over Na₂SO₄, filtered and concentrated to give the crude product which was purified by flash chromatography (silica gel, 0-50% EtOAc in PE) to afford the title compound (40 mg, 33% yield over two steps).

LC-MS (ESI): m/z (M+1)=578.66.

### Step 8: (2S)-ethyl 2-(tert-butoxy)-2-((7R)-2-(3,3-difluorocyclohexyl)-7-(8-fluoro-5-methylchroman-6-yl)-5,8-dimethyl-3-oxo-1,2,3,4-tetrahydroisoquinolin-6-yl)acetate

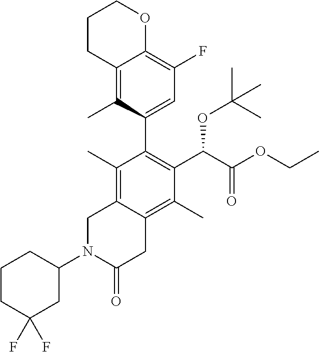

A mixture of ethyl (2S)-2-(tert-butoxy)-2-(2-(3,3-difluorocyclohexyl)-7-iodo-5,8-di methyl-3-oxo-1,2,3,4-tetrahydroisoquinolin-6-yl)acetate (40 mg, 0.069 mmol), (8-fluoro-5-methylchroman-6-yl) boronic acid (58 mg, 0.277 mmol), Pd(Pt-Bu₃)₂ (7.1 mg, 0.014 mmol) and Cs₂CO₃ (68 mg, 0.21 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (12 mg, 28% yield). LC-MS (ESI): m/z (M+1)=616.33.

### Step 9: (2S)-2-(tert-butoxy)-2-((7R)-2-(3,3-difluorocyclohexyl)-7-(8-fluoro-5-methylchroman-6-yl)-5,8-dimethyl-3-oxo-1,2,3,4-tetrahydroisoquinolin-6-yl)acetic Acid

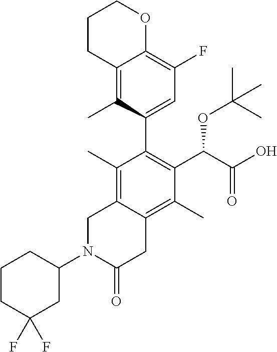

A mixture of ethyl (2S)-2-(tert-butoxy)-2-(2-(3,3-difluorocyclohexyl)-7-(8-fluoro-5-methylchroman-6-yl)-5,8-dimethyl-3-oxo-1,2,3,4-tetrahydroisoquinolin-6-yl)acetate (10 mg, 0.016 mmol) in EtOH (0.5 mL) and dioxane (1.0 mL) was added LiOH (2.5N, 0.06 mL, 0.15 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (4.6 mg, 48% yield). LC-MS (ESI): m/z (M+1)=588.26. ¹H NMR (400 MHz, CDCl₃) δ 9.67 (br, 1H), 6.68 (d, J=11.3 Hz, 1H), 5.07 (s, 1H), 4.75-4.65 (m, 1H), 4.41-4.30 (m, 2H), 4.29-4.25 (m, 2H), 3.68-3.57 (m, 2H), 2.76-2.64 (m, 2H), 2.29 (s, 3H), 2.26-2.21 (m, 1H), 2.19-2.07 (m, 4H), 1.91-1.79 (m, 8H), 1.73-1.62 (m, 3H), 1.12 (s, 9H).

### Example 118: ((S)-2-(tert-butoxy)-2-((R)-7-(8-chloro-5-methylchroman-6-yl)-2-cyclohexyl-5,8-dimethyl-3-oxo-1,2,3,4-tetrahydroisoquinolin-6-yl)acetic Acid

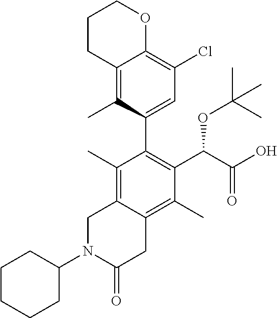

### Step 1: (S)-ethyl 2-(tert-butoxy)-2-((R)-7-(8-chloro-5-methylchroman-6-yl)-2-cyclohexyl-5,8-dimethyl-3-oxo-1,2,3,4-tetrahydroisoquinolin-6-yl)acetate

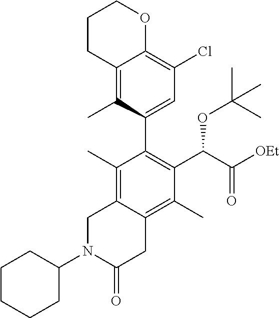

A mixture of ethyl (2S)-2-(tert-butoxy)-2-(2-(3,3-difluorocyclohexyl)-7-iodo-5,8-di methyl-3-oxo-1,2,3,4-tetrahydroisoquinolin-6-yl)acetate (42 mg, 0.078 mmol), (8-fluoro-5-methylchroman-6-yl) boronic acid (96 mg, 0.32 mmol), Pd(Pt-Bu₃)₂ (8 mg, 0.0155 mmol) and Cs₂CO₃ (76 mg, 0.233 mmol) in dioxane (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (30 mg, 67% yield). LC-MS (ESI): m/z (M/M+2)=596.53/598.50.

### Step 2: (S)-2-(tert-butoxy)-2-((R)-7-(8-chloro-5-methylchroman-6-yl)-2-cyclohexyl-5,8-dimethyl-3-oxo-1,2,3,4-tetrahydroisoquinolin-6-yl)acetic Acid

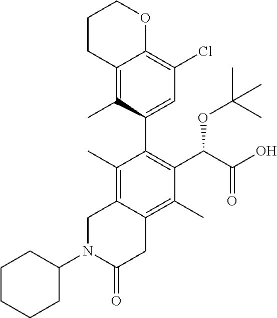

A mixture of ethyl (2S)-2-(tert-butoxy)-2-(7-(8-chloro-5-methylchroman-6-yl)-2-cyclo hexyl-5,8-dimethyl-3-oxo-1,2,3,4-tetrahydroisoquinolin-6-yl)acetate (30 mg, 0.05 mmol) in EtOH (0.5 mL) and dioxane (1.0 mL) was added LiOH (2.5N, 0.2 mL, 0.5 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (9.6 mg, 34% yield). LC-MS (ESI): m/z (M/M+2)=568.69/570.70. ¹H NMR (400 MHz, CDCl₃) δ 9.74 (br, 1H), 6.96 (s, 1H), 5.04 (s, 1H), 4.65-4.57 (m, 1H), 4.38-4.27 (m, 4H), 3.67-3.56 (m, 2H), 2.77-2.64 (m, 2H), 2.29 (s, 3H), 2.15-2.08 (m, 2H), 1.90-1.81 (m, 8H), 1.79-1.66 (m, 4H), 1.54-1.43 (m, 4H), 1.12 (s, 9H).

### Example 119: (S)-2-(tert-butoxy)-2-((R)-2-cyclohexyl-5,8-dimethyl-7-(5-methyl-3,4-dihydro-2H-benzo[b][1,4]oxazin-6-yl)-3-oxo-1,2,3,4-tetrahydroisoquinolin-6-yl)acetic Acid

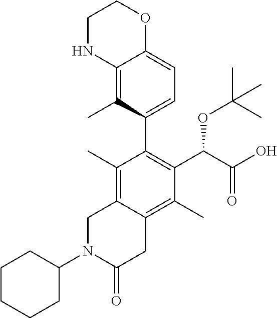

### Step 1: ethyl (2S)-2-(tert-butoxy)-2-((R)-2-cyclohexyl-5,8-dimethyl-7-(5-methyl-3,4-dihydro-2H-benzo[b][1,4]oxazin-6-yl)-3-oxo-1,2,3,4-tetrahydroisoquinolin-6-yl)acetate

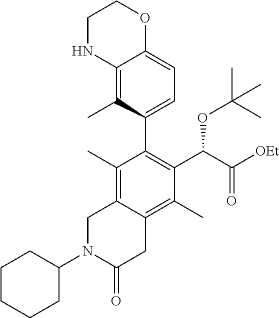

A mixture of ethyl (S)-2-(tert-butoxy)-2-(2-cyclohexyl-7-iodo-5,8-dimethyl-3-oxo-1,2,3,4-tetrahydroisoquinolin-6-yl)acetate (20 mg, 0.037 mmol), 5-methyl-6-(4,4,5,5-tetramethyl-1,3,2-dioxaborolan-2-yl)-3,4-dihydro-2H-benzo[b][1,4]oxazine (20 mg, 0.074 mmol), Pd₂(dba)₃ (6.4 mg, 0.007 mmol), MePhos (2.5 mg, 0.007 mmol) and K₃PO₄ (23 mg, 0.111 mmol) in DMF (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (9 mg, 45% yield). LC-MS (ESI): m/z (M+1)=563.53.

### Step 2: (S)-2-(tert-butoxy)-2-((R)-2-cyclohexyl-5,8-dimethyl-7-(5-methyl-3,4-dihydro-2H-benzo[b][1,4]oxazin-6-yl)-3-oxo-1,2,3,4-tetrahydroisoquinolin-6-yl)acetic Acid

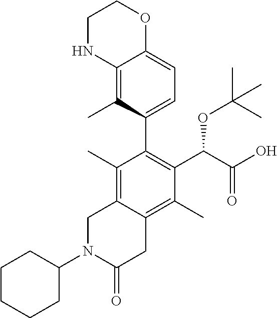

A mixture of ethyl (2S)-2-(tert-butoxy)-2-(2-cyclohexyl-5,8-dimethyl-7-(5-methyl-3,4-dihydro-2H-benzo[b][1,4]oxazin-6-yl)-3-oxo-1,2,3,4-tetrahydroisoquinolin-6-yl)acetate (9 mg, 0.016 mmol) in EtOH (0.25 mL) and dioxane (0.5 mL) was added LiOH (2.5N, 0.06 mL, 0.15 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (3.5 mg, 41% yield). LC-MS (ESI): m/z (M+1)=535.65. ¹H NMR (400 MHz, CDCl₃) δ 9.62 (br, 1H), 6.71 (d, J=8.2 Hz, 1H), 6.42 (d, J=8.1 Hz, 1H), 5.40-5.28 (m, 1H), 5.14 (s, 1H), 4.65-4.58 (m, 1H), 4.40-4.25 (m, 4H), 3.66-3.49 (m, 4H), 2.26 (s, 3H), 1.88-1.76 (m, 8H), 1.74-1.68 (m, 2H), 1.58-1.48 (m, 6H), 1.10 (s, 9H).

### Example 120: (S)-2-(tert-butoxy)-2-((R)-2-cyclohexyl-5,8-dimethyl-7-(5-methylchroman-6-yl)-3-oxo-1,2,3,4-tetrahydroisoquinolin-6-yl)acetic Acid

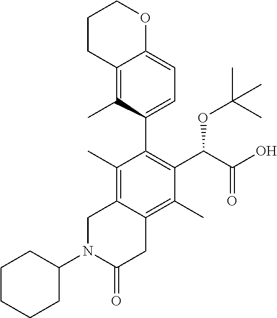

### Step 1: ethyl (2S)-2-(tert-butoxy)-2-(2-cyclohexyl-5,8-dimethyl-7-(5-methylchroman-6-yl)-3-oxo-1,2,3,4-tetrahydroisoquinolin-6-yl)acetate

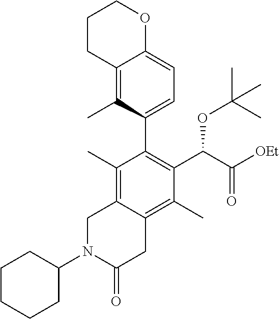

A mixture of ethyl (S)-2-(tert-butoxy)-2-(2-cyclohexyl-7-iodo-5,8-dimethyl-3-oxo-1,2,3,4-tetrahydroisoquinolin-6-yl)acetate (42 mg, 0.08 mmol), 4,4,5,5-tetramethyl-2-(5-methylchroman-6-yl)-1,3,2-dioxaborolane (44 mg, 0.16 mmol), Pd₂(dba)₃ (15 mg, 0.016 mmol), MePhos (6 mg, 0.016 mmol) and K₃PO₄ (51 mg, 0.24 mmol) in DMF (2 mL) was stirred at 90° C. under N₂ atmosphere for 15 min. The resulting mixture was partitioned between H₂O and EtOAc. The organic layer was washed with brine, dried over Na₂SO₄, filtered and concentrated to give the crude product which was purified by flash chromatography (silica gel, 0-50% EtOAc in PE) to afford the title compound (20 mg, 44% yield). LC-MS (ESI): m/z (M+1)=562.45.

### Step 2: (S)-2-(tert-butoxy)-2-((R)-2-cyclohexyl-5,8-dimethyl-7-(5-methylchroman-6-yl)-3-oxo-1,2,3,4-tetrahydroisoquinolin-6-yl)acetic Acid

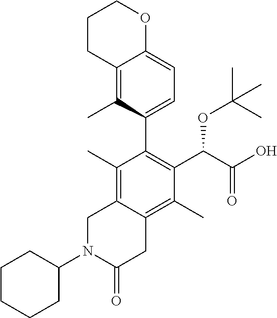

A mixture of ethyl (2S)-2-(tert-butoxy)-2-(2-cyclohexyl-5,8-dimethyl-7-(5-methyl chroman-6-yl)-3-oxo-1,2,3,4-tetrahydroisoquinolin-6-yl)acetate (20 mg, 0.036 mmol) in EtOH (0.5 mL) and dioxane (1.0 mL) was added LiOH (2.5N, 0.14 mL, 0.36 mmol). After stirred at 100° C. overnight, the resulting mixture was acidified with 1N HCl to pH 3-4 and extracted with EtOAc. The organic layer was washed with brine, dried over Na₂SO₄, filtered and concentrated to give the crude product which was purified by HPLC (C18, 0-100% MeCN in H₂O with 0.1% formic acid) to afford the title compound (7 mg, 37% yield). LC-MS (ESI): m/z (M+1)=534.56. ¹H NMR (400 MHz, CDCl₃) δ 9.67 (br, 1H), 6.82 (d, J=7.9 Hz, 1H), 6.71 (d, J=8.3 Hz, 1H), 5.12 (s, 1H), 4.66-4.57 (m, 1H), 4.40-4.28 (m, 2H), 4.25-4.12 (m, 2H), 3.68-3.56 (m, 2H), 2.75-2.60 (m, 2H), 2.28 (s, 3H), 2.14-2.04 (m, 2H), 1.90 (s, 3H), 1.82 (s, 3H), 1.79-1.67 (m, 3H), 1.62-1.44 (m, 7H), 1.10 (s, 9H).

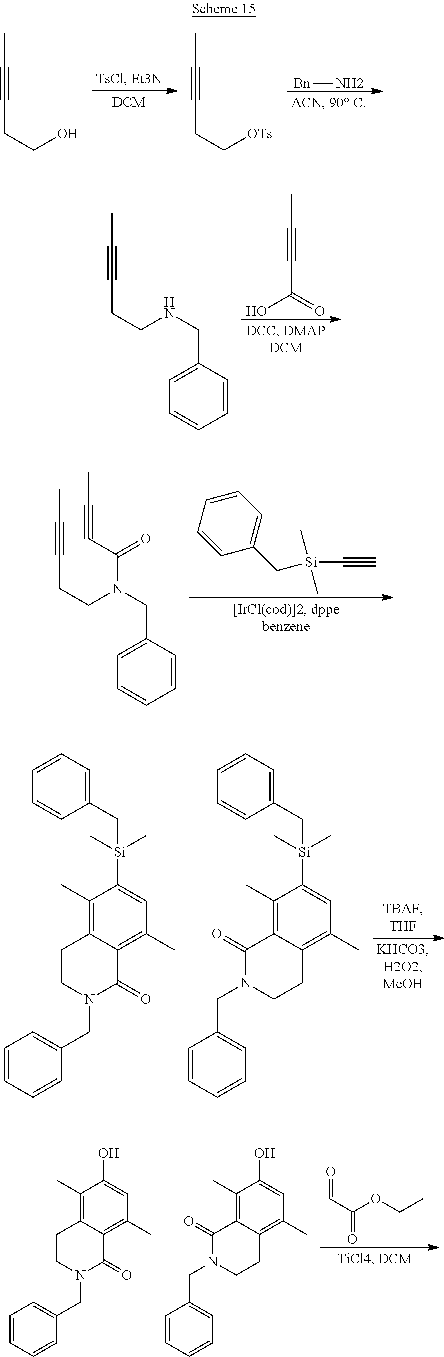

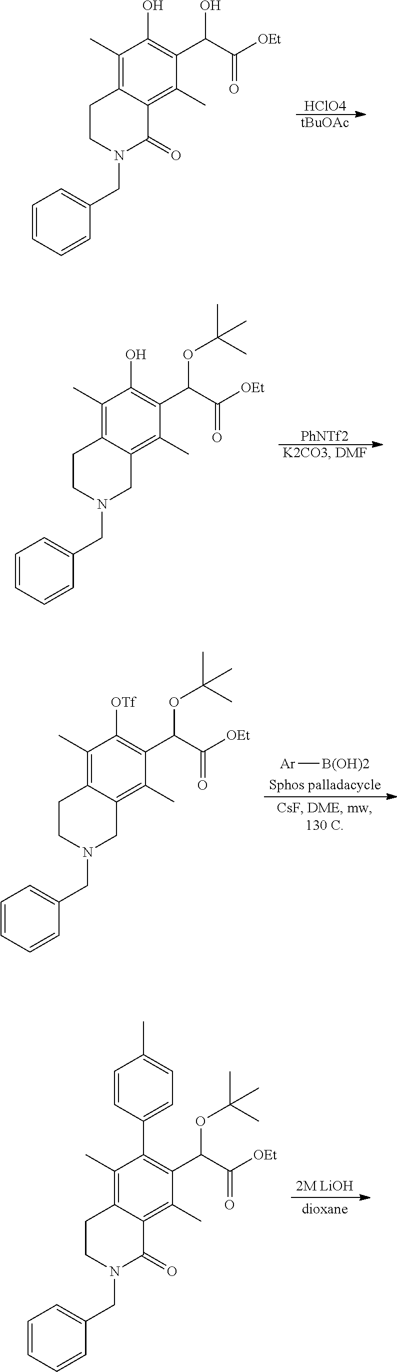

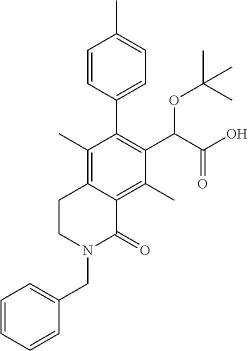 [SMILES: Cc1ccc(-c2c(C)c3c(c(C)c2C(OC(C)(C)C)C(=O)O)C(=O)N(Cc2ccccc2)CC3)cc1]

### Example 121: 2-(2-benzyl-5,8-dimethyl-1-oxo-6-(p-tolyl)-1,2,3,4-tetrahydroisoquinolin-7-yl)-2-(tert-butoxy)acetic Acid

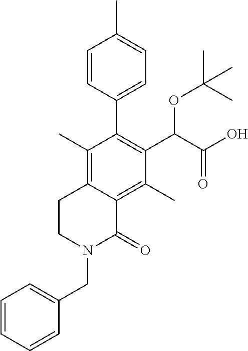 [SMILES: Cc1ccc(-c2c(C)c3c(c(C)c2C(OC(C)(C)C)C(=O)O)C(=O)N(Cc2ccccc2)CC3)cc1]

### Step 1: pent-3-yn-1-yl 4-methylbenzenesulfonate

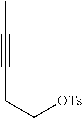 [SMILES: CC#CCCOS(=O)(=O)c1ccc(C)cc1]

An ice cold solution of pent-3-yn-1-ol (4 g, 47.6 mmol) in Dichloromethane (DCM) (90 ml) was treated with Et3N (9.94 ml, 71.3 mmol), pTsCl (9.07 g, 47.6 mmol), and then stirred at rt for 1 day. The reaction was diluted with water, extracted with DCM, washed with Brine, dried over Na2SO4, filtered, and concentrated to give the title compound (11.31 g, 47.5 mmol, 100% yield) as pale yellow liquid. ¹H NMR (400 MHz, CHLOROFORM-d) δ ppm 7.81 (d, J=8.0 Hz, 2H), 7.35 (d, J=8.0 Hz, 2H), 4.06 (t, J=7.2 Hz, 2H), 2.54-2.42 (m, 5H), 1.72 (t, J=2.4 Hz, 3H); LCMS (m/z) ES⁺=239 (M+1) (weak).

### Step 2: N-benzylpent-3-yn-1-amine

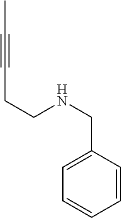

A solution of pent-3-yn-1-yl 4-methylbenzenesulfonate (4.5 g, 18.88 mmol) in Acetonitrile (45 mL) was treated with benzylamine (8.25 mL, 76 mmol) and heated in 90° C. bath in a sealed flask. After 2.5 hours, the reaction was cooled to rt overnight. The suspension was diluted with Et2O, filtered, and the filtrate was concentrated. The residue was diluted with sat. NaHCO₃, extracted with Et2O, washed with Brine, dried over Na2SO4, filtered, and concentrated to give 5.3684 g brown liquid. The residue was diluted with Et2O, washed with sat. NaHCO₃, Brine, dried over Na2SO4, filtered, and concentrated to give the title compound (3.0111 g, 17.38 mmol, 92% yield) as yellow liquid. ¹H NMR (400 MHz, CHLOROFORM-d) δ ppm 7.34 (d, J=4.8 Hz, 4H), 7.29-7.21 (m, 1H), 3.83 (s, 2H), 2.76 (t, J=6.5 Hz, 2H), 2.40-2.30 (m, 2H), 1.79 (t, J=2.5 Hz, 3H).

### Step 3: N-benzyl-N-(pent-3-yn-1-yl)but-2-ynamide

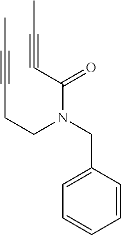 [SMILES: CC#CCCN(Cc1ccccc1)C(=O)C#CC]

A solution of N-benzylpent-3-yn-1-amine (3 g, 17.32 mmol) in Dichloromethane (DCM) (55 mL) was treated with DCC (4.11 g, 19.91 mmol) and DMAP (0.212 g, 1.732 mmol), cooled to 0° C., and then treated with a solution of but-2-ynoic acid (1.601 g, 19.05 mmol) in Dichloromethane (DCM) (10 mL). The reaction was stirred at rt for 18 hours, filtered, and the filtrate was concentrated. Purification with column chromatography (0-70% EtOAc/Hexane) afforded the title compound (3.4306 g, 14.34 mmol, 83% yield) as light yellow oil. NMR shows ˜1:1 mixture of rotomers. ¹H NMR (400 MHz, CHLOROFORM-d) δ ppm 7.44-7.18 (m, 5H), 4.90 (s, 1H), 4.69 (s, 1H), 3.61 (t, J=7.3 Hz, 1H), 3.42 (t, J=7.2 Hz, 1H), 2.38 (tddd, J=2.4, 4.7, 7.2, 14.3 Hz, 2H), 2.03 (d, J=17.3 Hz, 3H), 1.78 (td, J=2.6, 5.4 Hz, 3H); LCMS (m/z) ES⁺=240 (M+1).

### Step 4: 2-benzyl-6-(benzyldimethylsilyl)-5,8-dimethyl-3,4-dihydroisoquinolin-1(2H)-one and 2-benzyl-7-(benzyldimethylsilyl)-5,8-dimethyl-3,4-dihydroisoquinolin-1(2H)-one

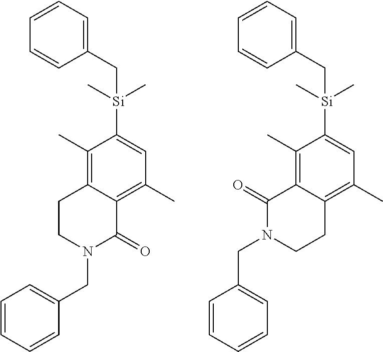 [SMILES: Cc1cc([Si](C)(C)Cc2ccccc2)c(C)c2c1C(=O)N(Cc1ccccc1)CC2 ; Cc1cc([Si](C)(C)Cc2ccccc2)c(C)c2c1CCN(Cc1ccccc1)C2=O]

A degassed solution of N-benzyl-N-(pent-3-yn-1-yl)but-2-ynamide (2 g, 8.36 mmol) in Benzene (83.6 mL) was treated with benzyl(ethynyl)dimethylsilane (2.91 g, 16.71 mmol), [IrCl(cod)]2 (0.561 g, 0.836 mmol), DPPE (0.666 g, 1.671 mmol), and stirred at rt for 18 hours. The reaction was bubbled with N2, treated with additional benzyl(ethynyl)dimethylsilane (493 mg), IrCl(cod) dimer (186 mg), dppe (235 mg), and then stirred for 1 day. The reaction was concentrated and purified with column chromatography (0-35% EtOAc/Hexane) to afford 1:1 mixture of 2-benzyl-6-(benzyldimethylsilyl)-5,8-dimethyl-3,4-dihydroisoquinolin-1(2H)-one and 2-benzyl-7-(benzyldimethylsilyl)-5,8-dimethyl-3,4-dihydroisoquinolin-1(2H)-one (2.1 g, 5.08 mmol, 60.7% yield) as brown oil. ¹H NMR (400 MHz, CHLOROFORM-d) δ ppm 7.40-7.28 (m, 5H), 7.25-7.16 (m, 3H), 7.11-7.05 (m, 1H), 6.99 (dd, J=7.3, 12.5 Hz, 2H), 4.80 (d, J=4.3 Hz, 2H), 3.44 (dt, J=3.5, 6.3 Hz, 2H), 2.84 (s, 1.5H), 2.78 (td, J=6.4, 16.6 Hz, 2H), 2.70 (s, 1.5H), 2.42 (d, J=14.6 Hz, 2H), 2.32-2.20 (m, 3H), 0.30 (d, J=1.8 Hz, 6H); LCMS (m/z) ES⁺=414 (M+1).

### Step 5: 2-benzyl-6-hydroxy-5,8-dimethyl-3,4-dihydroisoquinolin-1(2H)-one and 2-benzyl-7-hydroxy-5,8-dimethyl-3,4-dihydroisoquinolin-1(2H)-one

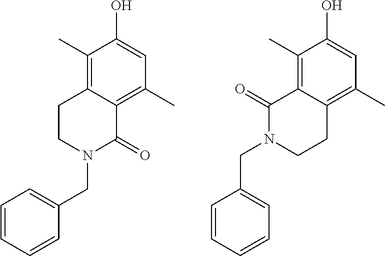 [SMILES: Cc1cc(O)c(C)c2c1C(=O)N(Cc1ccccc1)CC2 ; Cc1cc(O)c(C)c2c1CCN(Cc1ccccc1)C2=O]

An ice cold solution of 1:1 mixture of 2-benzyl-6-(benzyldimethylsilyl)-5,8-dimethyl-3,4-dihydroisoquinolin-1(2H)-one and 2-benzyl-7-(benzyldimethylsilyl)-5,8-dimethyl-3,4-dihydroisoquinolin-1(2H)-one (2.1 g, 5.08 mmol) in Tetrahydrofuran (THF) (12 mL) (open to air) was treated dropwise with TBAF (1M in THF) (20.32 mL, 20.32 mmol), and then stirred at rt for 20 min. The mixture was treated with Methanol (37 mL), KHCO3 (1.017 g, 10.16 mmol), H2O2 (30%) (5.19 mL, 50.8 mmol), and stirred at rt for 1.5 hours. The mixture was diluted with sat. aq. Na2S2O3, extracted with EtOAc, washed with Brine, dried over Na2SO4, filtered, and concentrated. Purification with column chromatography (0-60% EtOAc/Hexane) afforded ˜1:1 mixture of 2-benzyl-6-hydroxy-5,8-dimethyl-3,4-dihydroisoquinolin-1(2H)-one and 2-benzyl-7-hydroxy-5,8-dimethyl-3,4-dihydroisoquinolin-1(2H)-one (0.56 g, 1.990 mmol, 39.2% yield) as yellow solid. ¹H NMR (400 MHz, CHLOROFORM-d) δ ppm 7.40-7.28 (m, 5H), 6.85-6.50 (m, 1H), 5.48 (s, 0.5H), 4.95 (s, 0.5H), 4.84-4.73 (m, 2H), 3.48-3.30 (m, 2H), 2.83-2.76 (m, 1H), 2.73-2.67 (m, 1H), 2.63 (d, J=19.1 Hz, 3H), 2.21-2.09 (m, 3H); LCMS (m/z) ES⁺=282 (M+1).

### Step 6: ethyl 2-(2-benzyl-6-hydroxy-5,8-dimethyl-1-oxo-1,2,3,4-tetrahydroisoquinolin-7-yl)-2-hydroxyacetate

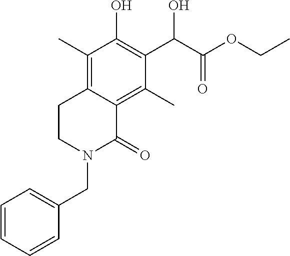 [SMILES: CCOC(=O)C(O)c1c(C)c2c(c(C)c1O)CCN(Cc1ccccc1)C2=O]

An ice cold solution of 1:1 mixture of 2-benzyl-6-hydroxy-5,8-dimethyl-3,4-dihydroisoquinolin-1(2H)-one and 2-benzyl-7-hydroxy-5,8-dimethyl-3,4-dihydroisoquinolin-1(2H)-one (0.56 g, 1.990 mmol) in Dichloromethane (DCM) (9 mL) was treated with TiCl4 (0.241 mL, 2.189 mmol). After stirring for 5 min, the reaction was treated with ethyl 2-oxoacetate (50% in toluene) (0.434 mL, 2.189 mmol) and stirred at rt for 2 hours. The reaction was treated with additional ethyl 2-oxoacetate (500 uL) and stirred at rt for 1.5 hours. Added additional TiCl4 (100 uL), ethyl 2-oxoacetate (500 uL), and stirred at rt for 18 hours. The reaction was quenched with cold 1N HCl, extracted with DCM, washed with Brine, dried over Na2SO4, filtered, and concentrated. Purification with column chromatography (0-60% EtOAc/DCM) afforded mix ethyl 2-(2-benzyl-6-hydroxy-5,8-dimethyl-1-oxo-1,2,3,4-tetrahydroisoquinolin-7-yl)-2-hydroxyacetate (202 mg, 0.527 mmol, 26.5% yield) as clear oil.

### Step 7: ethyl 2-(2-benzyl-6-hydroxy-5,8-dimethyl-1-oxo-1,2,3,4-tetrahydroisoquinolin-7-yl)-2-(tert-butoxy)acetate

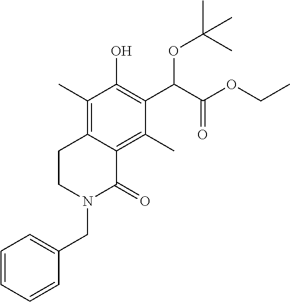 [SMILES: CCOC(=O)C(OC(C)(C)C)c1c(C)c2c(c(C)c1O)CCN(Cc1ccccc1)C2=O]

A solution of mix ethyl 2-(2-benzyl-6-hydroxy-5,8-dimethyl-1-oxo-1,2,3,4-tetrahydroisoquinolin-7-yl)-2-hydroxyacetate (200 mg, 0.522 mmol) in tert-butyl acetate (5 mL, 37.0 mmol) was treated dropwise with perchloric acid (0.179 mL, 2.086 mmol) and stirred at rt for 30 min. The mixture was quenched with aq. 15% NaOH and sat. NaHCO₃, extracted with EtOAc, washed with Brine, dried over Na2SO4, filtered, and concentrated to give crude ethyl 2-(2-benzyl-6-hydroxy-5,8-dimethyl-1-oxo-1,2,3,4-tetrahydroisoquinolin-7-yl)-2-(tert-butoxy)acetate (131.5 mg, 0.299 mmol, 57.4% yield) as pale yellow oil. LCMS (m/z) ES⁺=440 (M+1).

### Step 8: ethyl 2-(2-benzyl-5,8-dimethyl-1-oxo-6-(((trifluoromethyl)sulfonyl)oxy)-1,2,3,4-tetrahydroisoquinolin-7-yl)-2-(tert-butoxy)acetate

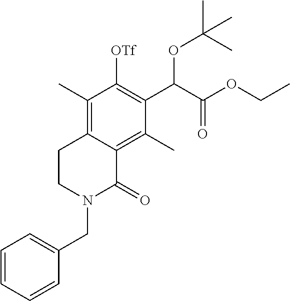 [SMILES: CCOC(=O)C(OC(C)(C)C)c1c(C)c2c(c(C)c1OS(=O)(=O)C(F)(F)F)CCN(Cc1ccccc1)C2=O]

A solution of crude ethyl 2-(2-benzyl-6-hydroxy-5,8-dimethyl-1-oxo-1,2,3,4-tetrahydroisoquinolin-7-yl)-2-(tert-butoxy)acetate (131.5 mg, 0.299 mmol) in N,N-Dimethylformamide (DMF) (2 mL) was treated with K2CO3 (49.1 mg, 0.3552 mmol), N-Phenyl-bis(trifluoromethanesulfonimide) (127 mg, 0.3552 mmol), and stirred at rt over the weekend. The reaction was diluted with water, extracted with EtOAc, washed with Brine, dried over Na2SO4, filtered, and concentrated. Purification with column chromatography (0-15% EtOAc/DCM) afforded ethyl 2-(2-benzyl-5,8-dimethyl-1-oxo-6-(((trifluoromethyl)sulfonyl)oxy)-1,2,3,4-tetrahydroisoquinolin-7-yl)-2-(tert-butoxy)acetate (52 mg, 0.091 mmol, 30.7% yield). NOE confirmed structure. ¹H NMR (400 MHz, CHLOROFORM-d) δ ppm 7.39-7.29 (m, 5H), 5.57 (s, 1H), 4.90-4.65 (m, 2H), 4.28-4.08 (m, 2H), 3.56-3.37 (m, 2H), 2.81 (t, J=6.3 Hz, 2H), 2.67 (s, 3H), 2.26 (s, 3H), 1.24-1.15 (m, 12H); LCMS (m/z) ES⁺=572 (M+1).

### Step 9: ethyl 2-(2-benzyl-5,8-dimethyl-1-oxo-6-(p-tolyl)-1,2,3,4-tetrahydroisoquinolin-7-yl)-2-(tert-butoxy)acetate

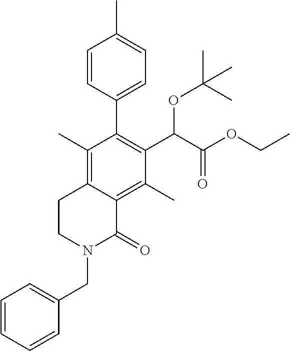 [SMILES: CCOC(=O)C(OC(C)(C)C)c1c(C)c2c(c(C)c1-c1ccc(C)cc1)CCN(Cc1ccccc1)C2=O]

A solution of ethyl 2-(2-benzyl-5,8-dimethyl-1-oxo-6-(((trifluoromethyl)sulfonyl)oxy)-1,2,3,4-tetrahydroisoquinolin-7-yl)-2-(tert-butoxy)acetate (52 mg, 0.091 mmol) in 1,2-Dimethoxyethane (DME) (2 mL) was treated with p-tolylboronic acid (16.08 mg, 0.118 mmol), CsF (55.3 mg, 0.364 mmol), and Sphos palladacycle (20.76 mg, 0.027 mmol). The reaction was bubbled with N2 for 5 min, and then irradiated in microwave at 130° C. for 20 min. The reaction was diluted with sat. NaHCO₃, extracted with EtOAc, washed with Brine, dried over Na2SO4, filtered, and concentrated to give crude ethyl 2-(2-benzyl-5,8-dimethyl-1-oxo-6-(p-tolyl)-1,2,3,4-tetrahydroisoquinolin-7-yl)-2-(tert-butoxy)acetate (56.8 mg, 0.111 mmol, 122% yield). LCMS (m/z) ES⁺=514 (M+1). Used crude for next step.

### Step 10: 2-(2-benzyl-5,8-dimethyl-1-oxo-6-(p-tolyl)-1,2,3,4-tetrahydroisoquinolin-7-yl)-2-(tert-butoxy)acetic Acid

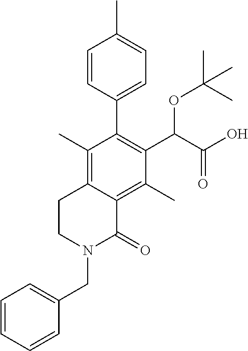 [SMILES: Cc1ccc(-c2c(C)c3c(c(C)c2C(OC(C)(C)C)C(=O)O)C(=O)N(Cc2ccccc2)CC3)cc1]

A solution of crude ethyl 2-(2-benzyl-5,8-dimethyl-1-oxo-6-(p-tolyl)-1,2,3,4-tetrahydroisoquinolin-7-yl)-2-(tert-butoxy)acetate (56.8 mg, 0.111 mmol) in 1,4-Dioxane (2 mL) was treated with 2M LiOH (0.455 mL, 0.910 mmol) and stirred at 80° C. for 11 hours. The reaction was treated with additional 2M LiOH (455 uL) and irradiated in microwave at 120° C. for 40 min. The mixture was concentrated and purified with reverse phase HPLC (30-100% MeCN/H2O-0.1% TFA) to afford the title compound (20.3 mg, 0.041 mmol, 45.0 yield) as white solid. ¹H NMR (400 MHz, METHANOL-d₄) δ ppm 7.42-7.33 (m, 4H), 7.32-7.24 (m, 4H), 7.10 (d, J=7.5 Hz, 1H), 5.11 (s, 1H), 4.82-4.77 (m, 2H), 3.55-3.44 (m, 2H), 2.84 (s, 2H), 2.65 (s, 3H), 2.42 (s, 3H), 1.89 (s, 3H), 0.95 (s, 9H); LCMS (m/z)

ES⁺=486 (M+1).

### Anti-HIV Activity

### MT4 Assay

Antiviral HIV activity and cytotoxicity values for compounds of the invention from Table 1 were measured in parallel in the HTLV-1 transformed cell line MT-4 based on the method previously described (Hazen et al., 2007, In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV (Hazen et al., “In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV”, Antimicrob. Agents Chemother. 2007, 51: 3147-3154; and Pauwels et al., “Sensitive and rapid assay on MT-4 cells for the detection of antiviral compounds against the AIDS virus”, J. of Virological Methods 1987, 16: 171-185).

Luciferase activity was measured 96 hours later by adding a cell titer glo (Promega, Madison, Wis.). Percent inhibition of cell protection data was plotted relative to no compound control. Under the same condition, cytotoxicity of the compounds was determined using cell titer Glo™ (Promega, Madison, Wis.). IC₅₀s were determined from a 10 point dose response curve using 3-4-fold serial dilution for each compound, which spans a concentration range >1000 fold.

These values are plotted against the molar compound concentrations using the standard four parameter logistic equation:

y=((Vmax*x̂n)/(K̂n+x̂n))+Y2

where:

Y2=minimum y n=slope factor

Vmax=maximum y x=compound concentration [M]

K=EC₅₀

When tested in the MT4 assay compounds were found to have IC₅₀ values listed in Table 1.

TABLE 1

| | IC₅₀ |
|---|---|
| Example | (uM) |
| 1 | 3.253 |
| 2 | 1.723 |
| 3 | 3.198 |
| 4 | 0.323 |
| 5 | 0.157 |
| 6 | 0.347 |
| 7 | 0.418 |
| 8 | 6.476 |
| 9 | 0.038 |
| 10 | 1.482 |
| 11 | 0.817 |
| 12 | 0.139 |
| 13 | 0.129 |
| 14 | 1.108 |
| 15 | 0.113 |
| 16 | 0.034 |
| 17 | 0.444 |
| 18 | 1.182 |
| 19 | 1.450 |
| 20 | 0.119 |
| 21 | 0.027 |
| 22 | 0.329 |
| 23 | 0.108 |
| 24 | 0.087 |
| 25 | 0.095 |
| 26 | 0.061 |
| 27 | 0.114 |
| 28 | 0.378 |
| 29 | 0.013 |
| 30 | 0.382 |
| 31 | 0.050 |
| 32 | 0.096 |
| 33 | 0.037 |
| 34 | 0.165 |
| 35 | 0.360 |
| 36 | 0.148 |
| 37 | 0.118 |
| 38 | 0.023 |
| 39 | 0.225 |
| 40 | 0.011 |
| 41 | 0.138 |
| 42 | 1.140 |
| 43 | 0.635 |
| 44 | 1.014 |
| 45 | 0.017 |
| 46 | 0.276 |
| 47 | 0.051 |
| 48 | 0.325 |
| 49 | 0.011 |
| 50 | 0.005 |
| 51 | 0.011 |
| 52 | 0.010 |
| 53 | 0.807 |
| 54 | 0.309 |
| 55 | 0.349 |
| 56 | 0.014 |
| 57 | 0.292 |
| 58 | 0.284 |
| 59 | 0.062 |
| | 1.030 |
| 60 | 0.005 |
| 61 | 0.141 |
| 62 | 0.011 |
| 63 | 0.119 |
| 64 | 0.089 |
| 65 | 0.012 |
| 66 | 0.048 |
| 67 | 0.005 |
| 68 | 0.050 |
| 69 | 0.006 |
| 70 | 0.130 |
| 71 | 0.014 |
| 72 | 0.023 |
| 73 | 0.042 |
| 74 | 0.012 |
| 75 | 0.051 |
| 76 | 0.041 |
| 77 | 0.040 |
| 78 | 0.109 |
| 79 | 0.035 |
| 80 | 0.013 |
| 81 | 0.004 |
| 82 | 0.020 |
| 83 | 0.046 |
| 84 | 0.005 |
| 85 | 0.014 |
| 86 | 0.038 |
| 87 | 0.015 |
| 88 | 0.036 |
| 89 | 0.012 |
| 90 | 0.067 |
| 91 | 0.048 |
| 92 | 0.153 |
| 93 | 0.178 |
| 94 | 0.384 |
| 95 | 0.009 |
| 96 | 0.109 |
| 97 | 0.036 |
| 98 | 0.014 |
| 99 | 3.301 |
| 100 | 1.363 |
| 101 | 0.004 |
| 102 | 0.003 |
| 103 | 0.002 |
| 104 | 0.002 |
| 105 | 0.002 |
| 106 | 0.003 |
| 107 | 0.002 |
| 108 | 0.013 |
| 109 | 0.004 |
| 110 | 0.003 |
| 111 | 0.003 |
| 112 | 1.039 |
| 113 | 0.014 |
| 114 | 0.003 |
| 115 | 0.116 |
| 116 | 0.003 |
| 117 | 0.002 |
| 118 | 0.004 |
| 119 | 0.008 |
| 120 | 0.002 |
| 121 | 0.474 |

1. A compound of Formula I or a pharmaceutically acceptable salt thereof: 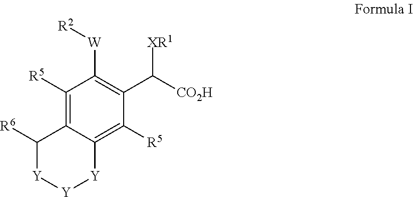
wherein:
each Y is independently C═O, CH—R⁶, or N-L-R³ R³ with the proviso that at least one Y must be CH—R⁶;
X is O or CH₂;
R¹ is C₁₋₆alkyl wherein said alkyl may contain cycloalkyl portions;
W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂(CO)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
each L is independently a bond, —CH₂(CO)—, —C₁₋₃alkylene-, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;
each R³ is independently H, CN, oxo, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, or an R³ may join together with an R⁶ or an R³ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;
R⁴ is CN, halo, —OC₁₋₆alkyl, C₁₋₆alkyl, C₃₋₇cycloalkyl, C₃₋₉heterocycle, or C₅₋₁₄aryl;
each R⁵ is independently H, C₁₋₃alkyl, C₃₋₆cycloalkyl, CH₂F, CHF₂, or CF₃;
each R⁶ is independently H, oxo, C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system, or R⁶ may represent a gem di-C₁₋₃alkyl;
and wherein each heterocycle, heteroaryl, heteroalkyl, and heteroalkylene comprises one to three heteroatoms selected from S, N, B, or O.

2. A compound or salt according to claim 1 wherein R¹ is C₁₋₆alkyl.

3. A compound or salt according to claim 1 wherein W is a bond.

4. A compound or salt according to claim 1 wherein each R⁶ is H.

5. A compound or salt according to claim 1 wherein R² is optionally substituted phenyl.

6. A compound or salt according to claim 5 wherein R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

7. A compound or salt according to claim 1 wherein each R³ is independently C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alky, C₁₋₃fluoroalkyl, or phenyl.

8. A compound or salt according to claim 1 wherein each R⁵ is methyl.

9. A compound or salt according to claim 1 wherein X is O.

10. A compound or salt according to claim 1 wherein one Y group is N-L-R³.

11. A compound or salt according to claim 1 wherein the stereochemistry on the carbon to which XR¹ is bound is as depicted below.

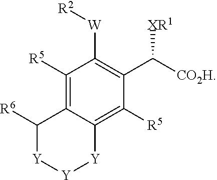

12. (canceled)

13. A pharmaceutical composition comprising a compound or salt according to claim 1.

14. A method for treating a viral infection in a patient mediated at least in part by a virus in the retrovirus family of viruses, comprising administering to said patient a composition according to claim 13.

15. The method of claim 14 wherein said viral infection is mediated by the HIV virus.

16-18. (canceled)
